(19) United States
(12) Patent Application Publication
(10) Pub. No.: US 2021/0023151 A1
(43) Pub. Date: Jan. 28, 2021

(54) ONCOLYTIC VACCINIA VIRUS EXPRESSING IMMUNE CHECKPOINT BLOCKADE FOR CANCER IMMUNOTHERAPY

(71) Applicant: MEMORIAL SLOAN KETTERING CANCER CENTER, New York, NY (US)

(72) Inventors: Liang DENG, New York, NY (US); Weiyi WANG, New York, NY (US); Stewart SHUMAN, New York, NY (US); Taha MERGHOUB, New York, NY (US); Jedd WOLCHOK, New York, NY (US); Wei YAN, Bothell, WA (US)

(73) Assignee: MEMORIAL SLOAN KETTERING CANCER CENTER, New York, NY (US)

(21) Appl. No.: 16/980,282

(22) PCT Filed: Mar. 12, 2019
(86) PCT No.: PCT/US2019/021853

§ 371 Date: Sep. 11, 2020

Related U.S. Application Data
(60) Provisional application No. 62/642,565, filed on Mar. 13, 2018.

(51) Int. Cl. A61K 35/76; C07K 16/28; A61K 9/00

(52) U.S. Cl. CPC A61K 35/76; C07K 16/2818; A61K 45/06; C07K 2317/565; A61K 9/0019

(57) ABSTRACT

Disclosed herein are methods and compositions related to the treatment, prevention, and/or amelioration of cancer in a subject in need thereof. In particular aspects, the present technology relates to the use of poxviruses, including an engineered attenuated vaccinia vims (VACV) strain comprising a disruption of the N-terminal DNA binding domain of the E3L gene (E3LΔA83N) with a deletion of thymidine kinase (E3LΔ83N-TK⁻) engineered to express an antibody specifically targeting cytotoxic T lymphocyte antigen (E3LΔ83N-TK⁻-anti-CTLA-4), alone or in combination with immune checkpoint blocking agents or immune stimulating agents, as an oncolytic and immunotherapeutic composition. In some aspects, the present technology relates to an E3LΔ83N-TK⁻-anti-CTLA-4 virus further engineered to express human Fms-like tyrosine kinase 3 ligand (hFlt3L) (E3LΔ83N-TK⁻-hFlt3L-anti-CTLA-4). In some embodiments, the engineered viruses are administered to a subject alone or in combination with immune checkpoint blocking agents or immune stimulating agents, as an oncolytic and immunotherapeutic composition.

### CROSS-REFERENCE TO RELATED APPLICATIONS

This application claims the benefit of and priority to U.S. Provisional Appl. No. 62/642,565, filed Mar. 13, 2018, the disclosure of which is incorporated by reference herein in its entirety.

### TECHNICAL FIELD

The present disclosure relates generally to the fields of oncology, virology, and immunotherapy. In particular, the present technology relates to the use of poxviruses, including an engineered attenuated vaccinia virus (VACV) strain comprising a disruption of the N-terminal DNA binding domain of the E3L gene (E3LΔ83N) with a deletion of thymidine kinase (E3LΔ83N-TK⁻) engineered to express an antibody that specifically targets cytotoxic T lymphocyte antigen 4 (E3LΔ83N-TK⁻-anti-CTLA-4) as an oncolytic and immunotherapeutic composition. In some embodiments, the technology of the present disclosure relates to the use of an E3LΔ83N-TK⁻-anti-CTLA-4 virus further engineered to express human Fms-like tyrosine kinase 3 ligand (hFlt3L) (E3LΔ83N-TK⁻-hFlt3L-anti-CTLA-4) as an oncolytic and immunotherapeutic composition. In some embodiments, the engineered E3LΔ83N viruses are administered to a subject in need thereof alone or in combination with immune checkpoint blocking agents or immune stimulating agents.

### BACKGROUND

The following description is provided to assist the understanding of the reader. None of the information provided or references cited is admitted to be prior art.

Malignant tumors such as melanoma are inherently resistant to conventional therapies and present significant therapeutic challenges. Immunotherapy is an evolving area of research and an additional option for the treatment of certain types of cancers. The immunotherapy approach rests on the rationale that the immune system may be stimulated to identify tumor cells, and target them for destruction. Despite presentation of antigens by cancer cells and the presence of immune cells that could potentially react against tumor cells, in many cases, the immune system is not activated or is affirmatively suppressed. Key to this phenomenon is the ability of tumors to protect themselves from immune response by coercing cells of the immune system to inhibit other cells of the immune system. Tumors develop a number of immunomodulatory mechanisms to evade antitumor immune responses. Thus, improved immunotherapeutic approaches are needed to enhance host antitumor immunity and target tumor cells for destruction.

### SUMMARY

In one aspect, the present disclosure provides an engineered E3LΔ83N-TK⁻-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding an anti-cytotoxic T lymphocyte-associated antigen (CTLA-4) antibody heavy chain (HC), and an anti-CTLA-4 antibody light chain (LC), wherein the HC and LC are separated by a nucleotide sequence that encodes, in the 5′ to 3′ direction, a protease cleavage site and a 2A peptide (Pep2A) sequence.

In some embodiments, the protease cleavage site is a furin cleavage site. In some embodiments, the expression cassette further comprises a promoter that is capable of directing expression of the open reading frame. In some embodiments, the heterologous nucleic acid sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene, a bioluminescent protein, a fluorescent protein, a chemiluminescent protein, or any combination thereof. In some embodiments, the virus does not produce a full-length thymidine kinase (TK) gene product. In some embodiments, the open reading frame comprises the nucleotide sequence set forth in SEQ ID NO: 1. In some embodiments, the open reading frame comprises one or more heavy chain CDR regions of anti-CTLA-4, one or more light chain CDR regions of anti-CTLA-4, and at least 95% sequence identity to the nucleotide sequence set forth in SEQ ID NO: 1. In some embodiments, the open reading frame encodes an anti-CTLA-4 antibody or antigen binding fragment thereof comprising a heavy chain immunoglobulin variable domain (V_{H}) and a light chain immunoglobulin variable domain (V_{L}), wherein: (a) the V_{H} comprises a V_{H}-CDR1 sequence of GYTFTDY (SEQ ID NO: 27), a V_{H}-CDR2 sequence of PYNG (SEQ ID NO: 28), and aV_{H}-CDR3 sequence of YGSWFA (SEQ ID NO: 29), and (b) the V_{L} comprises a V_{L}-CDR1 sequence of SQSIVHSNGNTY (SEQ ID NO: 30), a V_{L}-CDR2 sequence of KVS (SEQ ID NO: 31), and a V_{L}-CDR3 sequence of GSHVPY (SEQ ID NO: 32); and wherein the open reading frame is at least 95% identical to the nucleotide sequence set forth in SEQ ID NO: 1. In some embodiments, the open reading frame encodes (a) the heavy chain CDR regions of an anti-human CTLA-4 antibody (anti-huCTLA-4) and the light chain CDR regions of an anti-huCTLA-4, or (b) encodes the heavy chain variable regions of an anti-human CTLA-4 antibody (anti-huCTLA-4) and the light chain variable regions of an anti-huCTLA-4, wherein the anti-huCTLA-4 is optionally ipilimumab.

In some embodiments, mice infected with the engineered virus have an increased post-infection lifespan compared to mice infected with a vector control (E3LΔ83N-TK⁻) or E3LΔ83N-TK⁻ co-administered with anti-CTLA-4 (E3LΔ83N-TK⁻+ anti-CLTA-4).

In one aspect, the present disclosure provides an immunogenic composition comprising an engineered E3LΔ83N-TK⁻-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding an anti-cytotoxic T lymphocyte-associated antigen (CTLA-4) antibody heavy chain (HC), and an anti-CTLA-4 antibody light chain (LC), wherein the HC and LC are separated by a nucleotide sequence that encodes, in the 5′ to 3′ direction, a protease cleavage site and a 2A peptide (Pep2A) sequence.

In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of an engineered E3LΔ83N-TK⁻-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding an anti-cytotoxic T lymphocyte-associated antigen (CTLA-4) antibody heavy chain (HC), and an anti-CTLA-4 antibody light chain (LC), wherein the HC and LC are separated by a nucleotide sequence that encodes, in the 5′ to 3′ direction, a protease cleavage site and a 2A peptide (Pep2A) sequence.

In some embodiments, treatment comprises one or more of the following: inducing an immune response in the subject against the tumor or enhancing or promoting an ongoing immune response against the tumor in the subject, inducing increased cytotoxic CD8⁺ T cells and/or CD4⁺ T effector cells within the tumor; inducing increased cytotoxic CD8⁺ T cells within the spleen; reducing the volume of the tumor, eradicating the tumor, inhibiting growth of the tumor, inhibiting metastatic growth of the tumor, inducing apoptosis of tumor cells, or prolonging survival of the subject as compared to an untreated control subject. In some embodiments, the tumor includes tumor cells located at the site of the E3LΔ83N-TK⁻-anti-CTLA-4 vaccinia virus delivery, or tumor cells located both at the site of delivery and elsewhere in the body of the subject. In some embodiments, the composition is administered to the subject intratumorally, intravenously, or any combination thereof. In some embodiments, the tumor is melanoma, colon carcinoma, breast carcinoma, or prostate carcinoma.

In some embodiments, the method further comprises simultaneously or sequentially delivering one or more immune checkpoint blocking agents or immune stimulating agents to the subject, wherein the one or more immune checkpoint blocking agents is administered to the subject intratumorally, intravenously, or any combination thereof. In some embodiments, the one or more immune checkpoint blocking agents or immune stimulating agents is selected from the group consisting of: anti-PD-1 antibody, anti-PD-L1 antibody, anti-PD-L2 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, anti-GITR antibody, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, and any combination thereof.

In one aspect, the present disclosure provides an engineered E3LΔ83N-TK⁻- hFlt3L-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding human Fms-like tyrosine kinase 3 ligand (hFlt3L), an anti-cytotoxic T lymphocyte-associated antigen (CTLA-4) antibody heavy chain (HC), and an anti-CTLA-4 antibody light chain (LC), wherein the hFlt3L and the HC nucleotide sequences are separated by a nucleotide sequence that encodes, in the 5′ to 3′ direction, a protease cleavage site and a 2A peptide (Pep2A) sequence, and wherein the HC and LC are separated by a nucleotide sequence that encodes, in the 5′ to 3′ direction, a protease cleavage site and a Pep2A sequence.

In some embodiments, the protease cleavage site is a furin cleavage site. In some embodiments, the expression cassette further comprises a promoter that is capable of directing expression of the open reading frame. In some embodiments, the heterologous nucleic acid sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene, a bioluminescent protein, a fluorescent protein, a chemiluminescent protein, or any combination thereof. In some embodiments, the virus does not produce a full-length thymidine kinase (TK) gene product. In some embodiments, the open reading frame comprises the nucleotide sequence set forth in SEQ ID NO: 5. In some embodiments, the open reading frame comprises one or more heavy chain CDR regions of anti-CTLA-4, one or more light chain CDR regions of anti-CTLA-4, and at least 95% sequence identity to the nucleotide sequence set forth in SEQ ID NO: 5. In some embodiments, the open reading frame encodes an anti-CTLA-4 antibody or antigen binding fragment thereof comprising a heavy chain immunoglobulin variable domain (V_{H}) and a light chain immunoglobulin variable domain (V_{L}), wherein: (a) the V_{H} comprises a V_{H}-CDR1 sequence of GYTFTDY (SEQ ID NO: 27), a V_{H}-CDR2 sequence of PYNG (SEQ ID NO: 28), and aV_{H}-CDR3 sequence of YGSWFA (SEQ ID NO: 29), and (b) the V_{L} comprises a V_{L}-CDR1 sequence of SQSIVHSNGNTY (SEQ ID NO: 30), a V_{L}-CDR2 sequence of KVS (SEQ ID NO: 31), and a V_{L}-CDR3 sequence of GSHVPY (SEQ ID NO: 32); and wherein the open reading frame is at least 95% identical to the nucleotide sequence set forth in SEQ ID NO: 5. In some embodiments, the open reading frame encodes (a) the heavy chain CDR regions of an anti-human CTLA-4 antibody (anti-huCTLA-4) and the light chain CDR regions of an anti-huCTLA-4, or (b) encodes the heavy chain variable regions of an anti-human CTLA-4 antibody (anti-huCTLA-4) and the light chain variable regions of an anti-huCTLA-4, wherein the anti-huCTLA-4 is optionally ipilimumab.

In one aspect, the present disclosure provides an immunogenic composition comprising an engineered E3LΔ83N-TK⁻- hFlt3L-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding human Fms-like tyrosine kinase 3 ligand (hFlt3L), an anti-cytotoxic T lymphocyte-associated antigen (CTLA-4) antibody heavy chain (HC), and an anti-CTLA-4 antibody light chain (LC), wherein the hFlt3L and the HC nucleotide sequences are separated by a nucleotide sequence that encodes, in the 5′ to 3′ direction, a protease cleavage site and a 2A peptide (Pep2A) sequence, and wherein the HC and LC are separated by a nucleotide sequence that encodes, in the 5′ to 3′ direction, a protease cleavage site and a Pep2A sequence. In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of an engineered E3LΔ83N-TK⁻-hFlt3L-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding human Fms-like tyrosine kinase 3 ligand (hFlt3L), an anti-cytotoxic T lymphocyte-associated antigen (CTLA-4) antibody heavy chain (HC), and an anti-CTLA-4 antibody light chain (LC), wherein the hFlt3L and the HC nucleotide sequences are separated by a nucleotide sequence that encodes, in the 5′ to 3′ direction, a protease cleavage site and a 2A peptide (Pep2A) sequence, and wherein the HC and LC are separated by a nucleotide sequence that encodes, in the 5′ to 3′ direction, a protease cleavage site and a Pep2A sequence.

In some embodiments of the method, treatment comprises one or more of the following: inducing an immune response in the subject against the tumor or enhancing or promoting an ongoing immune response against the tumor in the subject, inducing increased cytotoxic CD8⁺ T cells and/or CD4⁺ T effector cells within the tumor; inducing increased cytotoxic CD8⁺ T cells within the spleen; reducing the volume of the tumor, eradicating the tumor, inhibiting growth of the tumor, inhibiting metastatic growth of the tumor, inducing apoptosis of tumor cells, or prolonging survival of the subject as compared to an untreated control subject. In some embodiments, the tumor includes tumor cells located at the site of the E3LΔ83N-TK⁻- hFlt3L-anti-CTLA-4 vaccinia virus delivery, or tumor cells located both at the site of delivery and elsewhere in the body of the subject. In some embodiments, the composition is administered to the subject intratumorally, intravenously, or any combination thereof. In some embodiments, the tumor is melanoma, colon carcinoma, breast carcinoma, or prostate carcinoma.

In some embodiments, the method further comprises simultaneously or sequentially delivering one or more immune checkpoint blocking agents or immune stimulating agents to the subject, wherein the one or more immune checkpoint blocking agents is administered to the subject intratumorally, intravenously, or any combination thereof. In some embodiments, the one or more immune checkpoint blocking agents or immune stimulating agents is selected from the group consisting of: anti-PD-1 antibody, anti-PD-L1 antibody, anti-PD-L2 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, anti-GITR antibody, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, and any combination thereof.

In one aspect, the present disclosure provides a recombinant E3LΔ83N-TK⁻-anti-CTLA-4 virus nucleic acid sequence, wherein the nucleic acid sequence between position 80,962 and 81,032 of the corresponding wild type vaccinia genome as set forth in SEQ ID NO: 7 is replaced with a heterologous nucleic acid comprising an expression cassette comprising an open reading frame encoding an anti-cytotoxic T lymphocyte-associated antigen (CTLA-4) antibody heavy chain (HC), and an anti-CTLA-4 antibody light chain (LC), wherein the HC and LC are separated by a nucleotide sequence that encodes, in the 5′ to 3′ direction, a protease cleavage site and a 2A peptide (Pep2A) sequence.

In one aspect, the present disclosure provides a recombinant E3LΔ83N-TK⁻- hFlt3L-anti-CTLA-4 vaccinia virus nucleic acid sequence, wherein the nucleic acid sequence between position 80,962 and 81,032 of the corresponding wild type vaccinia genome as set forth in SEQ ID NO: 7 is replaced with a heterologous nucleic acid sequence comprising an expression cassette comprising an open reading frame encoding human Fms-like tyrosine kinase 3 ligand (hFlt3L), an anti-cytotoxic T lymphocyte-associated antigen (CTLA-4) antibody heavy chain (HC), and an anti-CTLA-4 antibody light chain (LC), wherein the hFlt3L and the HC nucleotide sequences are separated by a nucleotide sequence that encodes, in the 5′ to 3′ direction, a protease cleavage site and a 2A peptide (Pep2A) sequence, and wherein the HC and LC are separated by a nucleotide sequence that encodes, in the 5′ to 3′ direction, a protease cleavage site and a Pep2A sequence.

### BRIEF DESCRIPTION OF THE DRAWINGS

FIG. 1 shows a schematic diagram of a single expression cassette designed to produce anti-muCTLA4 (9D9) using the vaccinia viral synthetic early and late promoter (PsE/L). The coding sequence of the heavy chain (muIgG2a) and light chain of 9D9 was separated by a cassette including a furin cleavage site followed by a Pep2A sequence, which enables ribosome skipping and the initiation of light chain protein synthesis. Human IgG kappa light chain leader sequence was used as the signal peptide for both the heavy chain and the light chain of 9D9. This construct allows for the generation of a single transcript, which can be translated into two protein precursors. The linker peptide was cleaved by furin resulting in the generation of the mature heavy chain, which was then paired with the light chain and secreted out as a fully assembled IgG. A separate construct was also generated to express a control IgG, anti-dinitrophenol (DNP) antibody, using the same design.

FIG. 2 shows a schematic diagram of homologous recombination between plasmid (pCB) DNA and viral genomic DNA at the thymidine kinase (TK) locus. pCB plasmid was used to insert specific gene of interest (SG) (e.g., anti-DNP muIgG2a, anti-muCTLA-4 muIgG2a, or human Flt3L-anti-muCTLA-4 fusion gene), under the control of the vaccinia synthetic early and late promoter (PsE/L), into the viral genomic DNA at the TK locus. The E. coli xanthine-guanine phosphoribosyl transferase gene (gpt) under the control of vaccinia P7.5 promoter was used as a drug selection marker. These two expression cassettes were flanked by partial sequence of TK gene and adjacent sequence (TK-L and TK-R) on either side. The plasmid DNA lacking SG was used as a vector control. Homologous recombination that occurred at the TK locus results in the insertion of SG and gpt expression cassettes or gpt alone into the viral genomic DNA to generate E3LΔ83N-TK⁻-DNP, E3LΔ83N-TK⁻-anti-muCTLA-4, E3LΔ83N-TK⁻-vector, and E3LΔ83N-TK⁻-hFlt3L-anti-muCTLA-4 (the construct is described in FIG. 9). The recombinant viruses were enriched in the presence of gpt selection medium including MPA, xanthine and hypoxanthine, and plaque purified for at least four rounds.

FIGS. 3A-3B show PCR analysis of purified recombinant vaccinia viruses, demonstrating the successful generation of E3LΔ83N-TK⁻-anti-DNP and E3LΔ83N-TK⁻-anti-muCTLA-4 recombinant viruses through homologous recombination at the thymidine kinase (TK) locus. FIG. 3A shows PCR analysis of viral genomic DNAs to verify the homologous recombinational insertions of transgenes at the TK locus and the existence of the transgenes. E3LΔ83N-TK⁻-vector was used as positive control. E3LΔ83N was used as negative control. FIG. 3B shows PCR analysis of viral genomic DNAs to verify the deletion of TK gene, and to make sure there were no contaminating parental E3LΔ83N viruses. E3LΔ83N was used as positive control, and water was used as negative control. The PCR products including the inserted transgenes were sequenced to make sure the inserted genes have the correct sequences.

FIGS. 4A-4F are a series of graphs showing a multi-step growth of the parental E3LΔ83N-TK⁺ virus, and recombinant viruses, including E3LΔ83N-TK⁻ vector, E3LΔ83N-TK⁻-DNP, and E3LΔ83N-TK⁻-anti-muCTLA-4 in murine and human melanoma cell line. Murine B16-F10 and human SK-MEL-28 and SK-MEL-146 melanoma cells were infected with the virus at a multiplicity of infection (MOI) of 0.1. Samples were collected at various time points post infection and viral yields (log pfu) were determined by titrating on BSC40 cells. Viral yields were plotted against hours post infection. The fold changes of viral yields at 72 h over those at 1 h post infection were calculated. FIGS. 4A-4B are graphs of the virus yield at 24, 48, and 72 h (FIG. 4A) and fold changes at 72 h over 1 h post infection in murine B16-F10 melanoma cells (FIG. 4B). FIGS. 4C-4D are graphs of the virus yield at 24, 48, and 72 h (FIG. 4C) and fold changes at 72 h (FIG. 4D) post infection in human SK-MEL-146 melanoma cells. FIGS. 4E-4F are graphs of the virus yield at 24, 48, and 72 h (FIG. 4E) and fold changes at 72 h over 1 h post infection in human SK-MEL-28 melanoma cells (FIG. 4F).

FIGS. 5A-5B show a Western blot analysis of antibody expression in E3LΔ83N-TK⁻, E3LΔ83N-TK⁻-anti-DNP, or E3LΔ83N-TK⁻-anti-muCTLA-4 viruses-infected murine B16-F10 melanoma cells. B16-F10 cells were infected or mock infected with E3LΔ83N-TK⁻, E3LΔ83N-TK⁻-anti-DNP, or E3LΔ83N-TK⁻-anti-muCTLA-4 viruses at a MOI of 10. Cell lysates and supernatant were collected at various time points post infection. FIG. 5A shows Western blot analysis of anti-DNP or anti-muCTLA-4 antibodies expression in cell pellet from E3LΔ83N-TK⁻, E3LΔ83N-TK⁻-anti-DNP, E3LΔ83N-TK⁻-anti-muCTLA-4, or mock-infected B16-F10 cells. Cell pellets were collected at 8, 24, 36, and 48 h after virus infection, and the polypeptides in cell lysates were separated using 10% SDS-PAGE. HRP-linked anti-mouse IgG (heavy and light chain) antibody was used to detect full-length and heavy chain of the anti-muCTLA-4 or anti-DNP antibodies. Antibody against the vaccinia D12 protein was used for detecting viral protein expression. GAPDH was used as loading control. FIG. 5B shows Western blot analysis of secreted anti-DNP and anti-muCTLA-4 antibodies in supernatant from B16-F10 cells infected with E3LΔ83N-TK⁻, E3LΔ83N-TK⁻-anti-DNP, or E3LΔ83N-TK⁻-anti-muCTLA-4 recombinant viruses. Supernatants were collected at 8, 24, and 48 h after virus infection, and the polypeptides were separated on the 8% native gel. HRP-linked anti-mouse IgG antibody was used to detect the secreted antibodies in the supernatant.

FIG. 6 shows a Western blot analysis of antibodies expressed in murine B16-F10 or human SK-MEL-28 melanoma cells after E3LΔ83N-TK⁻-anti-muCTLA-4 virus infection. B16-F10 or SK-MEL-28 cells were infected with E3LΔ83N-TK⁻-anti-muCTLA-4 virus at a MOI of 10. Cell lysates were collected at various time points post infection, and polypeptides were separated by using 10% SDS-PAGE. HRP-linked anti-mouse IgG (heavy and light chain) antibody was used to detect full-length, heavy chain, and light chain of anti-muCTLA-4 antibodies. GAPDH was used as a loading control.

FIGS. 7A-7D are graphical representations of the experimental scheme, Kaplan-Meier survival curves, and graphical representations of tumor volumes in mice treated with intratumoral injection of recombinant viruses in the presence or absence of systemic or intratumoral delivery of anti-muCTLA-4 antibody in a murine B16-F10 melanoma bilateral implantation model. FIG. 7A shows a schematic diagram of the experiment design. B16-F10 melanoma cells (5×10⁵ and 1×10⁵ cells) were intradermally implanted into the shaved skin on the right and left flank of C57BL/6J mice, respectively. At 7 or 8 days post implantation, the right side tumors (about 3 mm in diameter) were intratumorally injected twice per week with PBS, E3LΔ83N-TK⁻, E3LΔ83N-TK⁻ plus intraperitoneal (IP) injection of anti-muCTLA-4 antibody (100 μg/mouse), E3LΔ83N-TK⁻ plus intratumoral (IT) injection of anti-muCTLA-4 antibody (10 μg/mouse), or E3LΔ83N-TK⁻-anti-muCTLA-4. Tumor volumes were measured and the survival of the mice was monitored. FIG. 7B shows the Kaplan-Meier survival curve of the above experiment. The survival data were analyzed by log-rank (Mantel-Cox) test. (*, P<0.05; ***, P<0.001). FIGS. 7C-7D show graphical representations of tumor volumes. Tumor volumes were measured twice a week. FIG. 7C shows the volumes of the injected tumors at the right flank of the mice, and FIG. 7D are the volumes of non-injected tumors at the left flank of the mice.

FIGS. 8A-8D show a series of graphical representations of data demonstrating that E3LΔ83N-TK⁻-anti-muCTLA-4 is more effective than E3LΔ83N-TK⁻ virus in activating both CD8⁺ and CD4⁺ T cells in non-injected tumors in a bilateral B16-F10 melanoma model. FIG. 8A shows representative flow cytometry dot plots of Granzyme CD8⁺ cells from non-injected tumors of mice treated with PBS, E3LΔ83N-TK⁻, or E3LΔ83N-TK⁻-anti-muCTLA-4. FIG. 8B shows percentages of Granzyme CD8⁺ positive cells in non-injected side tumors of mice treated with PBS, E3LΔ83N-TK⁻, or E3LΔ83N-TK⁻-anti-muCTLA-4. FIG. 8C shows representative flow cytometry dot plots of Granzyme CD4⁺ cells from non-injected tumors of mice treated with PBS, E3LΔ83N-TK⁻, or E3LΔ83N-TK⁻-anti-muCTLA-4. FIG. 8D shows percentages of Granzyme CD4⁺ positive cells in non-injected side tumors of mice treated with PBS, E3LΔ83N-TK⁻, or E3LΔ83N-TK⁻-anti-muCTLA-4. Data are means±SEM (n=3). (*, p<0.05, **, p<0.01; ***, p<0.001).

FIG. 9 is a schematic diagram of a single expression cassette designed to produce both human Flt3L (hFlt3L) and anti-muCTLA4 (9D9) using the vaccinia viral synthetic early and late promoter (PsE/L). A cassette including a furin cleavage site followed by a Pep2A sequence was used to separate the coding sequence between the hFlt3L and the heavy chain of anti-muCTLA-4 (9D9). The same cassette was used to separate the heavy chain and light chain of 9D9. This cassette enables ribosome skipping and the initiation of the heavy chain or light chain protein synthesis. Human IgG kappa light chain leader sequence was used as the signal peptide for both heavy and light chain of 9D9. This construct allows the generation of a single transcript, which can be translated into three protein precursors. The linker peptide was cleaved by furin resulting in the generation of the hFlt3L, as well as the mature heavy chain, which was then paired with the light chain, and secreted out as a fully assembled IgG.

FIG. 10 shows a Western blot analysis of antibody expression in murine B16-F10 melanoma cells infected with E3LΔ83N-TK⁻ or E3LΔ83N-TK⁻-hFlt3L-anti-muCTLA-4 viruses. B16-F10 cells were infected with E3LΔ83N-TK⁻ or E3LΔ83N-TK⁻-hFlt3L-anti-muCTLA-4 viruses at a MOI of 10. Cell lysates were collected at 6, 20, and 36 h post infection, and the polypeptides in cell lysates were separated using 10% SDS-PAGE. HRP-linked anti-mouse IgG (heavy and light chain) antibody was used to detect full length, heavy chain, and light chain of the anti-muCTLA-4 antibodies. Antibody against human Flt3L (hFlt3L) was used to check the expression of hFlt3L protein. GAPDH was used as a loading control.

FIGS. 11A-11B show a series of graphical representations demonstrating that intratumoral injection of E3LΔ83N-TK⁻-anti-muCTLA-4 or E3LΔ83N-TK⁻-hFlt3L-anti-muCTLA-4 are more effective than E3LΔ83N-TK⁻ or E3LΔ83N-TK⁻-hFlt3L in generating antitumor CD8⁺ T cells in the spleens of treated mice in a murine B16-F10 melanoma bilateral implantation model. B16-F10 cells (5×10⁵ and 2.5×10⁵, respectively) were intradermally implanted into the shaved skin on the right and left flank of C57BL/6J mice. At 7 days post implantation, the tumors on the right flank (about 3 mm in diameter) were injected with PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4 twice, three days apart. Mice were euthanized 3 days after the second injection. ELISPOT was performed to assess the generation of antitumor specific CD8⁺ T cells in the spleens of mice treated with the recombinant viruses. Briefly, CD8⁺ T cells were isolated from splenocytes and 2.5×10⁵ cells were cultured with irradiated B16-F10 cells overnight at 37° C. in anti-IFN-γ-coated BD ELISPOT microwells plate. CD8⁺ T cells were stimulated with B16-F10 cells irradiated with a γ-irradiator and IFN-γ secretion was detected with an anti-IFN-γ antibody. FIG. 11A shows the numbers of IFN-γ⁺ spots per 250,000 CD8⁺ T cells from individual mouse treated with either PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4 (n=4 or 5; *, p<0.05, **, p<0.01). Data are means±SEM (n=4 or 5). FIG. 11B shows the numbers of IFN-γ⁺ spots per 250,000 CD8⁺ T cells pooled from mice in each group treated with either PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4 (*, p<0.05, **, p<0.01). Data are means±SEM (n=3 in triplicates).

FIGS. 12A-12B are graphical representations of tumor volumes in mice treated with intratumoral injection of recombinant viruses in the presence or absence of systemic delivery of anti-muPD-L1 antibody in a murine B16-F10 melanoma bilateral implantation model. Tumor volumes were measured twice a week. B16-F10 melanoma cells (5×10⁵ and 1×10⁵ cells) were intradermally implanted into the shaved skin on the right and left flank of C57BL/6J mice, respectively. At 8 days post implantation, the right side tumors (about 3-4 mm in diameter) were intratumorally injected twice per week with PBS, E3LΔ83N-TK⁻, E3LΔ83N-TK⁻-anti-muCTLA-4, E3LΔ83N-TK⁻-hFlt3L-anti-muCTLA-4, or each individual virus plus intraperitoneal (IP) injection of murine anti-PD-L1 antibody (250 μg/mouse). Tumor volumes were measured twice each week. FIG. 12A shows the individual tumor volumes of the non-injected tumors at the left flank of the mice, and FIG. 12B shows the individual tumor volumes of injected tumors at the right flank of the mice.

FIG. 13 shows the complete genome sequence of vaccinia virus Western Reserve (WR) (GenBank Accession No.: AY243312.1; SEQ ID NO: 7).

FIGS. 14A-14C are a series of graphs showing a multi-step growth of the parental E3LΔ83N-TK⁺ virus, and recombinant viruses, including E3LΔ83N-TK⁻ vector, E3LΔ83N-TK⁻-anti-muCTLA-4 and E3LΔ83N-TK⁻-hFlt3L-anti-muCTLA-4 in murine and human melanoma cell line. Murine B16-F10 and human SK-MEL-28 and SK-MEL-146 melanoma cells were infected with the virus at a multiplicity of infection (MOI) of 0.1. Samples were collected at various time points post infection and viral yields (log pfu) were determined by titrating on BSC40 cells. Viral yields were plotted against hours post infection. FIG. 14A is the graph of the virus yield at 24, 48, and 72 h in murine B16-F10 melanoma cells. FIG. 14B is the graph of the virus yield at 24, 48, and 72 h in human SK-MEL-28 melanoma cells. FIG. 14C is the graph of the virus yield at 24, 48, and 72 h in human SK-MEL-146 melanoma cells.

FIGS. 15A-15B show a Western blot analysis of antibody expression in E3LΔ83N-TK⁻, E3LΔ83N-TK⁻-anti-muCTLA-4, or E3LΔ83N-TK⁻-hFlt3L-anti-muCTLA-4 virus-infected murine B16-F10 melanoma cells and MC38 murine colon cancer cells. B16-F10 or MC38 cells were mock infected or infected with E3LΔ83N-TK⁻, E3LΔ83N-TK⁻-anti-muCTLA-4, or E3LΔ83N-TK⁻-hFlt3L-anti-muCTLA-4 viruses at a MOI of 10. Cell lysates and supernatant were collected at various time points post infection. FIG. 15A shows Western blot analysis of anti-muCTLA-4 antibody expression in cell pellet from E3LΔ83N-TK⁻, E3LΔ83N-TK⁻-anti-muCTLA-4, E3LΔ83N-TK⁻-hFlt3L-anti-muCTLA-4, or mock-infected B16-F10 cells. Cell pellets were collected at 8, 24, and 32 h after virus infection, and the polypeptides in cell lysates were separated using 10% SDS-PAGE. HRP-linked anti-mouse IgG (heavy and light chain) antibody was used to detect the un-processed full-length, heavy chain and light chain of the anti-muCTLA-4 antibody. Antibody against the vaccinia D12 protein was used for detecting viral protein expression. GAPDH was used as loading control. FIG. 15B shows Western blot analysis of anti-muCTLA-4 antibody expression in cell pellet from E3LΔ83N-TK⁻, E3LΔ83N-TK⁻-anti-muCTLA-4, E3LΔ83N-TK⁻-hFlt3L-anti-muCTLA-4, or mock-infected MC38 cells. Cell pellets were collected at 8, 24, and 32 h after virus infection, and the polypeptides in cell lysates were separated using 10% SDS-PAGE. HRP-linked anti-mouse IgG (heavy and light chain) antibody was used to detect the heavy chain and light chain of the anti-muCTLA-4 antibody. GAPDH was used as loading control.

FIG. 16 shows a Western blot analysis of antibody expression in E3LΔ83N-TK⁻, E3LΔ83N-TK⁻-anti-muCTLA-4, or E3LΔ83N-TK⁻-hFlt3L-anti-muCTLA-4 virus-infected human SK-MEL-28 melanoma cells. SK-MEL-28 cells were mock infected or infected with E3LΔ83N-TK⁻, E3LΔ83N-TK⁻-anti-muCTLA-4, or E3LΔ83N-TK⁻-hFlt3L-anti-muCTLA-4 viruses at a MOI of 10. Cell lysates were collected at 24 and 32 hours post infection, and the polypeptides in cell lysates were separated using 10% SDS-PAGE. HRP-linked anti-mouse IgG (heavy and light chain) antibody was used to detect the un-processed full-length, heavy chain and light chain of the anti-muCTLA-4 antibody. Antibody against the vaccinia D12 protein was used for detecting viral protein expression. GAPDH was used as loading control.

FIG. 17 shows a Western blot analysis of human Flt3L expression in E3LΔ83N-TK⁻, or E3LΔ83N-TK⁻-hFlt3L-anti-muCTLA-4 virus-infected murine B16-F10 melanoma cells or human SK-MEL-28 melanoma cells. B16-F10 cells or SK-MEL-28 cells were mock infected or infected with E3LΔ83N-TK⁻, or E3LΔ83N-TK⁻-hFlt3L-anti-muCTLA-4 viruses at a MOI of 10. Cell lysates were collected at 24 and 32 hours post infection, and the polypeptides in cell lysates were separated using 10% SDS-PAGE. Anti-human Flt3L antibody was used to detect the human Flt3L protein. GAPDH was used as loading control.

FIGS. 18A-18B show a Western blot analysis of antibody secretion from E3LΔ83N-TK⁻, E3LΔ83N-TK⁻-anti-muCTLA-4, or E3LΔ83N-TK⁻-hFlt3L-anti-muCTLA-4 virus-infected murine B16-F10 melanoma cells or human SK-MEL-28 melanoma cells. B16-F10 or SK-MEL-28 cells were mock infected or infected with E3LΔ83N-TK⁻, E3LΔ83N-TK⁻-anti-muCTLA-4, or E3LΔ83N-TK⁻-hFlt3L-anti-muCTLA-4 viruses at a MOI of 10. Cell culture supernatant was collected at various time points post infection. FIG. 18A shows a Western blot analysis of secreted anti-muCTLA-4 antibodies in supernatant from B16-F10 cells infected with E3LΔ83N-TK⁻, E3LΔ83N-TK⁻-anti-muCTLA-4, or E3LΔ83N-TK hFlt3L-anti-muCTLA-4 recombinant viruses. Supernatants were collected at 8, 24, and 48 h after virus infection, and the polypeptides were separated on 8% native gel. HRP-linked anti-mouse IgG antibody was used to detect the secreted antibodies in the supernatant. FIG. 18B shows a Western blot analysis of secreted anti-muCTLA-4 antibodies in supernatant from SK-MEL-28 cells infected with E3LΔ83N-TK⁻, E3LΔ83N-TK⁻-anti-muCTLA-4, or E3LΔ83N-TK⁻-hFlt3L-anti-muCTLA-4 recombinant viruses. Supernatants were collected at 8, 24, and 48 h after virus infection, and the polypeptides were separated on 8% native gel. HRP-linked anti-mouse IgG antibody was used to detect the secreted antibodies in the supernatant.

FIG. 19 shows a Western blot analysis of antibody secreted from E3LΔ83N-TK⁻-anti-muCTLA-4 or E3LΔ83N-TK⁻-hFlt3L-anti-muCTLA-4 virus-infected murine B16-F10 melanoma cells or human SK-MEL-28 melanoma cells can bind to recombinant murine CTLA-4 protein. B16-F10 or SK-MEL-28 cells were mock infected or infected with E3LΔ83N-TK⁻, E3LΔ83N-TK⁻-anti-muCTLA-4, or E3LΔ83N-TK⁻-hFlt3L-anti-muCTLA-4 viruses at a MOI of 10. Cell culture supernatant was collected at 24 hours post infection, and blotted against membrane strips containing murine recombinant CTLA-4 protein. HRP-linked anti-mouse IgG (heavy and light chain) antibody was used to detect the binding anti-muCTLA-4 antibody on the membrane.

FIGS. 20A-20B show the recombinant virus titer and the western blot analysis of antibody expression in implanted B16-F10 melanoma tumors injected with E3LΔ83N-TK⁻-hFlt3L-anti-muCTLA-4 virus in mouse. B16-F10 melanoma cells were intradermally implanted into the flank of C57BL/6J mice, and E3LΔ83N-TK⁻-hFlt3L-anti-muCTLA-4 virus was injected into the tumors 7-8 days after implantation. Tumor samples were collected and lysed at 24 or 48 hours after virus injection. FIG. 19A shows the E3LΔ83N-TK⁻-hFlt3L-anti-muCTLA-4 virus titer in tumor samples harvested at 24 and 48 hours after injection. Tumor samples were grinded and virus titers were examined using BSC-40 cells. FIG. 19B shows a western blot analysis of the antibody expression in implanted tumors injected with E3LΔ83N-TK⁻-hFlt3L-anti-muCTLA-4 virus. Tumor samples were collected at 24 and 48 hours after virus injection, and the polypeptide in lysed tumor samples were separated in 10% SDS-PAGE gel. Anti-FLAG antibody was used to detect the heavy chain of anti-muCTLA-4 antibody expressed by E3LΔ83N-TK⁻-hFlt3L-anti-muCTLA-4 virus. GAPDH was used as loading control.

FIGS. 21A-21B show a series of graphical representations of data demonstrating that E3LΔ83N-TK⁻-anti-muCTLA-4 and E3LΔ83N-TK⁻-hFlt3L-anti-muCTLA-4 viruses are more effective than E3LΔ83N-TK⁻ virus in enhancing specific anti-tumor CD8⁺ T cell activities in a B16-F10 melanoma model. B16-F10 melanoma cells were intradermally implanted into C57BL/6J mice. Tumors were mock injected or injected with E3LΔ83N-TK⁻, E3LΔ83N-TK⁻-anti-muCTLA-4, or E3LΔ83N-TK⁻-hFlt3L-anti-muCTLA-4 viruses at 7-8 days after tumor implantation. Mouse spleens were harvested and used to isolate CD8⁺ T cells. ELISPOT assay was used to measure the number of anti-tumor CD8⁺ T cells isolated from mouse spleen. FIG. 21A shows the number of IFN-γ positive CD8⁺ T cells in 250,000 CD8⁺ T cells isolated from mouse spleen. FIG. 21B shows representative images of IFN-γ spot in each wells of ELISPOT plate. Data are means±SEM (n=3). (*, p<0.05; **, p<0.01; ***, p<0.001; ****, p<0.0001).

FIGS. 22A-22D are graphical representations of the Kaplan-Meier survival curves, media survival time, and graphical representations of tumor volumes in mice treated with intratumoral injection of recombinant viruses in a murine B16-F10 melanoma bilateral implantation model. B16-F10 melanoma cells (5×10⁵ and 1×10⁵ cells) were intradermally implanted into the shaved skin on the right and left flank of C57BL/6J mice, respectively. At 7 or 8 days post implantation, the right side tumors (about 3 mm in diameter) were intratumorally injected twice per week with PBS, E3LΔ83N-TK⁻, E3LΔ83N-TK⁻-anti-muCTLA-4, or E3LΔ83N-TK⁻-hFlt3L-anti-muCTLA-4 viruses. Tumor volumes were measured and the survival of the mice was monitored. FIG. 22A shows the Kaplan-Meier survival curve of the above experiment. The survival data were analyzed by log-rank (Mantel-Cox) test. (*, P<0.05; ***, P<0.001). FIG. 22B shows the media survival time of this experiment. FIGS. 22C-22D shows graphical representations of tumor volumes. Tumor volumes were measured twice a week. FIG. 22C shows the volumes of the non-injected tumors at the left flank of the mice, and FIG. 22D shows the volumes of injected tumors at the right flank of the mice.

### DETAILED DESCRIPTION

It is to be appreciated that certain aspects, modes, embodiments, variations, and features of the present technology are described below in various levels of detail in order to provide a substantial understanding of the present technology.

### I. Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a,” “an,” and “the” include plural referents unless the content clearly dictates otherwise. For example, reference to “a cell” includes a combination of two or more cells, and the like.

As used herein, the term “about” in reference to a number is generally taken to include numbers that fall within a range of 1%-10% in either direction (greater than or less than) of the number unless otherwise stated or otherwise evident from the context (except where such number would be less than 0% or exceed 100% of a possible value).

As used herein, the term “antibody” collectively refers to immunoglobulins or immunoglobulin-like molecules including by way of example and without limitation, IgA, IgD, IgE, IgG and IgM, combinations thereof, “antigen binding fragments,” which are antibody fragments capable of binding antigen such as Fab, Fv, single chain Fv (scFv), Fab′, and (Fab′)₂, and similar molecules produced during an immune response in any vertebrate, for example, in mammals such as humans, goats, rabbits, and mice, as well as non-mammalian species, such as shark immunoglobulins. As used herein, “antibodies” (includes intact immunoglobulins) including “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy chain and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain may contain a constant region as well as a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds antigen will have a specific V_{H} region and the V_{L} region sequence, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, humanized antibodies, chimeric antibodies, recombinant antibodies, multispecific antibodies, bispecific antibodies, etc.,) as well as antigen binding fragments. An antibody or antigen binding fragment thereof specifically binds to an antigen.

In any of the above embodiments, the antibody further comprises a Fc domain of any isotype, e.g., but are not limited to, IgG (including IgG1, IgG2, IgG3, and IgG4), IgA (including IgA₁ and IgA₂), IgD, IgE, or IgM, and IgY. An antibody may, for example, comprise an IgG Fc domain, such as an IgG1, IgG2, IgG3, or IgG4. Non-limiting examples of constant region sequences include:

Human IgD constant region, Uniprot: P01880 (SEQ ID NO: 14), which may or may not include the C-terminal lysine amino acid:

APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK

Human IgG1 constant region, Uniprot: P01857 (SEQ ID NO: 15), which may or may not include the C-terminal lysine amino acid:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLEPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Human IgG2 constant region, Uniprot: P01859 (SEQ ID NO: 16), which may or may not include the C-terminal lysine amino acid:

ASTKGPSVFPLAPCSRSTSESTAALGCLVKDYFPEPVTVSWNSGALTSGV
HTFPAVLQSSGLYSLSSVVTVPSSNEGTQTYTCNVDHKPSNTKVDKTVER
KCCVECPPCPAPPVAGPSVFLEPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
EYPSDISVEWESNGQPENNYKTTPPMLDSDGSFFLYSKLTVDKSRWQQGN
VFSCSVMHEALHNHYTQKSLSLSPGK

Human IgG3 constant region, Uniprot: P01860 (SEQ ID NO: 17), which may or may not include the C-terminal lysine amino acid:

ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTERVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK

Human IgM constant region, Uniprot: P01871 (SEQ ID NO: 18):

GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFEGNPRKSKLICQATGESPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY

Human IgG4 constant region, Uniprot: P01861 (SEQ ID NO: 19), which may or may not include the C-terminal lysine amino acid:

ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLEPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

Human IgA1 constant region, Uniprot: P01876 (SEQ ID NO: 20):

ASPTSPKVFPLSLCSTQPDGNVVIACLVQGFFPQEPLSVTWSESGQGVTA
RNEPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTESCMVGHEALPLAFTQKTIDRLAGKPTHVNVSVVMAEVDG
TCY

Human IgA2 constant region, Uniprot: P01877 (SEQ ID NO: 21):

ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY

Human Ig kappa constant region, Uniprot: P01834 (SEQ ID NO: 22):

TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

As used herein, “attenuated,” as used in conjunction with a virus, refers to a virus having reduced virulence or pathogenicity as compared to a non-attenuated counterpart, yet is still viable or live. Typically, attenuation renders an infectious agent, such as a virus, less harmful or virulent to an infected subject compared to a non-attenuated virus. This is in contrast to a killed or completely inactivated virus.

As used herein, “conjoint administration” or “co-administration” refers to administration of a second therapeutic modality in combination with the engineered vaccinia viruses of the present technology, such as E3LΔ83N-TK⁻-anti-CTLA-4 or E3LΔ83N-TK⁻-hFlt3L-anti-CTLA-4. For example, an immune checkpoint blocking agent or immune stimulating agent may be administered in close temporal proximity with E3LΔ83N-TK⁻-anti-CTLA-4 or E3LΔ83N-TK⁻-hFlt3L-anti-CTLA-4. For example, a PD-1/PDL-1 inhibitor (in more specific embodiments, an antibody) can be administered simultaneously with E3LΔ83N-TK⁻-anti-CTLA-4 or E3LΔ83N-TK⁻-hFlt3L-anti-CTLA-4 (by intravenous or intratumoral injection when the E3LΔ83N-TK⁻-anti-CTLA-4 or E3LΔ83N-TK⁻-hFlt3L-anti-CTLA-4 is administered intratumorally or systemically as stated above) or before or after the E3LΔ83N-TK⁻-anti-CTLA-4 or E3LΔ83N-TK⁻-hFlt3L-anti-CTLA-4 administration. In some embodiments, if the E3LΔ83N-TK⁻-anti-CTLA-4 or E3LΔ83N-TK⁻-hFlt3L-anti-CTLA-4 administration and the immune checkpoint blocking agent or immune stimulating agent are administered 1-7 days apart or even up to three weeks apart, this would still be within “close temporal proximity” as stated herein, therefore such administration will qualify as “conjoint.”

The term “control” is used herein to refer to the E3LΔ83N-TK⁻ virus engineered to express a control IgG, anti-dinitrophenol (DNP) antibody (E3LΔ83N-TK⁻-anti-DNP) or a VACVΔC7L virus engineered to express a control IgG (e.g., DNP) antibody (VACVΔC7L-anti-DNP).

As used herein, the term “delivering” means depositing the engineered vaccinia viruses of the present technology, such as E3LΔ83N-TK⁻-anti-CTLA-4 or E3LΔ83N-TK⁻-hFlt3L-anti-CTLA-4, in the tumor microenvironment whether this is done by local administration to the tumor (intratumoral) or by, for example, intravenous route. The term focuses on E3LΔ83N-TK⁻-anti-CTLA-4 or E3LΔ83N-TK⁻-hFlt3L-anti-CTLA-4 that reaches the tumor itself. In some embodiments, “delivering” is synonymous with administering, but it is used with a particular administration locale in mind, e.g., intratumoral.

The terms “disruption” and “mutation” are used interchangeably herein to refer to a detectable and heritable change in the genetic material. Mutations may include insertions, deletions, substitutions (e.g., transitions, transversion), transpositions, inversions, knockouts and combinations thereof. Mutations may involve only a single nucleotide (e.g., a point mutation or a single nucleotide polymorphism) or multiple nucleotides. In some embodiments, mutations are silent, that is, no phenotypic effect of the mutation is detected. In other embodiments, the mutation causes a phenotypic change, for example, the expression level of the encoded product is altered, or the encoded product itself is altered. In some embodiments, a disruption or mutation may result in a disrupted gene with decreased levels of expression of a gene product (e.g., protein or RNA) as compared to the wild-type strain. In other embodiments, a disruption or mutation may result in an expressed protein with activity that is lower as compared to the activity of the expressed protein from the wild-type strain.

As used herein, an “effective amount” or “therapeutically effective amount” refers to a sufficient amount of an agent, which, when administered at one or more dosages and for a period of time, is sufficient to provide a desired biological result in alleviating, curing, or palliating a disease. In the present disclosure, an effective amount of E3LΔ83N-TK⁻-anti-CTLA-4 or E3LΔ83N-TK⁻-hFlt3L-anti-CTLA-4 is an amount that (when administered for a suitable period of time and at a suitable frequency) reduces the number of cancer cells; or reduces the tumor size or eradicates the tumor; or inhibits (i.e., slows down or stops) cancer cell infiltration into peripheral organs; inhibits (i.e., slows down or stops) metastatic growth; inhibits (stabilizes or arrests) tumor growth; allows for treatment of the tumor; and/or induces and promotes an immune response against the tumor. An appropriate therapeutic amount in any individual case may be determined by one of ordinary skill in the art using routine experimentation in light of the present disclosure. Such determination will begin with amounts found effective in vitro and amounts found effective in animals. The therapeutically effective amount will be initially determined based on the concentration or concentrations found to confer a benefit to cells in culture. Effective amounts can be extrapolated from data within the cell culture and can be adjusted up or down based on factors such as detailed herein. Effective amounts of the viral constructs are generally within the range of about 10⁵ to about 10¹⁰ plaque forming units (pfu), although a lower or higher dose may be administered. In some embodiments, the dosage is about 10⁶-10⁹ pfu. In some embodiments, a unit dosage is administered in a volume within the range from 1 to 10 mL. The equivalence of pfu to virus particles can differ according to the specific pfu titration method used. Generally, pfu is equal to about 5 to 100 virus particles. A therapeutically effective amount the anti-CTLA-4 or hFlt3L-anti-CTLA-4 transgene bearing viruses can be administered in one or more divided doses for a prescribed period of time and at a prescribed frequency of administration. For example, a therapeutically effective amount of anti-CTLA-4 or hFlt3L-anti-CTLA-4 bearing viruses in accordance with the present disclosure may vary according to factors such as the disease state, age, sex, weight, and general condition of the subject, and the potency of the viral constructs to elicit a desired immunological response in the particular subject for the particular cancer.

With particular reference to the viral-based immunostimulatory agents disclosed herein, an “effective amount” or “therapeutically effective amount” refers to an amount of a composition comprising an engineered vaccinia virus of the present technology, such as E3LΔ83N-TK⁻-anti-CTLA-4 or E3LΔ83N-TK⁻-hFlt3L-anti-CTLA-4, sufficient to reduce, inhibit, or abrogate tumor cell growth, thereby reducing or eradicating the tumor, or sufficient to inhibit, reduce or abrogate metastatic spread either in vitro, ex vivo, or in a subject or to elicit and promote an immune response against the tumor that will eventually result in one or more of metastatic spread reduction, inhibition, and/or abrogation as the case may be. The reduction, inhibition, or eradication of tumor cell growth may be the result of necrosis, apoptosis, or an immune response, or a combination of two or more of the foregoing (however, the precipitation of apoptosis, for example, may not be due to the same factors as observed with oncolytic viruses). The amount that is therapeutically effective may vary depending on such factors as the particular virus used in the composition, the age and condition of the subject being treated, the extent of tumor formation, the presence or absence of other therapeutic modalities, and the like. Similarly, the dosage of the composition to be administered and the frequency of its administration will depend on a variety of factors, such as the potency of the active ingredient, the duration of its activity once administered, the route of administration, the size, age, sex, and physical condition of the subject, the risk of adverse reactions and the judgment of the medical practitioner. The compositions are administered in a variety of dosage forms, such as injectable solutions.

With particular reference to combination therapy with an immune checkpoint inhibitor or immune stimulating agent, an “effective amount” or “therapeutically effective amount” for an immune checkpoint blocking agent or immune stimulating agent means an amount of an immune checkpoint blocking agent or immune stimulating agent sufficient to reverse or reduce immune suppression in the tumor microenvironment and to activate or enhance host immunity in the subject being treated. Immune checkpoint blocking agents or immune stimulating agents include, but are not limited to, inhibitory anti-PD-1 (programmed cell death 1) inhibitory antibodies (e.g., nivolumab, pembrolizumab, pidilizumab, lambrolizumab), anti-PD-L1 (Programmed death ligand 1) inhibitory antibodies (MPDL3280A, BMS-936559, MEDI4736, MSB 00107180, atezolizumab, avelumab, durvalumab), and antibodies against CD28 inhibitor such as CTLA-4 (cytotoxic T lymphocyte antigen 4) (e.g., ipilimumab), as well as inhibitory antibodies against LAG-3 (lymphocyte activation gene 3), TIM3 (T-cell immunoglobulin and mucin-3), B7-H3, and TIGIT (T-cell immunoreceptor with Ig and ITIM domains). Dosage ranges of the foregoing are known or readily within the skill in the art as several dosing clinical trials have been completed, making extrapolation to other agents possible.

Immune stimulating agents such as agonist antibodies have also been explored as immunotherapy for cancers. For example, anti-ICOS antibody binds to the extracellular domain of ICOS leading to the activation of ICOS signaling and T-cell activation. Anti-OX40 antibody can bind to OX40 and potentiate T-cell receptor signaling leading to T-cell activation, proliferation and survival. Other examples include agonist antibodies against 4-1BB (CD137), GITR.

The immune stimulating agonist antibodies can be used systemically in combination with intratumoral injection of the engineered vaccinia viruses of the present technology, such as E3LΔ83N-TK⁻-anti-CTLA-4 or E3LΔ83N-TK⁻-hFlt3L-anti-CTLA-4. Alternatively, the immune stimulating agonist antibodies can be used conjointly with the engineered vaccinia viruses of the present technology, such as E3LΔ83N-TK⁻-anti-CTLA-4 or E3LΔ83N-TK⁻-hFlt3L-anti-CTLA-4, via intratumoral delivery either simultaneously or sequentially.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcaR are involved in specific killing of target cells and presenting antigens to other components of the immune system, or binding to cells that present antigens.

The term “engineered” is used herein to refer to an organism that has been manipulated to be genetically altered, modified, or changed, e.g., by disruption of the genome. For example, an “engineered vaccinia virus strain” refers to a vaccinia strain that has been manipulated to be genetically altered, modified, or changed.

The term “expression cassette” or “gene cassette” is used herein to refer to a DNA sequence encoding and capable of expressing one or more specific genes of interest (e.g., anti-muCTLA-4 muIgG2a, hFlt3L) and/or a selectable marker (e.g., gpt) that can be inserted between one or more selected restriction sites of a DNA sequence. In some embodiments, insertion of a gene cassette results in a disrupted gene (e.g., a disrupted vaccinia virus thymidine kinase gene). In some embodiments, disruption of the gene involves replacement of at least a portion of the gene with a gene cassette or the insertion of a cassette, which includes a nucleotide sequence comprising an open reading frame encoding one or more of the following operatively linked sequences: specific genes of interest (e.g., an anti-muCTLA-4 muIgG2a heavy chain (HC) and light chain (LC), anti-PD-L1 antibody heavy chain (HC) and light chain (LC), hFlt3L), one or more promoters (e.g., PsE/L), suitable leader sequences, a protease cleavage site (e.g., furin cleavage site), 2A peptide (Pep2A), and a selectable marker. In some embodiments, the 2A peptide comprises one of the following: T2A having an amino acid sequence sequence (GSG) E G R G S L L T C G D V E E N P G P (SEQ ID NO: 23); P2A having an amino acid sequence (GSG) A T N F S L L K Q A G D V E E N P G P (SEQ ID NO: 24); E2A having an amino acid sequence (GSG) Q C T N Y A L L K L A G D V E S N P G P (SEQ ID NO: 25); or F2A having an amino acid sequence (GSG) V K Q T L N F D L L K L A G D V E S N P G P (SEQ ID NO: 26), where the N-terminal (GSG) for each 2A peptide is optional.

“Fc Modifications.” In some embodiments, the antibodies of the present technology comprise a variant Fc region, wherein said variant Fc region comprises at least one amino acid modification relative to a wild-type Fc region (or the parental Fc region), such that said molecule has an altered affinity for an Fc receptor (e.g., an FcγR), provided that said variant Fc region does not have a substitution at positions that make a direct contact with Fc receptor based on crystallographic and structural analysis of Fc-Fc receptor interactions such as those disclosed by Sondermann et al., Nature, 406:267-273 (2000). Examples of positions within the Fc region that make a direct contact with an Fc receptor such as an FcγR, include amino acids 234-239 (hinge region), amino acids 265-269 (B/C loop), amino acids 297-299 (C7E loop), and amino acids 327-332 (F/G) loop.

In some embodiments, an antibody of the present technology has an altered affinity for activating and/or inhibitory receptors, having a variant Fc region with one or more amino acid modifications, wherein said one or more amino acid modification is a N297 substitution with alanine, or a K322 substitution with alanine.

“Glycosylation Modifications.” In some embodiments, antibodies of the present technology have an Fc region with variant glycosylation as compared to a parent Fc region. In some embodiments, variant glycosylation includes the absence of fucose; in some embodiments, variant glycosylation results from expression in GnT1-deficient CHO cells.

In some embodiments, the antibodies of the present technology, may have a modified glycosylation site relative to an appropriate reference antibody that binds to an antigen of interest, without altering the functionality of the antibody, e.g., binding activity to the antigen. As used herein, “glycosylation sites” include any specific amino acid sequence in an antibody to which an oligosaccharide (i.e., carbohydrates containing two or more simple sugars linked together) will specifically and covalently attach.

Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. For example, an Fc-glycoform that lacks certain oligosaccharides including fucose and terminal N-acetylglucosamine may be produced in special CHO cells and exhibit enhanced ADCC effector function.

In some embodiments, the carbohydrate content of an immunoglobulin-related composition disclosed herein is modified by adding or deleting a glycosylation site. Methods for modifying the carbohydrate content of antibodies are well known in the art and are included within the present technology, see, e.g., U.S. Pat. No. 6,218,149; EP 0359096B1; U.S. Patent Publication No. US 2002/0028486; International Patent Application Publication WO 03/035835; U.S. Patent Publication No. 2003/0115614; U.S. Pat. Nos. 6,218,149; 6,472,511; all of which are incorporated herein by reference in their entirety. In some embodiments, the carbohydrate content of an antibody (or relevant portion or component thereof) is modified by deleting one or more endogenous carbohydrate moieties of the antibody. In some certain embodiments, the present technology includes deleting the glycosylation site of the Fc region of an antibody, by modifying position 297 from asparagine to alanine.

Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al., 1999, Nat. Biotechnol. 17: 176-180; Davies et al., 2001, Biotechnol. Bioeng. 74:288-294; Shields et al., 2002, 1 Biol. Chem. 277:26733-26740; Shinkawa et al., 2003, J Biol. Chem. 278:3466-3473; U.S. Pat. No. 6,602,684; U.S. patent application Ser. No. 10/277,370; U.S. patent application Ser. No. 10/113,929; International Patent Application Publications WO 00/61739A1; WO 01/292246A1; WO 02/311140A1; WO 02/30954A1; POTILLEGENT™ technology (Biowa, Inc. Princeton, N.J.); GLYCOMAB™ glycosylation engineering technology (GLYCART biotechnology AG, Zurich, Switzerland); each of which is incorporated herein by reference in its entirety. See, e.g., International Patent Application Publication WO 00/061739; U.S. Patent Application Publication No. 2003/0115614; Okazaki et al., 2004, JMB, 336: 1239-49.

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, “immune checkpoint inhibitor” or “immune checkpoint blocking agent” or “immune checkpoint blockade inhibitor” refers to molecules that completely or partially reduce, inhibit, interfere with or modulate the activity of one or more checkpoint proteins. Certain immune checkpoints act as immune stimulating agents. Checkpoint proteins regulate T-cell activation or function. Checkpoint proteins include, but are not limited to, PD-1 and its ligands PD-L1 and PD-L2; CD28 receptor family members, CTLA-4 and its ligands CD80 and CD86; LAG3, B7-H3, B7-H4, TIM3, ICOS, II DLBCL, BTLA or any combination of two or more of the foregoing. Non-limiting examples contemplated for use herein include inhibitors of PD-1 and its ligands PD-L1 and PD-L2, or any combination thereof (e.g., anti PD-1/PD-L1 therapy).

As used herein, “immune response” refers to the action of one or more of lymphocytes, antigen presenting cells, phagocytic cells, granulocytes, and soluble macromolecules produced by the above cells or the liver or spleen (including antibodies, cytokines, and complement) that results in selective damage to, destruction of, or elimination from the human body of cancerous cells, metastatic tumor cells, etc. An immune response may include a cellular response, such as a T-cell response that is an alteration (modulation, e.g., significant enhancement, stimulation, activation, impairment, or inhibition) of cellular, i.e., T-cell function. A T-cell response may include generation, proliferation or expansion, or stimulation of a particular type of T-cell, or subset of T-cells, for example, effector CD4⁺, CD4⁺ helper, effector CD8⁺, CD8⁺ cytotoxic, or natural killer (NK) cells. Such T-cell subsets may be identified by detecting one or more cell receptors or cell surface molecules (e.g., CD or cluster of differentiation molecules). A T-cell response may also include altered expression (statistically significant increase or decrease) of a cellular factor, such as a soluble mediator (e.g., a cytokine, lymphokine, cytokine binding protein, or interleukin) that influences the differentiation or proliferation of other cells. For example, Type I interferon (IFN-α/β) is a critical regulator of the innate immunity (Huber et al. Immunology 132(4):466-474 (2011)). Animal and human studies have shown a role for IFN-α/β in directly influencing the fate of both CD4⁺ and CD8⁺ T-cells during the initial phases of antigen recognition and anti-tumor immune response. IFN Type I is induced in response to activation of dendritic cells, in turn a sentinel of the innate immune system. An immune response may also include humoral (antibody) response.

The term “immunogenic composition” is used herein to refer to a composition that will elicit an immune response in a mammal that has been exposed to the composition. In some embodiments, an immunogenic composition comprises E3LΔ83N-TK⁻-anti-CTLA-4 or E3LΔ83N-TK⁻-hFlt3L-anti-CTLA-4, alone or in combination with immune checkpoint blockade inhibitors or immune stimulating agents.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

A “knocked out gene” or a “gene deletion” refers to a gene including a null mutation (e.g., the wild-type product encoded by the gene is not expressed, expressed at levels so low as to have no effect, or is non-functional). In some embodiments, the knocked out gene includes heterologous sequences or genetically engineered non-functional sequences of the gene itself, which renders the gene non-functional. In other embodiments, the knocked out gene is lacking a portion of the wild-type gene. For example, in some embodiments, at least about 10%, at least about 15%, at least about 20%, at least about 25%, at least about 30%, at least about 35%, at least about 40%, at least about 45%, at least about 50%, at least about 55% or at least about 60% of the wild-type gene sequence is deleted. In other embodiments, the knocked out gene is lacking at least about 65%, at least about 70%, at least about 75%, at least about 80%, at least about 85%, at least about 90%, at least about 95% or at least about 100% of the wild-type gene sequence. In other embodiments, the knocked out gene may include up to 100% of the wild-type gene sequence (e.g., some portion of the wild-type gene sequence may be deleted) but also include one or more heterologous and/or non-functional nucleic acid sequences inserted therein.

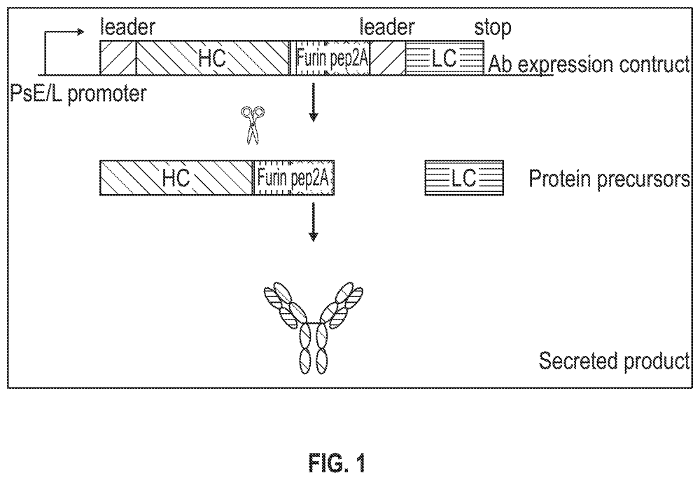

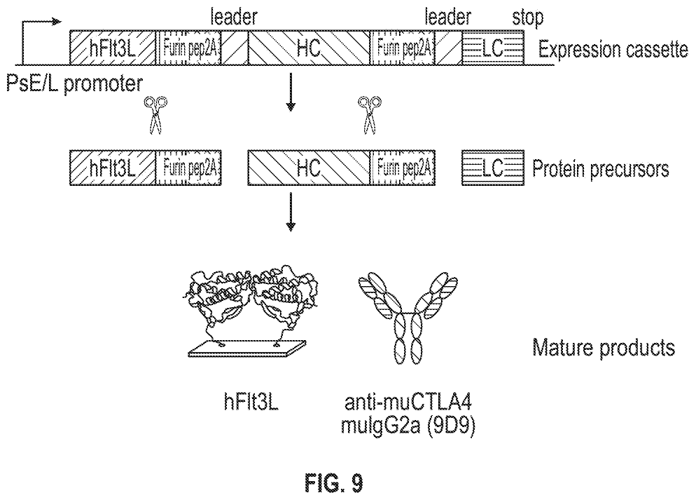

The terms “E3LΔ83N-TK⁻-anti-CTLA-4,” “E3LΔ83N-TK⁻-anti-muCTLA-4 muIgG2a,” “E3LΔ83N-TK⁻-anti-muCTLA-4,” or “E3LΔ83N-TK⁻-anti-huCTLA-4” are used herein to refer to a recombinant vaccinia virus or a vaccine comprising the virus, in which the thymidine kinase (TK) gene, through homologous recombination, has been engineered to contain a disruption comprising a heterologous nucleic acid sequence comprising one or more expression cassettes, which results in a TK gene knockout such that the TK gene is not expressed, expressed at levels so low as to have no effect, or the expressed protein is non-functional (e.g., is a null-mutation). The resulting engineered virus comprises one or more expression cassettes that are flanked by a partial sequence of the TK gene (TK-L and TK-R) on either side. In some embodiments, the expression cassette comprises a single open reading frame that encodes a specific gene of interest (SG), such as anti-muCTLA-4 (“9D9”) or anti-huCTLA-4, using the vaccinia viral synthetic early and late promoter (PsE/L). The anti-CTLA-4 antibody described herein includes murine CTLA-4 (muCTLA-4), human or humanized CTLA-4 (huCTLA-4) antibodies, and anti-CTLA-4 antibodies such as ipilimumab. The coding sequence of the heavy chain (muIgG2a) and light chain of 9D9 is separated by a cassette including a furin cleavage site followed by a Pep2A sequence, which enables ribosome skipping and the initiation of light chain protein synthesis. Human IgG kappa light chain leader sequence is used as the signal peptide for both the heavy and light chain of 9D9. This construct allows for the generation of a single transcript that can be translated into two protein precursors. The linker peptide is cleaved by furin resulting in the generation of a mature heavy chain, which is then paired with the light chain and secreted as a fully assembled IgG (FIG. 1). In some embodiments, the open reading frame further encodes a human Fms-like tyrosine kinase 3 ligand (hFlt3L) gene (“E3LΔ83N-TK⁻-hFlt3L-anti-CTLA-4,” “E3LΔ83N-TK⁻-hFlt3L-anti-muCTLA-4,” “E3LΔ83N-TK⁻-hFlt3L-anti-huCTLA-4”), wherein the nucleotide sequence encoding hFlt3L and the nucleotide sequence encoding the heavy chain of anti-muCTLA-4 (muIgG2a) (9D9) is separated by a cassette including a furin cleavage site followed by a Pep2a sequence (FIG. 9). Again, human IgG kappa light chain leader sequence is used as the signal peptide for both the heavy and light chain of 9D9. This construct also allows for the generation of a single transcript, which can be translated into three protein precursors. The linker peptide is cleaved by furin resulting in the generation of hFlt3L, as well as the mature heavy chain, which is then paired with the light chain, and secreted as a fully assembled IgG. In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene. A non-limiting example of a 9D9 antibody expression construct open reading frame according to the present technology is shown in SEQ ID NO: 1 (Table 1). A non-limiting example of an hFlt3L-9D9 antibody expression construct open reading frame according to the present technology is shown in SEQ ID NO: 5 (Table 1).

As used herein, “metastasis” refers to the spread of cancer from its primary site to neighboring tissues or distal locations in the body. Cancer cells (including cancer stem cells) can break away from a primary tumor, penetrate lymphatic and blood vessels, circulate through the bloodstream, and grow in normal tissues elsewhere in the body. Metastasis is a sequential process, contingent on tumor cells (or cancer stem cells) breaking off from the primary tumor, traveling through the bloodstream or lymphatics, and stopping at a distant site. Once at another site, cancer cells re-penetrate through the blood vessels or lymphatic walls, continue to multiply, and eventually form a new tumor (metastatic tumor). In some embodiments, this new tumor is referred to as a metastatic (or secondary) tumor.

As used herein, “oncolytic virus” refers to a virus that preferentially infects cancer cells, replicates in such cells, and induces lysis of the cancer cells through its replication process. Nonlimiting examples of naturally occurring oncolytic viruses include vesicular stomatitis virus, reovirus, as well as viruses engineered to be oncoselective such as adenovirus, Newcastle disease virus and herpes simplex virus (See, e.g., Nemunaitis, J. Invest New Drugs. 17(4):375-86 (1999); Kim, D H et al. Nat Rev Cancer. 9(1):64-71(2009); Kim et al. Nat. Med. 7:781 (2001); Coffey et al. Science 282:1332 (1998)). Vaccinia virus infects many types of cells but replicates preferentially in tumor cells due to the fact that tumor cells have a metabolism that favors replication, exhibit activation of certain pathways that also favor replication and create an environment that evades the innate immune system, which also favors viral replication.

As used herein, “parenteral,” when used in the context of administration of a therapeutic substance or composition, includes any route of administration other than administration through the alimentary tract. Particularly relevant for the methods disclosed herein are intravenous (including, for example, through the hepatic portal vein for hepatic delivery), intratumoral, or intrathecal administration.

As used herein, “pharmaceutically acceptable carrier and/or diluent” or “pharmaceutically acceptable excipient” includes without limitation any and all solvents, dispersion media, coatings, antibacterial and antifungal agents, isotonic and absorption delaying agents, and the like. The use of such media and agents for biologically active substances is well known in the art. Further details of excipients are provided below. Supplementary active ingredients, such as antimicrobials, for example antifungal agents, can also be incorporated into the compositions.

As used herein, “pharmaceutically acceptable excipient” refers to substances and compositions that do not produce an adverse, allergic, or other untoward reaction when administered to an animal or a human. As used herein, the term includes all inert, non-toxic, liquid or solid fillers or diluents, as long as they do not react with the therapeutic substance of the present technology in an inappropriate negative manner, solvents, dispersion media, coatings, antibacterial and antifungal agents, isotonic and absorption delaying agents, preservatives and the like, for example liquid pharmaceutical carriers e.g., sterile water, saline, sugar solutions, Tris buffer, ethanol and/or certain oils.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, “solid tumor” refers to all neoplastic cell growth and proliferation, and all pre-cancerous and cancerous cells and tissues, except for hematologic cancers such as lymphomas, leukemias, and multiple myeloma. Examples of solid tumors include, but are not limited to: soft tissue sarcoma, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor and other bone tumors (e.g., osteosarcoma, malignant fibrous histiocytoma), leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, brain/CNS tumors (e.g., astrocytoma, glioma, glioblastoma, childhood tumors, such as atypical teratoid/rhabdoid tumor, germ cell tumor, embryonal tumor, ependymoma) medulloblastoma, craniopharyngioma, ependymoma, pinealoma, hemangioblastoma, acoustic neuroma, oligodendroglioma, meningioma, melanoma, neuroblastoma, and retinoblastoma. Some of the most common solid tumors for which the compositions and methods of the present disclosure would be useful include: head-and-neck cancer, rectal adenocarcinoma, glioma, medulloblastoma, urothelial carcinoma, pancreatic adenocarcinoma, uterine (e.g., endometrial cancer, fallopian tube cancer) ovarian cancer, cervical cancer prostate adenocarcinoma, non-small cell lung cancer (squamous and adenocarcinoma), small cell lung cancer, melanoma, breast carcinoma, ductal carcinoma in situ, renal cell carcinoma, and hepatocellular carcinoma. adrenal tumors (e.g., adrenocortical carcinoma), esophageal, eye (e.g., melanoma, retinoblastoma), gallbladder, gastrointestinal, Wilms' tumor, heart, head and neck, laryngeal and hypopharyngeal, oral (e.g., lip, mouth, salivary gland), nasopharyngeal, neuroblastoma, peritoneal, pituitary, Kaposi's sarcoma, small intestine, stomach, testicular, thymus, thyroid, parathyroid, vaginal tumor, and the metastases of any of the foregoing.

As used herein, the terms “subject,” “individual,” or “patient” can be an individual organism, a vertebrate, a mammal, or a human. In some embodiments, “subject” means any animal (mammalian, human, or other) patient that can be afflicted with cancer and when thus afflicted is in need of treatment.

As used herein, a “synergistic therapeutic effect” refers to a greater-than-additive therapeutic effect which is produced by a combination of at least two agents, and which exceeds that which would otherwise result from the individual administration of the agents. For example, lower doses of one or more agents may be used in treating a disease or disorder, resulting in increased therapeutic efficacy and decreased side-effects.

“Treating,” “treat,” “treated,” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission. In some embodiments, “inhibiting,” means reducing or slowing the growth of a tumor. In some embodiments, the inhibition of tumor growth may be, for example, by 5% or more, 10% or more, 20% or more, 30% or more, 40% or more, 50% or more, 60% or more, 70% or more, 80% or more, or 90% or more. In some embodiments, the inhibition may be complete.

It is also to be appreciated that the various modes of treatment or prevention of medical diseases and conditions as described are intended to mean “substantial,” which includes total but also less than total treatment or prevention, and wherein some biologically or medically relevant result is achieved.

As used herein, “tumor immunity” refers to one or more processes by which tumors evade recognition and clearance by the immune system. Thus, as a therapeutic concept, tumor immunity is “treated” when such evasion is attenuated or eliminated, and the tumors are recognized and attacked by the immune system (the latter being termed herein “anti-tumor immunity”). An example of tumor recognition is tumor binding, and examples of tumor attack are tumor reduction (in number, size, or both) and tumor clearance.

As used herein, “T-cell” refers to a thymus derived lymphocyte that participates in a variety of cell-mediated adaptive immune reactions.

As used herein, “helper T-cell” refers to a CD4⁺ T-cell; helper T-cells recognize antigen bound to MHC Class II molecules. There are at least two types of helper T-cells, Th1 and Th2, which produce different cytokines.

As used herein, “cytotoxic T-cell” refers to a T-cell that usually bears CD8 molecular markers on its surface (CD8⁺) and that functions in cell-mediated immunity by destroying a target cell having a specific antigenic molecule on its surface. Cytotoxic T-cells also release Granzyme, a serine protease that can enter target cells via the perforin-formed pore and induce apoptosis (cell death). Granzyme serves as a marker of cytotoxic phenotype. Other names for cytotoxic T-cell include CTL, cytolytic T-cell, cytolytic T lymphocyte, killer T-cell, or killer T lymphocyte. Targets of cytotoxic T-cells may include virus-infected cells, cells infected with bacterial or protozoal parasites, or cancer cells. Most cytotoxic T-cells have the protein CD8 present on their cell surfaces. CD8 is attracted to portions of the Class I MHC molecule. Typically, a cytotoxic T-cell is a CD8⁺ cell.

As used herein, “tumor-infiltrating leukocytes” refers to white blood cells of a subject afflicted with a cancer (such as melanoma), that are resident in or otherwise have left the circulation (blood or lymphatic fluid) and have migrated into a tumor.

As used herein, “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene. The term “expression vector” or “expression construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or protein precursor from a transcribed gene.

The term “virulence” as used herein to refer to the relative ability of a pathogen to cause disease. The term “attenuated virulence” or “reduced virulence” is used herein to refer to a reduced relative ability of a pathogen to cause disease.

### II. Immune System and Cancer

Malignant tumors are inherently resistant to conventional therapies and present significant therapeutic challenges. Immunotherapy has become an evolving area of research and an additional option for the treatment of certain types of cancers. The immunotherapy approach rests on the rationale that the immune system may be stimulated to identify tumor cells, and target them for destruction.

Numerous studies support the importance of the differential presence of immune system components in cancer progression (Jochems et al., Exp Biol Med, 236(5): 567-579 (2011)). Clinical data suggest that high densities of tumor-infiltrating lymphocytes are linked to improved clinical outcome (Mlecnik et al., Cancer Metastasis Rev.; 30: 5-12, (2011)). The correlation between a robust lymphocyte infiltration and patient survival has been reported in various types of cancer, including melanoma, ovarian, head and neck, breast, urothelial, colorectal, lung, hepatocellular, gallbladder, and esophageal cancer (Angell et al., Current Opinion in Immunology, 25:1-7, (2013)). Tumor immune infiltrates include macrophages, dendritic cells (DC), monocytes, neutrophils, natural killer (NK) cells, naïve and memory lymphocytes, B cells and effector T-cells (T lymphocytes), primarily responsible for the recognition of antigens expressed by tumor cells and subsequent destruction of the tumor cells by cytotoxic T-cells.

Despite presentation of antigens by cancer cells and the presence of immune cells that could potentially react against tumor cells, in many cases the immune system does not get activated or is affirmatively suppressed. Key to this phenomenon is the ability of tumors to protect themselves from immune response by coercing cells of the immune system to inhibit other cells of the immune system. Tumors develop a number of immunomodulatory mechanisms to evade antitumor immune responses. For example, tumor cells secrete immune inhibitory cytokines (such as TGF-β) or induce immune cells, such as CD4⁺ T regulatory cells and macrophages, in tumor lesions to secrete these cytokines. Tumors also have the ability to bias CD4⁺ T-cells to express the regulatory phenotype. The overall result is impaired T-cell responses and impaired induction of apoptosis or reduced anti-tumor immune capacity of CD8⁺ cytotoxic T-cells. Additionally, tumor-associated altered expression of MHC class I on the surface of tumor cells makes them “invisible” to the immune response (Garrido et al. Cancer Immunol. Immunother. 59(10), 1601-1606 (2010)). Inhibition of antigen-presenting functions and dendritic cell (DC) additionally contributes to the evasion of anti-tumor immunity (Gerlini et al. Am. J. Pathol. 165(6), 1853-1863 (2004)).

Moreover, the local immunosuppressive nature of the tumor microenvironment, along with immune editing, can lead to the escape of cancer cell subpopulations that do not express the target antigens. Thus, finding an approach that would promote the preservation and/or restoration of anti-tumor activities of the immune system would be of considerable therapeutic benefit.

Immune checkpoints have been implicated in the tumor-mediated downregulation of anti-tumor immunity and used as therapeutic targets. It has been demonstrated that T-cell dysfunction occurs concurrently with an induced expression of the inhibitory receptors, CTLA-4 and programmed cell death 1 polypeptide (PD-1), members of the CD28 family of receptors. PD-1 is an inhibitory member of the CD28 family of receptors that in addition to PD-1 includes CD28, CTLA-4, ICOS and BTLA. However, while promise regarding the use of immunotherapy in the treatment of melanoma has been underscored by the clinical use and even regulatory approval of anti-CTLA-4 (ipilimumab) and anti-PD-1 drugs (e.g., pembrolizumab and nivolumab), the response of patients to these immunotherapies has been limited. Clinical trials, focused on blocking these inhibitory signals in T-cells (e.g., CTLA-4, PD-1, and the ligand of PD-1, PD-L1), have shown that reversing T-cell suppression is critical for successful immunotherapy (Sharma et al., Science 348(6230), 56-61 (2015); Topalian et al., Curr Opin Immunol. 24(2), 202-217 (2012)). These observations highlight the need for development of novel therapeutic approaches for harnessing the immune system against cancer.

### III. Poxviruses

Poxviruses, such as engineered vaccinia viruses, are in the forefront as oncolytic therapy for metastatic cancers (Kim et al., Nature Review Cancer 9, 64-71 (2009)). Vaccinia viruses are large DNA viruses, which have a rapid life cycle and efficient hematogenous spread to distant tissues (Moss, In Fields Virology (Lippincott Williams & Wilkins, 2007), pp. 2905-2946). Poxviruses are well-suited as vectors to express multiple transgenes in cancer cells and thus to enhance therapeutic efficacy (Breitbach et al., Current pharmaceutical biotechnology 13, 1768-1772 (2012)). Preclinical studies and clinical trials have demonstrated efficacy of using oncolytic vaccinia viruses and other poxviruses for treatment of advanced cancers refractory to conventional therapy (Park et al., Lacent Oncol 9, 533-542 (2008); Kim et al., PLoS Med 4, e353 (2007); Thorne et al., J Clin Invest 117, 3350-3358 (2007)). Poxvirus-based oncolytic therapy has the advantage of killing cancer cells through a combination of cell lysis, apoptosis, and necrosis. It also triggers innate immune sensing pathway that facilitates the recruitment of immune cells to the tumors and the development of anti-tumor adaptive immune responses. The current oncolytic vaccinia strains in clinical trials (JX-594, for example) are replicative strains. They use wild-type vaccinia with deletion of thymidine kinase to enhance tumor selectivity, and with expression of transgenes such as granulocyte macrophage colony stimulating factor (GM-CSF) to stimulate immune responses (Breitbach et al., Curr Pharm Biotechnol 13, 1768-1772 (2012)). Many studies have shown, however, that wild-type vaccinia has immune suppressive effects on antigen presenting cells (APCs) (Engelmayer et al., J Immunol 163, 6762-6768 (1999); Jenne et al., Gene therapy 7, 1575-1583 (2000); P. Li et al., J Immunol 175, 6481-6488 (2005); Deng et al., J Virol 80, 9977-9987 (2006)), and thus adds to the immunosuppressive and immunoevasive effects of tumors themselves.

### IV. E3LΔ83N Virus

Poxviruses are extraordinarily adept at evading and antagonizing multiple innate immune signaling pathways by encoding proteins that interdict the extracellular and intracellular components of those pathways (Seet et al. Annu. Rev. Immunol. 21377-423 (2003)). Chief among the poxvirus antagonists of intracellular innate immune signaling is the vaccinia virus duel Z-DNA and dsRNA-binding protein E3, which can inhibit the PKR and NF-κB pathways (Cheng et al. Proc. Natl. Acad. Sci. USA 894825-4829 (1992); Deng et al. J. Virol. 809977-9987 (2006)) that would otherwise be activated by vaccinia virus infection. A mutant vaccinia virus lacking the E3L gene (ΔE3L) has a restricted host range, is highly sensitive to IFN, and has greatly reduced virulence in animal models of lethal poxvirus infection (Beattie et al. Virus Genes. 1289-94 (1996); Brandt et al. Virology 333263-270 (2004)). Recent studies have shown that infection of cultured cell lines with ΔE3L virus elicits proinflammatory responses that are masked during infection with wild-type vaccinia virus (Deng et al. J. Virol. 809977-9987 (2006); Langland et al. J. Virol. 8010083-10095). Infection of a mouse epidermal dendritic cell line with wild-type vaccinia virus attenuated proinflammatory responses to the TLR agonists lipopolysaccharide (LPS) and poly(I:C), an effect that was diminished by deletion of E3L. Moreover, infection of the dendritic cells with ΔE3L virus triggered NF-κB activation in the absence of exogenous agonists (Deng et al. J. Virol. 809977-9987 (2006)). Whereas wild-type vaccinia virus infection of murine keratinocytes does not induce the production of proinflammatory cytokines and chemokines, infection with ΔE3L virus does induce the production of IFN-β, IL-6, CCL4 and CCL5 from murine keratinocytes, which is dependent on the cytosolic dsRNA-sensing pathway mediated by the mitochondrial antiviral signaling protein (MAVS; an adaptor for the cytosolic RNA sensors RIG-I and MDAS) and the transcription factor IRF3 (Deng et al., J Virol. 2008 November; 82(21): 10735-10746.).

E3LΔ83N virus with deletion of the Z-DNA-binding domain is 1,000-fold more attenuated than wild-type vaccinia virus in an intranasal infection model (Brandt et al., 2001). E3LΔ83N also has reduced neurovirulence compared with wild-type vaccinia in an intra-cranial inoculation model (Brandt et al., 2005). A mutation within the Z-DNA binding domain of E3 (Y48A) resulting in decreased Z-DNA-binding leads to decreased neurovirulence (Kim et al., 2003). Although the N-terminal Z-DNA binding domain of E3 is important in viral pathogenesis, how it affects host innate immune sensing of vaccinia virus is not well understood. Myxoma virus but not wild-type vaccinia infection of murine plasmacytoid dendritic cells induces type I IFN production via the TLR9/MyD88/IRF5/IRF7-dependent pathway (Dai et al., 2011). Myxoma virus E3 ortholog M029 retains the dsRNA-binding domain of E3 but lacks the Z-DNA binding domain of E3. It was found that the Z-DNA-binding domain of E3 (but probably not Z-DNA-binding activity per se) plays an important role in inhibiting poxviral sensing in murine and human pDCs (Dai et al., 2011; Cao et al., 2012).

Deletion of E3L sensitizes vaccinia virus replication to IFN inhibition in permissive RK13 cells and results in a host range phenotype, whereby ΔE3L cannot replicate in HeLa or BSC40 cells (Chang et al., 1995). The C-terminal dsRNA-binding domain of E3 is responsible for the host range effects, whereas E3LΔ83N virus with deletion of the N-terminal Z-DNA-binding domain is replication competent in HeLa and BSC40 cells (Brandt et al., 2001).

Vaccinia virus (Western Reserve strain; WR) with deletion of thymidine kinase is highly attenuated in non-dividing cells but is replicative in transformed cells (Buller et al., 1988). TK-deleted vaccinia virus selectively replicates in tumor cells in vivo (Puhlmann et al., 2000). Thorne et al. showed that compared with other vaccinia strains, WR strain has the highest burst ratio in tumor cell lines relative to normal cells (Thorne et al., 2007). The derivative of this strain, vaccinia E3LΔ83N WR strain, was selected for further modification in the present disclosure.

### V. Fms-Like Tyrosine Kinase 3 Ligand (Flt3L)

Human Flt3L (Fms-like tyrosine kinase 3 ligand) is a type I transmembrane protein that stimulates the proliferation of bone marrow cells. The use of hFlt3L has been explored in various preclinical and clinical settings including stem cell mobilization in preparation for bone marrow transplantation, cancer immunotherapy such as expansion of dendritic cells, as well as a vaccine adjuvant. Recombinant human Flt3L (rhuFlt3L) has been tested in more than 500 human subjects and is bioactive, safe, and well tolerated (Fong et al., 1998; Maraskovsky et al., 2000; Shackleton et al., 2004; He et al., 2014; Anandasabapathy et al., 2015).

### VI. Engineered Vaccinia Virus Strains of the Present Technology

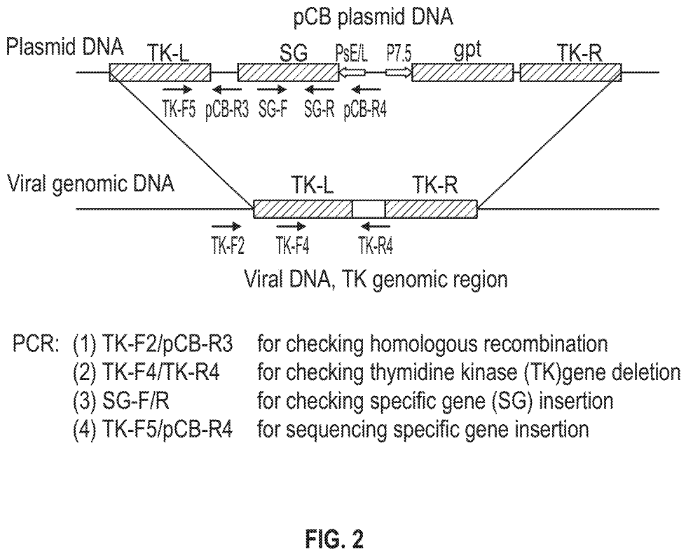

The disclosure of the present technology relates to recombinant vaccinia E3LΔ83N-TK⁻ viruses, or vaccines comprising the viruses, engineered to express one or more specific genes of interest (SG), such as anti-CLTA-4 antibody or hFlt3L, for use as an oncolytic therapy. In some embodiments, the thymidine kinase (TK) gene of the E3LΔ83N virus, through homologous recombination, has been engineered to contain a disruption comprising a heterologous nucleic acid sequence comprising one or more expression cassettes, which results in a TK gene knockout such that the TK gene is not expressed, expressed at levels so low as to have no effect, or the expressed protein is non-functional (e.g., is a null-mutation). The resulting E3LΔ83N-TK⁻ virus is further engineered to comprise one or more expression cassettes that are flanked by a partial sequence of the TK gene (TK-L and TK-R) on either side (FIG. 2). In some embodiments, the expression cassette comprises a single open reading frame that encodes a specific gene of interest (SG), such as anti-muCTLA-4 (“9D9”) or anti-huCTLA-4 using the vaccinia viral synthetic early and late promoter (PsE/L), resulting in E3LΔ83N-TK⁻-anti-CTLA-4, E3LΔ83N-TK⁻-anti-muCTLA-4, or E3LΔ83N-TK⁻-anti-huCTLA-4.

In some embodiments, the open reading frame comprises one or more of the heavy chain CDR regions of anti-CTLA-4 as described in Table 1, and/or one or more of the light chain CDR regions of anti-CTLA-4 as described in Table 1. In some embodiments, the open reading frame comprises all six heavy and light chain CDR regions of anti-CTLA-4 as described in Table 1. In some embodiments, the open reading frame encodes an anti-CTLA-4 antibody or antigen binding fragment thereof comprising a heavy chain immunoglobulin variable domain (V_{H}) and a light chain immunoglobulin variable domain (V_{L}), wherein (a) the V_{H} comprises a V_{H}-CDR1 sequence of GYTFTDY (SEQ ID NO: 27), a V_{H}-CDR2 sequence of PYNG (SEQ ID NO: 28), and aV_{H}-CDR3 sequence of YGSWFA (SEQ ID NO: 29), and (b) the V_{L} comprises a V_{L}-CDR1 sequence of SQSIVHSNGNTY (SEQ ID NO: 30), a V_{L}-CDR2 sequence of KVS (SEQ ID NO: 31), and a V_{L}-CDR3 sequence of GSHVPY (SEQ ID NO: 32). In some embodiments, the open reading frame encodes an anti-CTLA-4 antibody or antigen binding fragment thereof comprising a V_{H} and a V_{L}, wherein (a) the V_{H} comprises a V_{H}-CDR1 sequence of GYTFTDY (SEQ ID NO: 27), a V_{H}-CDR2 sequence of PYNG (SEQ ID NO: 28), and aV_{H}-CDR3 sequence of YGSWFA (SEQ ID NO: 29), and (b) the V_{L} comprises a V_{L}-CDR1 sequence of SQSIVHSNGNTY (SEQ ID NO: 30), a V_{L}-CDR2 sequence of KVS (SEQ ID NO: 31), and a V_{L}-CDR3 sequence of GSHVPY (SEQ ID NO: 32), and wherein the open reading frame is at least 80%, at least 85%, at least 90%, at least 95%, at least 99%, or is 100% identical to the nucleotide sequence set forth in SEQ ID NO: 1. In some embodiments, the open reading frame encodes an anti-CTLA-4 antibody or antigen binding fragment thereof comprising a V_{H} and a V_{L}, wherein (a) the V_{H} comprises a V_{H}-CDR1 sequence of GYTFTDY (SEQ ID NO: 27), a V_{H}-CDR2 sequence of PYNG (SEQ ID NO: 28), and aV_{H}-CDR3 sequence of YGSWFA (SEQ ID NO: 29), and (b) the V_{L} comprises a V_{L}-CDR1 sequence of SQSIVHSNGNTY (SEQ ID NO: 30), a V_{L}-CDR2 sequence of KVS (SEQ ID NO: 31), and a V_{L}-CDR3 sequence of GSHVPY (SEQ ID NO: 32), and wherein the open reading frame is at least 80%, at least 85%, at least 90%, at least 95%, at least 99%, or is 100% identical to the nucleotide sequence set forth in SEQ ID NO: 5.

In some embodiments, the open reading frame comprises a nucleotide sequence that comprises the six heavy and light chain CDR regions of anti-CTLA-4 encoded by SEQ ID NO: 1, optionally further comprising nucleotide sequence that is at least 95% identical to the nucleotide sequence encoded by SEQ ID NO: 5. In some embodiments, the open reading frame comprises one or more of the heavy chain CDR regions of anti-CTLA-4 as described in Table 1, one or more of the light chain CDR regions of anti-CTLA-4 as described in Table 1, and at least 95% sequence identity to the nucleotide sequence set forth in SEQ ID NO: 1. In some embodiments, the open reading frame comprises one or more of the heavy chain CDR regions of anti-CTLA-4 as described in Table 1, one or more of the light chain CDR regions of anti-CTLA-4 as described in Table 1, and at least 95% sequence identity to the nucleotide sequence set forth in SEQ ID NO: 5.

In some embodiments, the open reading frame encodes an anti-huCTLA-4 antibody. In some embodiments, the open reading frame encodes the heavy chain CDR regions of an anti-huCTLA-4, and/or the light chain CDR regions of an anti-huCTLA-4 such as ipilimumab. In some embodiments the open reading frame encodes the heavy chain and/or light chain variable regions of an anti-huCTLA-4 such as ipilimumab. In some embodiments the open reading frame encodes the heavy chain and/or light chain of an anti-huCTLA-4 such as ipilimumab.

In some embodiments, the open reading frame encodes a heavy chain variable region that is at least 95% identical to the amino acid sequence of the heavy chain variable region of an anti-huCTLA-4 such as ipilimumab. In some embodiments, the open reading frame encodes a light chain variable region that is at least 95% identical to the amino acid sequence of the light chain variable region of an anti-huCTLA-4 such as ipilimumab. In some embodiments, the open reading frame encodes both a heavy chain variable region and a light chain variable region that is at least 95% identical to the amino acid sequence of the heavy chain variable region and the light chain variable region of an anti-huCTLA-4 such as ipilimumab. In such embodiments, the heavy and light chain CDRs may be unmodified.

In some embodiments, the coding sequence of the heavy chain (muIgG2a) and light chain of 9D9 is separated by a cassette including a furin cleavage site followed by a Pep2A sequence, which enables ribosome skipping and the initiation of light chain protein synthesis. In some embodiments, the Pep2A comprises one of the following: T2A having an amino acid sequence sequence (GSG) E G R G S L L T C G D V E E N P G P (SEQ ID NO: 23); P2A having an amino acid sequence (GSG) A T N F S L L K Q A G D V E E N P G P (SEQ ID NO: 24); E2A having an amino acid sequence (GSG) Q C T N Y A L L K L A G D V E S N P G P (SEQ ID NO: 25); or F2A having an amino acid sequence (GSG) V K Q T L N F D L L K L A G D V E S N P G P (SEQ ID NO: 26), where the N-terminal (GSG) for each 2A peptide is optional. Human IgG kappa light chain leader sequence is used as the signal peptide for both the heavy and light chain of 9D9. This construct allows for the generation of a single transcript that can be translated into two protein precursors. The linker peptide is cleaved by furin resulting in the generation of a mature heavy chain, which is then paired with the light chain and secreted as a fully assembled IgG (FIG. 1).

In some embodiments, the open reading frame further encodes an hFlt3L gene (E3LΔ83N-TK⁻-hFlt3L-anti-CTLA-4 or E3LΔ83N-TK⁻-hFlt3L-anti-muCTLA-4 or E3LΔ83N-TK⁻-hFlt3L-anti-huCTLA-4), wherein the nucleotide sequence encoding hFlt3L and the nucleotide sequence encoding the heavy chain of anti-CTLA-4 (e.g., anti-muCTLA-4 muIgG2a) (9D9) is separated by a cassette including a furin cleavage site followed by a Pep2a sequence. Again, human IgG kappa light chain leader sequence is used as the signal peptide for both the heavy and light chain of 9D9. This construct also allows for the generation of a single transcript, which can be translated into three protein precursors. The linker peptide is cleaved by furin resulting in the generation of hFlt3L, as well as the mature heavy chain, which is then paired with the light chain, and secreted as a fully assembled IgG (FIG. 9).

In some embodiments, the disclosure of the present technology relates to recombinant E3LΔ83N-TK⁻ virus as described above, wherein the specific gene of interest (SG) is an anti-programmed death-ligand 1 (PD-L1) antibody, thereby resulting in the following viruses: E3LΔ83N-TK⁻-anti-PD-L1 or E3LΔ83N-TK⁻-hFlt3L-anti-PD-L1.

In some embodiments, the heterologous nucleotide sequence further comprises an additional expression cassette comprising an open reading frame that encodes a selectable marker operably linked to a promoter that is capable of directing expression of the selectable marker (FIG. 2). In some embodiments, the selectable marker is a xanthine-guanine phosphoribosyl transferase (gpt) gene.

A non-limiting example of a 9D9 antibody expression construct open reading frame according to the present technology is shown in SEQ ID NO: 1 (Table 1). A non-limiting example of an hFlt3L-9D9 antibody expression construct according to the present technology is shown in SEQ ID NO: 5 (Table 1).

In some embodiments, the disclosure of the present technology relates to a recombinant vaccinia strain comprising a disruption of a C7L gene (VACVΔC7L) and engineered to express one or more specific genes of interest (SG), such as anti-CLTA-4 antibody or hFlt3L, for use as an oncolytic therapy. In some embodiments, the vaccinia host range factor C7L gene, through homologous recombination, has been engineered to contain a disruption comprising a heterologous nucleic acid sequence comprising one or more expression cassettes, which results in a C7L gene knockout such that the C7L gene is not expressed, expressed at levels so low as to have no effect, or the expressed protein is non-functional (e.g., is a null-mutation). The resulting VACVΔC7L virus is further engineered to comprise one or more expression cassettes that are flanked by a partial sequence of the C7L gene (C7-L and C7-R) on either side. In some embodiments, the expression cassette comprises a single open reading frame that encodes a specific gene of interest (SG), such as anti-CTLA-4 (“9D9”) using the vaccinia viral synthetic early and late promoter (PsE/L), resulting in VACVΔC7L-anti-CTLA-4 or VACVΔC7L-anti-muCTLA-4 or VACVΔC7L-anti-huCTLA-4. In some embodiments, the coding sequence of the heavy chain and light chain of 9D9 is separated by a cassette including a furin cleavage site followed by a Pep2A sequence, which enables ribosome skipping and the initiation of light chain protein synthesis. Human IgG kappa light chain leader sequence is used as the signal peptide for both the heavy chain and the light chain of 9D9. This construct allows for the generation of a single transcript that can be translated into two protein precursors. The linker peptide is cleaved by furin resulting in the generation of a mature heavy chain, which is then paired with the light chain and secreted as a fully assembled IgG.

In some embodiments, the open reading frame further encodes an hFlt3L gene (VACVΔC7L-hFlt3L-anti-CTLA-4 or VACVΔC7L-hFlt3L-anti-muCTLA-4 or VACVΔC7L-hFlt3L-anti-huCTLA-4), wherein the nucleotide sequence encoding hFlt3L and the nucleotide sequence encoding the heavy chain of anti-CTLA-4 (9D9) is separated by a cassette including a furin cleavage site followed by a Pep2a sequence. Again, human IgG kappa light chain leader sequence is used as the signal peptide for both the heavy and light chain of 9D9. This construct also allows for the generation of a single transcript, which can be translated into three protein precursors. The linker peptide is cleaved by furin resulting in the generation of hFlt3L, as well as the mature heavy chain, which is then paired with the light chain, and secreted as a fully assembled IgG.

In some embodiments, the disclosure of the present technology relates to recombinant VACVΔC7L virus as described above, wherein the specific gene of interest (SG) is an anti-programmed death-ligand 1 (PD-L1) antibody, thereby resulting in the following viruses: VACVΔC7L-anti-PD-L1 or VACVΔC7L-hFlt3L-anti-PD-L1.

In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene.

In some embodiments, the disclosure of the present technology relates to recombinant vaccinia virus comprising the E3LΔ83N-TK⁻ and ΔC7L modifications as described above, engineered to express a specific gene of interest (SG), such as an anti-CTLA-4 antibody, anti PD-L1 antibody, hFlt3L, or any combination thereof.

Exemplary nucleotide and amino acid sequences for the open reading frames of the vaccinia virus constructs of the present technology are provided in Table 1, anti-muCTLA4-muIgG2a nucleotide sequence (SEQ ID NO: 1), anti-muCTLA4-muIgG2a amino acid sequence (SEQ ID NO: 2), anti-DNPmuIgG2a nucleotide sequence (SEQ ID NO: 3), anti-DNPmuIgG2a amino acid sequence (SEQ ID NO: 4), hFltL3_PEP2A_anti-CTLA-4HC_PEP2A_anti-CTLA-4LC nucleotide sequence (SEQ ID NO: 5), and hFltL3_PEP2A_anti-CTLA-4HC_PEP2A_anti-CTLA-4LC amino acid sequence (SEQ ID NO: 6).

TABLE 1
Exemplary nucleotide and amino acid sequences
for the open reading frames of the vaccinia
virus constructs of the present technology.

anti-muCTLA4-muIgG2a nucleotide sequence (SEQ ID
NO: 1).
5′ATGGAATGGTCCTTTGTCTTTCTTTTTTTCTTGTCCGCAGCTGCCGGA
GTACATTCGGAGGCGAAGTTGCAAGAGTCCGGACCTGTACTTGTTAAGCC
CGGAGCTTCAGTGAAAATGTCCTGTAAAGCATCCGGATATACCTTTACAG
ATTATTATATGAATTGGGTGAAGCAAAGTCATGGAAAGAGTCTTGAATGG
ATAGGAGTAATTAATCCTTATAACGGAGATACATCTTATAATCAAAAGTT
CAAAGGAAAAGCTACACTAACTGTTGATAAATCCTCAAGTACTGCTTATA
TGGAACTAAACTCACTAACTAGTGAAGATTCTGCAGTTTATTATTGTGCT
CGTTATTATGGTTCGTGGTTTGCATATTGGGGACAGGGAACCTTAATAAC
TGTAAGTACAGCAAAAACAACGGCGCCTTCTGTTTATCCATTAGCGCCTG
TATGTGGAGATACAACTGGTTCTTCTGTTACATTAGGATGTCTAGTCAAA
GGATATTTCCCAGAACCTGTTACATTAACCTGGAACTCCGGTTCGCTATC
ATCAGGTGTACACACTTTCCCGGCGGTTCTACAATCTGATTTGTATACAT
TATCATCTTCCGTTACAGTTACTTCTTCCACTTGGCCATCGCAAAGTATC
ACATGTAACGTAGCGCACCCAGCTTCATCAACAAAAGTCGATAAAAAAAT
AGAGCCGCGAGGTCCCACTATAAAGCCGTGTCCACCTTGTAAATGTCCAG
CTCCTAATTTATTAGGAGGACCCAGTGTATTTATTTTCCCTCCTAAAATT
AAAGATGTATTGATGATTTCTTTATCTCCAATTGTTACATGCGTGGTTGT
AGATGTATCCGAAGACGATCCGGATGTGCAAATATCGTGGTTCGTTAATA
ATGTGGAAGTTCACACCGCGCAAACTCAAACTCACAGAGAGGATTACAAT
TCTACCTTGCGTGTAGTGTCGGCTCTACCTATACAACACCAAGATTGGAT
GTCTGGAAAAGAATTTAAATGCAAAGTTAATAACAAAGACCTTCCAGCGC
CAATAGAAAGAACAATATCCAAACCTAAAGGTAGTGTAAGAGCTCCTCAA
GTATACGTTTTACCGCCTCCTGAAGAAGAAATGACGAAAAAACAAGTTAC
ATTAACCTGTATGGTGACAGATTTTATGCCAGAGGATATTTATGTGGAGT
GGACTAATAATGGAAAAACGGAATTGAATTACAAAAATACTGAACCTGTA
TTAGATAGTGATGGATCATATTTTATGTACAGTAAATTGAGAGTGGAAAA
AAAGAATTGGGTTGAAAGAAATTCGTACTCTTGTTCAGTTGTACATGAGG
GACTACATAATCATCATACCACTAAGAGTTTTTCAAGAACCCCTGGTAAA
CGTAGAAGGCGTAGGAGATCTGGTGCTACTAATTTCTCCTTGTTAAAACA
AGCCGGTGACGTCGAAGAAAACCCTGGTCCTATGATGACATGGACTCTAC
TATTCCTTGCCTTCCTTCATCACTTAACAGGGTCATGTGCC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ITALICS UPPER CASE = human IgG kappa leader
sequence
UPPER CASE UNDERLINED = anti-CTLA-4 Heavy Chain
(HC or variable heavy (V_{H}) region)
BOLD UPPER CASE = Furin cleavage site
BOLD UPPER CASE UNDERLINED = Pep2A [symbol] [symbol] [symbol] [symbol]
= anti-CTLA-4 Light Chain
(LC or variable light (V_{L}) region)
anti-muCTLA4-muIgG2a amino acid sequence (SEQ ID
NO: 2).
5′MEWSFVFLFFLSAAAGVHSEAKLQESGPVLVKPGASVKMSCKASGYTF
TDYYMNWVKQSHGKSLEWIGVINPYNGDTSYNQKFKGKATLTVDKSSSTA
YMELNSLTSEDSAVYYCARYYGSWFAYWGQGTLITVSTAKTTAPSVYPLA
PVCGDTTGSSVTLGCLVKGYFPEPVTLTWNSGSLSSGVHTFPAVLQSDLY
TLSSSVTVTSSTWPSQSITCNVAHPASSTKVDKKIEPRGPTIKPCPPCKC
PAPNLLGGPSVFIFPPKIKDVLMISLSPIVTCVVVDVSEDDPDVQISWFV
NNVEVHTAQTQTHREDYNSTLRVVSALPIQHQDWMSGKEFKCKVNNKDLP
APIERTISKPKGSVRAPQVYVLPPPEEEMTKKQVTLTCMVTDFMPEDIYV
EWTNNGKTELNYKNTEPVLDSDGSYFMYSKLRVEKKNWVERNSYSCSVVH
EGLHNHHTTKSFSRTPGKRRRRRRSGATNFSLLKQAGDVEENPGPMMTWT
LLFLAFLHHLTGSCA

[symbol]

[symbol]

[symbol]

[symbol]

[symbol]

[symbol]

[symbol]

[symbol]

[symbol]

[symbol]

[symbol]

[symbol]

[symbol]

ITALICS UPPER CASE = human IgG kappa leader
sequence
UPPER CASE UNDERLINED = anti-CTLA-4 Heavy Chain
(HC or variable heavy (V_{H}) region)
BOLD UPPER CASE = Furin cleavage site
BOLD UPPER CASE UNDERLINED = Pep2A [symbol] [symbol] [symbol] [symbol]
= anti-CTLA-4 Light Chain
(LC or variable light (V_{L}) region)
anti-CTLA-4 heavy chain immunoglobulin variable
domain (V_{H}) and a light chain immunoglobulin
variable domain (V_{L}) CDR Sequences.
V_{H}-CDR1: GYTFTDY (SEQ ID NO: 27)
V_{H}-CDR2: PYNG (SEQ ID NO: 28)
V_{H}-CDR3: YGSWFA (SEQ ID NO: 29)
V_{L}-CDR1: SQSIVHSNGNTY (SEQ ID NO: 30)
V_{L}-CDR2: KVS (SEQ ID NO: 31)
V_{L}-CDR3: GSHVPY (SEQ ID NO: 32)
anti-DNPmuIgG2a nucleotide sequence (SEQ ID NO: 3).
5′ATGGAATGGAGCTTTGTCTTTCTCTTCTTCCTGTCAGCAGCTGCAGGT
GTCCACTCCCAGGTGCAGCTGCAGGAGTCGGGCCCAGGACTGGTGAAGCC
TTTACAGACCCTGTCCCTCACCTGCACTGTCTCTGGTGGCTCCATCAGCA
GTGGTGGTTATTACTGGAGCTGGATCCGCCAGCACCCAGGGAAGGGCCTG
GAGTGGATTGGGTACATCTATTACAGTAGGAGCACCTACTACAACCCGTC
CCTCAAGAGTCGAGTTACCATATCAGTAGACACGTCTAAGAACCAGTTCT
CCCTGAAGCTGAGCTCTGTGACAGCCGCGGACACGGCCGTGTATTACTGT
GCGAGAACCGGGTATAGCAGTGGCTGGTACCCTTTTGACTACTGGGGCCA
GGGAACCCTGGTCACCGTCTCTAGTGCAAAAACAACGGCGCCTTCTGTTT
ATCCATTAGCGCCTGTATGTGGAGATACAACTGGTTCTTCTGTTACATTA
GGATGTCTAGTCAAAGGATATTTCCCAGAACCTGTTACATTAACCTGGAA
CTCCGGTTCGCTATCATCAGGTGTACACACTTTCCCGGCGGTTCTACAAT
CTGATTTGTATACATTATCATCTTCCGTTACAGTTACTTCTTCCACTTGG
CCATCGCAAAGTATCACATGTAACGTAGCGCACCCAGCTTCATCAACAAA
AGTCGATAAAAAAATAGAGCCGCGAGGTCCCACTATAAAGCCGTGTCCAC
CTTGTAAATGTCCAGCTCCTAATTTATTAGGAGGACCCAGTGTATTTATT
TTCCCTCCTAAAATTAAAGATGTATTGATGATTTCTTTATCTCCAATTGT
TACATGCGTGGTTGTAGATGTATCCGAAGACGATCCGGATGTGCAAATAT
CGTGGTTCGTTAATAATGTGGAAGTTCACACCGCGCAAACTCAAACTCAC
AGAGAGGATTACAATTCTACCTTGCGTGTAGTGTCGGCTCTACCTATACA
ACACCAAGATTGGATGTCTGGAAAAGAATTTAAATGCAAAGTTAATAACA
AAGACCTTCCAGCGCCAATAGAAAGAACAATATCCAAACCTAAAGGTAGT
GTAAGAGCTCCTCAAGTATACGTTTTACCGCCTCCTGAAGAAGAAATGAC
GAAAAAACAAGTTACATTAACCTGTATGGTGACAGATTTTATGCCAGAGG
ATATTTATGTGGAGTGGACTAATAATGGAAAAACGGAATTGAATTACAAA
AATACTGAACCTGTATTAGATAGTGATGGATCATATTTTATGTACAGTAA
ATTGAGAGTGGAAAAAAAGAATTGGGTTGAAAGAAATTCGTACTCTTGTT
CAGTTGTACATGAGGGACTACATAATCATCATACCACTAAGAGTTTTTCA
AGAACCCCTGGTAAAGGTAGTTCCGACTACAAAGACGATGACGACAAGCG
TAGAAGGCGTAGGAGATCTGGTGCTACTAATTTCTCCTTGTTAAAACAAG
CCGGTGACGTCGAAGAAAACCCTGGTCCTATG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ITALICS UPPER CASE = human IgG kappa leader
sequence
UPPER CASE UNDERLINED = anti-DNP Heavy Chain (HC)
UPPER CASE = FLAG
BOLD UPPER CASE = Furin cleavage site
BOLD UPPER CASE UNDERLINED = Pep2A [symbol] [symbol] [symbol] [symbol]
= anti-DNP Light Chain
(LC or variable light (V_{L}) region)
anti-DNPmuIgG2a amino acid sequence (SEQ ID
NO: 4).
5′MEWSFVFLFFLSAAAGVHSQVQLQESGPGLVKPLQTLSLTCTVSGGSI
SSGGYYWSWIRQHPGKGLEWIGYIYYSRSTYYNPSLKSRVTISVDTSKNQ
FSLKLSSVTAADTAVYYCARTGYSSGWYPFDYWGQGTLVTVSSAKTTAPS
VYPLAPVCGDTTGSSVTLGCLVKGYFPEPVTLTWNSGSLSSGVHTFPAVL
QSDLYTLSSSVTVTSSTWPSQSITCNVAHPASSTKVDKKIEPRGPTIKPC
PPCKCPAPNLLGGPSVFIFPPKIKDVLMISLSPIVTCVVVDVSEDDPDVQ
ISWFVNNVEVHTAQTQTHREDYNSTLRVVSALPIQHQDWMSGKEFKCKVN
NKDLPAPIERTISKPKGSVRAPQVYVLPPPEEEMTKKQVTLTCMVTDFMP
EDIYVEWTNNGKTELNYKNTEPVLDSDGSYFMYSKLRVEKKNWVERNSYS
CSVVHEGLHNHHTTKSFSRTPGKGSSDYKDDDDKRRRRRRSGATNFSLLK
QAGDVEENPGPMMTWTLLFLAFLHHLTGSCA

[symbol]

[symbol]

[symbol]

[symbol]

[symbol]

[symbol]

[symbol]

[symbol]

[symbol]

[symbol]

[symbol]

[symbol]

[symbol]

ITALICS UPPER CASE = human IgG kappa leader
sequence
UPPER CASE UNDERLINED = anti-DNP Heavy Chain (HC)
UPPER CASE = FLAG
BOLD UPPER CASE = Furin cleavage site
BOLD UPPER CASE UNDERLINED = Pep2A [symbol] [symbol] [symbol] [symbol]
= anti-DNP Light Chain
(LC)
hFltL3_PEP2A_anti-CTLA-4HC_PEP2A_anti-CTLA-4LC
nucleotide sequence (SEQ ID NO: 5).
5′ATGACAGTGCTGGCGCCAGCCTGGAGCCCAACGACCTATCTCCTCCTG

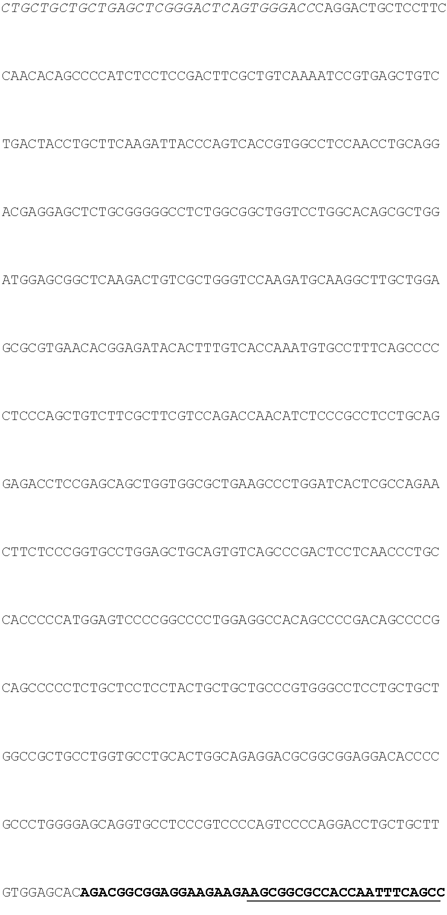

TGCTGAAACAGGCTGGCGACGTGGAAGAGAACCCCGGACCTATGATGGAA
TGGTCCTTTGTCTTTCTTTTTTTCTTGTCCGCAGCTGCCGGAGTACATTC
GGAGGCGAAGTTGCAAGAGTCCGGACCTGTACTTGTTAAGCCCGGAGCTT
CAGTGAAAATGTCCTGTAAAGCATCCGGATATACCTTTACAGATTATTAT
ATGAATTGGGTGAAGCAAAGTCATGGAAAGAGTCTTGAATGGATAGGAGT
AATTAATCCTTATAACGGAGATACATCTTATAATCAAAAGTTCAAAGGAA
AAGCTACACTAACTGTTGATAAATCCTCAAGTACTGCTTATATGGAACTA
AACTCACTAACTAGTGAAGATTCTGCAGTTTATTATTGTGCTCGTTATTA
TGGTTCGTGGTTTGCATATTGGGGACAGGGAACCTTAATAACTGTAAGTA
CAGCAAAAACAACGGCGCCTTCTGTTTATCCATTAGCGCCTGTATGTGGA
GATACAACTGGTTCTTCTGTTACATTAGGATGTCTAGTCAAAGGATATTT
CCCAGAACCTGTTACATTAACCTGGAACTCCGGTTCGCTATCATCAGGTG
TACACACTTTCCCGGCGGTTCTACAATCTGATTTGTATACATTATCATCT
TCCGTTACAGTTACTTCTTCCACTTGGCCATCGCAAAGTATCACATGTAA
CGTAGCGCACCCAGCTTCATCAACAAAAGTCGATAAAAAAATAGAGCCGC
GAGGTCCCACTATAAAGCCGTGTCCACCTTGTAAATGTCCAGCTCCTAAT
TTATTAGGAGGACCCAGTGTATTTATTTTCCCTCCTAAAATTAAAGATGT
ATTGATGATTTCTTTATCTCCAATTGTTACATGCGTGGTTGTAGATGTAT
CCGAAGACGATCCGGATGTGCAAATATCGTGGTTCGTTAATAATGTGGAA
GTTCACACCGCGCAAACTCAAACTCACAGAGAGGATTACAATTCTACCTT
GCGTGTAGTGTCGGCTCTACCTATACAACACCAAGATTGGATGTCTGGAA
AAGAATTTAAATGCAAAGTTAATAACAAAGACCTTCCAGCGCCAATAGAA
AGAACAATATCCAAACCTAAAGGTAGTGTAAGAGCTCCTCAAGTATACGT
TTTACCGCCTCCTGAAGAAGAAATGACGAAAAAACAAGTTACATTAACCT
GTATGGTGACAGATTTTATGCCAGAGGATATTTATGTGGAGTGGACTAAT
AATGGAAAAACGGAATTGAATTACAAAAATACTGAACCTGTATTAGATAG
TGATGGATCATATTTTATGTACAGTAAATTGAGAGTGGAAAAAAAGAATT
GGGTTGAAAGAAATTCGTACTCTTGTTCAGTTGTACATGAGGGACTACAT
AATCATCATACCACTAAGAGTTTTTCAAGAACCCCTGGTAAAGGTAGTTC
CGACTACAAAGACGATGACGACAAGCGTAGAAGGCGTAGGAGATCTGGTG
CTACTAATTTCTCCTTGTTAAAACAAGCCGGTGACGTCGAAGAAAACCCT
GGTCCTATGATGACATGGACTCTACTATTCCTTGCCTTCCTTCATCACTT
AACAGGGTCATGTGCC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ITALICS UPPER CASE = human IgG kappa leader
sequence

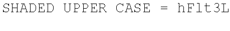

BOLD UPPER CASE = Furin cleavage site
BOLD UPPER CASE UNDERLINED = Pep2A
UPPER CASE UNDERLINED = anti-CTLA-4 Heavy Chain
(HC or variable heavy (V_{H}) region)
UPPER CASE = FLAG [symbol] [symbol] [symbol] [symbol]
= anti-CTLA-4 Light Chain
(LC or variable light (V_{L}) region)
hFltL3_PEP2A_anti-CTLA-4HC_PEP2A_anti-CTLA-4LC
amino acid sequence (SEQ ID NO: 6).

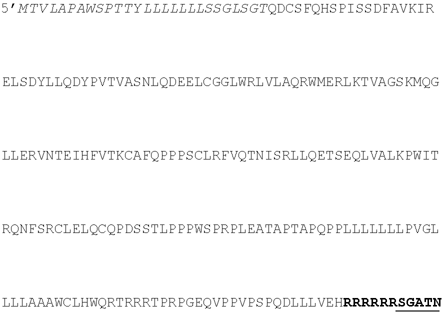

FSLLKQAGDVEENPGPMMEWSFVFLFFLSAAAGVHSEAKLQESGPVLVKP
GASVKMSCKASGYTFTDYYMNWVKQSHGKSLEWIGVINPYNGDTSYNQKF
KGKATLTVDKSSSTAYMELNSLTSEDSAVYYCARYYGSWFAYWGQGTLIT
VSTAKTTAPSVYPLAPVCGDTTGSSVTLGCLVKGYFPEPVTLTWNSGSLS
SGVHTFPAVLQSDLYTLSSSVTVTSSTWPSQSITCNVAHPASSTKVDKKI
EPRGPTIKPCPPCKCPAPNLLGGPSVFIFPPKIKDVLMISLSPIVTCVVV
DVSEDDPDVQISWFVNNVEVHTAQTQTHREDYNSTLRVVSALPIQHQDWM
SGKEFKCKVNNKDLPAPIERTISKPKGSVRAPQVYVLPPPEEEMTKKQVT
LTCMVTDFMPEDIYVEWTNNGKTELNYKNTEPVLDSDGSYFMYSKLRVEK
KNWVERNSYSCSVVHEGLHNHHTTKSFSRTPGKGSSDYKDDDDKRRRRRR
SGATNFSLLKQAGDVEENPGPMMTWTLLFLAFLHHLTGSCA

[symbol]

[symbol]

[symbol]

[symbol]

[symbol]

[symbol]

[symbol]

[symbol]

[symbol]

[symbol]

[symbol]

[symbol]

[symbol]

ITALICS UPPER CASE = human IgG kappa leader
sequence

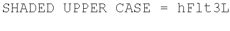

BOLD UPPER CASE = Furin cleavage site
BOLD UPPER CASE UNDERLINED = Pep2A
UPPER CASE UNDERLINED = anti-CTLA-4 Heavy Chain
(HC or variable heavy (V_{H}) region)
UPPER CASE = FLAG [symbol] [symbol] [symbol] [symbol]
= anti-CTLA-4 Light
Chain (LC or variable light (V_{L}) region)

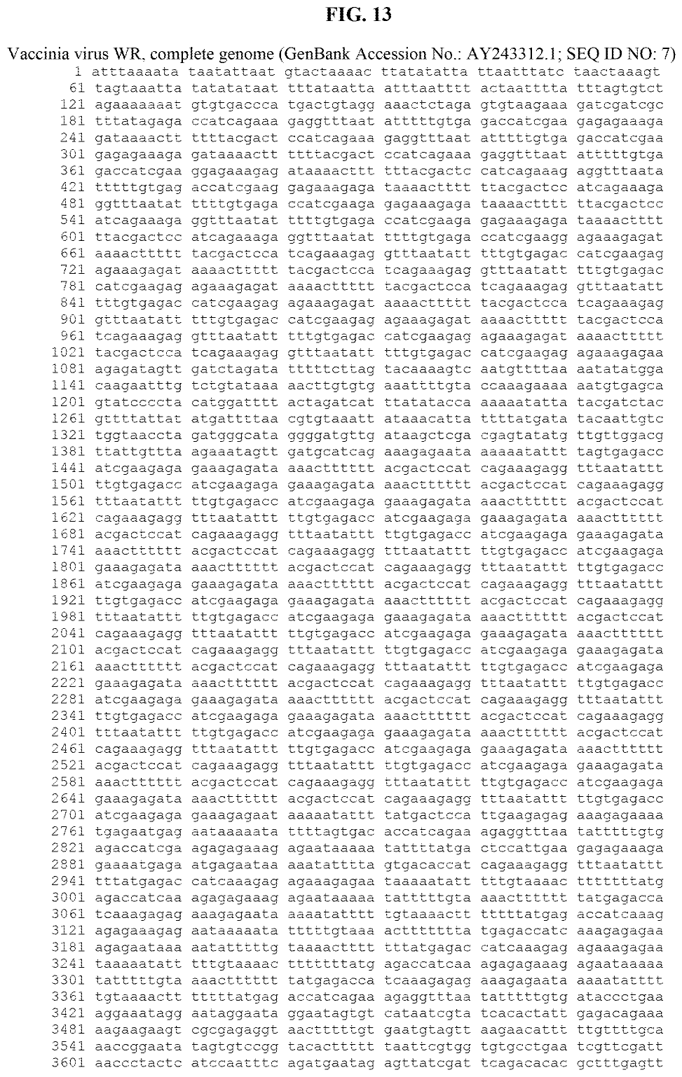

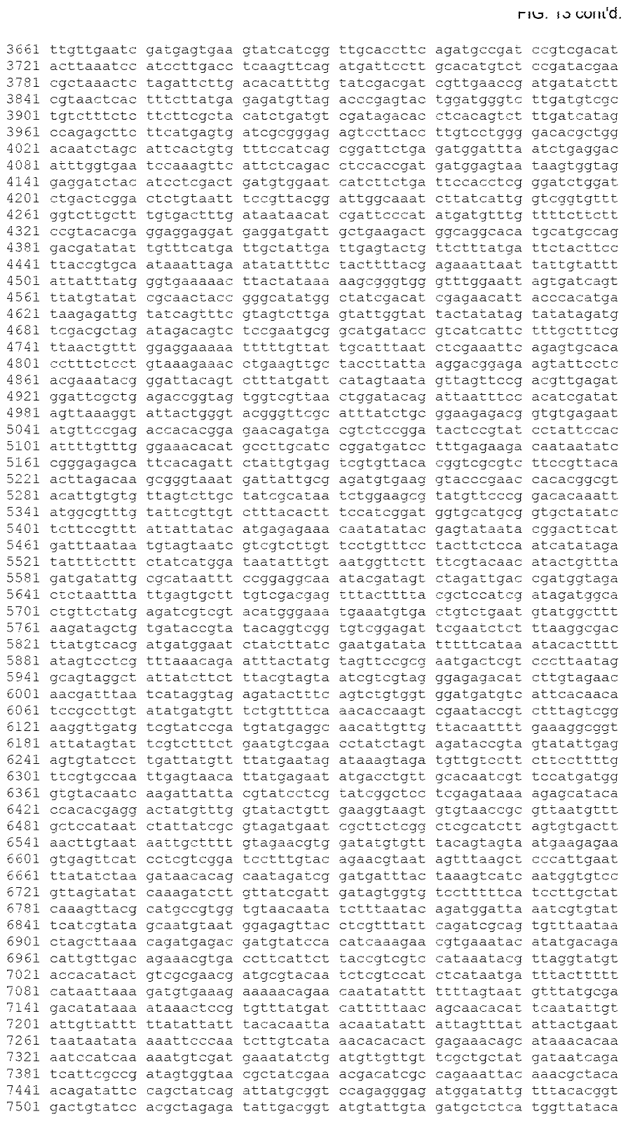

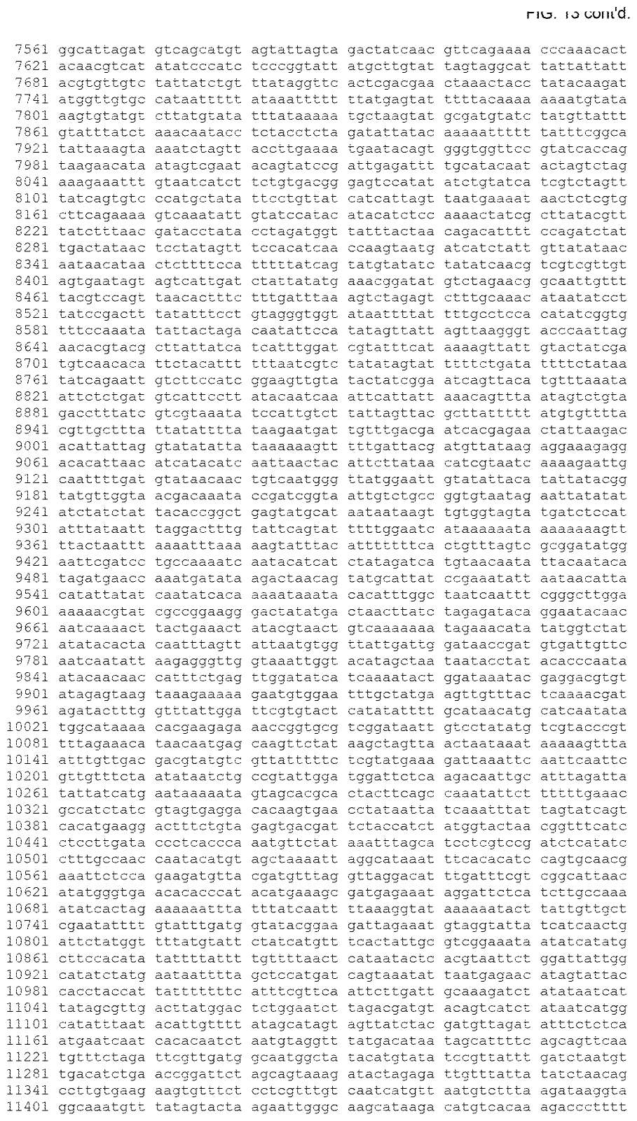

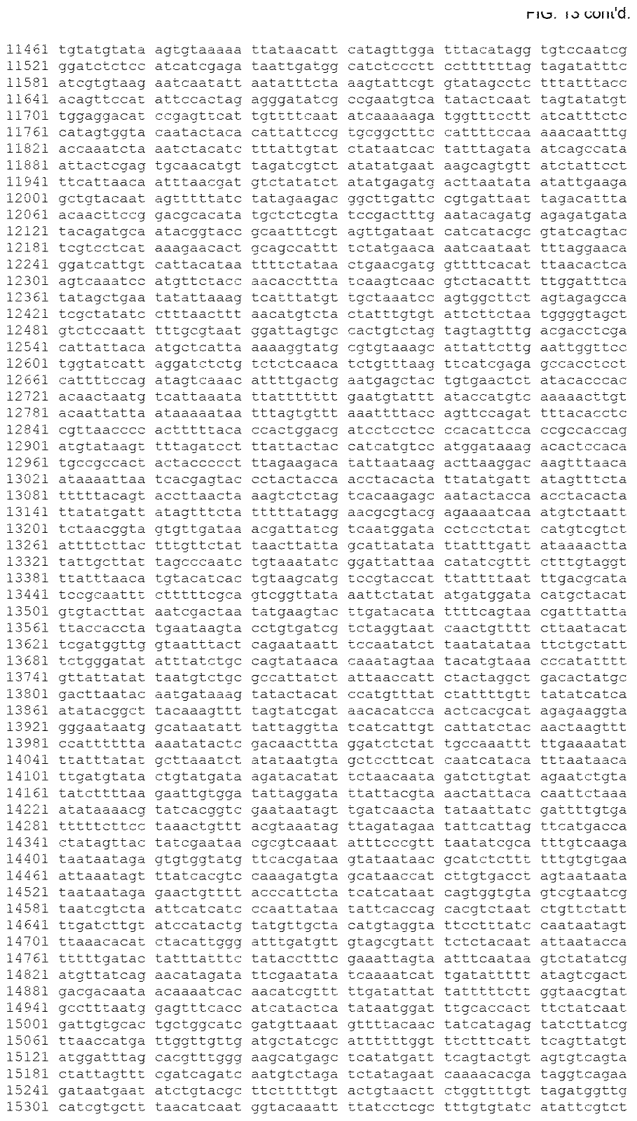

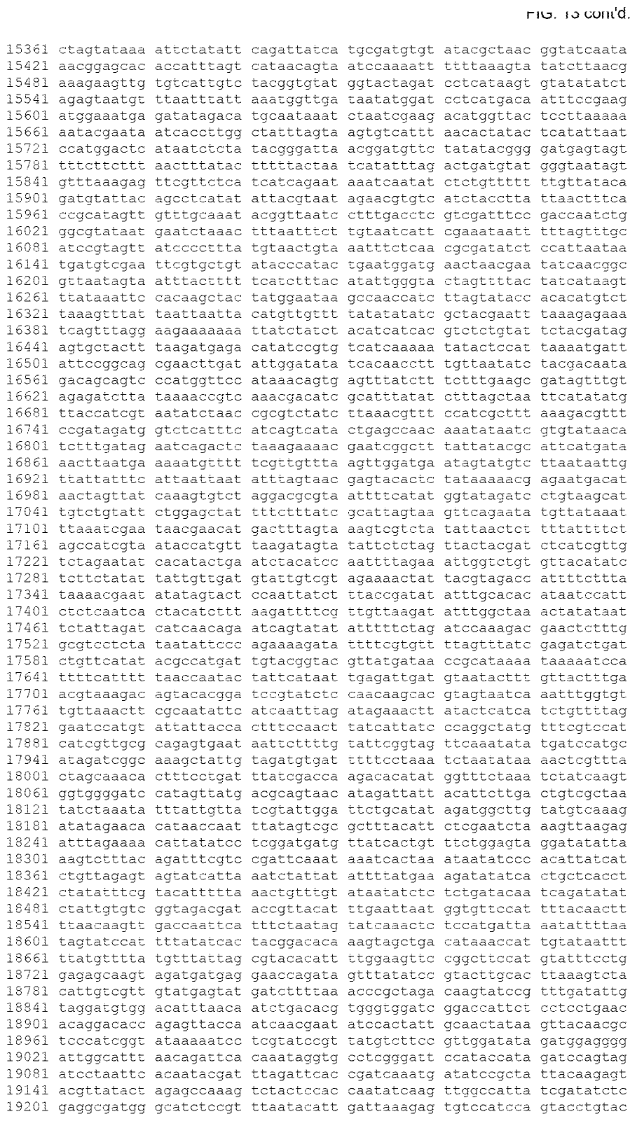

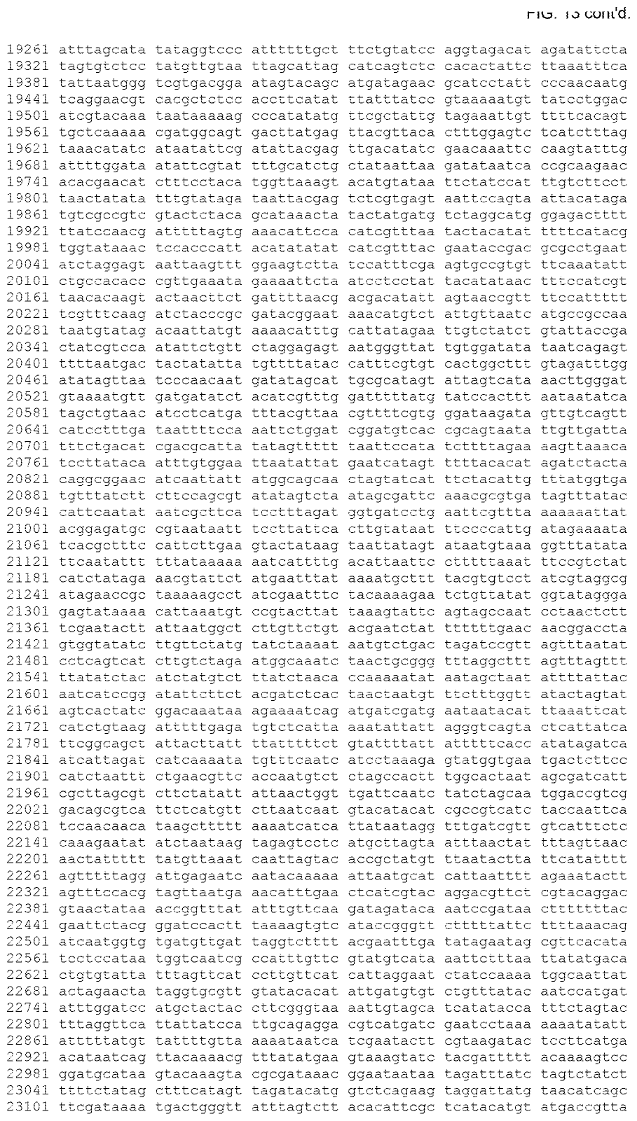

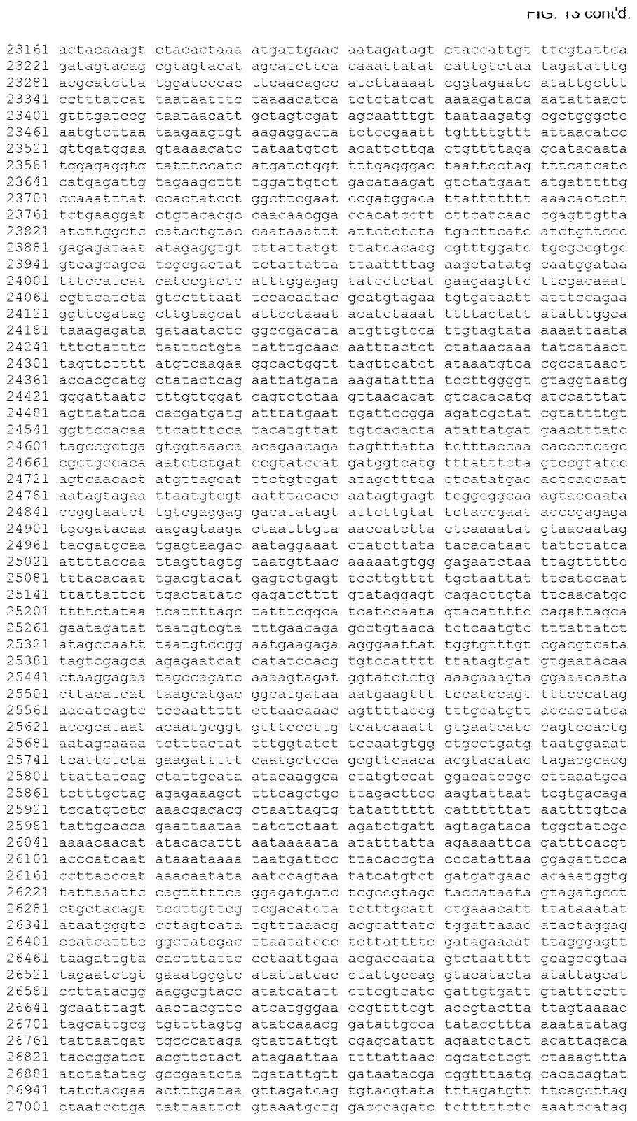

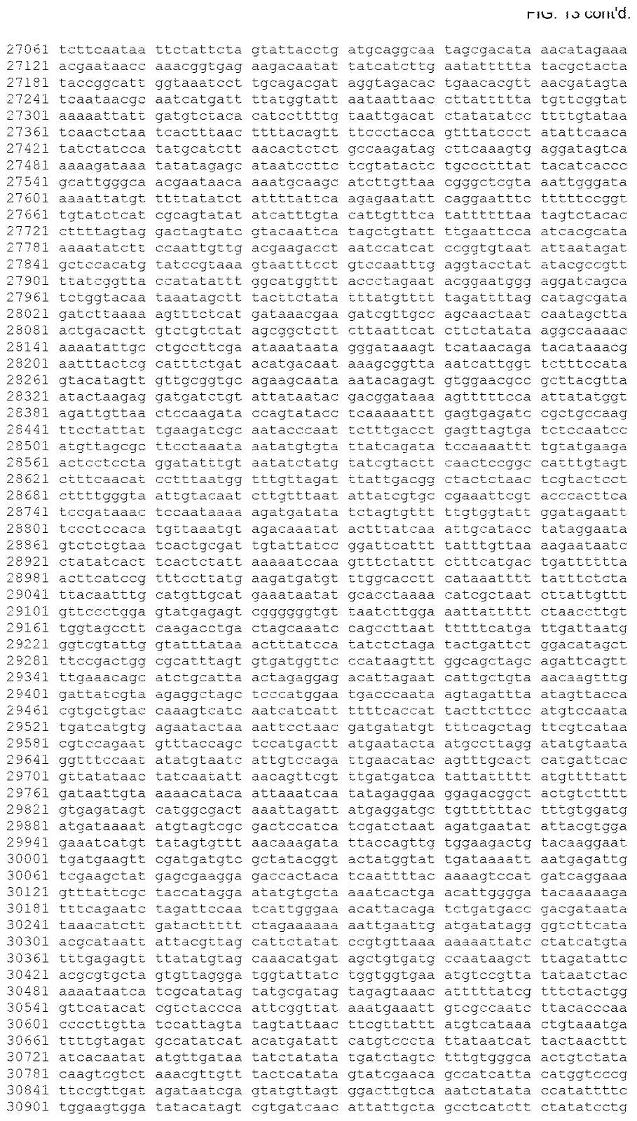

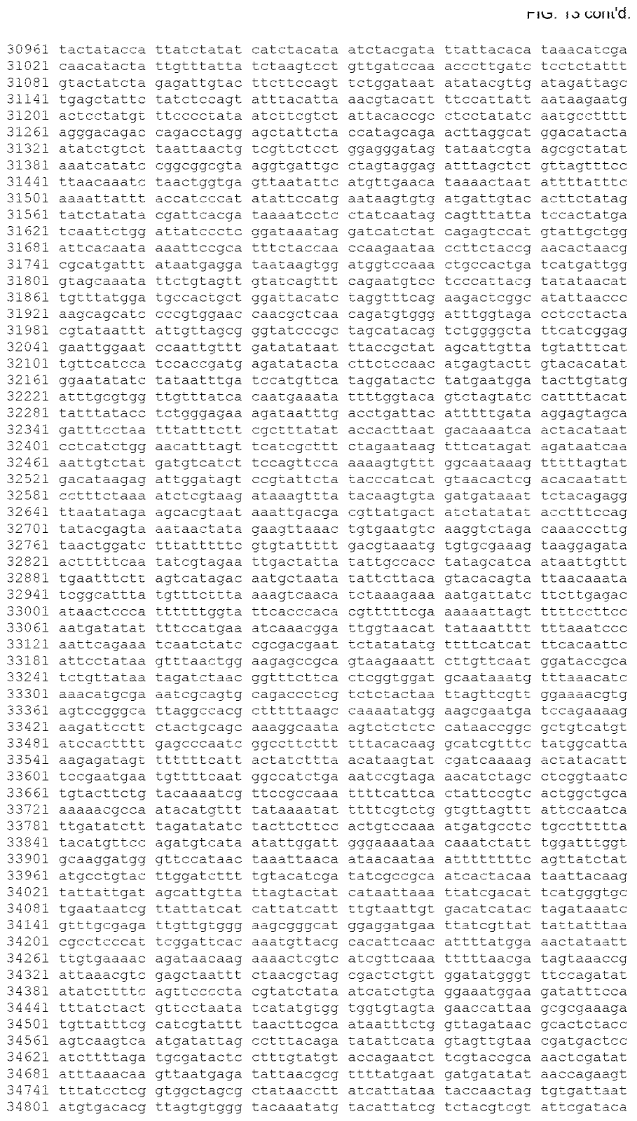

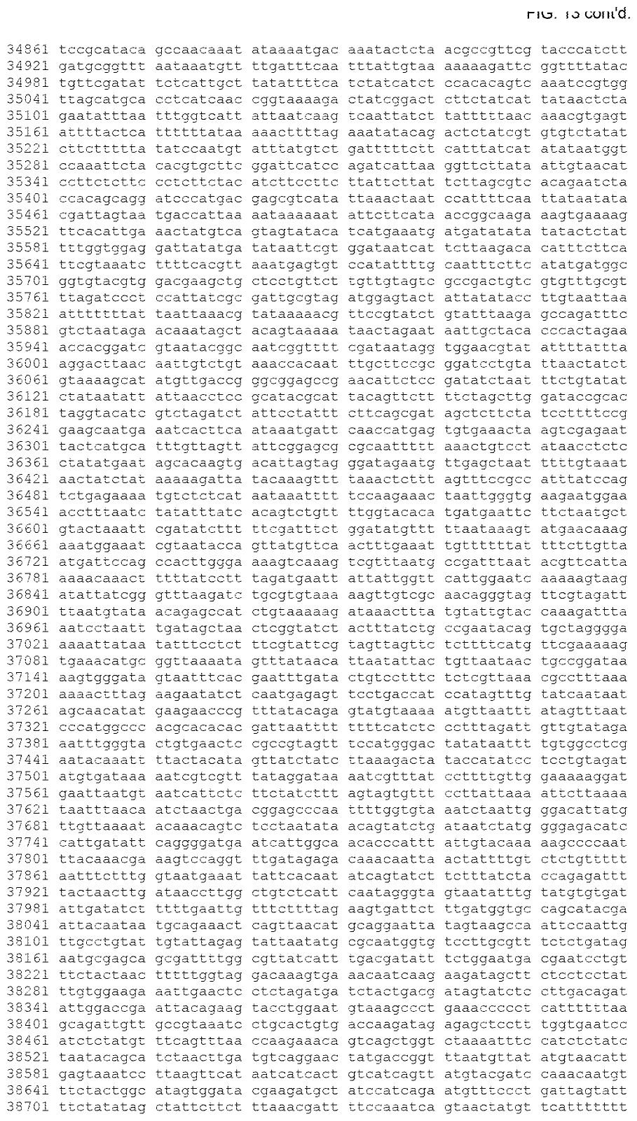

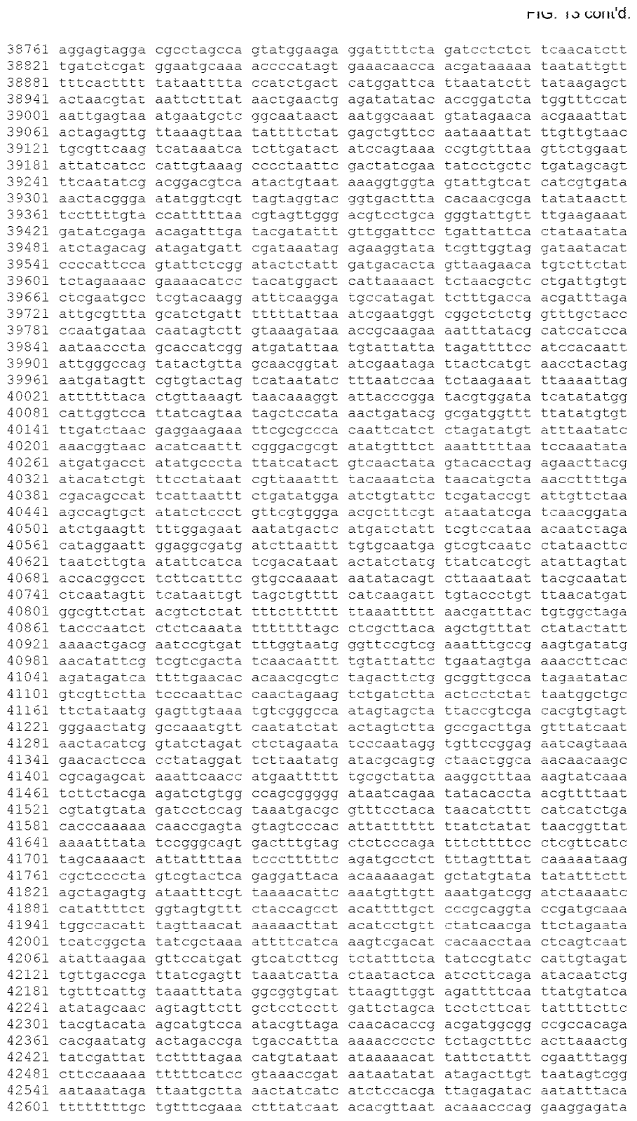

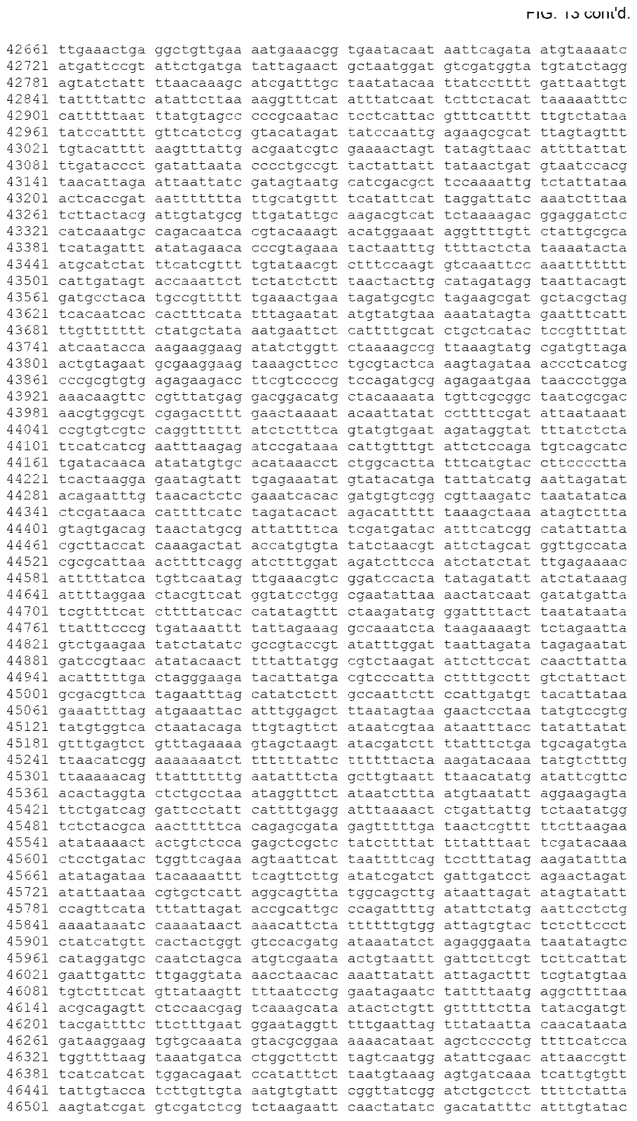

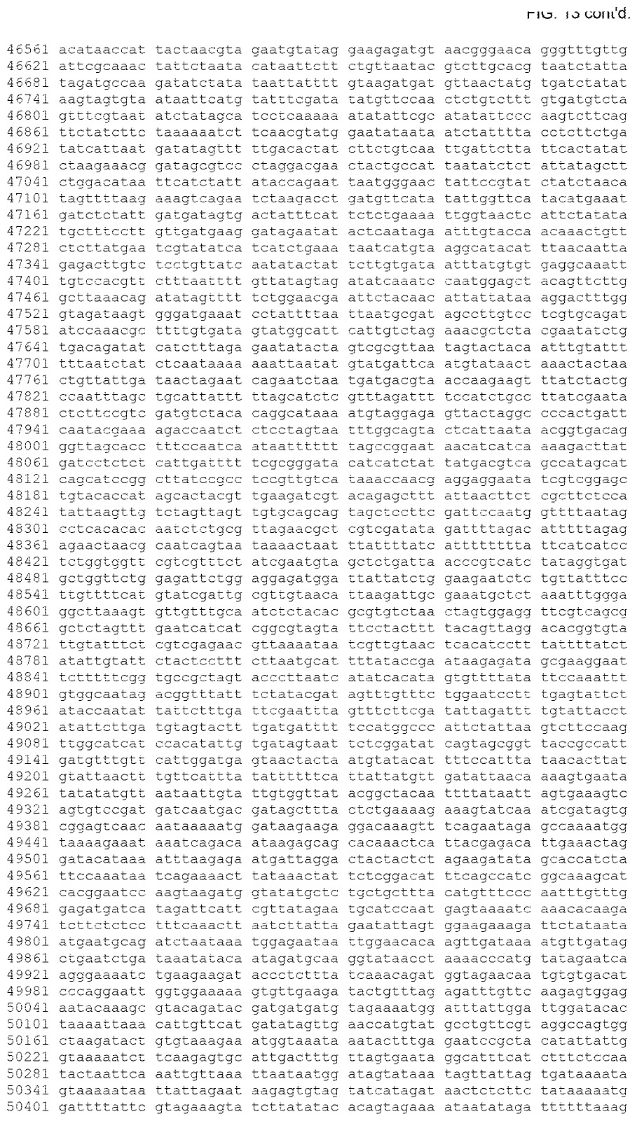

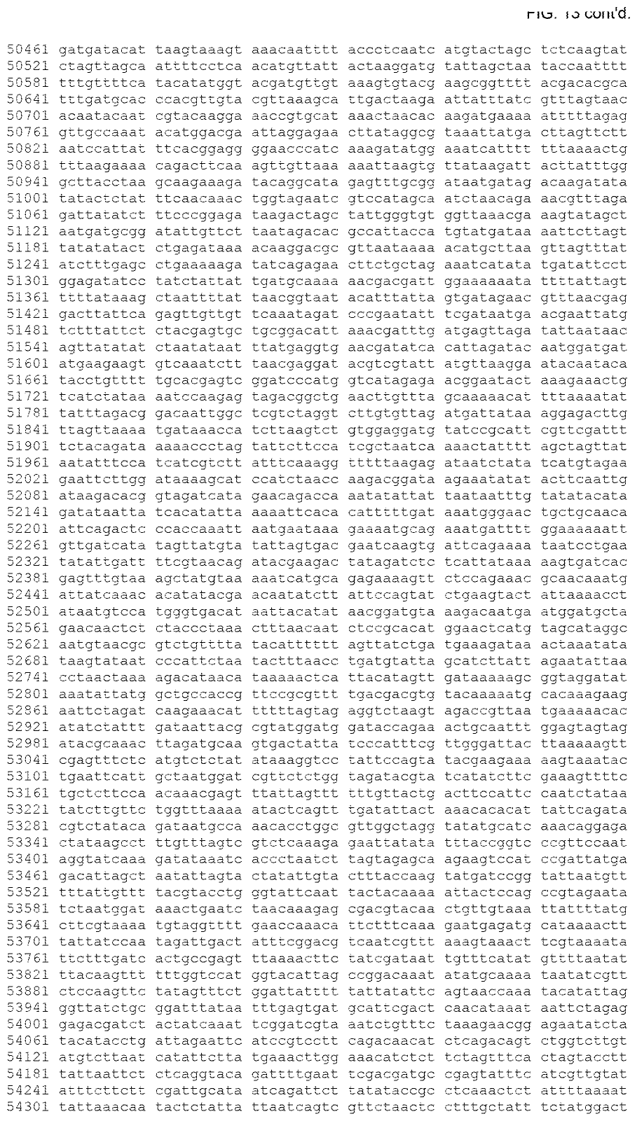

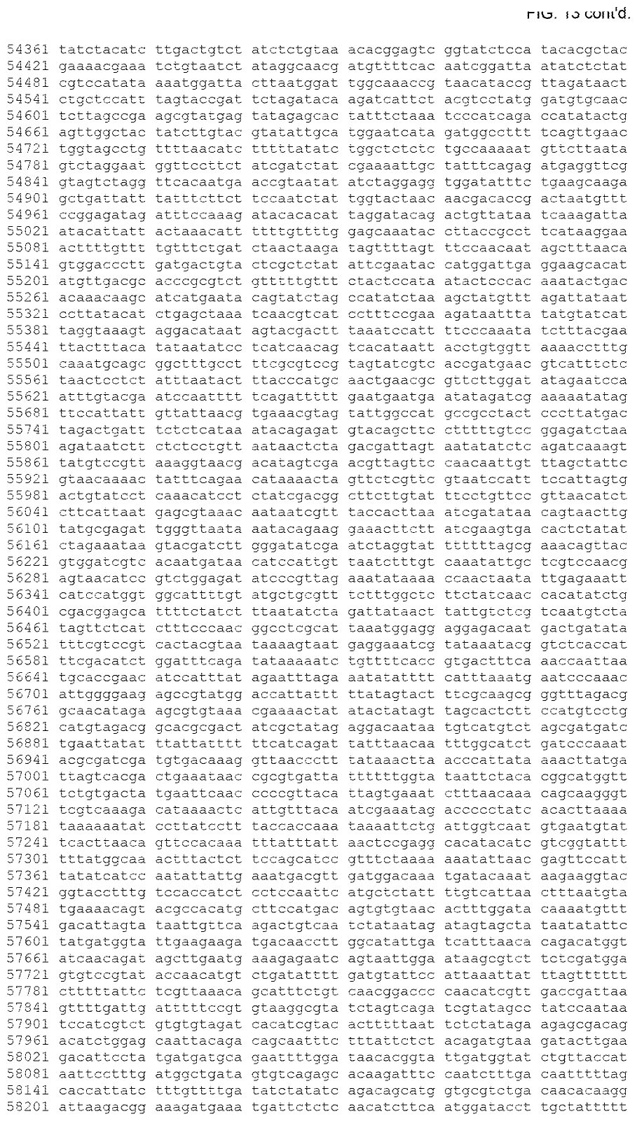

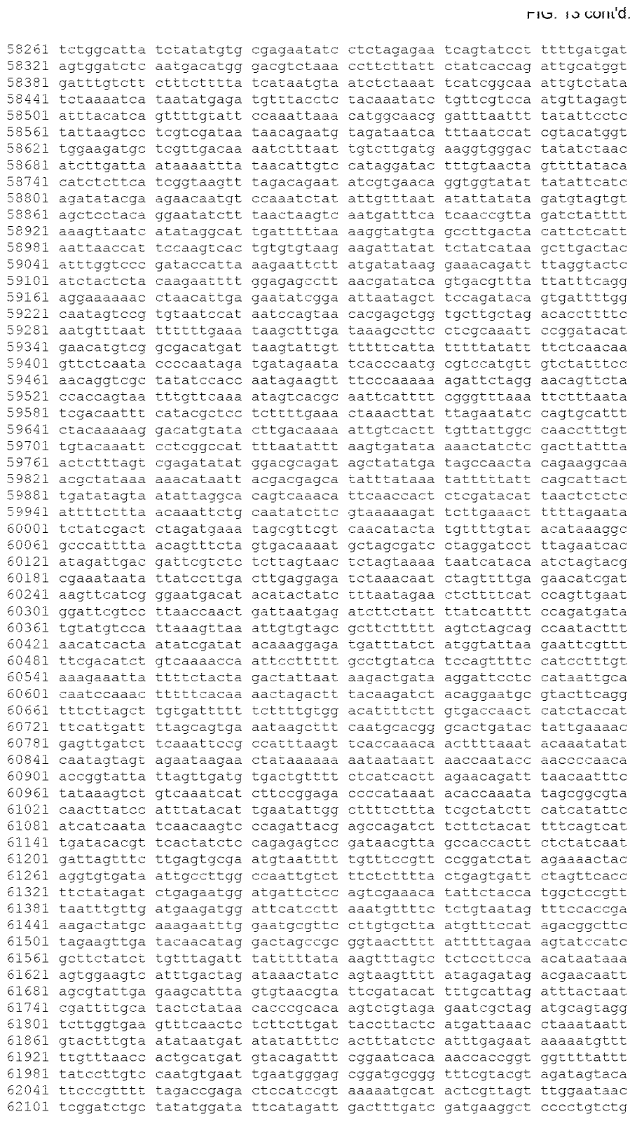

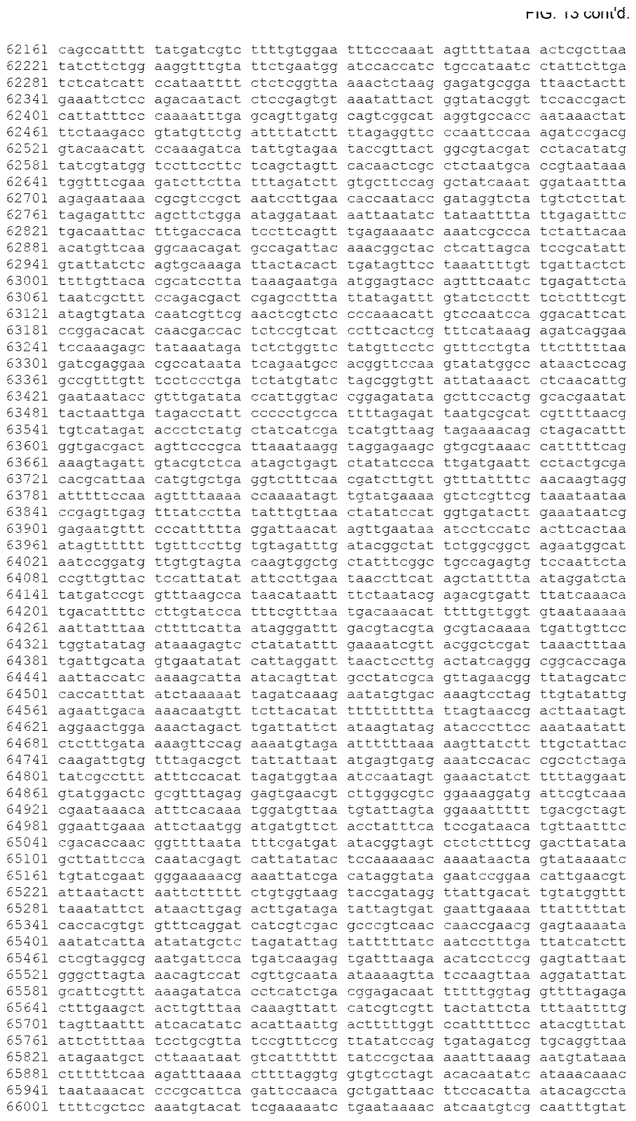

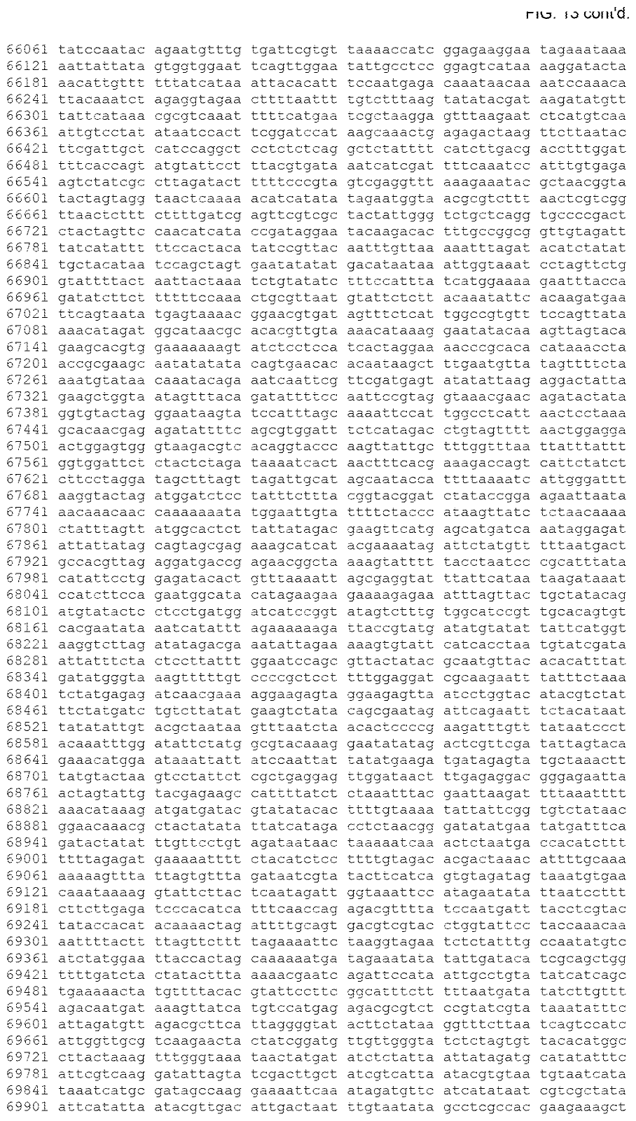

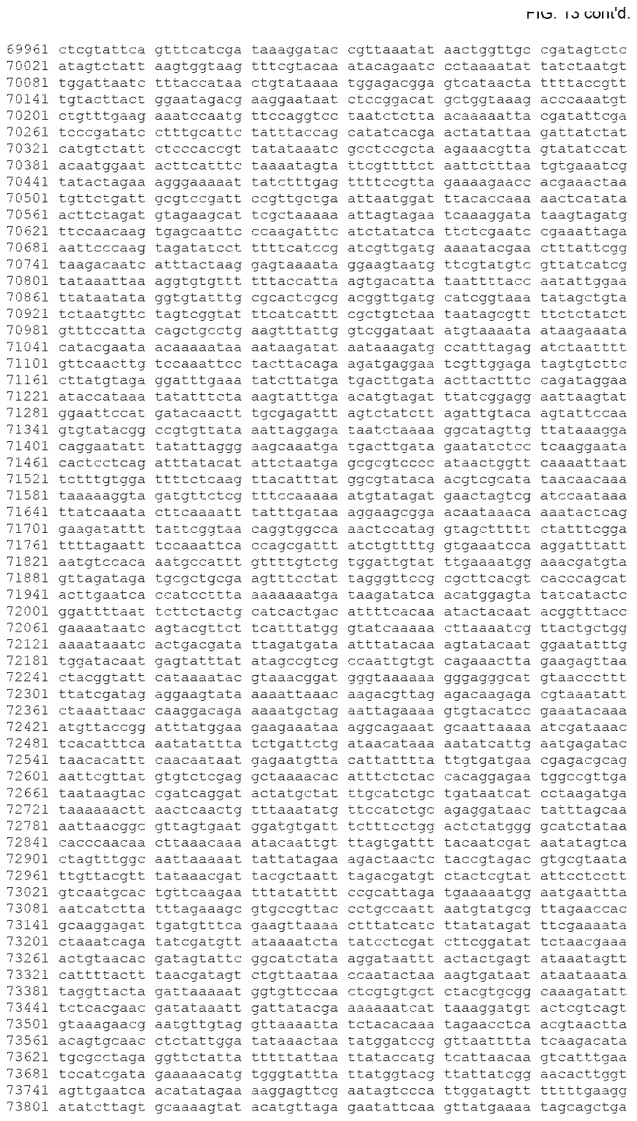

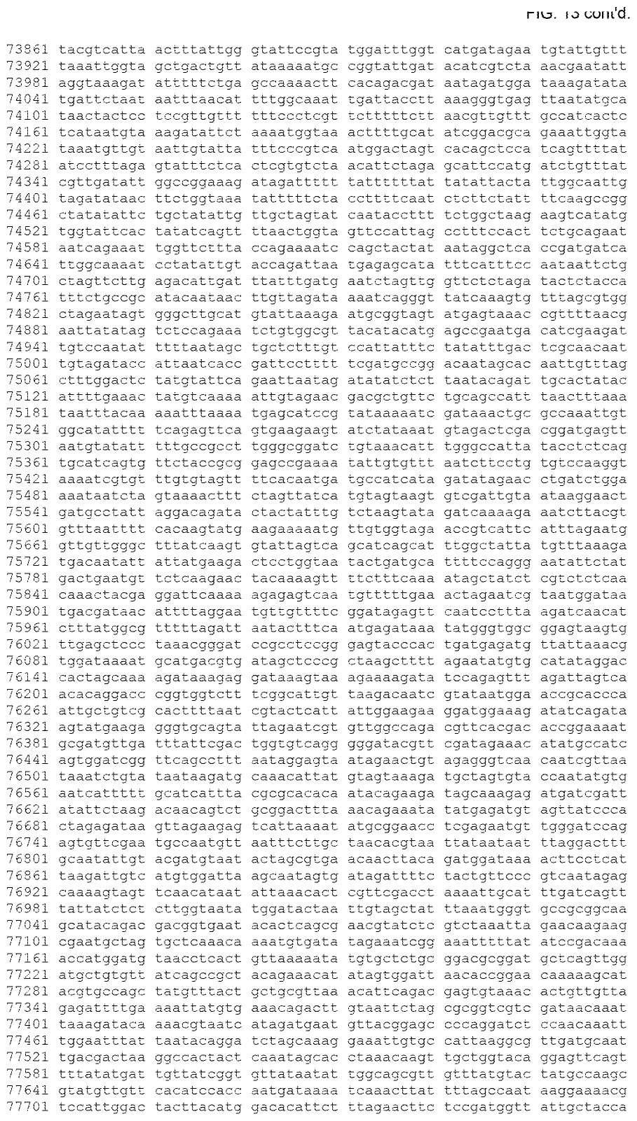

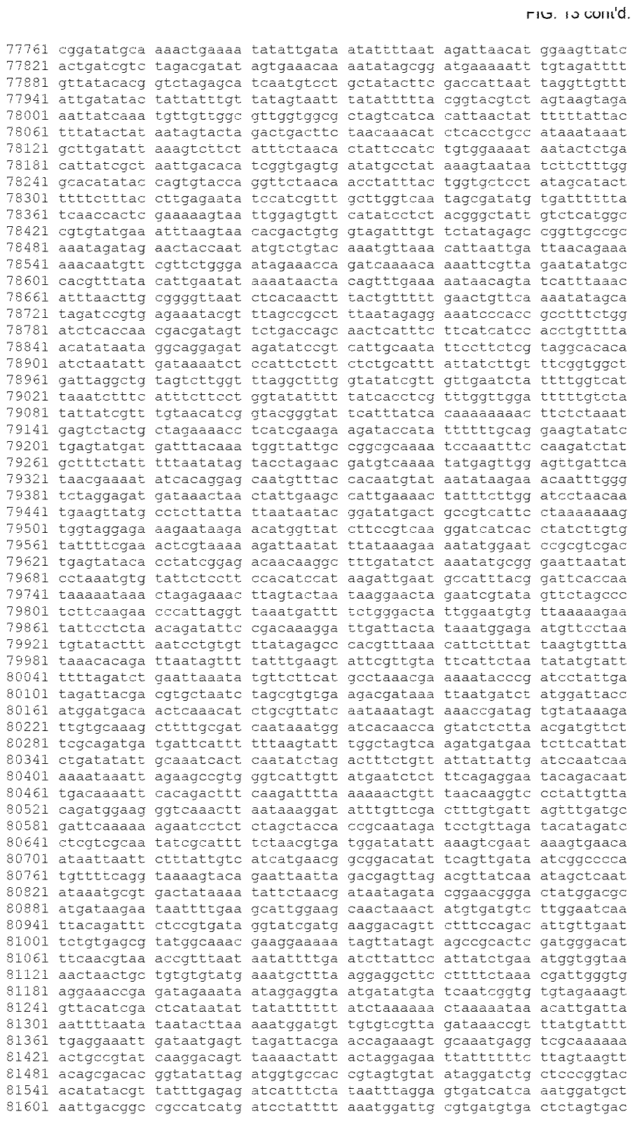

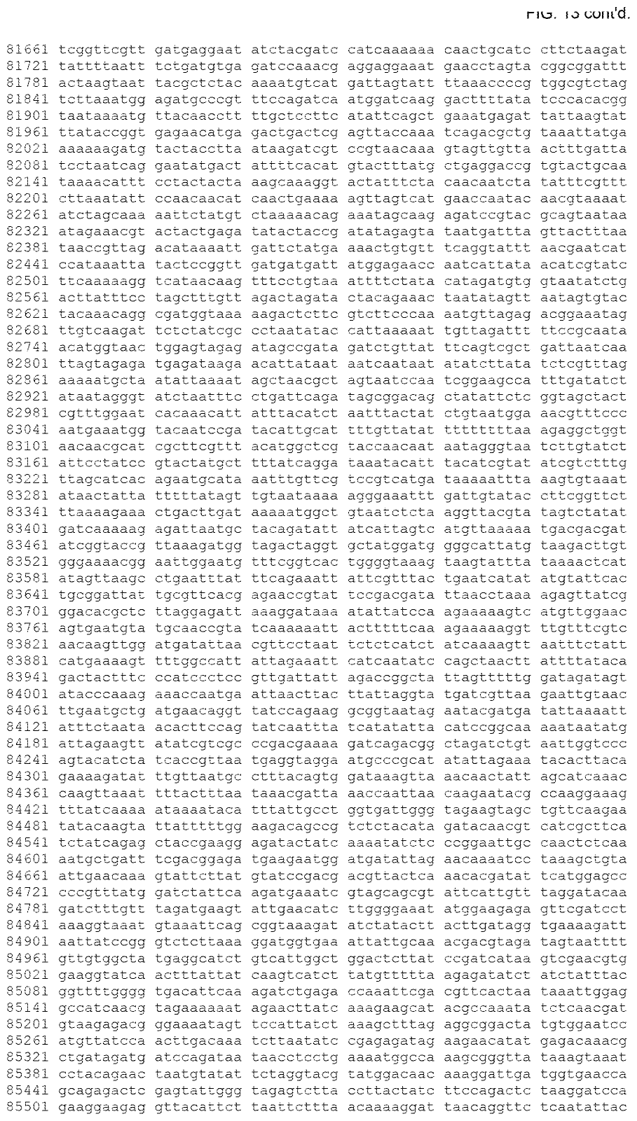

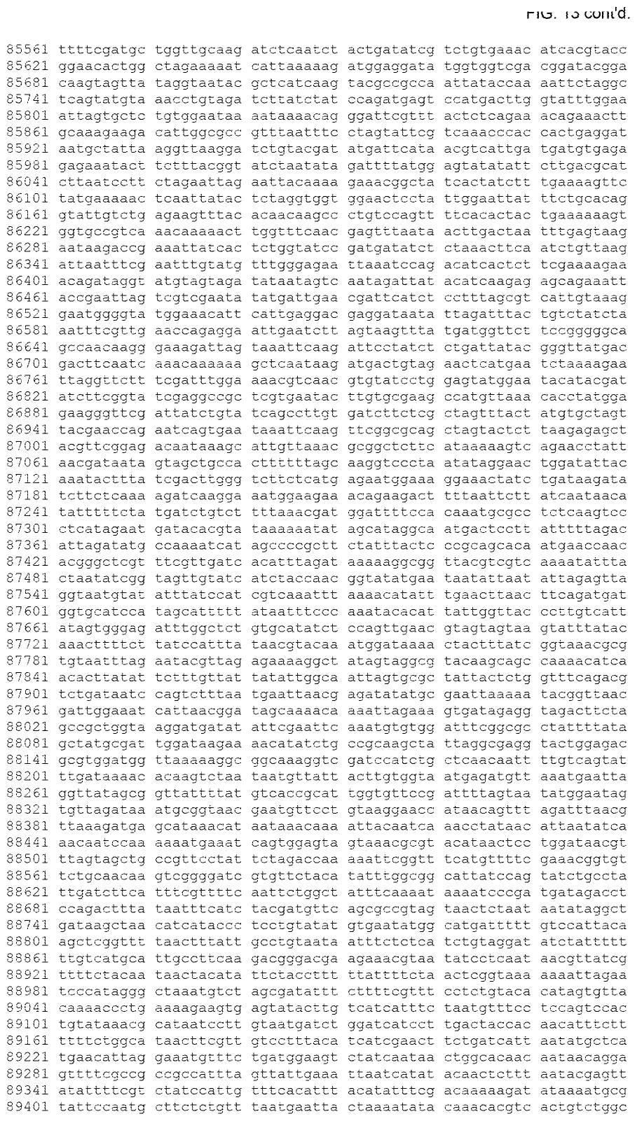

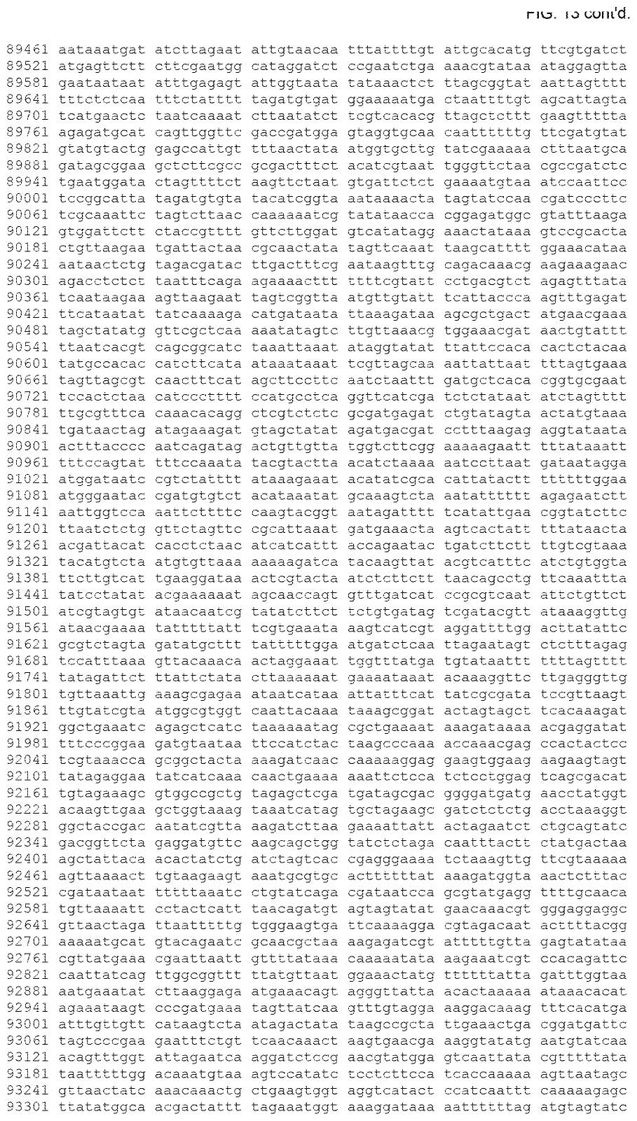

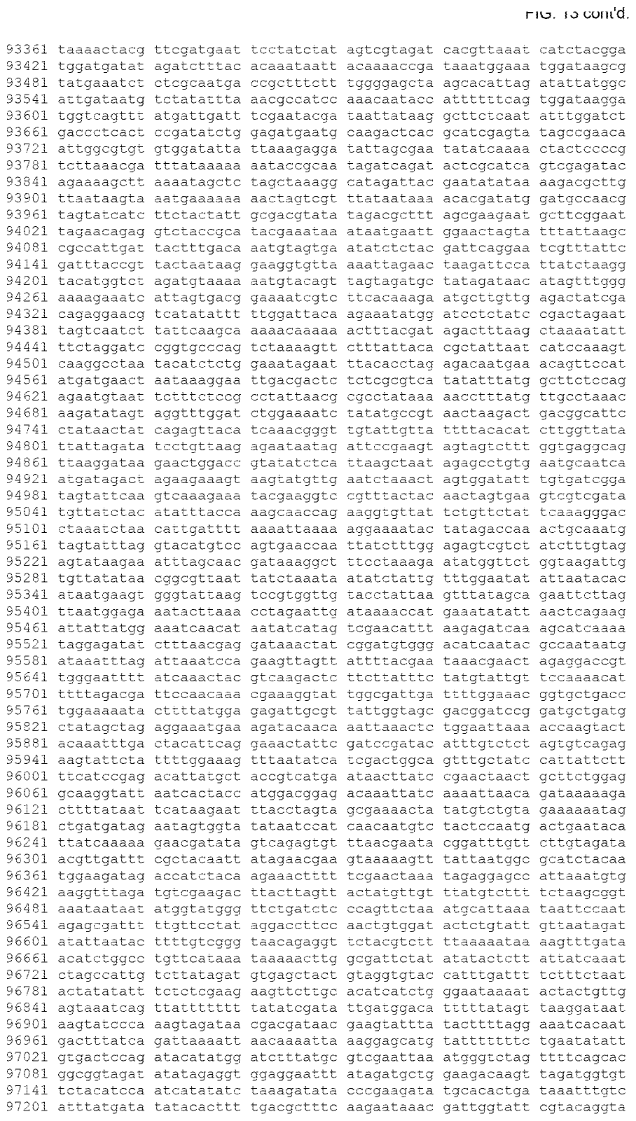

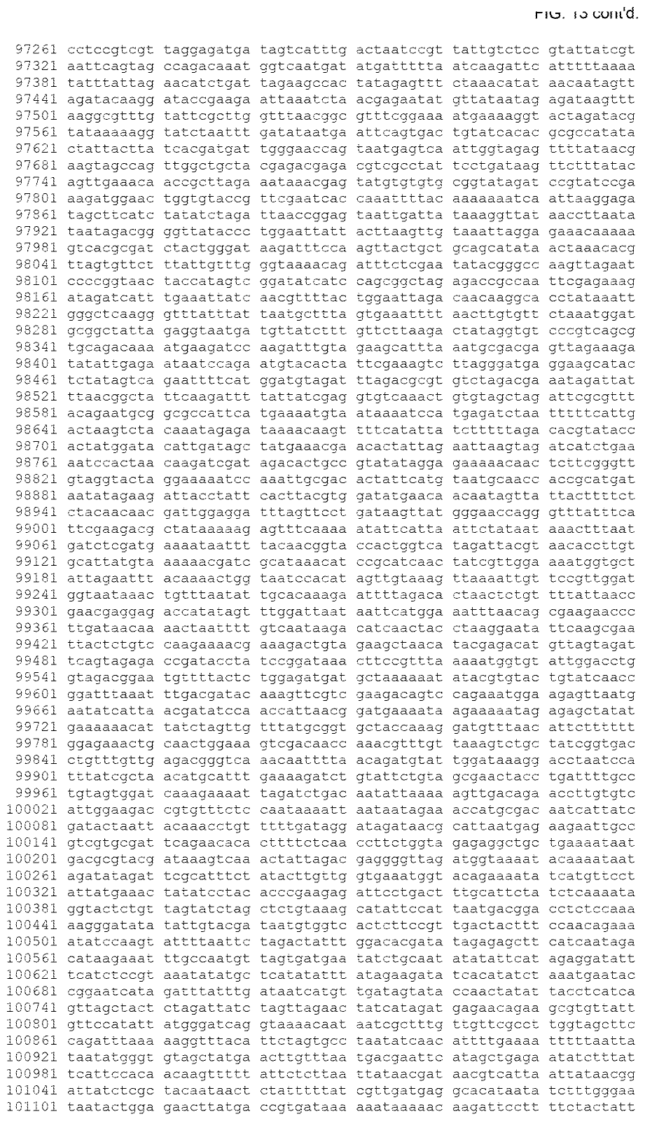

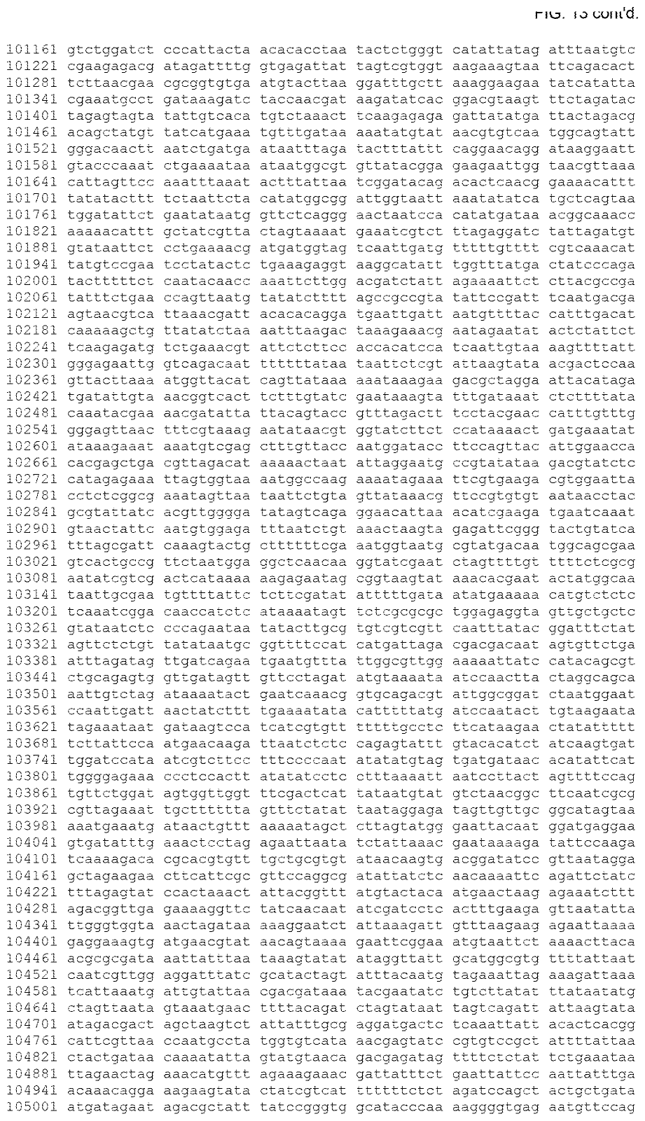

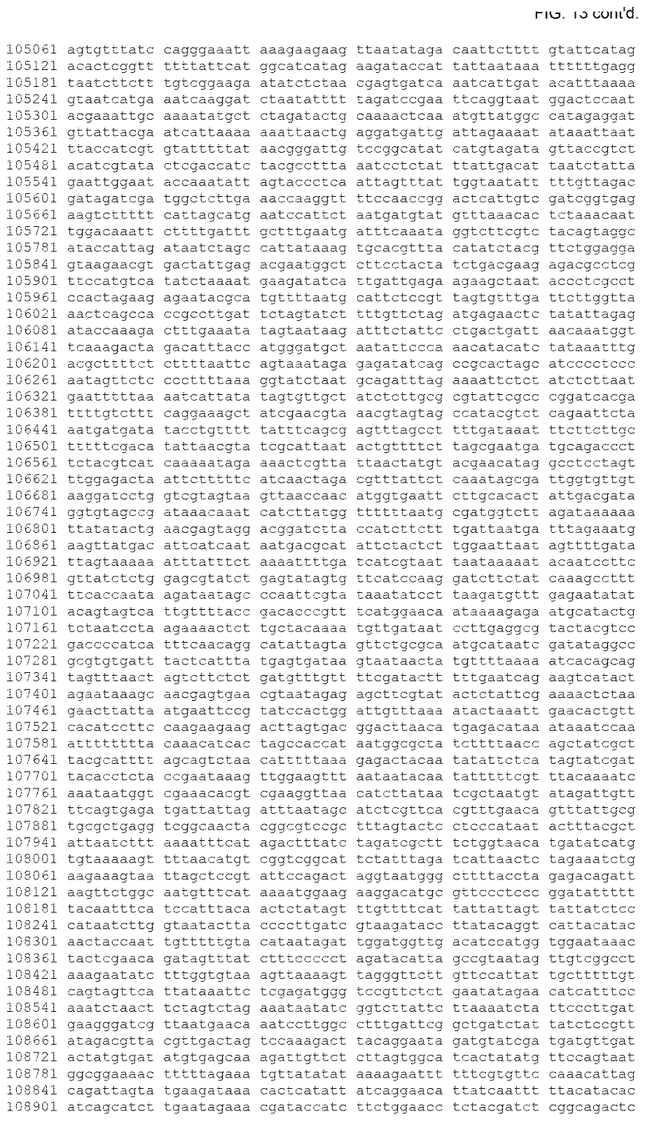

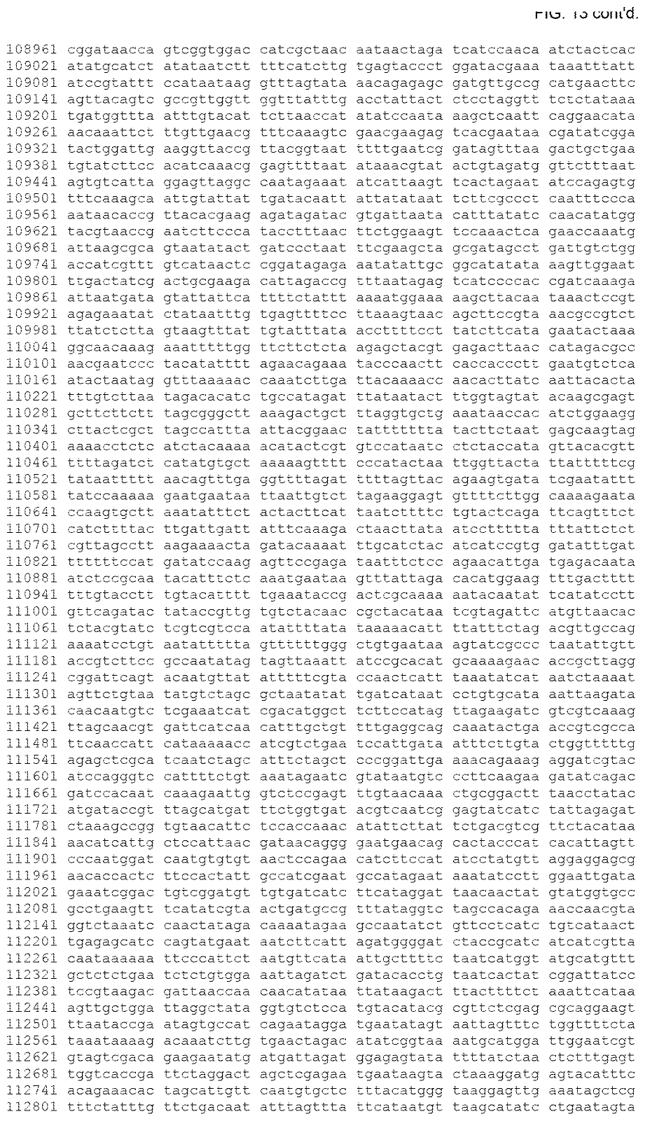

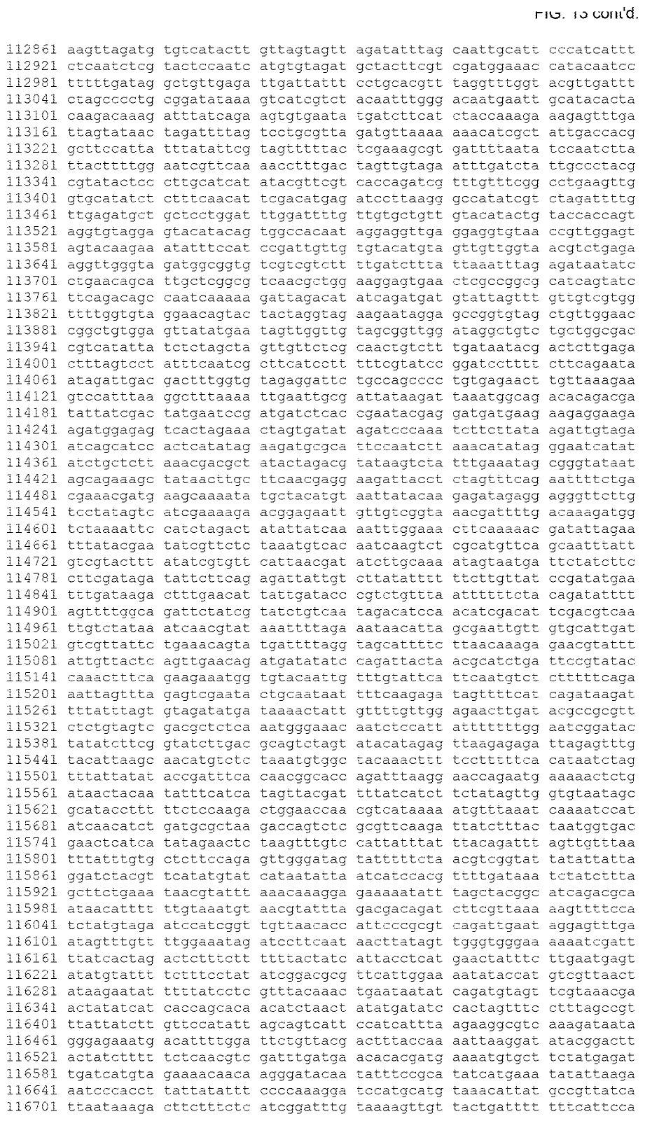

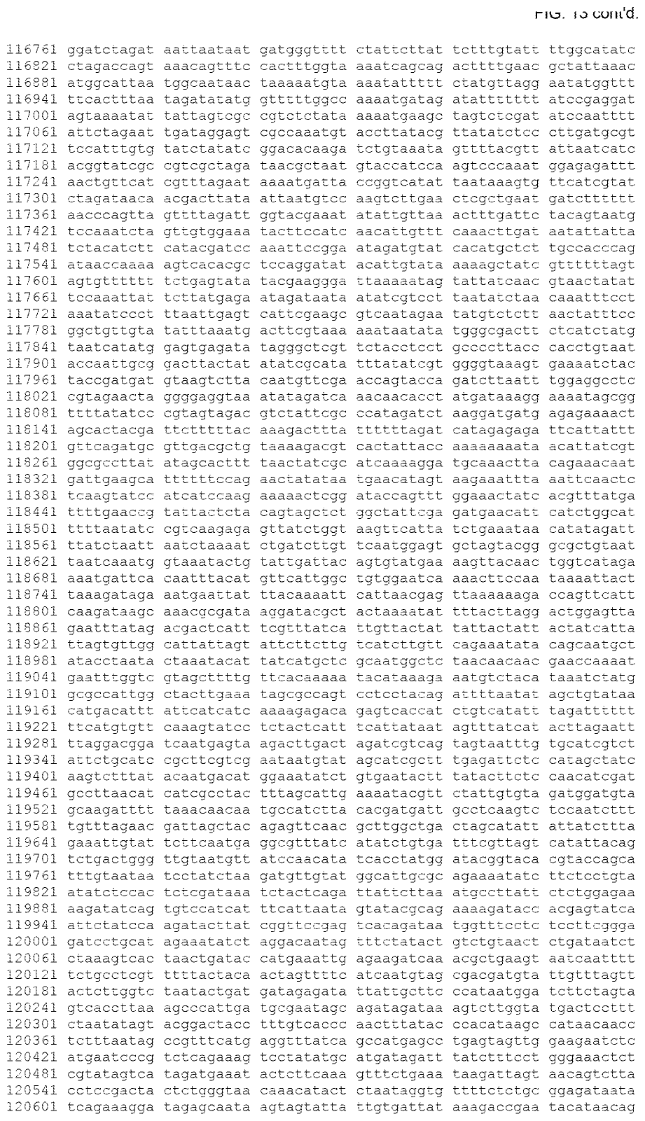

The vaccinia virus (Western Reserve strain; WR) genome sequence (SEQ ID NO: 7) given by GenBank Accession No. AY243312.1 is provided in FIG. 13. In some embodiments, the engineered E3LΔ83N-TK⁻ viruses described above are generated by inserting the expression constructs set forth in SEQ ID NOs: 1-6 into the E3LΔ83N-TK⁻ genomic region that corresponds to base pair positions 80,962 and 81,032 of the wild type vaccinia WR genome.

### VII. Melanoma

Melanoma, one of the deadliest cancers, is the fastest growing cancer in the U.S. and worldwide. In most cases, advanced melanoma is resistant to conventional therapies, including chemotherapy and radiation. As a result, people with metastatic melanoma have a very poor prognosis, with a life expectancy of only 6 to 10 months. The discovery that about 50% of melanomas have mutations in BRAF (a key tumor-promoting gene) opened the door for targeted therapy of this disease. Early clinical trials with BRAF inhibitors showed remarkable, but unfortunately not sustainable, responses in patients with melanomas with BRAF mutations. Therefore, alternative treatment strategies for these patients, as well as others with melanoma without BRAF mutations, are urgently needed.

Human pathological data indicate that the presence of T-cell infiltrates within melanoma lesions correlates positively with longer patient survival (Oble et al. Cancer Immun. 9, 3 (2009)). The importance of the immune system in protection against melanoma is further supported by partial success of immunotherapies, such as the immune activators IFN-α2b and IL-2 (Lacy et al. Expert Rev Dermatol 7(1):51-68 (2012)) as well as the unprecedented clinical responses of patients with metastatic melanoma to immune checkpoint therapy, including anti-CTLA-4 and anti-PD-1/PD-L1 either agent alone or in combination therapy (Sharma and Allison, Science 348(6230), 56-61 (2015); Hodi et al., NEJM 363(8), 711-723 (2010); Wolchok et al., Lancet Oncol. 11(6), 155-164 (2010); Topalian et al., NEJM 366(26), 2443-2454 (2012); Wolchok et al., NEJM 369(2), 122-133 (2013); Hamid et al., NEJM 369(2), 134-144 (2013); Tumeh et al., Nature 515(7528), 568-571 (2014)). However, many patients fail to respond to immune checkpoint blockade therapy alone.

### VIII. Pharmaceutical Compositions and Preparations of the Present Technology

Disclosed herein are pharmaceutical compositions comprising the engineered vaccinia viruses of the present technology, such as E3LΔ83N-TK⁻-anti-CTLA-4 or E3LΔ83N-TK⁻-hFlt3L-anti-CTLA-4, that may contain a carrier or diluent, which can be a solvent or dispersion medium containing, for example, water, saline, Tris buffer, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be effected by various antibacterial and antifungal agents and preservatives, for example, parabens, chlorobutanol, phenol, sorbic acid, thimerosal, and the like. In some embodiments, isotonic agents, for example, sugars or sodium chloride, and buffering agents are included. Prolonged absorption of the injectable compositions can be brought about by the use in the compositions of agents delaying absorption, for example, aluminum monostearate and gelatin or carrier molecules. Other excipients may include wetting or emulsifying agents. In general, excipients suitable for injectable preparations can be included as apparent to those skilled in the art.

Pharmaceutical compositions and preparations comprising the engineered vaccinia viruses of the present technology, such as E3LΔ83N-TK⁻-anti-CTLA-4 or E3LΔ83N-TK⁻-hFlt3L-anti-CTLA-4, may be manufactured by means of conventional mixing, dissolving, granulating, emulsifying, encapsulating, entrapping or lyophilizing processes. Pharmaceutical viral compositions may be formulated in conventional manner using one or more physiologically acceptable carriers, diluents, excipients or auxiliaries that facilitate formulating virus preparations suitable for in vitro, in vivo, or ex vivo use. The compositions can be combined with one or more additional biologically active agents (for example parallel administration of an immune checkpoint inhibitor, such as a PD-1 inhibitor or anti PD-1/PD-L1 therapy) and may be formulated with a pharmaceutically acceptable carrier, diluent or excipient to generate pharmaceutical (including biologic) or veterinary compositions of the instant disclosure suitable for parenteral or intra-tumoral administration.

Many types of formulation are possible as is appreciated by those skilled in the art. The particular type chosen is dependent upon the route of administration chosen, as is well-recognized in the art. For example, systemic formulations will generally be designed for administration by injection, e.g., intravenous, as well as those designed for intratumoral delivery. In some embodiments, the systemic or intratumoral formulation is sterile.

Sterile injectable solutions are prepared by incorporating the engineered vaccinia viruses of the present technology, such as E3LΔ83N-TK⁻-anti-CTLA-4 or E3LΔ83N-TK⁻-hFlt3L-anti-CTLA-4, in the required amount of the appropriate solvent with various other ingredients enumerated herein, as required, followed by suitable sterilization means. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum drying and freeze-drying techniques, which yield a powder of the virus plus any additional desired ingredient from a previously sterile-filtered solution thereof.

In some embodiments, the engineered vaccinia virus compositions of the present disclosure may be formulated in aqueous solutions, or in physiologically compatible solutions or buffers such as Hanks's solution, Ringer's solution, mannitol solutions or physiological saline buffer. In certain embodiments, any of the engineered vaccinia virus compositions of the present technology may contain formulator agents, such as suspending, stabilizing, penetrating or dispersing agents, buffers, lyoprotectants or preservatives such as polyethylene glycol, polysorbate 80, 1-dodecylhexahydro-2H-azepin-2-one (laurocapran), oleic acid, sodium citrate, Tris HCl, dextrose, propylene glycol, mannitol, polysorbate polyethylene-sorbitan monolaurate (Tween®-20), isopropyl myristate, benzyl alcohol, isopropyl alcohol, ethanol sucrose, trehalose and other such generally known in the art may be used in any of the compositions of the instant disclosure. (Pramanick et al., Pharma Times 45(3), 65-76 (2013)).

The biologic or pharmaceutical compositions of the present disclosure can be formulated to allow the virus contained therein to be available to infect tumor cells upon administration of the composition to a subject. The level of virus in serum, tumors, and if desired other tissues after administration can be monitored by various well-established techniques, such as antibody-based assays (e.g., ELISA, immunohistochemistry, etc.).

The recombinant viruses of the present technology can be stored at −80° C. with a titer of 3.5×10⁷ PFU/ml formulated in about 10 mM Tris, 140 mM NaCl pH 7.7. For the preparation of vaccine shots, e.g., 10²-10⁸ or 10²-10⁹ viral particles can be lyophilized in 100 ml of phosphate-buffered saline (PBS) in the presence of 2% peptone and 1% human albumin in an ampoule, preferably a glass ampoule. Alternatively, the injectable preparations can be produced by stepwise freeze-drying of the recombinant virus in a formulation. This formulation can contain additional additives such as mannitol, dextran, sugar, glycine, lactose or polyvinylpyrrolidone or other additives such as antioxidants or inert gas, stabilizers or recombinant proteins (e.g., human serum albumin) suitable for in vivo administration. The glass ampoule is then sealed and can be stored between 4° C. and room temperature for several months. In some embodiments, the ampoule is stored at temperatures below −20° C.

For therapy, the lyophilisate can be dissolved in an aqueous solution, such as physiological saline or Tris buffer, and administered either systemically or intratumorally. The mode of administration, the dose, and the number of administrations can be optimized by those skilled in the art.

The pharmaceutical composition according to the present disclosure may comprise an additional adjuvant. As used herein, an “adjuvant” refers to a substance that enhances, augments or potentiates the host's immune response to tumor antigens. A typical adjuvant may be aluminum salts, such as aluminum hydroxide or aluminum phosphate, Quil A, bacterial cell wall peptidoglycans, virus-like particles, polysaccharides, toll-like receptors, nano-beads, etc. (Aguilar et al. (2007), Vaccine 25: 3752-3762).

### IX. Kits Comprising the Recombinant Vaccinia Viruses of the Present Technology, Such as E3LΔ83N-TK⁻-Anti-CTLA-4 or E3LΔ83N-TK⁻-hFlt3L-Anti-CTLA-4 Viruses

The present disclosure provides for kits comprising one or more compositions comprising one or more of the recombinant vaccinia viruses of the present technology, such as E3LΔ83N-TK⁻-anti-CTLA-4 or E3LΔ83N-TK⁻-hFlt3L-anti-CTLA-4. The kit can comprise one or multiple containers or vials of the recombinant E3LΔ83N-TK⁻-anti-CTLA-4 or E3LΔ83N-TK⁻-hFlt3L-anti-CTLA-4 together with instructions for the administration of the recombinant E3LΔ83N-TK⁻-anti-CTLA-4 or E3LΔ83N-TK⁻-hFlt3L-anti-CTLA-4 to a subject to be treated. The instructions may indicate a dosage regimen for administering the composition or compositions as provided below.

In some embodiments, the kit may also comprise an additional composition comprising a checkpoint inhibitor for conjoint administration with the recombinant E3LΔ83N-TK⁻-anti-CTLA-4 or E3LΔ83N-TK⁻-hFlt3L-anti-CTLA-4 composition.

### X. Effective Amount and Dosage of the Recombinant Vaccinia Viruses of the Present Technology, Such as E3LΔ83N-TK⁻-Anti-CTLA-4 or E3LΔ83N-TK⁻-hFlt3L-Anti-CTLA-4

In general, the subject is administered a dosage of the engineered vaccinia viruses of the present technology, such as E3LΔ83N-TK⁻-anti-CTLA-4 or E3LΔ83N-TK⁻-hFlt3L-anti-CTLA-4, in the range of about 10⁶ to about 10¹⁰ plaque forming units (pfu), although a lower or higher dose may be administered. In some embodiments, the dosage ranges from about 10² to about 10¹⁰ pfu. In some embodiments, the dosage ranges from about 10³ to about 10¹⁰ pfu. In some embodiments, the dosage ranges from about 10⁴ to about 10¹⁰ pfu. In some embodiments, the dosage ranges from about 10⁵ to about 10¹⁰ pfu. In some embodiments, the dosage ranges from about 10⁶ to about 10¹⁰ pfu. In some embodiments, the dosage ranges from about 10⁷ to about 10¹⁰ pfu. In some embodiments, the dosage ranges from about 10⁸ to about 10¹⁰ pfu. In some embodiments, the dosage ranges from about 10⁹ to about 10¹⁰ pfu. In some embodiments, dosage is about 10⁷ to about 10⁹ pfu. The equivalence of pfu to virus particles can differ according to the specific pfu titration method used. Generally, a pfu is equal to about 5 to 100 virus particles and 0.69 PFU is about 1 TCID50. A therapeutically effective amount of E3LΔ83N-TK⁻-anti-CTLA-4 or E3LΔ83N-TK⁻-hFlt3L-anti-CTLA-4 can be administered in one or more divided doses for a prescribed period of time and at a prescribed frequency of administration.

For example, as is apparent to those skilled in the art, a therapeutically effective amount of E3LΔ83N-TK⁻-anti-CTLA-4 or E3LΔ83N-TK⁻-hFlt3L-anti-CTLA-4 in accordance with the present disclosure may vary according to factors such as the disease state, age, sex, weight, and general condition of the subject, and the ability of E3LΔ83N-TK⁻-anti-CTLA-4 or E3LΔ83N-TK⁻-hFlt3L-anti-CTLA-4 to elicit a desired immunological response in the particular subject (the subject's response to therapy). In delivering E3LΔ83N-TK⁻-anti-CTLA-4 or E3LΔ83N-TK⁻-hFlt3L-anti-CTLA-4 to a subject, the dosage will also vary depending upon such factors as the general medical condition, previous medical history, disease type and progression, tumor burden, the presence or absence of tumor infiltrating immune cells in the tumor, and the like.

In some embodiments, it may be advantageous to formulate compositions of the present disclosure in dosage unit form for ease of administration and uniformity of dosage. “Dosage unit form as used herein” refers to physically discrete units suited as unitary dosages for the mammalian subjects to be treated; each unit containing a predetermined quantity of active material calculated to produce the desired therapeutic effect in association with the required pharmaceutically or veterinary acceptable carrier.

### XI. Administration and Therapeutic Regimen of the Engineered Vaccinia Viruses of the Present Technology, Such as E3LΔ83N-TK⁻-Anti-CTLA-4 or E3LΔ83N-TK⁻-hFlt3L-Anti-CTLA-4

Pharmaceutical compositions are typically formulated to be compatible with its intended route of administration. Administration of the engineered vaccinia viruses of the present technology, such as E3LΔ83N-TK⁻-anti-CTLA-4 or E3LΔ83N-TK⁻-hFlt3L-anti-CTLA-4, can be achieved using more than one route. Examples of routes of administration include, but are not limited to parenteral (e.g., intravenous, intramuscular, intraperitoneal, intradermal, subcutaneous), intratumoral, intrathecal, intranasal, systemic, transdermal, iontophoretic, intradermal, intraocular, or topical administration. In one embodiment, E3LΔ83N-TK⁻-anti-CTLA-4 or E3LΔ83N-TK⁻-hFlt3L-anti-CTLA-4 is administered directly into the tumor, e.g. by intratumoral injection, where a direct local reaction is desired. Additionally, administration routes of E3LΔ83N-TK⁻-anti-CTLA-4 or E3LΔ83N-TK⁻-hFlt3L-anti-CTLA-4 can vary, e.g., first administration using an intratumoral injection, and subsequent administration via an intravenous injection, or any combination thereof. A therapeutically effective amount of E3LΔ83N-TK⁻-anti-CTLA-4 or E3LΔ83N-TK⁻-hFlt3L-anti-CTLA-4 injection can be administered for a prescribed period of time and at a prescribed frequency of administration. In certain embodiments, E3LΔ83N-TK⁻-anti-CTLA-4 or E3LΔ83N-TK⁻-hFlt3L-anti-CTLA-4 can be used in conjunction with other therapeutic treatments. For example, E3LΔ83N-TK⁻-anti-CTLA-4 or E3LΔ83N-TK⁻-hFlt3L-anti-CTLA-4 can be administered in a neoadjuvant (preoperative) or adjuvant (postoperative) setting for subjects inflicted with bulky primary tumors. It is anticipated that such optimized therapeutic regimen will induce an immune response against the tumor, and reduce the tumor burden in a subject before or after primary therapy, such as surgery. Furthermore, E3LΔ83N-TK⁻-anti-CTLA-4 or E3LΔ83N-TK⁻-hFlt3L-anti-CTLA-4 can be administered in conjunction with other therapeutic treatments such as chemotherapy or radiation.

In some embodiments, the E3LΔ83N-TK⁻-anti-CTLA-4 or E3LΔ83N-TK⁻-hFlt3L-anti-CTLA-4 virus is co-administered with an immune checkpoint blocking agent, such as a PD-1 and/or PD-L1 inhibitor (e.g., pembrolizumab, nivolumab, atezolizumab, avelumab, or durvalumab). In some embodiments, the E3LΔ83N-TK⁻-anti-CTLA-4 or E3LΔ83N-TK⁻-hFlt3L-anti-CTLA-4 virus is administered intratumorally either simultaneously or sequentially with the systemic administration of the immune checkpoint blocking agent.

In certain embodiments, the E3LΔ83N-TK⁻-anti-CTLA-4 or E3LΔ83N-TK⁻-hFlt3L-anti-CTLA-4 virus is administered at least once weekly or monthly but can be administered more often if needed, such as two times weekly for several weeks, months, years or even indefinitely as long as benefits persist. More frequent administrations are contemplated if tolerated and if they result in sustained or increased benefits. Benefits of the present methods include but are not limited to the following: reduction of the number of cancer cells, reduction of the tumor size, eradication of tumor, inhibition of cancer cell infiltration into peripheral organs, inhibition or stabilization or eradication of metastatic growth, inhibition or stabilization of tumor growth, and stabilization or improvement of quality of life. Furthermore, the benefits may include induction of an immune response against the tumor, activation of effector CD4 T-cells, an increase of effector CD8⁺ T-cells, or reduction of regulatory CD4⁺ cells. For example, in the context of melanoma or, a benefit may be a lack of recurrences or metastasis within one, two, three, four, five or more years of the initial diagnosis of melanoma. Similar assessments can be made for colon cancer and other solid tumors.

In certain other embodiments, the tumor mass or tumor cells are treated with E3LΔ83N-TK⁻-anti-CTLA-4 or E3LΔ83N-TK⁻-hFlt3L-anti-CTLA-4 in vivo, ex vivo, or in vitro.

### XII. Vectors

In some embodiments, a pCB plasmid-based vector is used to insert a specific gene of interest (SG), such as murine CTLA-4 (muCTLA-4) or human Flt3L (hFlt3L) under the control of the vaccinia synthetic early and late promoter (PsE/L). The methodology for constructing the vector has been described (See M. Puhlmann, C. K. Brown, M. Gnant, J. Huang, S. K. Libutti, H. R. Alexander, D. L. Bartlett, Vaccinia as a vector for tumor-directed gene therapy: Biodistribution of a thymidine kinase-deleted mutant. Cancer Gene Therapy, 7(1), 66-73 (2000)). In some embodiments, the CTLA-4 heavy chain (HC) and light chain (LC) sequences are separated by a cassette comprising a furin cleavage site and Pep2A. Human IgG kappa light chain leader sequences are used as the signal peptide for both the heavy and light chain of CTLA-4. In some embodiments, the CTLA-4 heavy chain is separated from an upstream nucleotide sequence encoding hFlt3L by another cassette comprising a furin cleavage site and Pep2A. A xanthine-guanine phophoribosyl transferase gene (gpt) gene under the control of vaccinia P7.5 promoter is used as a selectable marker. In some embodiments, these expression cassettes are flanked by a partial sequence of TK or C7L gene on each side. Homologous recombination that occurs at the TK locus of the plasmid DNA and E3LΔ83N genomic DNA results in the insertion of SG and gpt expression cassettes into the E3LΔ83N genomic DNA TK locus to generate, e.g., E3LΔ83N-TK⁻-anti-CTLA-4 or E3LΔ83N-TK⁻-hFlt3L-anti-CTLA-4. In some embodiments, the E3LΔ83N-TK⁻ base pair positions corresponding to base pair positions 80,962 to 81,032 of the wild type vaccinia WR genomic sequence (SEQ ID NO: 7) are replaced with a heterologous nucleic acid sequence comprising one or more open reading frames that encode for and a gene of interest (SG) and a selectable marker, such as gpt.

It will be appreciated, that any other expression vector suitable for integration into the E3LΔ83N genome could be used as well as alternative promoters, regulatory elements, selectable markers, cleavage sites, leader sequences, and nonessential insertion regions of E3LΔ83N.

### EXPERIMENTAL EXAMPLES

The present technology is further illustrated by the following examples, which should not be construed as limiting in any way.

### General Materials and Methods

Viruses and Cell Lines.

E3LΔ83N viruses were kindly provided by B. L. Jacobs (Arizona State University, Tempe, Ariz.). They were propagated in BSC40 cells and viral titers were determined by plaque assay using BSC40 cells. Alternatively, E3LΔ83N can be generated by homologous recombination at the E3L-Z-DNA binding domain locus using strategies similar to what is described for homologous recombination at the TK locus below. E3LΔ83N-TK⁻-anti-muCTLA-4 and E3LΔ83N-TK⁻-hFlt3L-anti-muCTLA-4 viruses were generated through homologous recombination at the thymidine kinase (TK) locus (see Example 1). These recombinant viruses were enriched through culturing in gpt selection medium and plaque purified in the presence of selection medium through more than three rounds. The pure recombinant clones were amplified in the absence of selection medium. After validation, the viruses were purified through a 36% sucrose cushion. BSC40 cells were cultured in Dulbecco's modified Eagle's medium (DMEM) supplemented containing 5% fetal bovine serum (FBS), 100 Units/ml penicillin, and 100 μg/ml streptomycin. The murine melanoma cell line B16-F10 was originally obtained from I. Fidler (MD Anderson Cancer Center, Houston, Tex.). B16-F10 cells were maintained in complete RPMI 1640 medium including RPMI plus 10% FBS, 100 Units/ml penicillin, 100 μg/ml streptomycin, 0.1 mM non-essential amino acid (NEAA), 2 mM L-glutamine, 1 mM sodium pyruvate, and 10 mM HEPES buffer. The human melanoma SK-MEL-28, and SK-MEL-146 cells were cultured in complete RPMI 1640 medium. All cells were grown at 37° C. in a 5% CO2 incubator.

PCR Verification of Recombinant Virus.

PCR reactions were used to verify the purity of E3LΔ83N-TK⁻-DNP and E3LΔ83N-TK⁻-anti-muCTLA-4 recombinant viruses. The primer sequences used for the PCR reactions are:

TK-F2:
pCB-R3:
(SEQ ID NO: 8)
5′-TGTGAAGACGATAAATTAATGATC-3′,
TK-F4:
(SEQ ID NO: 9)
5′-ACCTGATGGATAAAAAGGCG-3′,
TK-R4:
(SEQ ID NO: 10)
5′-TTGTCATCATGAACGGCGGA-3′,
GS-F:
(SEQ ID NO: 11)
5′-TCCTTCGTTTGCCATACGCT-3′,
GS-R:
(SEQ ID NO: 12)
5′-AGGAGACCAGGCATCCATCT-3′,
(SEQ ID NO: 13)
5′-GTTCTGACGACGGTGGGAAT-3′.

Multi-Step Growth in Cell Culture.

Murine B16-F10, and human SK-MEL-28 and SK-MEL-146 melanoma cells were cultured overnight prior to infection with viruses, including E3LΔ83N-TK⁺, E3LΔ83N-TK⁻, E3LΔ83N-TK⁻-DNP, and E3LΔ83N-TK⁻-anti-muCTLA-4 at a MOI (multiplicity of infection) of 0.1. The inoculum was removed after 60 min; the cells were washed twice with PBS and then overlaid with medium. The cells were harvested at 1, 24, 48, and 72 hours after initial infection by scraping the cells and collect all the medium. After three cycles of freezing and thawing, the samples were sonicated and virus titers were determined by serial dilution and infection of BSC40 cell monolayers. Plaques were visualized by staining with 0.1% crystal violet in 20% ethanol.

Western Blot Analysis.

Murine B16-F10 melanoma cells or human melanoma cells SK-MEL-28 (1×10⁶) were infected with E3LΔ83N-TK⁺, E3LΔ83N-TK⁻, E3LΔ83N-TK⁻-anti-muCTLA-4, or E3LΔ83N-TK⁻-hFlt3L-anti-muCTLA-4 viruses at a MOI of 10. At various times post-infection, the supernatants and cell lysates were collected. For cell lysates, equal amounts of proteins were loaded onto 10% sodium dodecyl sulfate-polyacrylamide gel electrophoresis (SDS-PAGE) gel, and the polypeptides were separated and transferred to a nitrocellulose membrane. The level of anti-muCTLA-4 and hFlt3L expression was determined by Western blot analysis using HRP-linked anti-mouse IgG (Cell Signaling Technology, Danvers, Mass.) and anti-hFlt3L (R&D Systems Inc., Minneapolis, Minn.) antibody. Anti-glyceraldehyde-3-phosphate dehydrogenase (GADPH) antibody (Cell Signaling Technology, Danvers, Mass.) was used for detecting the levels of GAPDH as a loading control. For detecting the secreted anti-muCTLA-4 antibody, equal amount of supernatant was loaded onto an 8% non-denaturing (native) PAGE gel. The polypeptides were separated and transferred to a nitrocellulose membrane. The level of secreted anti-muCTLA-4 antibody expression was determined by Western blot analyses using HRP-linked anti-mouse IgG.

Tumor Implantation and Intratumoral Injection with Viruses.

A bilateral tumor implantation model was used in these experiments. B16-F10 melanoma cells were implanted intradermally into the shaved skin on the right (5×10⁵ cells) and left (1×10⁵ cells) flanks of a C57BL/6J mouse. After 7 to 8 days post implantation, the larger tumors on the right flank (about 3 mm or larger in diameter) were injected twice per week with PBS, E3LΔ83N-TK⁻, E3LΔ83N-TK⁻ plus intraperitoneal (IP) injection of anti-muCTLA-4 antibody (100 μg/mouse), E3LΔ83N-TK⁻ plus intratumoral (IT) injection of anti-muCTLA-4 antibody (10 μg/mouse), or E3LΔ83N-TK⁻-anti-muCTLA-4 when the mice were under anesthesia. Mice were monitored for survival and the tumor sizes were measured twice a week.

Flow Cytometry Analysis of Tumor Infiltrating Immune Cells.

B16-F10 melanoma cells were implanted intradermally to the right and left flanks of C57B/6J mice (5×10⁵ cells to the right flank and 2.5×10⁵ cells to the left flank). PBS, E3LΔ83N-TK⁻, or E3LΔ83N-TK⁻-anti-muCTLA-4 were injected into the tumors on the right flanks 7 days after tumor implantation. The injections were repeated once 3 days later. Tumors were harvested 3 days after the second injection with forceps and surgical scissors and were weighed. They were then minced prior to incubation with Liberase (1.67 Wunsch U/ml) and DNase (0.2 mg/ml) in serum free RPMI medium for 30 minutes at 37° C. Cell suspensions were generated by mashing through a 70 μm nylon filter, and then washed with the complete RPMI medium. Cells were processed for surface labeling with anti-CD3, CD45, CD4, and CD8 antibodies. Live cells are distinguished from dead cells by using fixable dye eFluor506 (eBioscience, Thermo Fisher Scientific, Waltham, Mass.). They were further permeabilized using permeabilization kit (eBioscience, Thermo Fisher Scientific, Waltham, Mass.), and stained for Granzyme B. Data were acquired using the LSRII Flow cytometer (Becton-Dickinson Biosciences, Franklin Lakes, N.J.). Data were analyzed with FlowJo software (FlowJo, Becton-Dickinson, Franklin Lakes, N.J.).

IFN-γ Elispot Assay.

B16-F10 melanoma cells were implanted intradermally to the right (5×10⁵ cells) and left (2.5×10⁵ cells) flanks of C57B/6J mice. Seven days after tumor implantation, the tumors on the right flanks were injected with PBS, E3LΔ83N-TK⁻, E3LΔ83N-TK⁻ hFlt3L, E3LΔ83N-TK⁻-anti-muCTLA-4, or E3LΔ83N-TK⁻-hFlt3L-anti-muCTLA-4. The injections were repeated once 3 days later. Three days after the second injection, spleens were harvested from mice treated with different viruses, and were mashed through a 70 μm strainer (Thermo Fisher Scientific, Waltham, Mass.). Red blood cells were lysed using ACK Lysis Buffer (Life Technologies, Carlsbad, Calif.) and the cells were re-suspended in complete RPMI medium. CD8⁺ T cells were purified using CD8a (Ly-2) MicroBeads from Miltenyi Biotechnology. Enzyme-linked ImmunoSpot (ELISPOT) assay was performed to measure tumor specific IFN-γ⁺ CD8⁺ T cell activities according to the manufacturer's protocol (Becton-Dickinson Biosciences, Franklin Lakes, N.J.). CD8⁺ T cells were mixed with irradiated B16 cells at 1:1 ratio (250,000 cells each) in RPMI medium, and the ELISPOT plate was incubated at 37° C. for 16 hours before staining.

Reagents.

The commercial sources for reagents were as follows: anti-hFlt3L antibody was purchased from R & D Systems Inc. (Minneapolis, Minn.). Therapeutic anti-CTLA4 (clone 9D9) antibody was purchased from BioXcell (West Lebanon, N.H.). HRP-linked anti-mouse IgG and anti-GADPH antibodies were from Cell Signaling Technology (Danvers, Mass.). Anti-CD3, -CD45, -CD8, and -Granzyme B antibodies were purchased form eBioscience (Thermo Fisher Scientific, Waltham, Mass.). CD8a microbeads was from Miltenyi Biotechnology (Somerville, Mass.). ELISPOT assay kit was purchased from Becton-Dickinson Biosciences (Franklin Lakes, N.J.).

Statistics.

Two-tailed unpaired Student's t test was used for comparisons of two groups in the studies. Survival data were analyzed by log-rank (Mantel-Cox) test. The p values deemed significant are indicated in the figures as follows: *, P<0.05; **, P<0.01; ***, P<0.001; ****, P<0.0001.

### Example 1: Generation of Recombinant Vaccinia Virus with a TK-Deletion and Disruption of the E3L Gene Expressing or without Expressing an Antibody that Selectively Targets Cytotoxic T Lymphocyte Antigen 4 (E3LΔ83N-TK⁻-Anti-muCTLA-4)

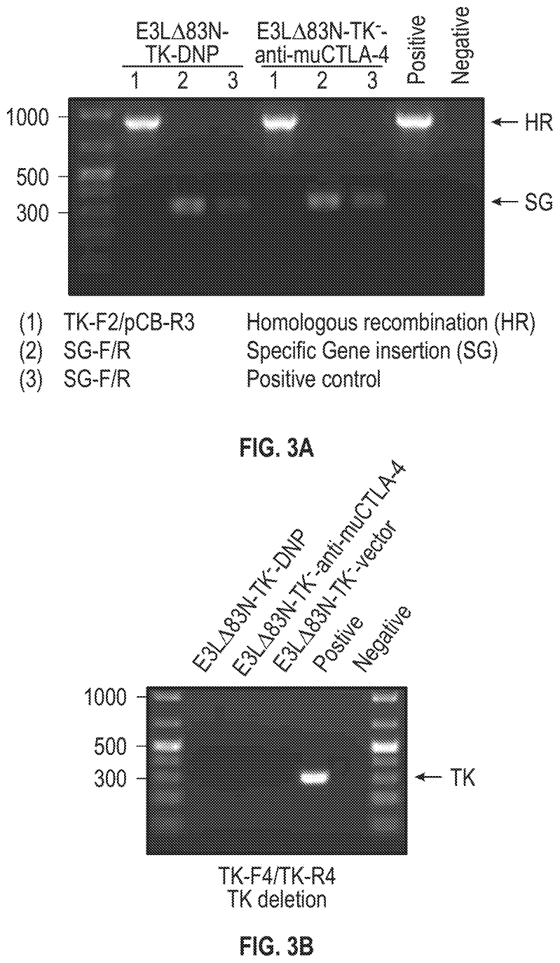

This example describes the generation of a recombinant vaccinia E3LΔ83N virus comprising a TK-deletion expressing or without expressing a murine antibody that specifically targets cytotoxic T lymphocyte antigen 4 (anti-muCTLA-4 (9D9)). FIG. 1 shows the schematic diagram of a single expression cassette designed to express the heavy chain and light of the antibody using the vaccinia viral synthetic early and late promoter (PsE/L). The coding sequence of the heavy chain (muIgG2a) and the light chain of 9D9 was separated by a cassette including a furin cleavage site followed by a 2A peptide (Pep2A) sequence, which enables ribosome skipping. A plasmid containing a specific gene of interest (SG) under the control of the vaccinia PsE/L as well as the E. coli xanthine-guanine phosphoribosyl transferase gene (gpt) under the control of vaccinia P7.5 promoter flanked by the thymidine kinase (TK) gene on either side was constructed using standard recombinant virus technology through homologous recombination at the TK locus between pCB plasmid DNA and viral genomic DNA (FIG. 2). BSC40 cells were infected with recombinant vaccinia virus at a multiplicity of infection (MOI) of 0.05 for 1 h, and then were transfected with the plasmid DNAs described above. The infected cells were collected at 48 h. Recombinant viruses were selected through further culturing in gpt selection medium including MPA, xanthine and hypoxanthine, and plaque purified. PCR analysis was performed to identify recombinant viruses with loss of part of the TK gene and with and without anti-muCTLA-4, (FIG. 3A). Homologous recombination that occurred at the TK locus results in the insertion of SG and gpt expression cassettes or gpt alone into the viral genomic DNA to generate E3LΔ83N-TK⁻-DNP, E3LΔ83N-TK⁻-anti-muCTLA-4, E3LΔ83N-TK vector, and E3LΔ83N-TK⁻-hFlt3L-anti-muCTLA-4 (the construct is described in FIG. 9). FIG. 3B shows PCR analysis of viral genomic DNAs to verify the deletion of TK gene, and to make sure there were no contaminating parental viruses (E3LΔ83N).

### Example 2: VACV E3LΔ83N-TK⁺, E3LΔ83N-TK⁻-vector, E3LΔ83N-TK⁻-DNP, and E3LΔ83N-TK⁻-Anti-muCTLA-4 are Replication Competent

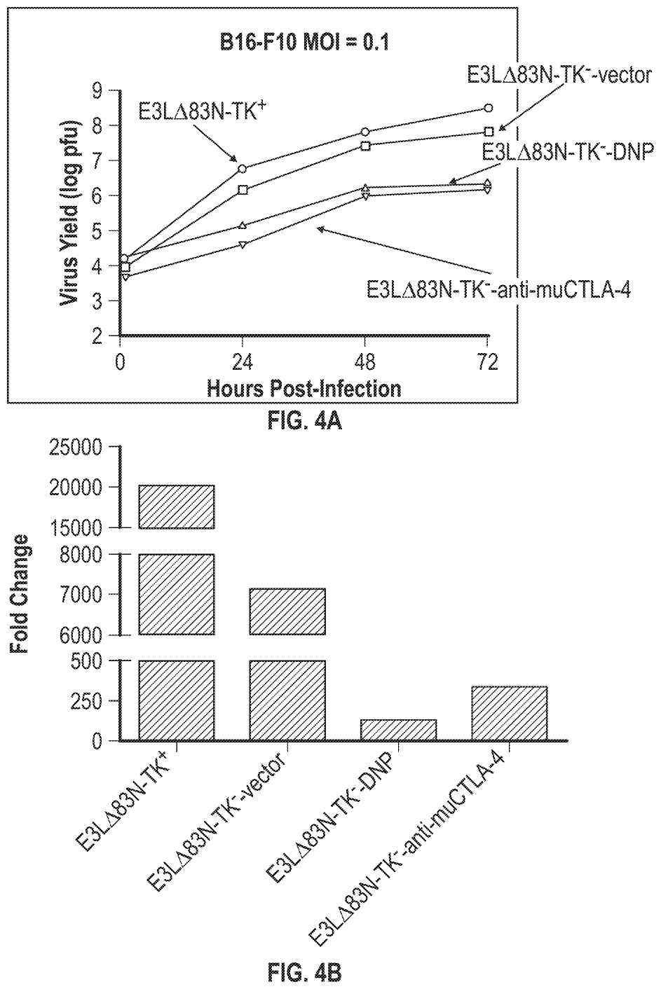

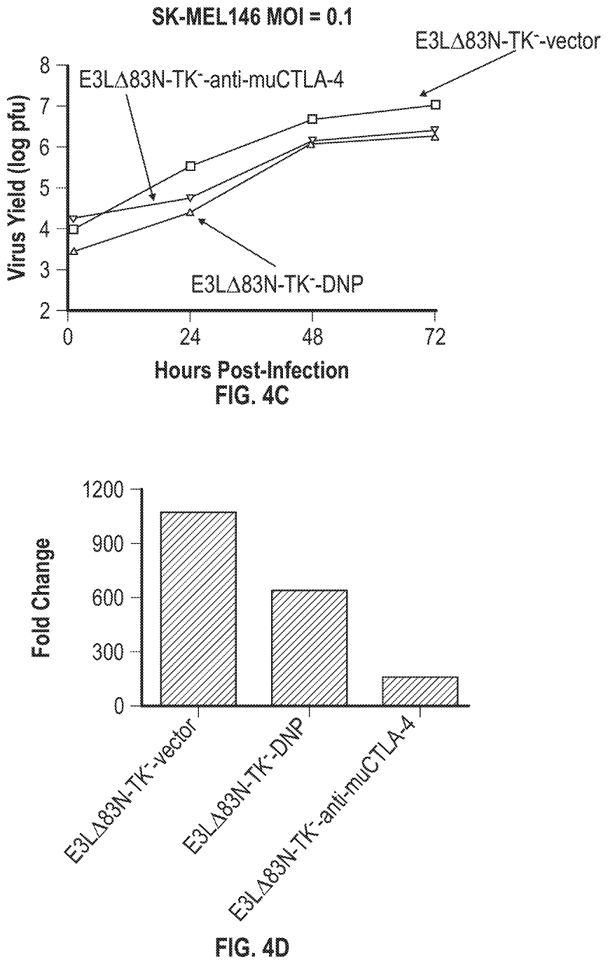

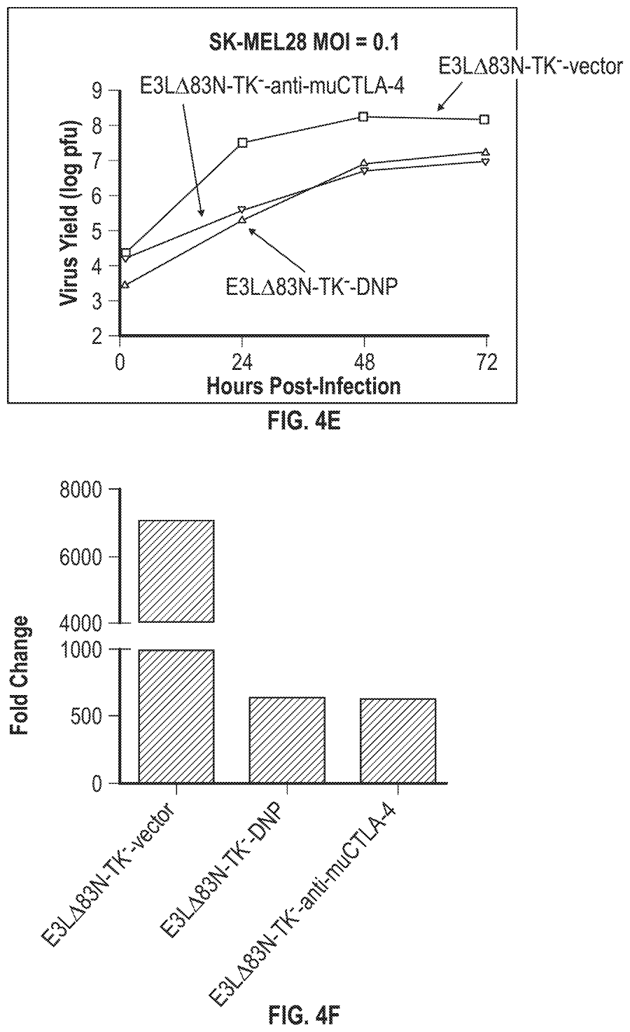

The replication capacities of E3LΔ83N-TK⁺, E3LΔ83N-TK⁻-vector, E3LΔ83N-TK⁻-DNP, and E3LΔ83N-TK⁻-anti-muCTLA-4 were determined in murine B16-F10 melanoma cells by infecting them at a MOI of 0.1. Cells were collected at various time points post infection and viral yields (log pfu) were determined by titrating on BSC40 cells. FIG. 4A shows the graphs of viral yields plotted against hours post infection. E3LΔ83N-TK⁺ replicated efficiently in B16-F10 cells with viral titers increasing by 20,000-fold at 72h post-infection. Deletion of the TK gene resulted in the 3-fold decrease in viral replication in B16-F10 cells compared with E3LΔ83N-TK⁺. The fold changes of viral yields at 72 h over those at 1 h post infection were calculated (FIG. 4B). Similarly, the replication capacities of E3LΔ83N-TK⁻-vector, E3LΔ83N-TK⁻-DNP, and E3LΔ83N-TK⁻-anti-muCTLA-4 were determined in human melanoma cell lines SK-MEL-146 and SK-MEL-28 by infecting them at a MOI of 0.1. Cells were collected at various time points post infection and viral yields (log pfu) were determined by titrating on BSC40 cells. FIGS. 4C-4F show a series of graphs of viral yields plotted against hours post infection. All VACV strains, E3LΔ83N-TK⁻-vector, E3LΔ83N-TK⁻-DNP, and E3LΔ83N-TK⁻-anti-muCTLA-4, have replicated efficiently in human melanoma cell lines SK-MEL-146 and SK-MEL-28.

TABLE 2
Quantitative data (fold change) for results shown in FIGS. 4B, 4D,
and 4F

| | | E3LΔ83N- | | E3LΔ83N- |
|---|---|---|---|---|
| | E3LΔ83N- | TK⁻- | E3LΔ83N- | TK⁻-anti- |
| | TK+ | vector | TK⁻⁻DNP | muCTLA-4 |
| B16-F10 | 20000 | 7103 | 124 | 328 |
| (FIG. 4B) | | | | |
| SK-mel- | | 1066 | 633 | 150 |
| 146 | | | | |
| (FIG. 4D) | | | | |
| SK-mel- | | 7000 | 629 | 615 |
| 28 | | | | |

### Example 3: Expression of Anti-DNP and Anti-muCTLA-4 in B16-F10 Melanoma Cells Via Infection of E3LΔ83N-TK⁻-Anti-muCTLA-4

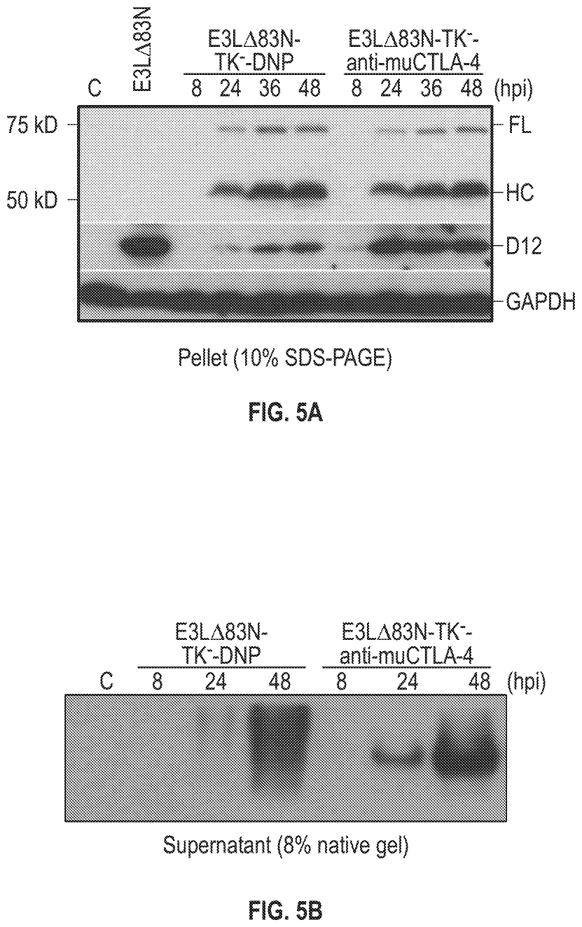

To determine whether E3LΔ E3LΔ83N-TK⁻ recombinant viruses are capable of expressing desired anitbodies, B16-F10 murine melanoma cells were infected with E3LΔ83N-TK⁻, E3LΔ83N-TK⁻-DNP, and E3LΔ83N-TK⁻-anti-muCTLA-4 at a MOI of 10, and the expression of anti-DNP and anti-muCTLA-4 was measured. Cell lysates and supernatants were collected at various times (8, 24, 36, and 48 hours) post infection. Western blot analyses were performed to determine the levels of the antibody expression. As shown in FIGS. 5A-B, Western blot analysis reveals abundant levels of both antibodies, anti-DNP and anti-muCTLA-4, in both the cell lysates and the supernatants. Accordingly, these results demonstrate that the recombinant viruses of the present technology have the capacity to express specific genes of interest in infected cells and are useful in methods for delivering the desired antibodies to cells.

### Example 4: Expression of Anti-muCTLA-4 in Human SK-MEL-28 Melanoma Cells

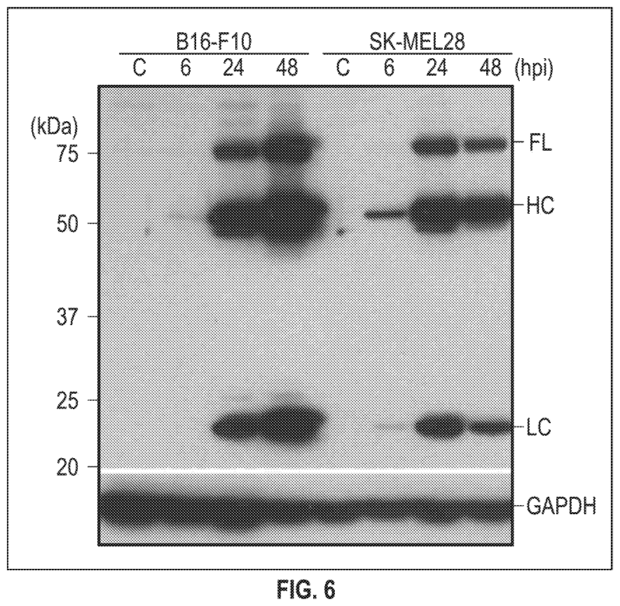

To determine whether recombinant E3LΔ83N-TK⁻-anti-muCTLA-4 virus infection results in the production of anti-CTLA-4 antibodies, human SK-MEL-28 melanoma cells and murine B16-F10 cells were infected with E3LΔ83N-TK⁻-anti-muCTLA-4 at a MOI of 10. Cell lysates were collected at 6, 24, and 48 hours post-infection, and polypeptides were separated using 10% SDS-PAGE. HRP-linked anti-mouse IgG (heavy and light chain) antibody was used to detect full-length (FL), heavy chain (HC), and light chain (LC) of anti-muCTLA-4 antibodies. GAPDH was used as a loading control. As shown in FIG. 6, Western blot analysis shows the expression of the full-length (FL), heavy chain (HC), and light chain (LC) of anti-CTLA-4 antibodies in both the B16-F10 and SK-MEL-28 melanoma cell lines. anti-muCTLA-4, in both the cell lysates and the supernatants. Accordingly, these results demonstrate that the recombinant viruses of the present technology have the capacity to express anti-CTLA-4 antibodies in infected cells and are useful in methods for delivering the antibodies to cells.

### Example 5: Intratumoral Injection of E3LΔ83N-TK⁻-Anti-muCTLA-4 is More Effective than E3LΔ83N-TK⁻ in a Bilateral B16-F10 Tumor Implantation Model

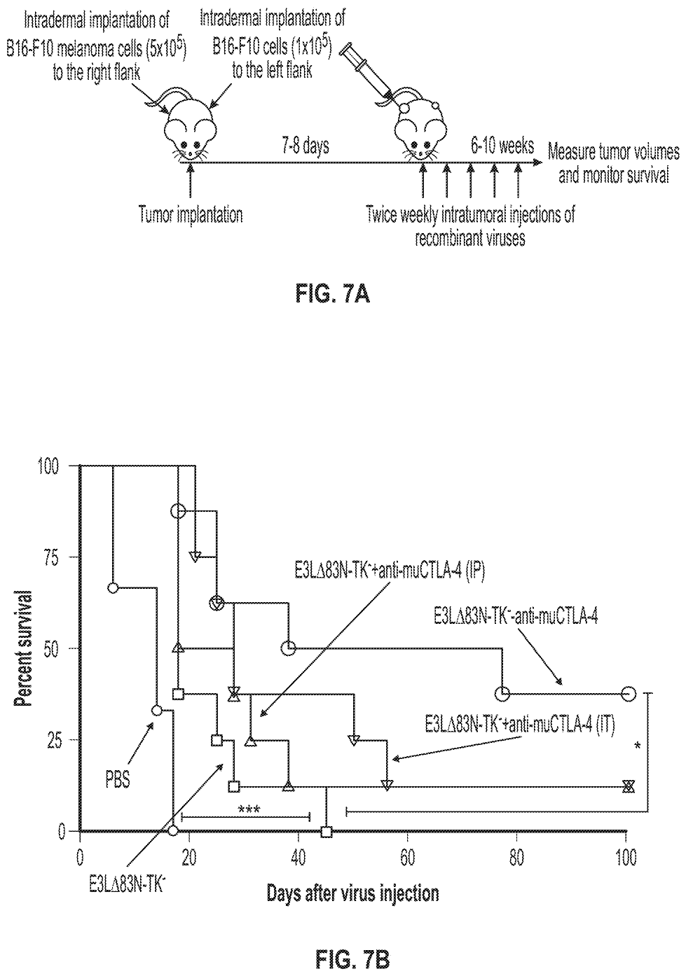

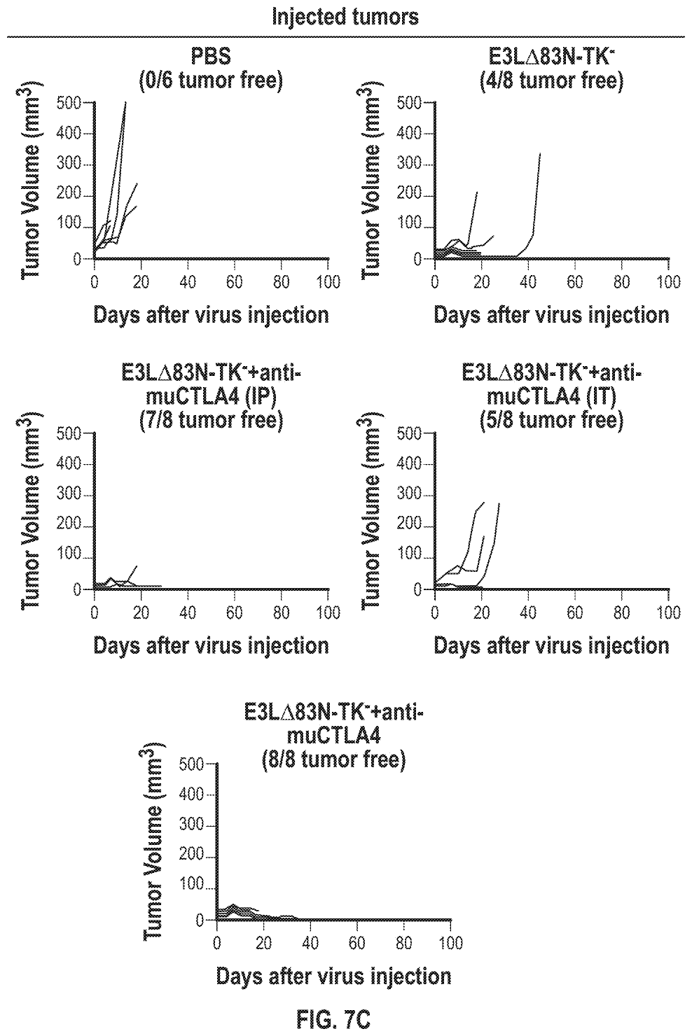

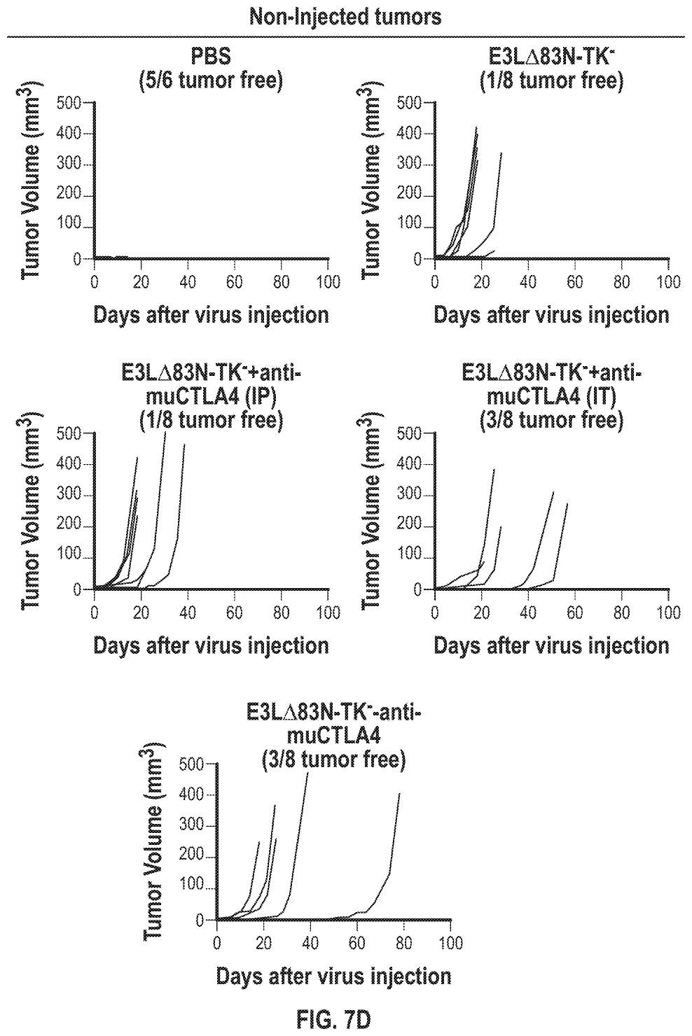

To test the in vivo tumor killing activities of the recombinant viruses and vector control, a bilateral tumor implantation model was used. B16-F10 melanoma cells were implanted intradermally into the shaved skin on the right (5×10⁵ cells) and left (1×10⁵ cells) flanks of a C57BL/6J mouse. After 7 to 8 days post implantation, the larger tumors on the right flank (about 3 mm or larger in diameter) were injected twice per week with PBS, E3LΔ83N-TK⁻, E3LΔ83N-TK⁻ plus intraperitoneal (IP) injection of anti-muCTLA-4 antibody (100 μg/mouse), E3LΔ83N-TK⁻ plus intratumoral (IT) injection of anti-muCTLA-4 antibody (10 μg/mouse), or E3LΔ83N-TK⁻-anti-muCTLA-4 when the mice were under anesthesia. Mice were monitored for survival and the tumor sizes were measured twice a week. The experimental scheme is shown in FIG. 7A. Tumor volumes were measured and the survival of the mice was monitored. FIG. 7B shows the Kaplan-Meier survival curve of the experiment. The data demonstrate that mice with PBS mock-treated tumors grew very quickly and the mice died with a median survival of 14 days. The injection of E3LΔ83N-TK⁻ into the tumors extended the median survival day to 18 days. Co-injection of E3LΔ83N-TK⁻ plus intraperitoneal (IP) injection of anti-muCTLA-4 antibody (100 μg/mouse), increased the median survival to 23 days. Co-injection of E3LΔ83N-TK⁻ plus intratumoral (IT) injection of anti-muCTLA-4 antibody (10 μg/mouse), increased the median survival to 28 days. The injection of E3LΔ83N-TK⁻-anti-muCTLA-4 into the tumors extended the median survival to 57.5 days. FIG. 7C-D demonstrate the measured tumor volume over time for injected tumors and non-injected tumors. These results demonstrate that the expression of anti-muCTLA-4 by the engineered E3LΔ83N-TK⁻-anti-muCTLA-4 is capable of reducing tumor volume and slowing tumor growth in both injected and non-injected tumors, thereby demonstrating an abscopal effect. In addition, these results show that the use of a recombinant E3LΔ83N-TK⁻ virus expressing the anti-CLTA-4 antibody is more efficacious than co-administration of the E3LΔ83N-TK⁻ plus IT or IP injection of anti-muCTLA-4.

### Example 6: Intratumoral Injection of E3LΔ83N-TK⁻-Anti-muCTLA-4 is More Effective than E3LΔ83N-TK⁻ in the Proliferation and Activation of CD8⁺ and CD4⁺ T Cells in the Non-Injected Tumors

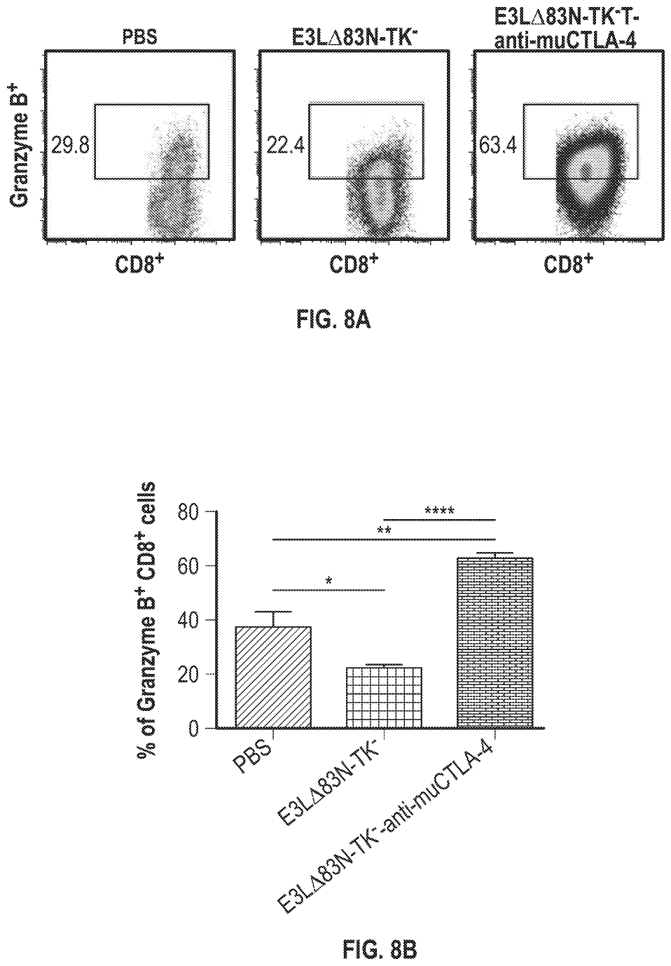

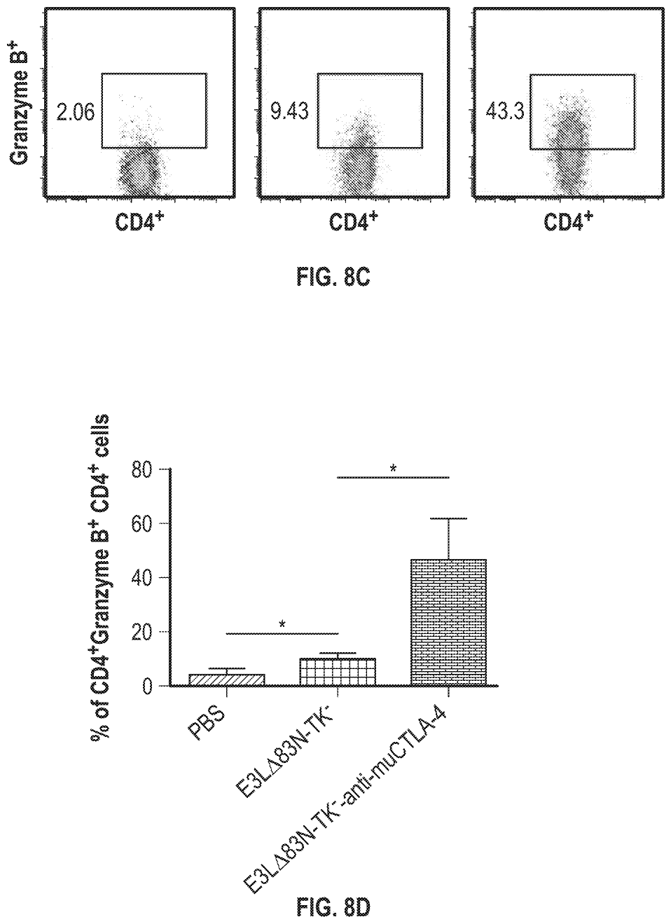

To assess whether intratumoral injection of E3LΔ83N-TK⁻ or E3LΔ83N-TK⁻-anti-muCTLA-4 in B16-F10 melanomas leads to activation and proliferation of CD8⁺ and CD4⁺ T cells, B16-F10 melanoma cells were implanted intradermally to the right and left flanks of C57B/6J mice (5×10⁵ cells to the right flank and 2.5×10⁵ cells to the left flank). Seven days after tumor implantation, PBS, E3LΔ83N-TK⁻, or E3LΔ83N-TK⁻-anti-muCTLA-4 were injected into the tumors on the right flanks. The injections were repeated three days later. Three days after the second injection, tumors were harvested and cells were processed for surface labeling with anti-CD3, CD45, CD4, and CD8 antibodies, and also for intracellular Granzyme B staining. The live immune cell infiltrates in the non-injected tumors were analyzed by FACS. There was a dramatic increase in CD8⁺ T cells expressing Granzyme B in the injected tumors, from 37% in tumors of PBS-treated mice to 62% in the tumors of E3LΔ83N-TK⁻-anti-muCTLA-4-treated mice, whereas the percentage of Granzyme CD8⁺ T cell were decreased to 22% in the tumors of E3LΔ83N-TK⁻-treated mice (FIG. 8A-B). These results indicate that intratumoral injection of E3LΔ83N-TK⁻-anti-muCTLA-4 led to significantly increased levels of activated CD8⁺ T cells in the non-injected tumors. Similar changes were observed for the CD4⁺ T cells expressing Granzyme B in the injected tumors. There was a dramatic increase in CD4⁺ T cells expressing Granzyme B in the non-injected tumors, from 3.7% in tumors of PBS-treated mice to 10% in the tumors of E3LΔ83N-TK⁻-treated mice, to 46% in the tumors of in E3LΔ83N-TK⁻-anti-muCTLA-4-treated mice (FIG. 8C-D). These results demonstrate that intratumoral injection of E3LΔ83N-TK⁻-anti-muCTLA-4 is capable of inducing an immune response in the subject, including increasing cytotoxic CD8⁺ T cells and/or CD4⁺ T cells within non-injected tumors.

### Example 7: Generation of Recombinant Vaccinia Virus with with a TK-Deletion and Disruption of the E3L Gene Expressing or without Expressing Human Fms-Like Tyrosine Kinase 3 Ligand (hFlt3L) and an Antibody that Selectively Targets Cytotoxic T Lymphocyte Antigen 4 (E3LΔ83N-TK⁻-hFlt3L-Anti-muCTLA-4)

A recombinant vaccinia viruses comprising a TK-deletion expressing or without expressing human Fms-like tyrosine kinase 3 ligand (hFlt3L) and a murine antibody that specifically targets cytotoxic T lymphocyte antigen 4 (anti-muCTLA-4 (9D9)) was generated. FIG. 9 shows the schematic diagram of a single expression cassette designed to express hFlt3L and the heavy chain and light of anti-muCTLA-4 using the vaccinia viral synthetic early and late promoter (PsE/L). The coding sequence of the hFlt3L and the heavy chain (muIgG2a) was separated by a cassette including a furin cleavage site followed by a 2A peptide (Pep2A) sequence, which enables ribosome skipping. The heavy chain (muIgG2a) and the light chain of 9D9 was also separated by a cassette including a furin cleavage site followed by Pep2A sequence.

### Example 8: Expression of hFlt3L and Anti-muCTLA-4 in B16-F10 Melanoma Cells Via Infection of E3LΔ83N-TK⁻-hFlt3L-Anti-muCTLA-4

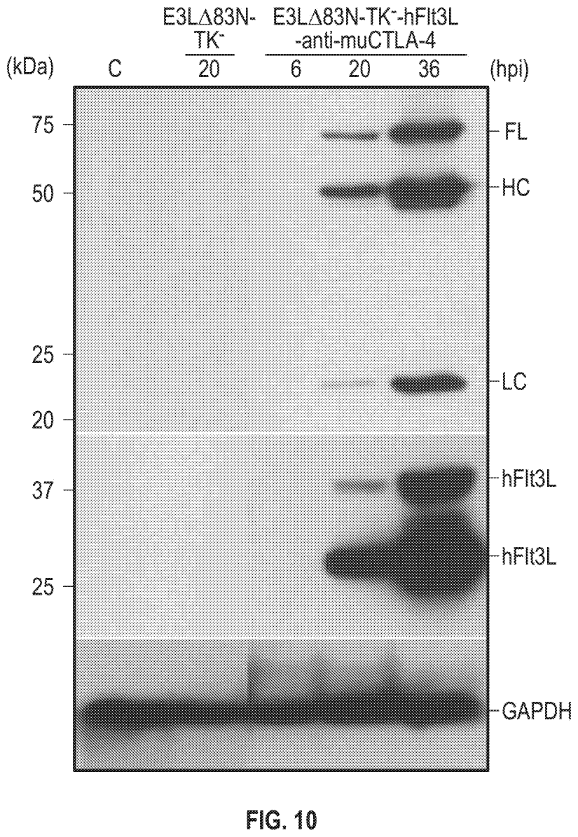

To test the expression of hFlt3L and anti-muCTLA-4 from the E3LΔ83N-TK⁻-hFlt3L-anti-muCTLA-4 recombinant virus, B16-F10 melanoma cells were mock infected or infected with E3LΔ83N-TK⁻ or E3LΔ83N-TK⁻-hFlt3L-anti-muCTLA-4 at a MOI of 10. Cell lysates and supernatants were collected at various times (6, 20, and 36 hours) post-infection. Western blot analyses were performed to determine the levels of the protein and antibody expression. As shown in FIG. 10, Western blot analysis reveals abundant levels of both hFlt3L and anti-muCTLA-4 (full-length (FL), heavy chain (HC), and light chain (LC)) in the cell lysates. Accordingly, these results demonstrate that the E3LΔ83N-TK⁻-hFlt3L-anti-muCTLA-4 recombinant viruses of the present technology are capable of expressing hF1T3L and anti-CLTA-4 antibody in infected cells and are useful in methods for expressing these proteins in tumor cells.

### Example 9: Intratumoral Injection with E3LΔ83N-TK⁻-Anti-muCTLA-4 or E3LΔ83N-TK⁻-hFlt3L-Anti-muCTLA-4 Leads to the Generation of Antitumor CD8⁺ T-Cell Immunity

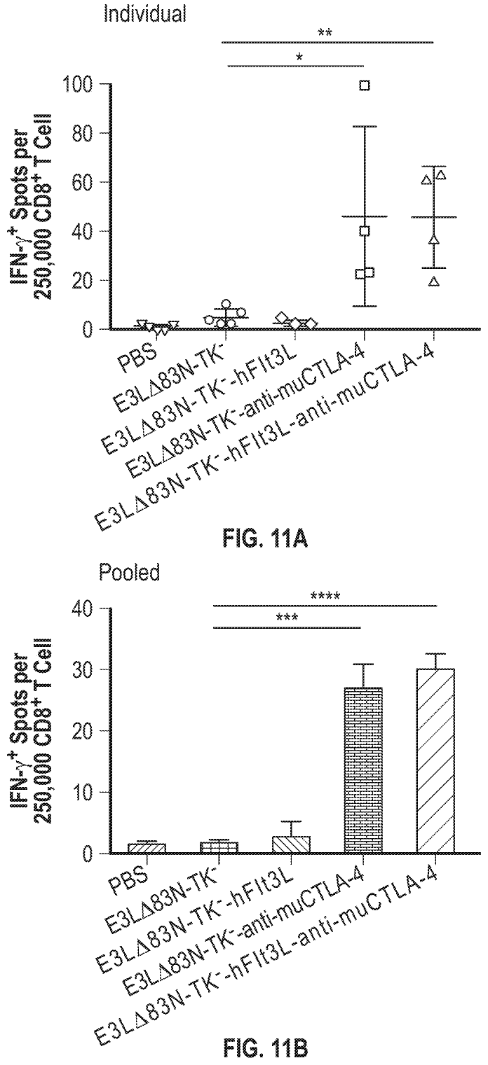

To assess whether mice gained antitumor memory T-cell immunity against the murine B16-F10 melanoma cancer after treatment with intratumoral injection of PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4, Enzyme-linked ImmunoSpot (ELISpot) was used. B16-F10 cells (5×10⁵ and 2.5×10⁵, respectively) were intradermally implanted into the shaved skin on the right and left flank of C57BL/6J mice. Seven days after tumor implantation the tumors on the right flank (about 3 mm in diameter) were injected with PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4. The injections were repeated three days later, followed by euthanization three days after the second injection. ELISpot was performed to assess the generation of antitumor specific CD8⁺ T cells in the spleens of mice treated with the recombinant viruses. Briefly, CD8⁺ T cells were isolated from splenocytes and 2.5×10⁵ cells were cultured with irradiated B16-F10 cells overnight at 37° C. in anti-IFN-γ-coated BD ELISpot plate microwells. CD8⁺ T cells were stimulated with B16-F10 cells irradiated with an γ-irradiator and IFN-γ secretion was detected with an anti-IFN-γ antibody. FIG. 11A shows the numbers of IFN-γ⁺ spots per 250,000 CD8⁺ T cells from individual mouse treated with either PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4. FIG. 11B shows the numbers of IFN-γ⁺ spots per 250,000 CD8⁺ T cells pooled from mice in each group treated with either PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4. These results demonstrate that intratumoral injection of E3LΔ83N-TK⁻-anti-muCTLA-4 or E3LΔ83N-TK⁻-hFlt3L-anti-muCTLA-4 increases antitumor CD8⁺ T cells in treated mice. In addition, the results demonstrate that the intratumoral injection of E3LΔ83N-TK⁻-anti-muCTLA-4 or E3LΔ83N-TK⁻-hFlt3L-anti-muCTLA-4 is more effective than E3LΔ83N-TK⁻ or E3LΔ83N-TK⁻-hFlt3L in generating antitumor CD8⁺ T cells in treated mice in a murine B16-F10 melanoma bilateral implantation model. Accordingly, these results demonstrate that the recombinant E3LΔ83N-TK⁻-anti-muCTLA-4 and E3LΔ83N-TK⁻-hFlt3L-anti-muCTLA-4 viruses of the present technology are effective in enhancing or promoting an immune response in the subject and in increased cytotoxic CD8⁺ T cells within of a subject.

### Example 10: Combination Therapy with Intratumoral Injection of E3LΔ83N-TK⁻-Anti-muCTLA-4 or E3LΔ83N-TK⁻-hFlt3L-Anti-muCTLA-4 and Systemic Administration of Anti-PD1/PD-L1 Therapy

This example demonstrates the use of recombinant viruses of the present technology, such as E3LΔ83N-TK⁻-anti-huCTLA-4 or E3LΔ83N-TK⁻-hFlt3L-anti-huCTLA-4 in combination with the use of PD1/PD-L1 therapy in the treatment of solid tumors, such as melanoma.

### Methods

Reagents.

Murine anti-PD-L1 (clone 10F.9G2) antibody was purchased from BioXcell.

Tumor Implantation and Intratumoral Injection with Viruses.

A bilateral tumor implantation model was used. B16-F10 melanoma cells were implanted intradermally into the shaved skin on the right (5×10⁵ cells) and left (1×10⁵ cells) flanks of a C57BL/6J mouse. After 8 days post implantation, the larger tumors on the right flank (about 3 mm or larger in diameter) were injected twice per week with PBS, E3LΔ83N-TK⁻, E3LΔ83N-TK⁻-anti-muCTLA-4, E3LΔ83N-TK⁻-hFlt3L-anti-muCTLA-4, or E3LΔ83N-TK⁻ plus intraperitoneal (IP) injection of anti-muPD-L1 antibody (250 μg/mouse), E3LΔ83N-TK⁻-anti-muCTLA-4 plus IP injection of anti-muPD-L1 antibody (250 μg/mouse), E3LΔ83N-TK⁻-hFlt3L-anti-muCTLA-4 plus IP injection of anti-muPD-L1 antibody (250 μg/mouse), when the mice were under anesthesia. Mice were monitored for survival and the tumor sizes were measured twice a week.

### Results

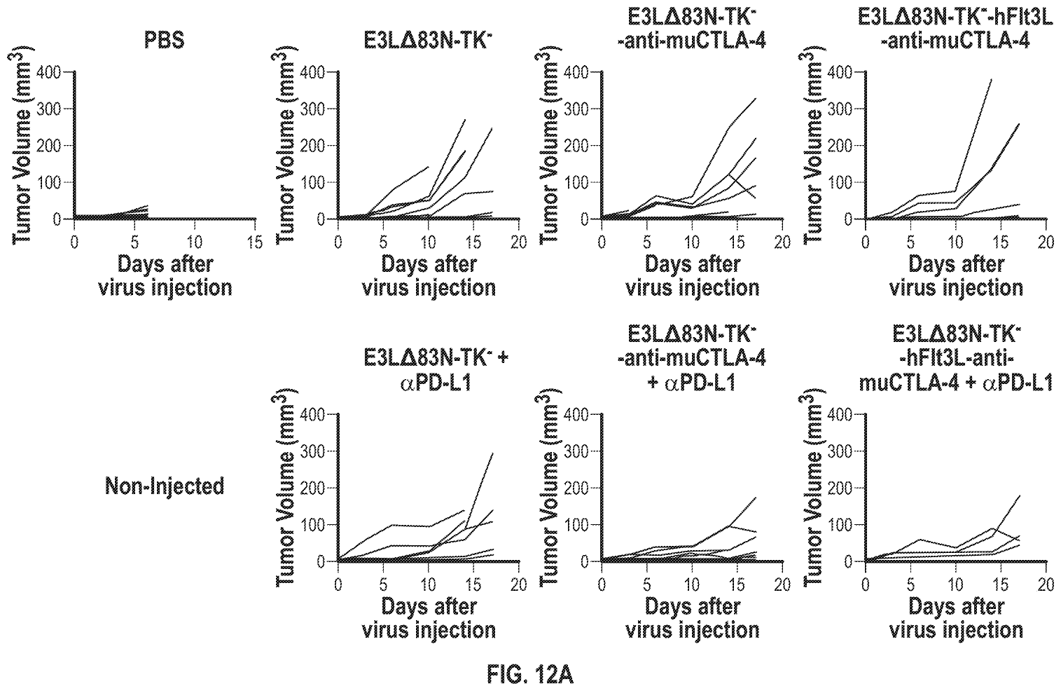

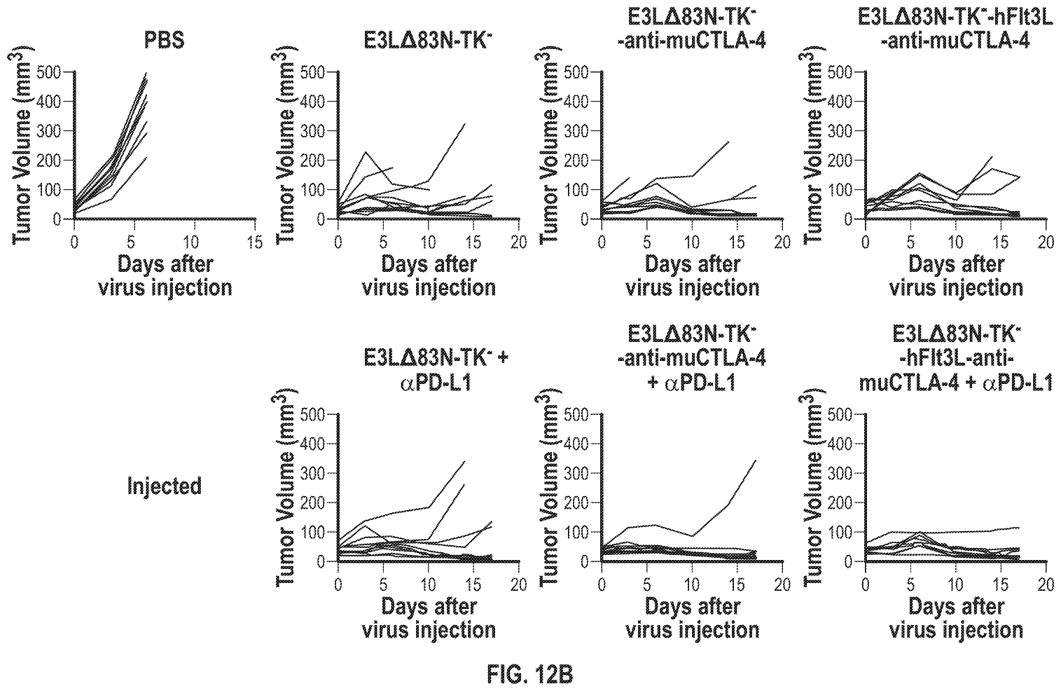

As shown in FIGS. 12A-12B, combination therapy delayed tumor growth on the non-injected side of the mice when compared with intratumoral injection of the virus alone. Also, combination therapy with recombinant viruses expressing anti-muCTLA-4 delayed the tumor growth when compared with control virus combination therapy. Accordingly, these results demonstrate that the compositions of the present technology are useful in methods for treating a solid tumor.

### Example 11: Use of E3LΔ83N-TK⁻-Anti-huCTLA-4 or E3LΔ83N-TK⁻-hFlt3L-Anti-huCTLA-4 in the Treatment of a Solid Tumor in Humans

This example demonstrates the use of the recombinant viruses of the present technology, such as E3LΔ83N-TK⁻-anti-huCTLA-4 or E3LΔ83N-TK⁻-hFlt3L-anti-huCTLA-4, in the treatment of solid tumors, such as melanoma.

### Methods

Subjects diagnosed as having a solid tumor, such as melanoma, receive administrations of 4×10⁶-4×10⁸ pfu of E3LΔ83N-TK⁻-anti-huCTLA-4 or E3LΔ83N-TK⁻-hFlt3L-anti-huCTLA-4 every two to three weeks. The recombinant virus is administered intratumorally according to methods know in the art. Subjects are evaluated every two to three weeks for measurements of tumor volume. Treatments are maintained until such a time when tumor volume decreases or the tumor is eradicated, or one or more signs or symptoms indicative of a solid tumor is ameliorated or eliminated.

It is anticipated that subjects having been diagnosed with a solid tumor, such as melanoma, receiving therapeutically effective amounts of a recombinant virus of the present technology, such as E3LΔ83N-TK⁻-anti-huCTLA-4 or E3LΔ83N-TK⁻-hFlt3L-anti-huCTLA-4, will display reduced tumor volume or tumor eradication, and/or reduced severity or elimination of one or more signs or symptoms indicative of a solid tumor.

Results

These results will show that recombinant viruses of the present technology, such as E3LΔ83N-TK⁻-anti-huCTLA-4 or E3LΔ83N-TK⁻-hFlt3L-anti-huCTLA-4, are useful in the treatment of solid tumors, such as melanoma.

### Example 12: E3LΔ83N-TK⁻-Vector, E3LΔ83N-TK⁻-Anti-muCTLA-4, and E3LΔ83N-TK⁻-hFlt3L-Anti-muCTLA-4 Recombinant Viruses are Replication Competent

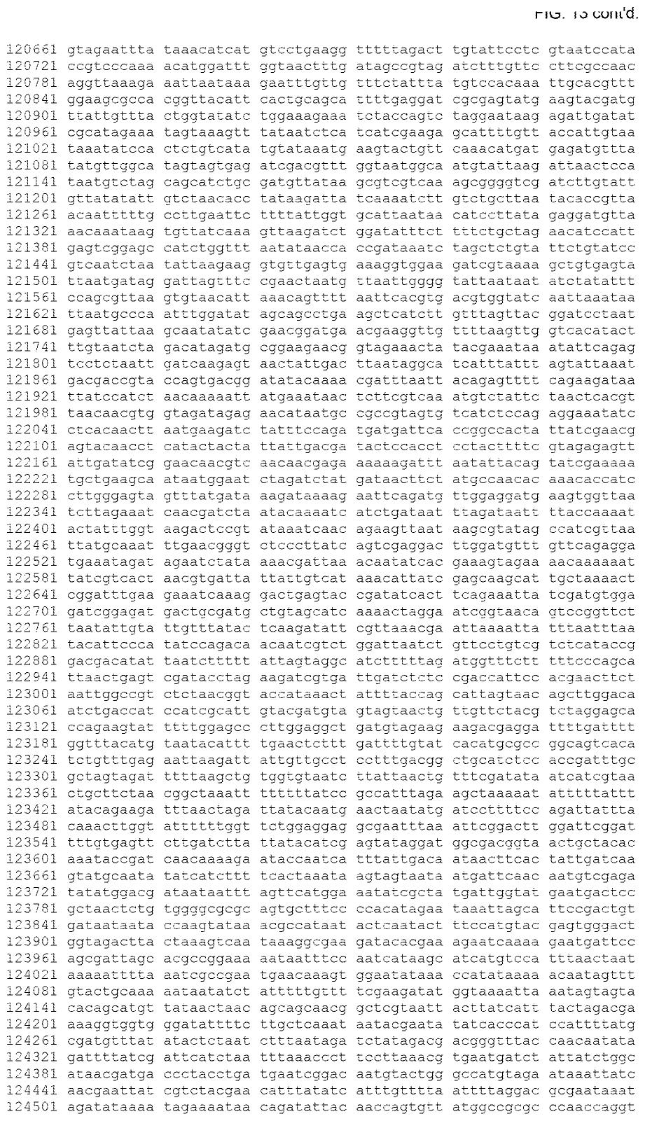

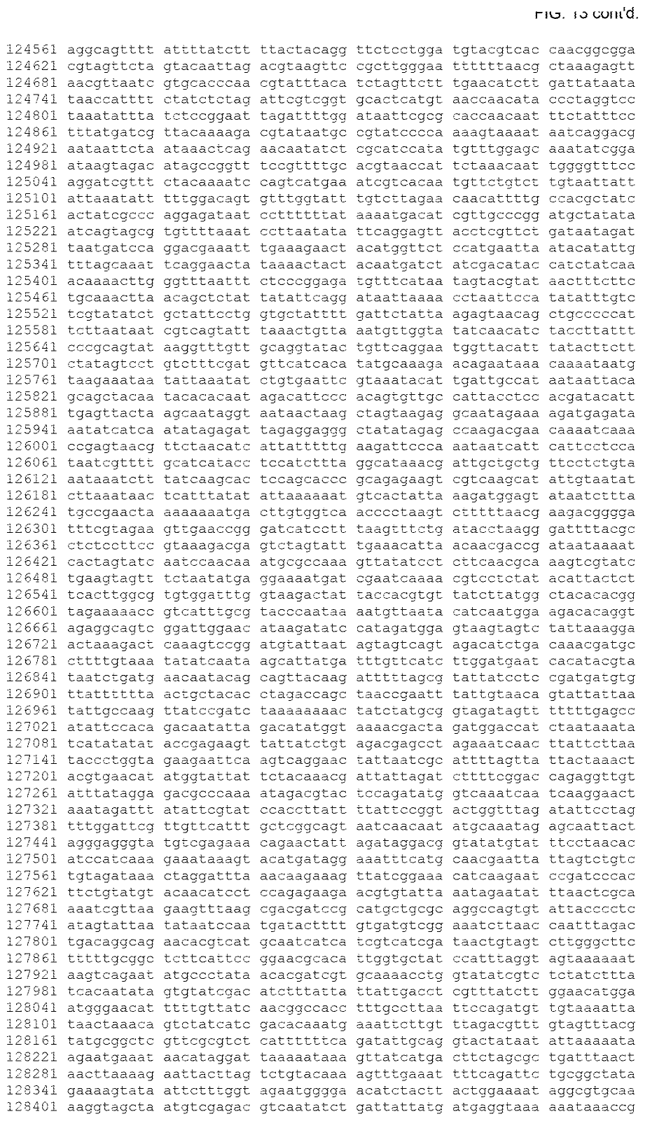

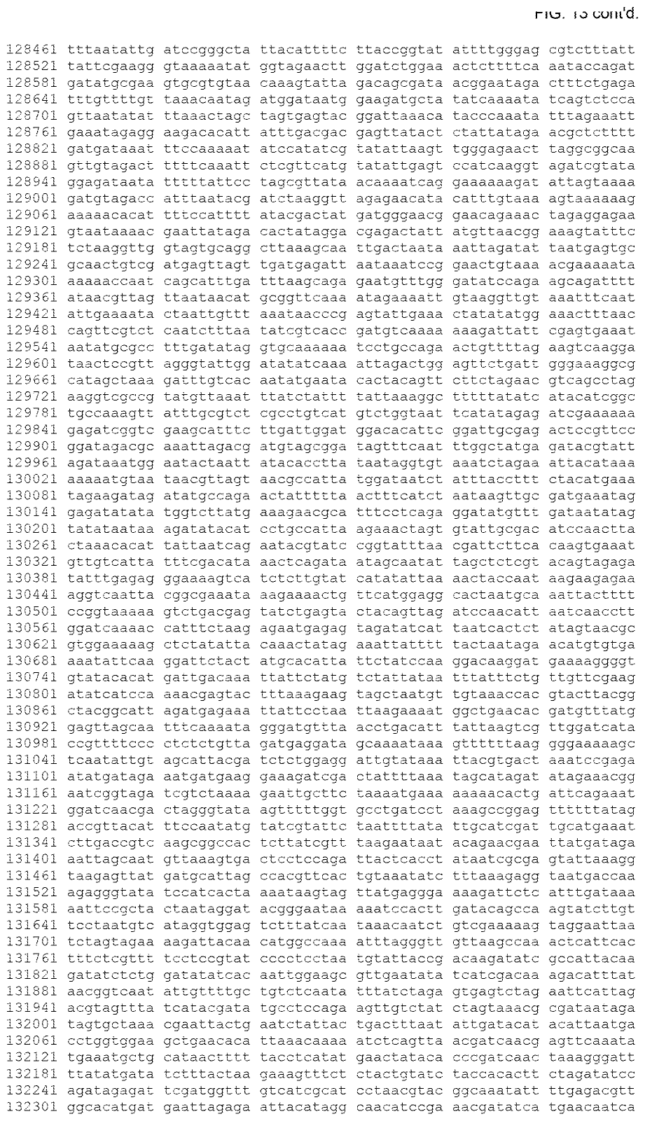

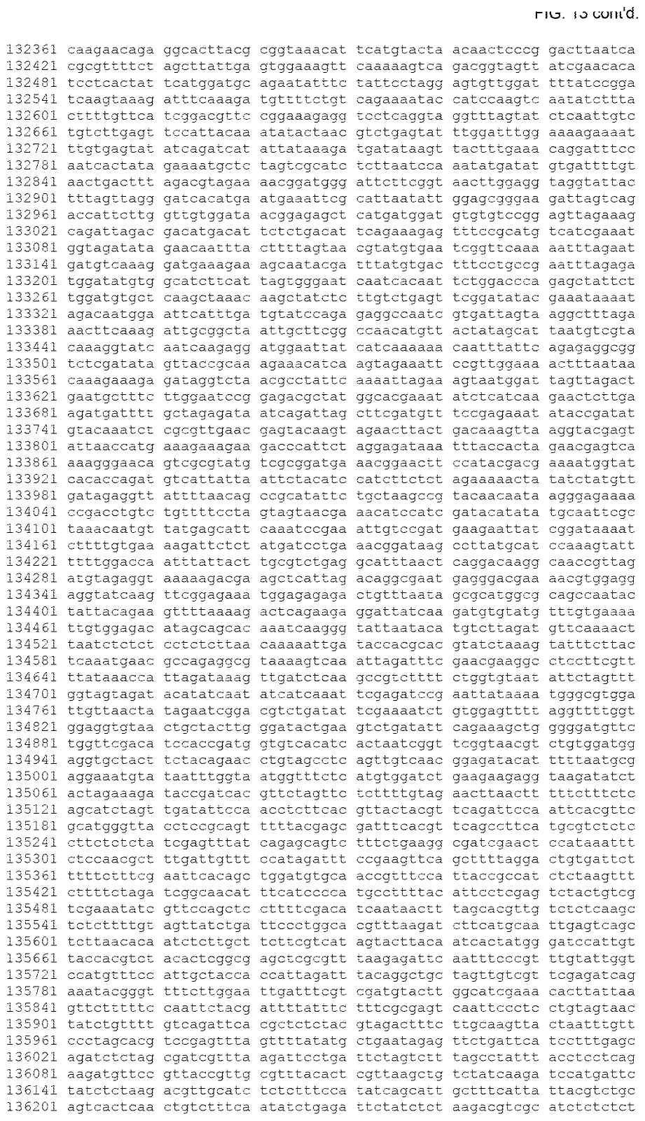

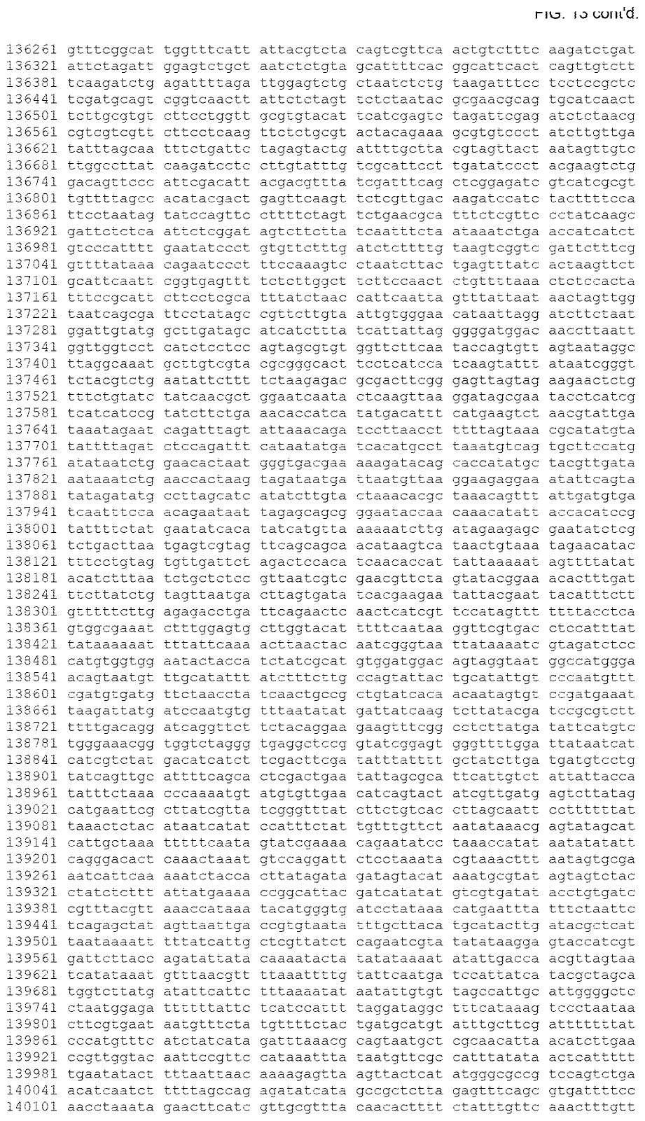

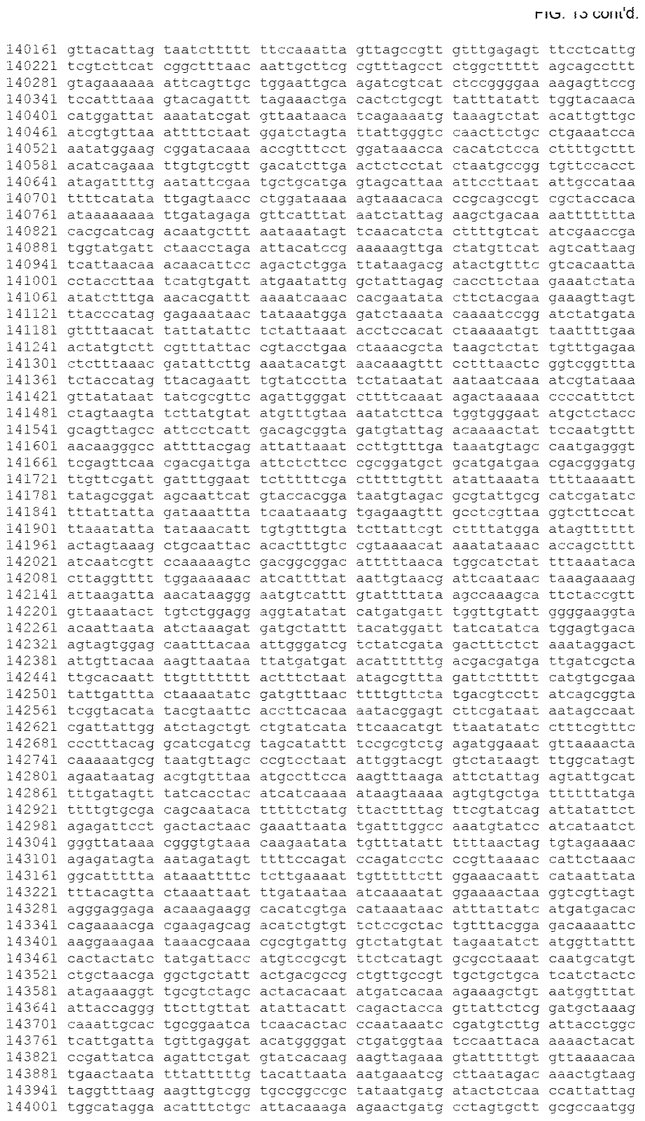

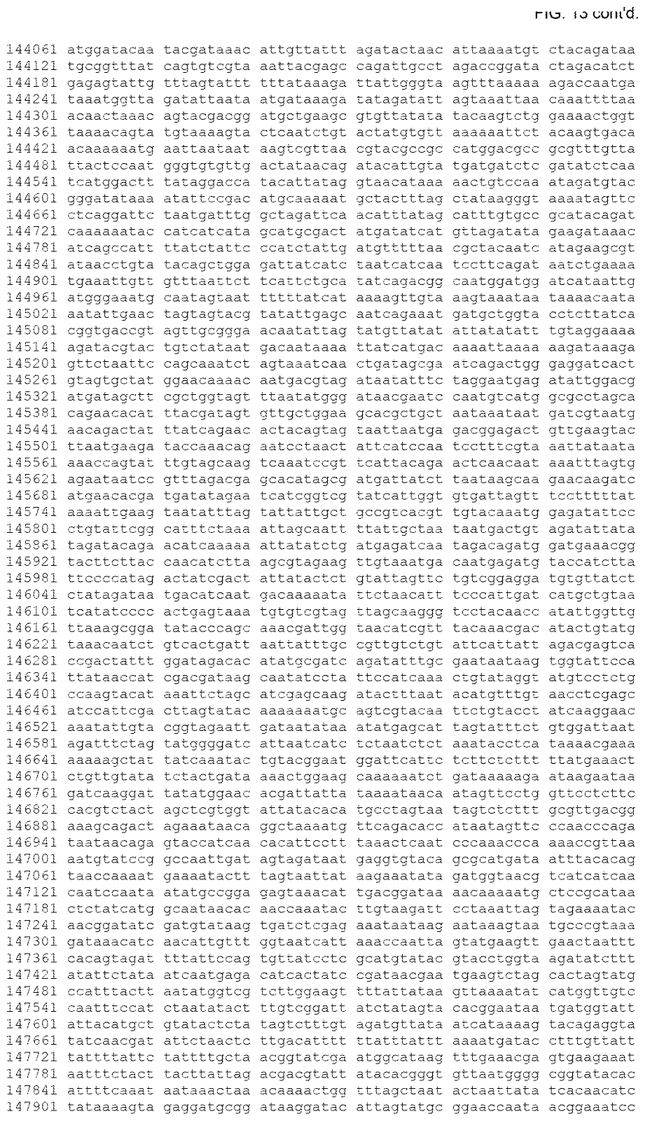

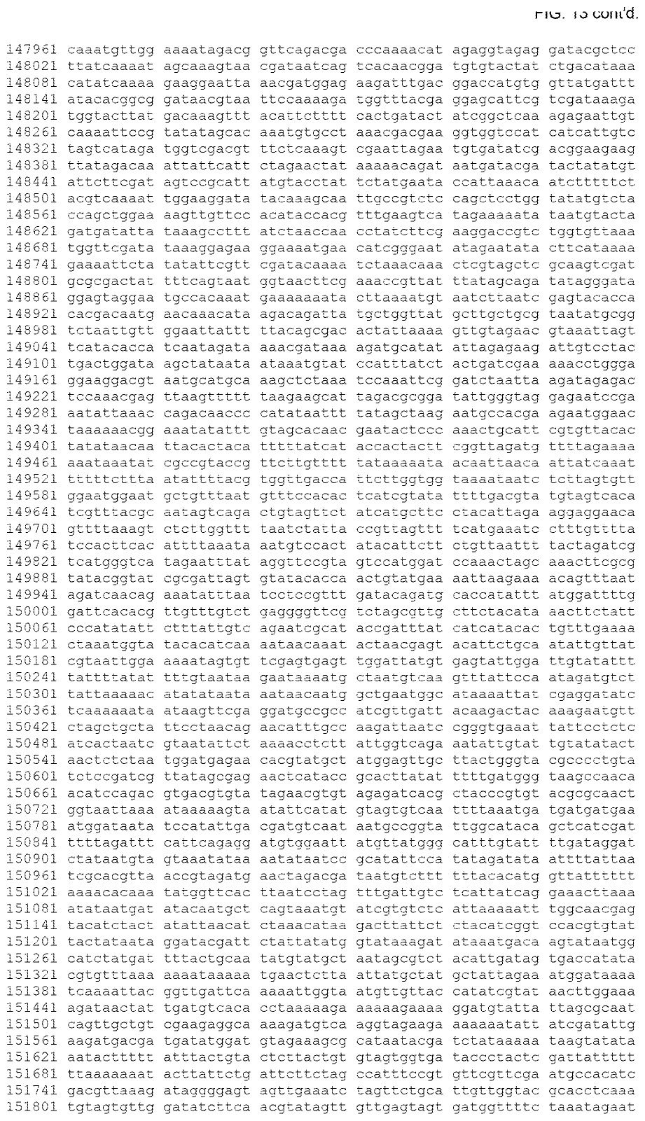

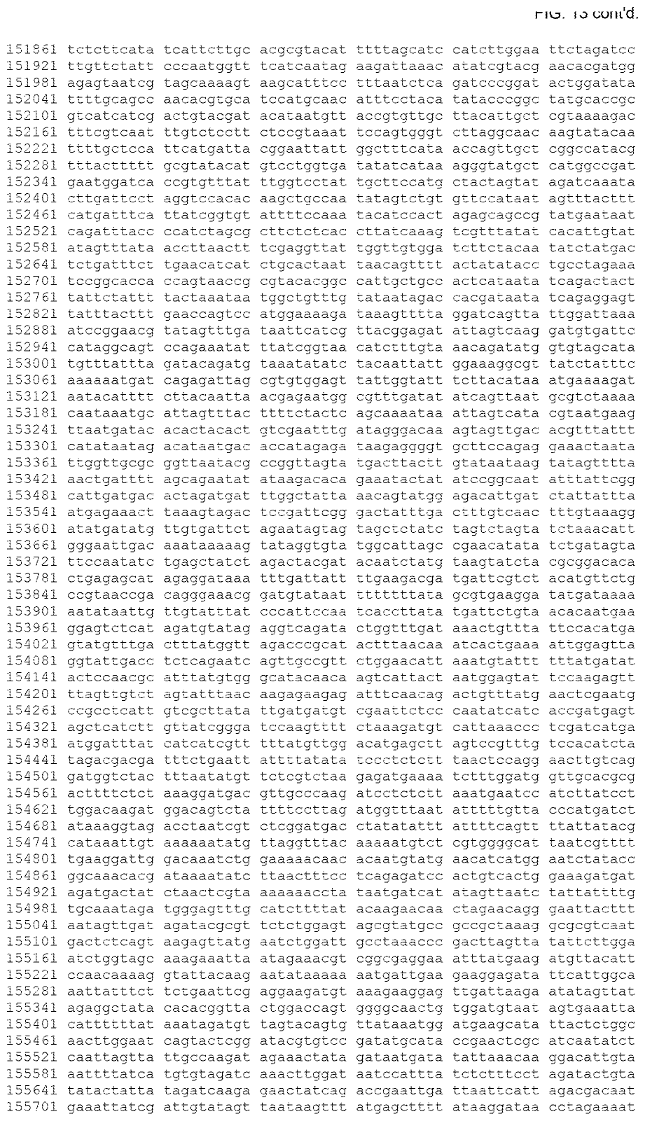

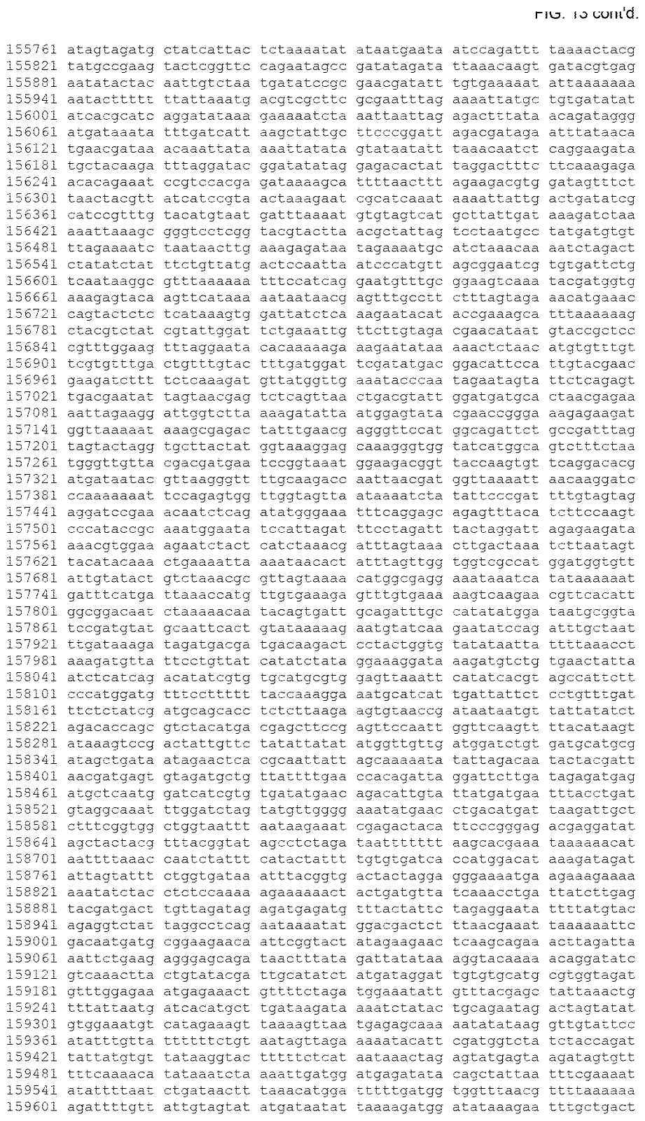

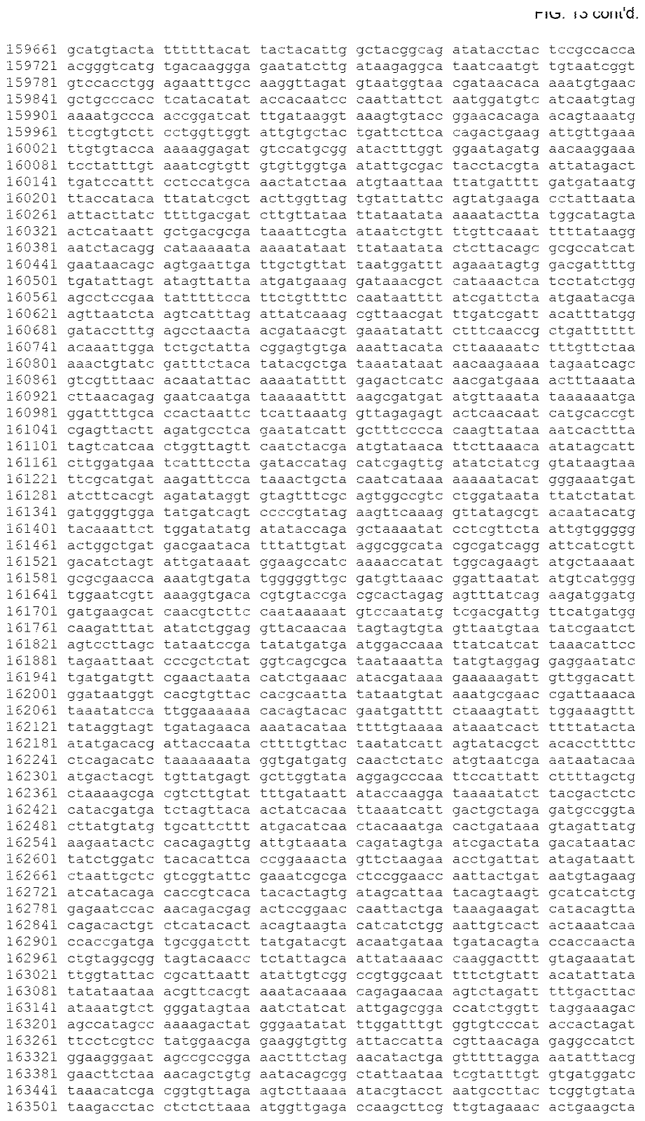

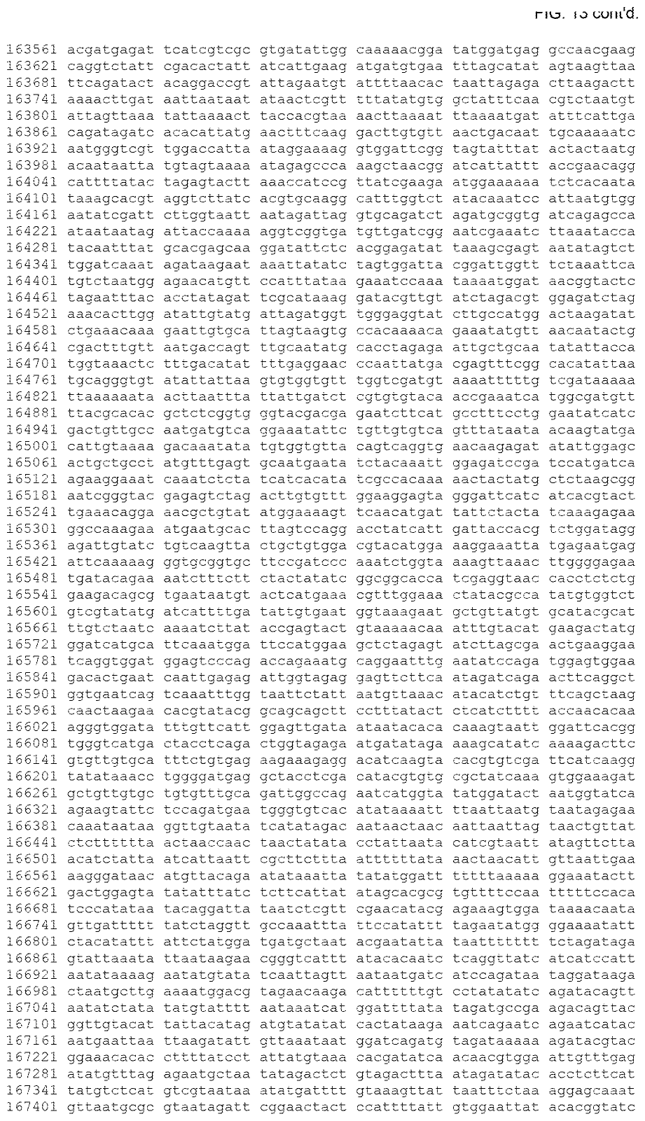

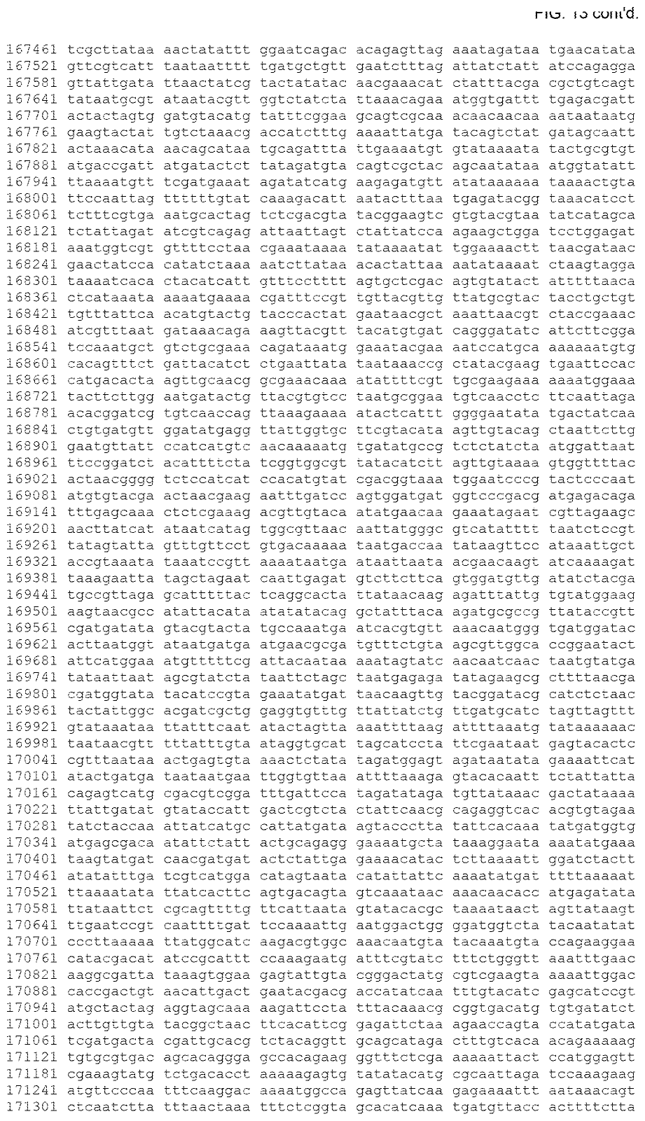

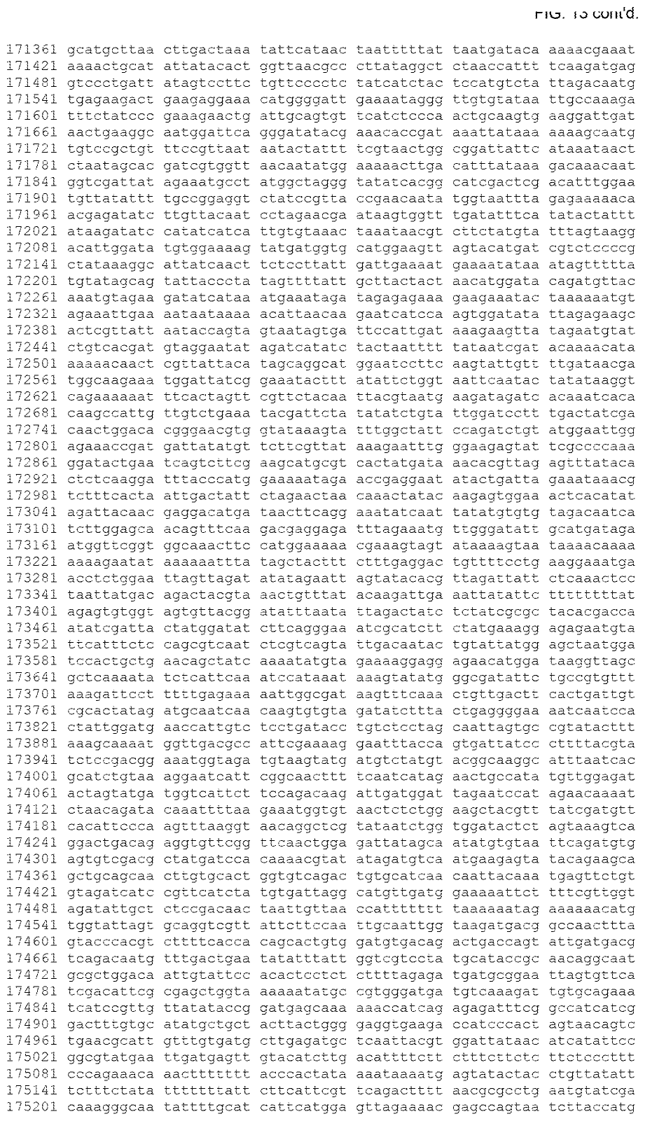

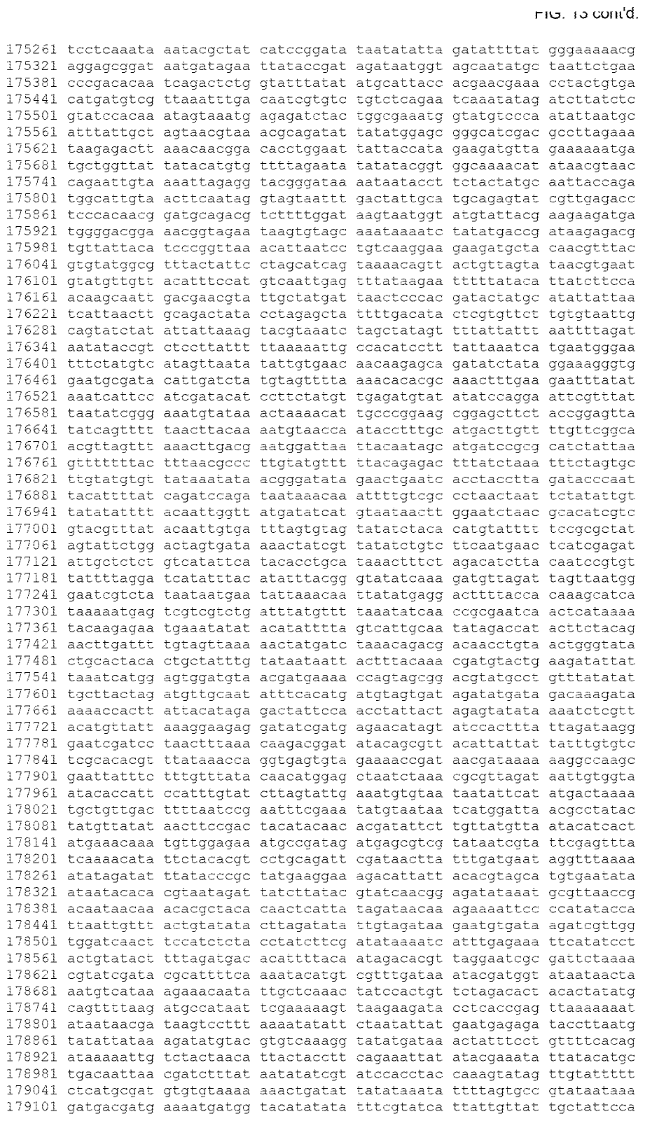

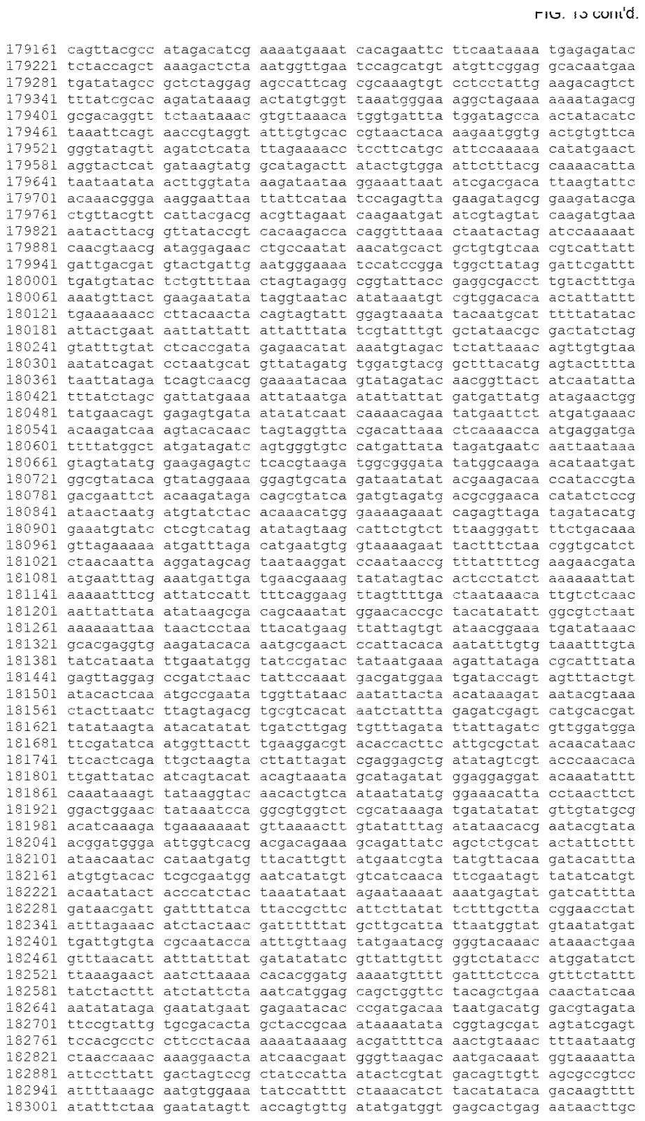

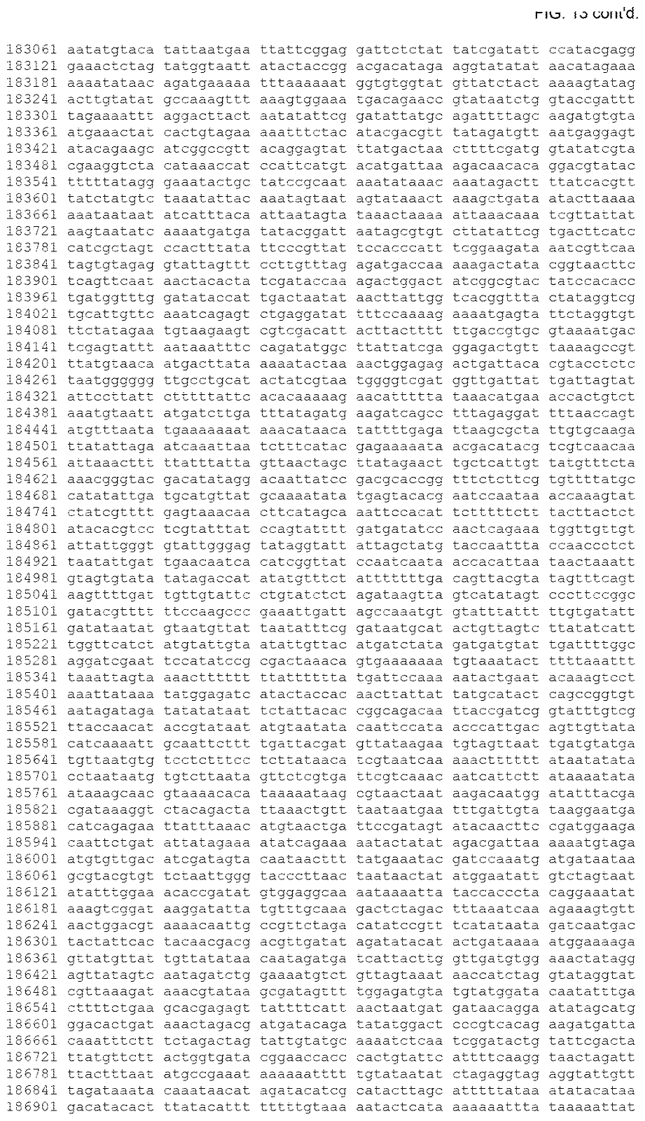

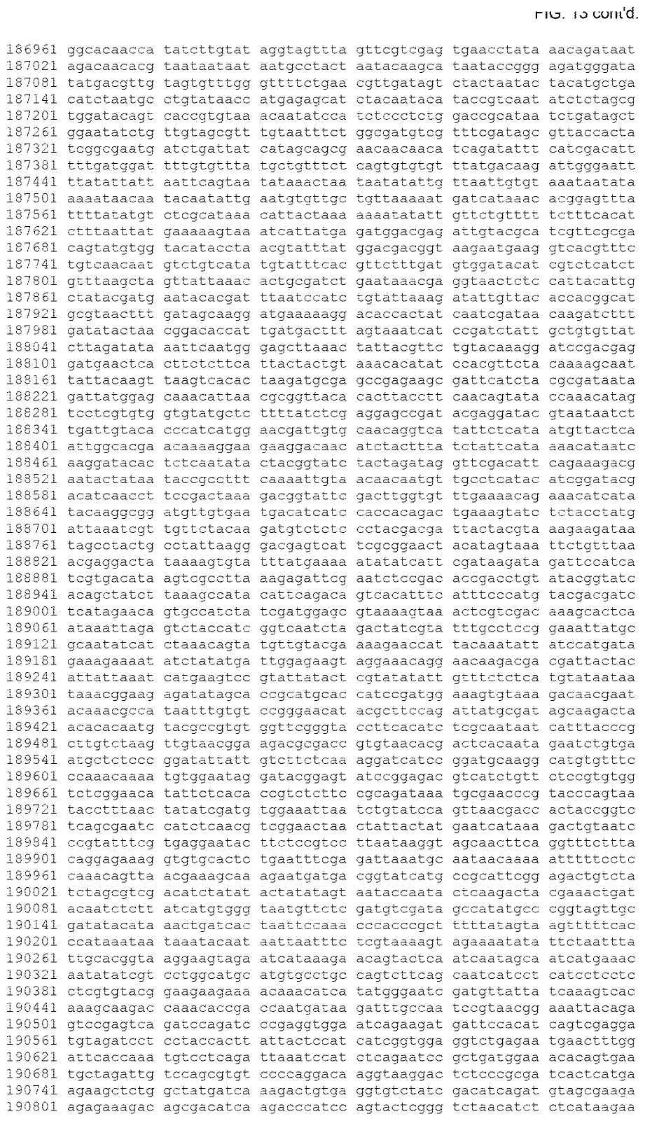

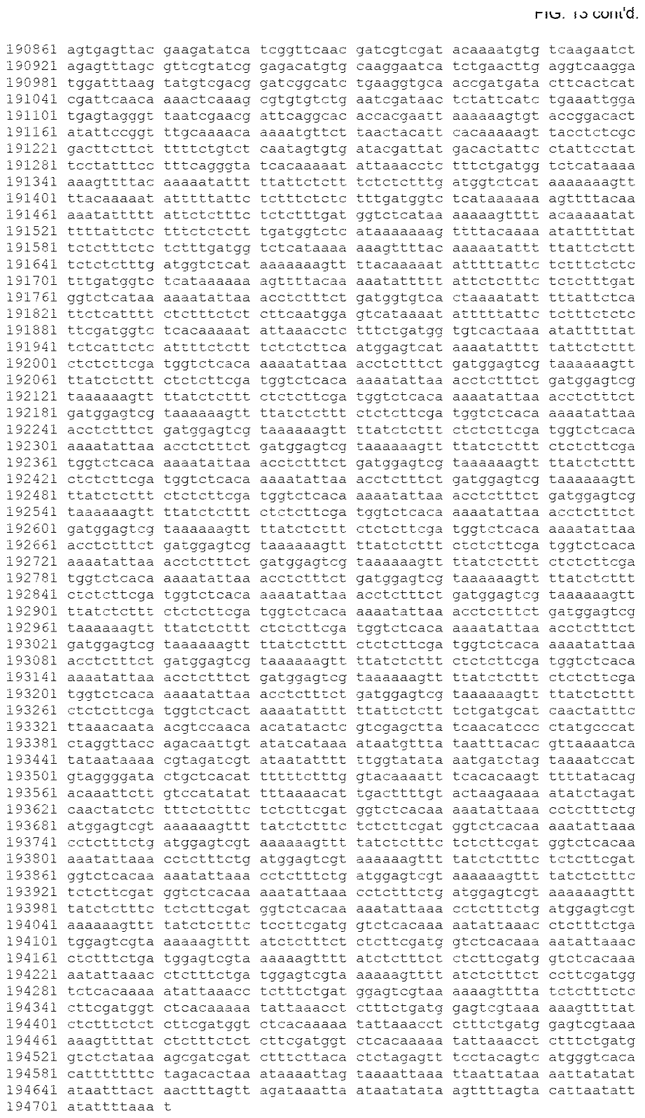

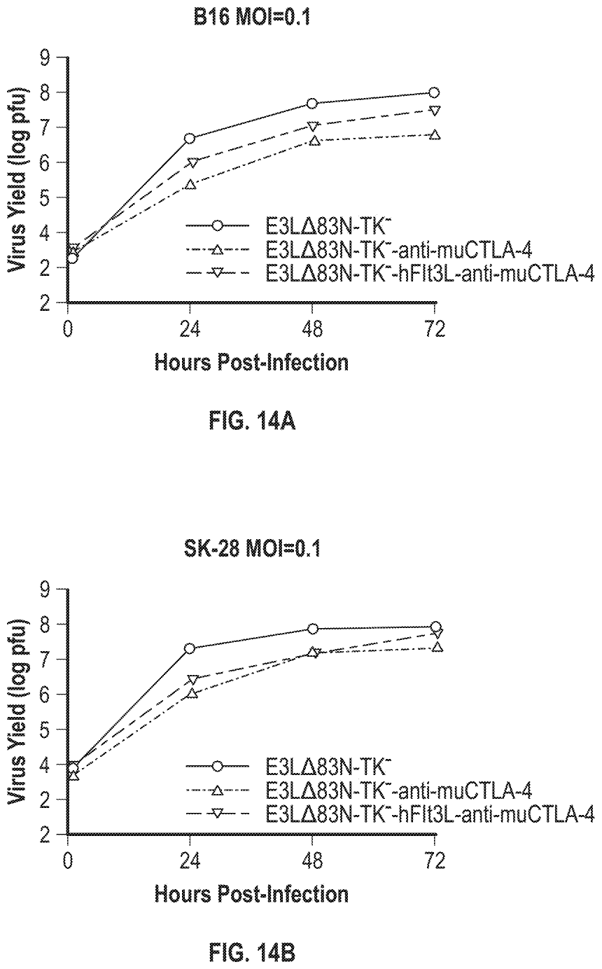

The replication capacities of E3LΔ83N-TK⁻-vector, E3LΔ83N-TK⁻-anti-muCTLA-4, and E3LΔ83N-TK⁻-hFlt3L-anti-muCTLA-4 were determined in murine B16-F10 melanoma cells and human SK-MEL-28 and SK-MEL-146 melanoma cells by infecting them at a MOI of 0.1. Cells were collected at various time points post infection (e.g., 1, 24, 48, and 72 hours) and viral yields (log pfu) were determined by titrating on BSC40 cells. FIG. 14A shows the graphs of viral yields plotted against hours post infection. E3LΔ83N-TK⁻-vector replicated efficiently in B16-F10 cells with viral titers increasing by more than 50,000-fold at 72h post-infection. E3LΔ83N-TK⁻-anti-muCTLA-4 and E3LΔ83N-TK⁻-hFlt3L-anti-muCTLA-4 viruses also replicated in B16-F10 cells, albeit with reduced efficiency compared to E3LΔ83N-TK⁻-vector. The fold changes of E3LΔ83N-TK⁻-anti-muCTLA-4 or E3LΔ83N-TK⁻-hFlt3L-anti-muCTLA-4 viral yields at 72 h over those at 1 h post infection were calculated to be about 2700 and 11500 folds, respectively. Similarly, the replication capacities of E3LΔ83N-TK⁻-vector, E3LΔ83N-TK⁻-anti-muCTLA-4, and E3LΔ83N-TK⁻-hFlt3L-anti-muCTLA-4 were determined in human melanoma cell lines SK-MEL-28 (FIG. 14B) and SK-MEL-146 (FIG. 14C) by infecting them at a MOI of 0.1. Cells were collected at various time points post infection and viral yields (log pfu) were determined by titrating on BSC40 cells. FIGS. 14B and 14C show the graphs of viral yields plotted against hours post infection. All recombinant viruses, E3LΔ83N-TK⁻-vector, E3LΔ83N-TK⁻-anti-muCTLA-4, and E3LΔ83N-TK⁻-hFlt3L-anti-muCTLA-4 have replicated efficiently in human melanoma cell lines SK-MEL-28 and SK-MEL-146.

TABLE 3
Quantitative data (fold change at 72 hpi) for results shown in FIGS.
12A, 12B, and 12C

| | | E3LΔ83N- | E3LΔ83N-TK⁻- |
|---|---|---|---|
| | E3LΔ83N-TK⁻- | TK⁻-anti- | hFlt3L-anti- |
| | vector | muCTLA-4 | muCTLA-4 |
| B16-F10 | 57142 | 2364 | 11538 |
| SK-MEL-28 | 12667 | 4615 | 6562 |
| SK-MEL-146 | 808 | 696 | 1074 |

### Example 13: Expression of Anti-muCTLA-4 in B16-F10 Melanoma Cells and MC38 Colon Cancer Cells Via Infection of E3LΔ83N-TK⁻-Anti-muCTLA-4 or E3LΔ83N-TK⁻-hFlt3L-Anti-muCTLA-4 Viruses

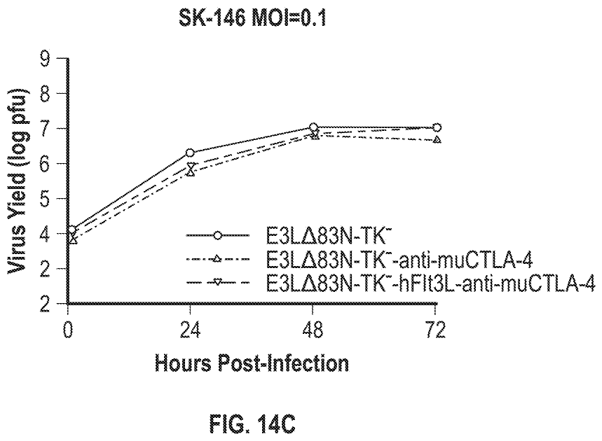

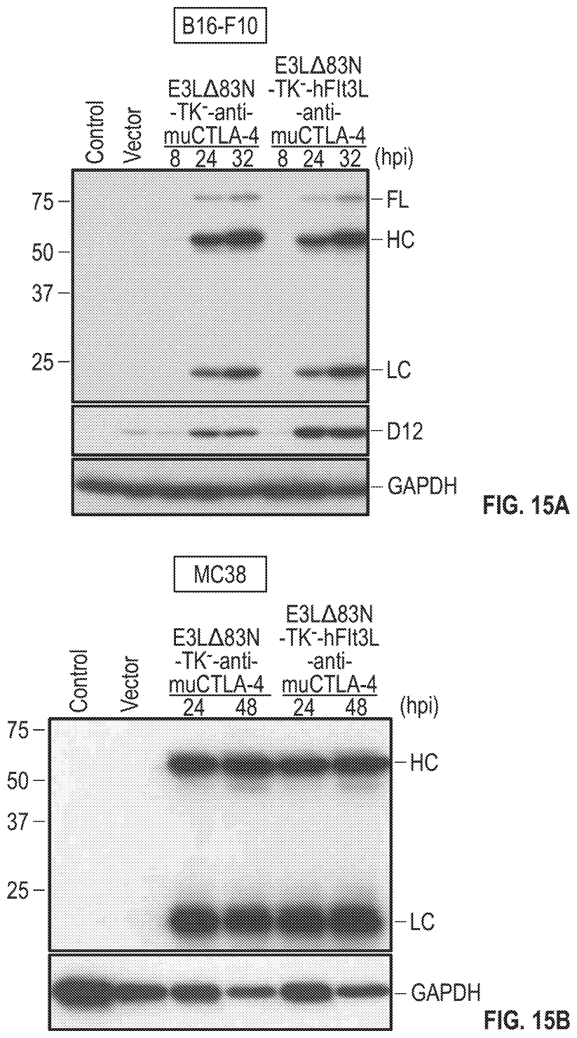

To determine whether E3LΔ83N-TK⁻ recombinant viruses are capable of expressing desired antibodies, B16-F10 murine melanoma cells or MC38 colon cancer cells were infected with E3LΔ83N-TK⁻, E3LΔ83N-TK⁻-anti-muCTLA-4, or E3LΔ83N-TK⁻-hFlt3L-anti-muCTLA-4 at a MOI of 10, and the expression of anti-muCTLA-4 antibody was measured. Cell lysates were collected at various times (e.g., 8, 24, and 32 hours) post infection. Western blot analyses were performed to determine the levels of the antibody expression. As shown in FIGS. 15A-15B, Western blot analysis reveals abundant expression levels of anti-muCTLA-4 antibody in both B16-F10 and MC38 cell infected with E3LΔ83N-TK⁻-anti-muCTLA-4 or E3LΔ83N-TK⁻-hFlt3L-anti-muCTLA-4 viruses. Accordingly, these results demonstrate that the recombinant viruses of the present technology have the capacity to express specific genes of interest in infected cells and are useful in methods for delivering the desired antibodies to cells.

### Example 14: Expression of Anti-muCTLA-4 in Human SK-MEL-28 Melanoma Cells

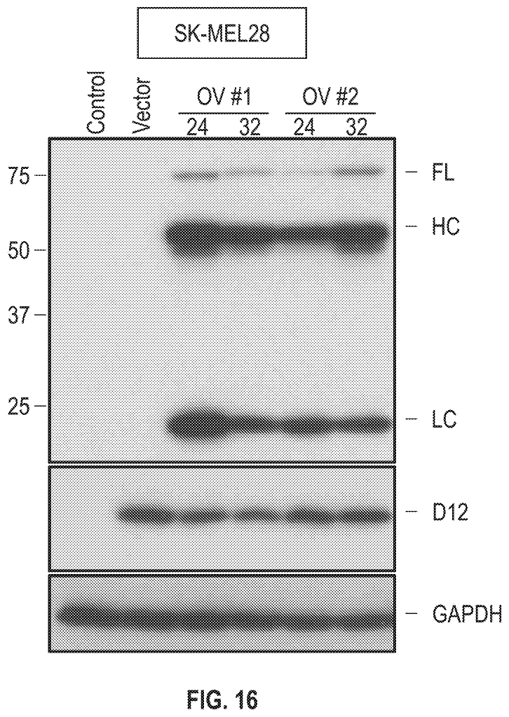

To determine whether recombinant E3LΔ83N-TK⁻-anti-muCTLA-4 or E3LΔ83N-TK⁻-hFlt3L-anti-muCTLA-4 virus infection results in the production of anti-CTLA-4 antibodies, human SK-MEL-28 melanoma cells were infected with E3LΔ83N-TK⁻-anti-muCTLA-4 or E3LΔ83N-TK⁻-hFlt3L-anti-muCTLA-4 at a MOI of 10. Cell lysates were collected at various times (e.g., 24 and 32 hours) post-infection, and polypeptides were separated using 10% SDS-PAGE. HRP-linked anti-mouse IgG (heavy and light chain) antibody was used to detect full-length (FL), heavy chain (HC), and light chain (LC) of anti-muCTLA-4 antibody. Anti-vaccinia-D12 antibody was used to check the expression of viral protein, and GAPDH was used as a loading control. As shown in FIG. 16, Western blot analysis shows the expression of the full-length (FL), heavy chain (HC), and light chain (LC) of anti-muCTLA-4 antibody in SK-MEL-28 melanoma cell lines. Accordingly, these results demonstrate that the recombinant viruses of the present technology have the capacity to express anti-CTLA-4 antibodies in infected cells and are useful in methods for delivering the antibodies to cells.

### Example 15: Expression of hFlt3L in Murine B16-F10 and Human SK-MEL-28 Melanoma Cells Via Infection of E3LΔ83N-TK⁻-hFlt3L-Anti-muCTLA-4

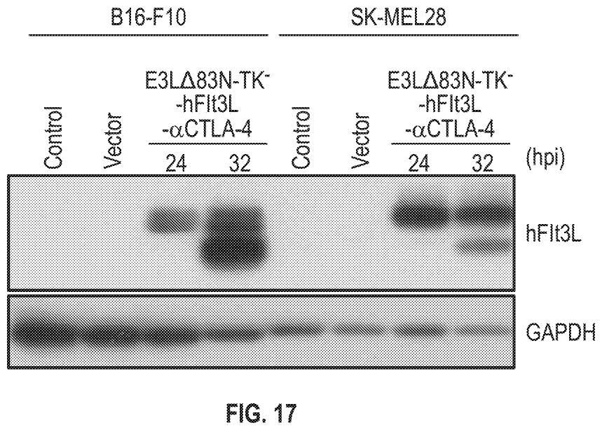

To test the expression of hFlt3L from the E3LΔ83N-TK⁻-hFlt3L-anti-muCTLA-4, murine B16-F10 or human SK-MEL-28 melanoma cells were mock infected or infected with E3LΔ83N-TK⁻-hFlt3L-anti-muCTLA-4 at a MOI of 10. Cell lysates were collected at various times (e.g., 24 and 32 hours) post-infection. Western blot analyses were performed to determine the levels of hFlt3L protein expression. As shown in FIG. 17, Western blot analysis reveals abundant levels of hFlt3L protein in the cell lysates. Accordingly, these results demonstrate that the E3LΔ83N-TK⁻-hFlt3L-anti-muCTLA-4 recombinant viruses of the present technology are capable of expressing hF1T3L proteins in infected cells and are useful in methods for expressing these proteins in tumor cells.

### Example 16: Secretion of Anti-muCTLA-4 from Murine B16-F10 Melanoma Cells and Human SK-MEL-28 Melanoma Cells Via Infection of E3LΔ83N-TK⁻-Anti-muCTLA-4 or E3LΔ83N-TK⁻-hFlt3L-Anti-muCTLA-4 Viruses

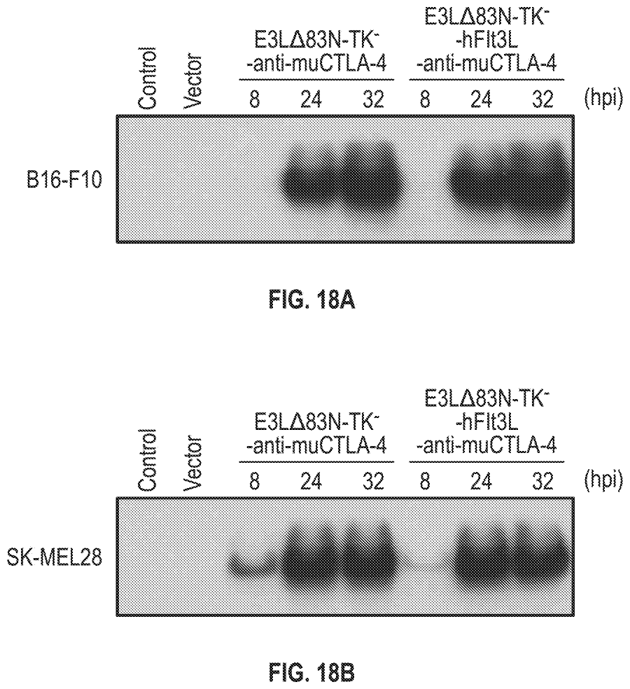

To determine whether recombinant E3LΔ83N-TK⁻-anti-muCTLA-4 or E3LΔ83N-TK⁻-hFlt3L-anti-muCTLA-4 virus infection results in the secretion of anti-CTLA-4 antibodies, murine B16-F10 melanoma cells or human SK-MEL-28 melanoma cells were infected with E3LΔ83N-TK⁻, E3LΔ83N-TK⁻-anti-muCTLA-4, or E3LΔ83N-TK⁻-hFlt3L-anti-muCTLA-4 at a MOI of 10. The cell culture supernatant was collected at various times (e.g., 8, 24, and 32 hours) post infection. Western blot analyses were performed to determine the levels of secreted anti-muCTLA-4 in culture supernatant. As shown in FIGS. 18A-18B, Western blot analysis reveals increased secretion of anti-muCTLA-4 antibody in the cell culture supernatant from murine B16-F10 and human SK-MEL-28 melanoma cells infected with recombinant E3LΔ83N-TK⁻-anti-muCTLA-4 or E3LΔ83N-TK⁻-hFlt3L-anti-muCTLA-4 viruses. Accordingly, these results demonstrate that the E3LΔ83N-TK⁻-anti-muCTLA-4 and E3LΔ83N-TK⁻-hFlt3L-anti-muCTLA-4 recombinant viruses of the present technology are capable of expressing and secreting anti-CTLA-4 antibodies in infected cells and are useful in methods for expressing these proteins in tumor cells and are useful in methods for delivering and secretion the desired antibodies to cells.

### Example 17: Secreted Anti-muCTLA-4 from Murine B16-F10 Melanoma Cells and Human SK-MEL-28 Melanoma Cells Via Infection of E3LΔ83N-TK⁻-Anti-muCTLA-4 or E3LΔ83N-TK⁻-hFlt3L-Anti-muCTLA-4 Viruses can Bind to Recombinant Murine CTLA-4 Protein

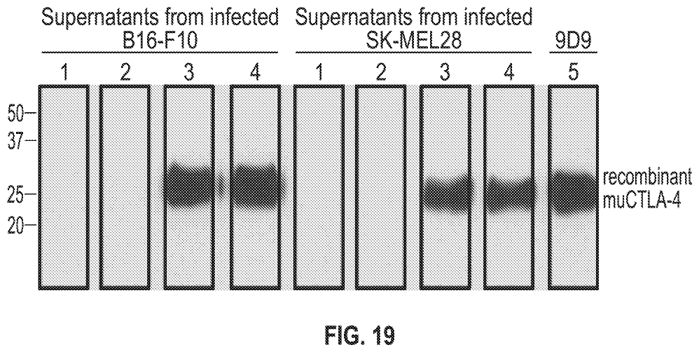

To determine whether secreted antibody from virus infected cells can bind to its intended target, murine B16-F10 melanoma cells or human SK-MEL-28 melanoma cells were infected with E3LΔ83N-TK⁻, E3LΔ83N-TK⁻-anti-muCTLA-4, or E3LΔ83N-TK⁻-hFlt3L-anti-muCTLA-4 at a MOI of 10. The cell culture supernatant was collected 24 hours post infection. The supernatant was incubated with membrane strips containing recombinant murine CTLA-4 protein to examine the binding of anti-muCTLA-4 antibody. As shown in FIG. 19, secreted anti-muCTLA-4 antibody in the supernatant from murine B16-F10 and human SK-MEL-28 melanoma cells infected with recombinant E3LΔ83N-TK⁻-anti-muCTLA-4 or E3LΔ83N-TK⁻-hFlt3L-anti-muCTLA-4 viruses can bind to the target CTLA-4 protein on the membrane strips. Accordingly, these data demonstrate that the secreted anti-muCTLA-4 antibodies murine B16-F10 or human SK-MEL-28 melanoma cells that were infected with the recombinant E3LΔ83N-TK⁻-anti-muCTLA-4 or E3LΔ83N-TK⁻-hFlt3L-anti-muCTLA-4 viruses of the present technology are capable of binding to their intended target and are useful in methods for generating functional antibodies.

### Example 18: Intratumorally Injected E3LΔ83N-TK⁻-hFlt3L-Anti-muCTLA-4 has the Capacity to Replicate and Express Desired Specific Gene in Implanted Tumors In Vivo

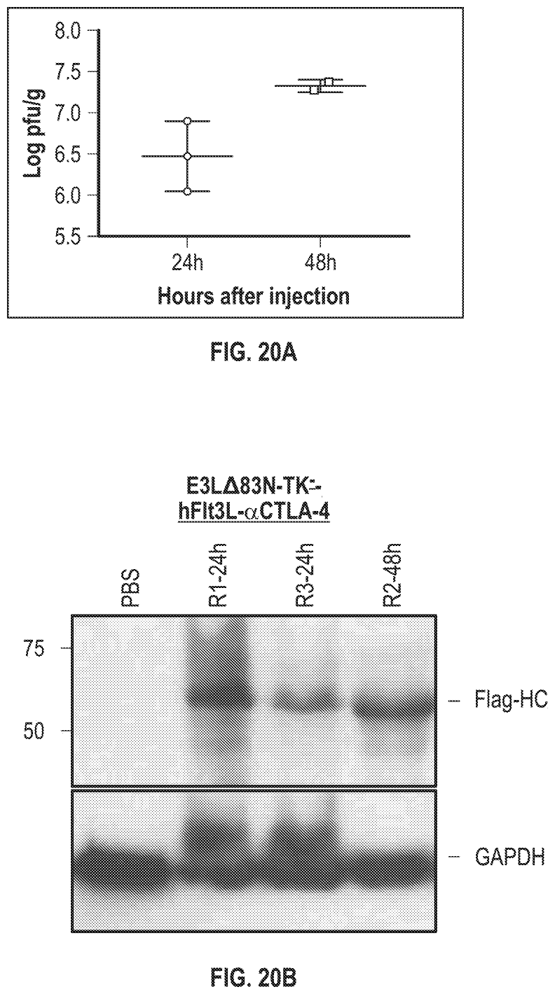

To assess whether intratumoral injection of E3LΔ83N-TK⁻-hFlt3L-anti-muCTLA-4 in B16-F10 melanomas leads to replication and expression of specific genes in implanted tumors in vivo, a unilateral tumor implantation model was used. Briefly, B16-F10 melanoma cells (5×10⁵ cells) were implanted intradermally into the shaved skin on the right flank of C57BL/6J mice. Seven days after tumor implantation, the tumors (about 3 mm in diameter) were injected with E3LΔ83N-TK⁻-hFlt3L-anti-muCTLA-4. Tumor samples were collected at various times (e.g., 24 and 48 hours) after virus injection. Viral yields of the E3LΔ83N-TK⁻-hFlt3L-anti-muCTLA-4 virus in the tumors were determined by titrating on BSC40 cells, and the expression of anti-muCTLA-4 antibody was examined by western blot. As show in FIGS. 20A-20B, there was modest virus replication in tumors at 48 hours after virus injection. The injected virus also expressed anti-muCTLA-4 antibody in tumors. Accordingly, these results demonstrate that the recombinant E3LΔ83N-TK⁻-hFlt3L-anti-muCTLA-4 virus of the present technology is replication competent in a subject and is capabale of expressing desired specific genes within of a subject.

### Example 19: Intratumoral Injection with E3LΔ83N-TK⁻-Anti-muCTLA-4 or E3LΔ83N-TK⁻-hFlt3L-Anti-muCTLA-4 Leads to the Generation of Antitumor CD8⁺ T-Cell Immunity

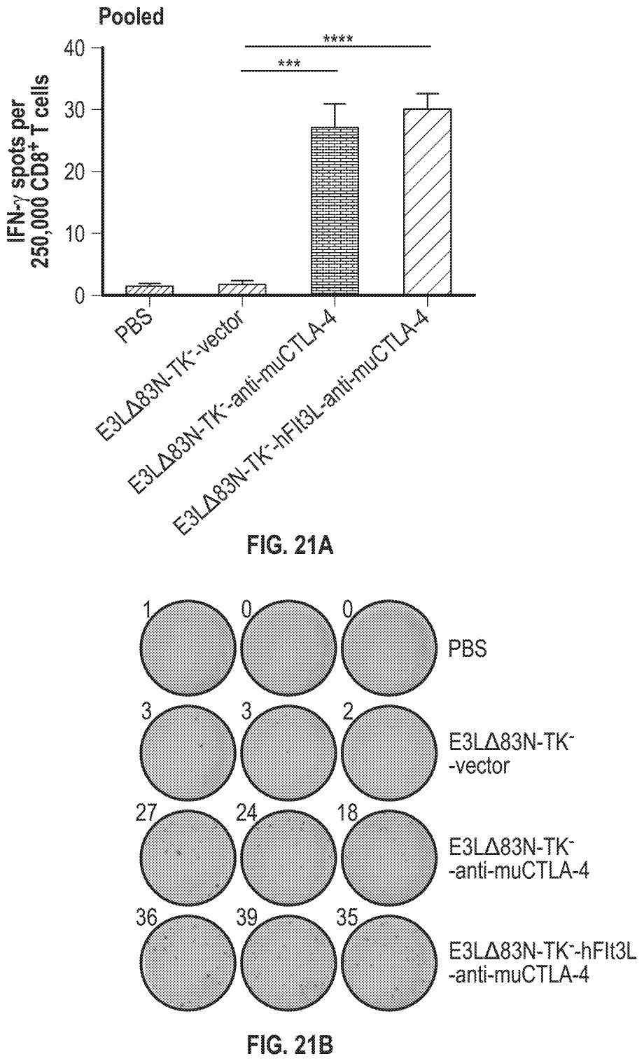

To assess whether mice gained antitumor memory T-cell immunity against the murine B16-F10 melanoma cancer after treatment with intratumoral injection of PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4, Enzyme-linked ImmunoSpot (ELISpot) was used. B16-F10 cells (5×10⁵ and 2.5×10⁵, respectively) were intradermally implanted into the shaved skin on the right and left flank of C57BL/6J mice. Seven days after tumor implantation the tumors on the right flank (about 3 mm in diameter) were injected with PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4. The injections were repeated three days later, followed by euthanization three days after the second injection. ELISpot was performed to assess the generation of antitumor specific CD8⁺ T cells in the spleens of mice treated with the recombinant viruses. Briefly, CD8⁺ T cells were isolated from splenocytes and 2.5×10⁵ cells were cultured with irradiated B16-F10 cells overnight at 37° C. in anti-IFN-γ-coated BD ELISpot plate microwells. CD8⁺ T cells were stimulated with B16-F10 cells irradiated with an γ-irradiator and IFN-γ secretion was detected with an anti-IFN-γ antibody. FIG. 21A shows the numbers of IFN-γ⁺ spots per 250,000 CD8⁺ T cells from individual mouse treated with either PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4. FIG. 21B shows the numbers of IFN-γ⁺ spots per 250,000 CD8⁺ T cells pooled from mice in each group treated with either PBS, E3LΔ83N-TK⁻, E3LΔ83N-TK⁻-hFlt3L, E3LΔ83N-TK⁻-anti-muCTLA-4, or E3LΔ83N-TK⁻-hFlt3L-anti-muCTLA-4. These results demonstrate that intratumoral injection of E3LΔ83N-TK⁻-anti-muCTLA-4 or E3LΔ83N-TK⁻-hFlt3L-anti-muCTLA-4 increases antitumor CD8⁺ T cells in treated mice. In addition, the results demonstrate that the intratumoral injection of E3LΔ83N-TK⁻-anti-muCTLA-4 or E3LΔ83N-TK⁻-hFlt3L-anti-muCTLA-4 is more effective than E3LΔ83N-TK⁻ or E3LΔ83N-TK⁻-hFlt3L in generating antitumor CD8⁺ T cells in treated mice in a murine B16-F10 melanoma bilateral implantation model. Accordingly, these results demonstrate that the recombinant E3LΔ83N-TK⁻-anti-muCTLA-4 and E3LΔ83N-TK⁻-hFlt3L-anti-muCTLA-4 viruses of the present technology are effective in enhancing or promoting an immune response in the subject and in increased cytotoxic CD8⁺ T cells within of a subject.

### Example 20: Viral Therapy of Intratumoral Injection of E3LΔ83N-TK⁻-Anti-muCTLA-4 or E3LΔ83N-TK⁻-hFlt3L-Anti-muCTLA-4

This example demonstrates the use of recombinant viruses of the present technology, such as E3LΔ83N-TK⁻-anti-huCTLA-4 or E3LΔ83N-TK⁻-hFlt3L-anti-huCTLA-4 in the treatment of solid tumors, such as melanoma.

### Methods

Tumor implantation and intratumoral injection with viruses. A bilateral tumor implantation model was used. B16-F10 melanoma cells were implanted intradermally into the shaved skin on the right (5×10⁵ cells) and left (1×10⁵ cells) flanks of a C57BL/6J mouse. After 8 days post implantation, the larger tumors on the right flank (about 3 mm or larger in diameter) were injected twice per week with PBS, E3LΔ83N-TK⁻, E3LΔ83N-TK⁻-anti-muCTLA-4, E3LΔ83N-TK⁻-hFlt3L-anti-muCTLA-4, when the mice were under anesthesia. Mice were monitored for survival and the tumor sizes were measured twice a week.

### Results

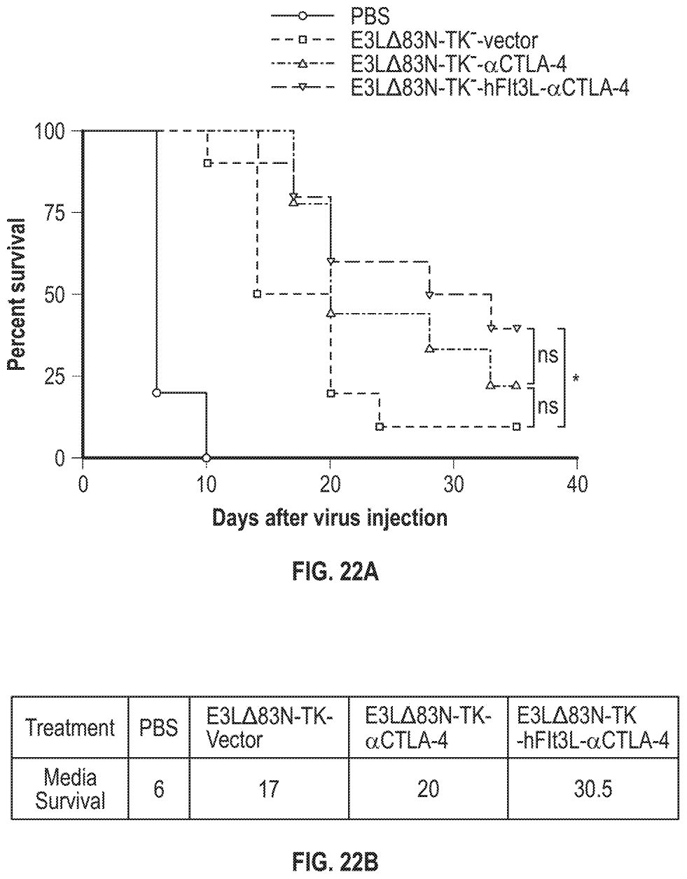

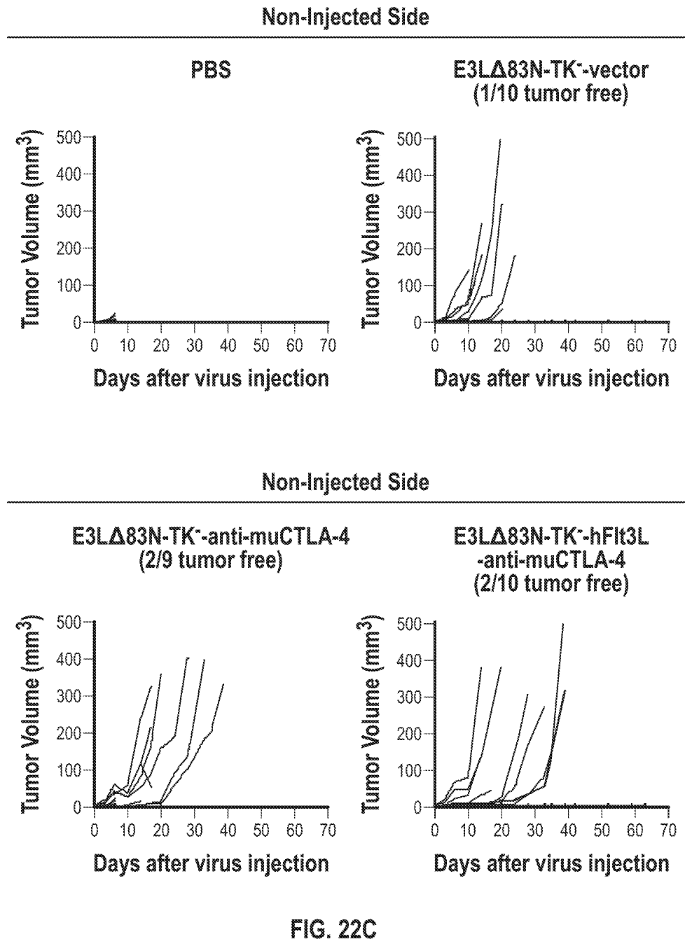

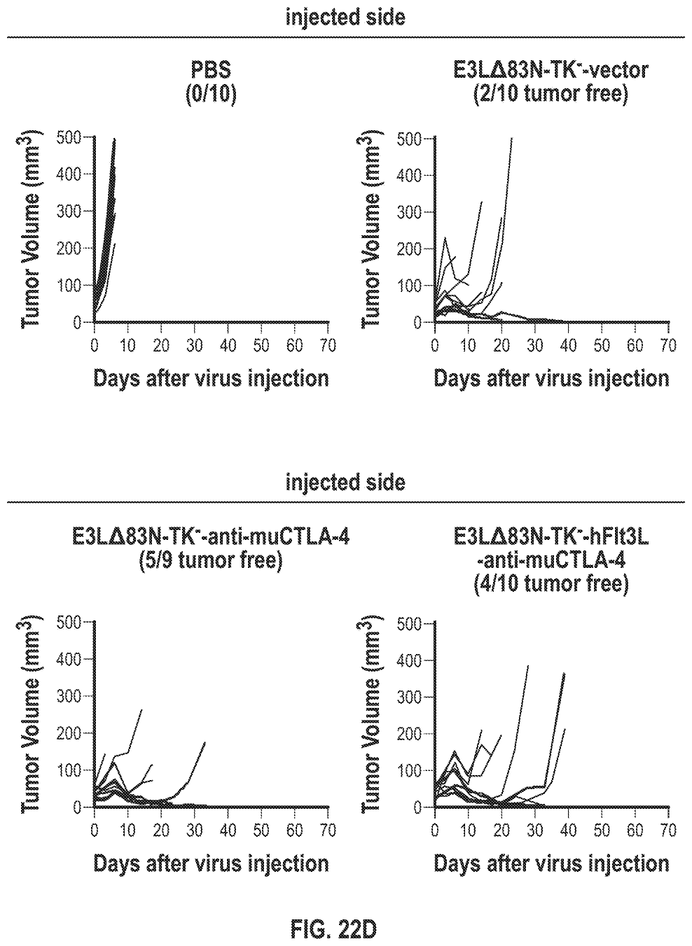

As shown in FIGS. 22A-22D, viral therapy delayed tumor growth on the non-injected side of the mice when compared with PBS control. Also, viruses expressing anti-muCTLA-4 and human Flt3L delayed the tumor growth when compared with control virus. Accordingly, these results demonstrate that the compositions of the present technology are useful in methods for treating a solid tumor.

### REFERENCES

1. C. Jochems, J. Schlom, Tumor-infiltrating immune cells and prognosis: the potential link between conventional cancer therapy and immunity. Exp Biol Med (Maywood) 236, 567-579 (2011).
2. B. Mlecnik, G. Bindea, F. Pages, J. Galon, Tumor immunosurveillance in human cancers. Cancer Metastasis Rev 30, 5-12 (2011).
3. H. Angell, J. Galon, From the immune contexture to the Immunoscore: the role of prognostic and predictive immune markers in cancer. Curr Opin Immunol 25, 261-267 (2013).
4. F. Garrido, I. Algarra, A. M. Garcia-Lora, The escape of cancer from T lymphocytes: immunoselection of MHC class I loss variants harboring structural-irreversible “hard” lesions. Cancer Immunol Immunother 59, 1601-1606 (2010).
5. G. Gerlini et al., Metastatic melanoma secreted IL-10 down-regulates CD1 molecules on dendritic cells in metastatic tumor lesions. Am J Pathol 165, 1853-1863 (2004).
6. P. Sharma, J. P. Allison, The future of immune checkpoint therapy. Science 348, 56-61 (2015). 7. S. L. Topalian, C. G. Drake, D. M. Pardoll, Targeting the PD-1/B7-H1(PD-L1) pathway to activate anti-tumor immunity. Curr Opin Immunol 24, 207-212 (2012).
8. D. H. Kim, S. H. Thorne, Targeted and armed oncolytic poxviruses: a novel multi-mechanistic therapeutic class for cancer. Nature reviews. Cancer 9, 64-71 (2009).
9. B. Moss, Poxviridae: The viruses and their replication. e. D. M. Knipe, Ed., In Fields Virology (Lippincott Williams & Wilkins, 2007), pp. pp. 2905-2946.
10. C. J. Breitbach, S. H. Thorne, J. C. Bell, D. H. Kim, Targeted and armed oncolytic poxviruses for cancer: the lead example of JX-594. Current pharmaceutical biotechnology 13, 1768-1772 (2012).
11. B. H. Park et al., Use of a targeted oncolytic poxvirus, JX-594, in patients with refractory primary or metastatic liver cancer: a phase I trial. Lancet Oncol 9, 533-542 (2008).
12. D. H. Kim, Y. Wang, F. Le Boeuf, J. Bell, S. H. Thorne, Targeting of interferon-beta to produce a specific, multi-mechanistic oncolytic vaccinia virus. PLoS Med 4, e353 (2007).
13. S. H. Thorne et al., Rational strain selection and engineering creates a broad-spectrum, systemically effective oncolytic poxvirus, JX-963. J Clin Invest 117, 3350-3358 (2007).
14. J. Engelmayer et al., Vaccinia virus inhibits the maturation of human dendritic cells: a novel mechanism of immune evasion. J Immunol 163, 6762-6768 (1999).
15. L. Jenne, C. Hauser, J. F. Arrighi, J. H. Saurat, A. W. Hugin, Poxvirus as a vector to transduce human dendritic cells for immunotherapy: abortive infection but reduced APC function. Gene therapy 7, 1575-1583 (2000).
16. P. Li et al., Disruption of MHC class II-restricted antigen presentation by vaccinia virus. J Immunol 175, 6481-6488 (2005).
17. L. Deng, P. Dai, W. Ding, R. D. Granstein, S. Shuman, Vaccinia virus infection attenuates innate immune responses and antigen presentation by epidermal dendritic cells. J Virol 80, 9977-9987 (2006).
18. R. Drillien, D. Spehner, D. Hanau, Modified vaccinia virus Ankara induces moderate activation of human dendritic cells. J Gen Virol 85, 2167-2175 (2004).
19. P. Dai et al., Modified vaccinia virus Ankara triggers type I IFN production in murine conventional dendritic cells via a cGAS/STING-mediated cytosolic DNA-sensing pathway. PLoS Pathog 10, e1003989 (2014).
20. G. Sutter, C. Staib, Vaccinia vectors as candidate vaccines: the development of modified vaccinia virus Ankara for antigen delivery. Current drug targets. Infectious disorders 3, 263-271 (2003).
21. C. E. Gomez, J. L. Najera, M. Krupa, M. Esteban, The poxvirus vectors MVA and NYVAC as gene delivery systems for vaccination against infectious diseases and cancer. Curr Gene Ther 8, 97-120 (2008).
22. C. E. Gomez, J. L. Najera, M. Krupa, B. Perdiguero, M. Esteban, MVA and NYVAC as vaccines against emergent infectious diseases and cancer. Curr Gene Ther 11, 189-217 (2011).
23. P. A. Goepfert et al., Phase 1 safety and immunogenicity testing of DNA and recombinant modified vaccinia Ankara vaccines expressing HIV-1 virus-like particles. J Infect Dis 203, 610-619 (2011).
24. L. S. Wyatt, I. M. Belyakov, P. L. Earl, J. A. Berzofsky, B. Moss, Enhanced cell surface expression, immunogenicity and genetic stability resulting from a spontaneous truncation of HIV Env expressed by a recombinant MVA. Virology 372, 260-272 (2008).
25. F. Garcia et al., Safety and immunogenicity of a modified pox vector-based HIV/AIDS vaccine candidate expressing Env, Gag, Pol and Nef proteins of HIV-1 subtype B (MVA-B) in healthy HIV-1-uninfected volunteers: A phase I clinical trial (RISVACO2). Vaccine 29, 8309-8316 (2011).
26. M. Tagliamonte, A. Petrizzo, M. L. Tornesello, F. M. Buonaguro, L. Buonaguro, Antigen-specific vaccines for cancer treatment. Human vaccines & immunotherapeutics 10, 3332-3346 (2014).
27. P. H. Verardi, A. Titong, C. J. Hagen, A vaccinia virus renaissance: new vaccine and immunotherapeutic uses after smallpox eradication. Human vaccines & immunotherapeutics 8, 961-970 (2012).
28. S. Hornemann et al., Replication of modified vaccinia virus Ankara in primary chicken embryo fibroblasts requires expression of the interferon resistance gene E3L. J Virol 77, 8394-8407 (2003).
29. H. Ludwig et al., Role of viral factor E3L in modified vaccinia virus ankara infection of human HeLa Cells: regulation of the virus life cycle and identification of differentially expressed host genes. J Virol 79, 2584-2596 (2005).
30. S. F. Fischer et al., Modified vaccinia virus Ankara protein F1L is a novel BH3-domain-binding protein and acts together with the early viral protein E3L to block virus-associated apoptosis. Cell Death Differ 13, 109-118 (2006).
31. J. C. Castle et al., Exploiting the mutanome for tumor vaccination. Cancer Res 72, 1081-1091 (2012).
32. T. N. Schumacher, R. D. Schreiber, Neoantigens in cancer immunotherapy. Science 348, 69-74 (2015).
33. I. Mellman, G. Coukos, G. Dranoff, Cancer immunotherapy comes of age. Nature 480, 480-489 (2011).
34. K. S. Peggs, S. A. Quezada, C. A. Chambers, A. J. Korman, J. P. Allison, Blockade of CTLA-4 on both effector and regulatory T-cell compartments contributes to the antitumor activity of anti-CTLA-4 antibodies. J Exp Med 206, 1717-1725 (2009).
35. K. Wing et al., CTLA-4 control over Foxp3+ regulatory T-cell function. Science 322, 271-275 (2008).
36. D. R. Leach, M. F. Krummel, J. P. Allison, Enhancement of antitumor immunity by CTLA-4 blockade. Science 271, 1734-1736 (1996).
37. F. S. Hodi et al., Improved survival with ipilimumab in patients with metastatic melanoma. The New England journal of medicine 363, 711-723 (2010).
38. C. Robert et al., Ipilimumab plus dacarbazine for previously untreated metastatic melanoma. The New England journal of medicine 364, 2517-2526 (2011).
39. S. L. Topalian, C. G. Drake, D. M. Pardoll, Immune checkpoint blockade: a common denominator approach to cancer therapy. Cancer Cell 27, 450-461 (2015).
40. D. A. Oble, R. Loewe, P. Yu, M. C. Mihm, Jr., Focus on TILs: prognostic significance of tumor infiltrating lymphocytes in human melanoma. Cancer immunity 9, 3 (2009).
41. K. E. Lacy, S. N. Karagiannis, and F. O. Nestle, Immunotherapy for Melanoma. Expert Rev Dermatol 7, 51-68 (2012).
42. J. D. Wolchok et al., Ipilimumab monotherapy in patients with pretreated advanced melanoma: a randomised, double-blind, multicentre, phase 2, dose-ranging study. Lancet Oncol 11, 155-164 (2010).
43. J. D. Wolchok et al., Nivolumab plus ipilimumab in advanced melanoma. The New England journal of medicine 369, 122-133 (2013).
44. O. Hamid et al., Safety and Tumor Responses with Lambrolizumab (Anti-PD-1) in Melanoma. The New England journal of medicine, (2013).
45. P. C. Tumeh et al., PD-1 blockade induces responses by inhibiting adaptive immune resistance. Nature 515, 568-571 (2014).
46. D. Zamarin et al., Localized oncolytic virotherapy overcomes systemic tumor resistance to immune checkpoint blockade immunotherapy. Science translational medicine 6, 226ra232 (2014).
47. M. B. Fuertes, S. R. Woo, B. Burnett, Y. X. Fu, T. F. Gajewski, Type I interferon response and innate immune sensing of cancer. Trends Immunol 34, 67-73 (2013).
48. M. S. Diamond et al., Type I interferon is selectively required by dendritic cells for immune rejection of tumors. J Exp Med 208, 1989-2003 (2011).
49. M. B. Fuertes et al., Host type I IFN signals are required for antitumor CD8⁺ T-cell responses through CD8{alpha}⁺ dendritic cells. J Exp Med 208, 2005-2016 (2011).
50. S. R. Woo et al., STING-dependent cytosolic DNA sensing mediates innate immune recognition of immunogenic tumors. Immunity 41, 830-842 (2014).
51. L. Deng et al., STING-Dependent Cytosolic DNA Sensing Promotes Radiation-Induced Type I Interferon-Dependent Antitumor Immunity in Immunogenic Tumors. Immunity 41, 843-852 (2014).
52. J. P. Huber, J. D. Farrar, Regulation of effector and memory T-cell functions by type I interferon. Immunology 132, 466-474 (2011).
53. D. M. Pardoll, The blockade of immune checkpoints in cancer immunotherapy. Nature reviews. Cancer 12, 252-264 (2012).
54. J. Nemunaitis, Oncolytic viruses. Invest New Drugs 17, 375-386 (1999).
55. D. Kim, R. L. Martuza, J. Zwiebel, Replication-selective virotherapy for cancer: Biological principles, risk management and future directions. Nat Med 7, 781-787 (2001).
56. M. C. Coffey, J. E. Strong, P. A. Forsyth, P. W. Lee, Reovirus therapy of tumors with activated Ras pathway. Science 282, 1332-1334 (1998).
57. A. Mayr, H. Stickl, H. K. Muller, K. Danner, H. Singer, [The smallpox vaccination strain MVA: marker, genetic structure, experience gained with the parenteral vaccination and behavior in organisms with a debilitated defence mechanism (author's transl)]. Zentralbl Bakteriol B 167, 375-390 (1978).
58. C. Verheust, M. Goossens, K. Pauwels, D. Breyer, Biosafety aspects of modified vaccinia virus Ankara (MVA)-based vectors used for gene therapy or vaccination. Vaccine 30, 2623-2632 (2012).
59. G. Antoine, F. Scheiflinger, F. Dorner, F. G. Falkner, The complete genomic sequence of the modified vaccinia Ankara strain: comparison with other orthopoxviruses. Virology 244, 365-396 (1998).
60. A. Mayr, Hochstein-Mintzel V, Stickl H., Passage history, properties, and applicability of the attenuated vaccinia virus strain MVA [in German]. Infection 3, 6-14 (1975).
61. H. Meyer, G. Sutter, A. Mayr, Mapping of deletions in the genome of the highly attenuated vaccinia virus MVA and their influence on virulence. J Gen Virol 72 (Pt 5), 1031-1038 (1991).
62. S. Brandler et al., Preclinical studies of a modified vaccinia virus Ankara-based HIV candidate vaccine: antigen presentation and antiviral effect. J Virol 84, 5314-5328 (2010).
63. A. Takaoka, T. Taniguchi, New aspects of IFN-alpha/beta signalling in immunity, oncogenesis and bone metabolism. Cancer Sci 94, 405-411 (2003).
64. D. Nagorsen, E. Wang, F. M. Marincola, J. Even, Transcriptional analysis of tumor-specific T-cell responses in cancer patients. Crit Rev Immunol 22, 449-462 (2002).
65. S. Pramanick, Singodia, D., and Chandel, V., Excipient selection in parenteral formulation development. Pharma Times 45, 65-77 (2013).
66. J. R. Weaver et al., The identification and characterization of a monoclonal antibody to the vaccinia virus E3 protein. Virus Res 130, 269-274 (2007).
67. M. Sato et al., Distinct and essential roles of transcription factors IRF-3 and IRF-7 in response to viruses for IFN-alpha/beta gene induction. Immunity 13, 539-548 (2000).
68. H. Ishikawa, G. N. Barber, STING is an endoplasmic reticulum adaptor that facilitates innate immune signalling. Nature 455, 674-678 (2008).
69. G. N. Barber, Innate immune DNA sensing pathways: STING, AIMII and the regulation of interferon production and inflammatory responses. Curr Opin Immunol 23, 10-20 (2011).
70. J. D. Sauer et al., The N-ethyl-N-nitrosourea-induced Goldenticket mouse mutant reveals an essential function of Sting in the in vivo interferon response to Listeria monocytogenes and cyclic dinucleotides. Infection and immunity 79, 688-694 (2011).
71. L. Sun, J. Wu, F. Du, X. Chen, Z. J. Chen, Cyclic GMP-AMP synthase is a cytosolic DNA sensor that activates the type I interferon pathway. Science 339, 786-791 (2013).
72. X. D. Li et al., Pivotal roles of cGAS-cGAMP signaling in antiviral defense and immune adjuvant effects. Science 341, 1390-1394 (2013).
73. J. Wu et al., Cyclic GMP-AMP is an endogenous second messenger in innate immune signaling by cytosolic DNA. Science 339, 826-830 (2013).
74. P. Gao et al., Structure-function analysis of STING activation by c[G(2′,5′)pA(3′,5′)p] and targeting by antiviral DMXAA. Cell 154, 748-762 (2013).
75. K. V. Kibler et al., Double-stranded RNA is a trigger for apoptosis in vaccinia virus-infected cells. J Virol 71, 1992-2003 (1997).
76. S. B. Lee, M. Esteban, The interferon-induced double-stranded RNA-activated protein kinase induces apoptosis. Virology 199, 491-496 (1994).
77. D. Tormo et al., Targeted activation of innate immunity for therapeutic induction of autophagy and apoptosis in melanoma cells. Cancer Cell. 2009 Aug. 4; 16(2):103-14. doi: 10.1016/j.ccr.2009.07.004.
78. L. Gitlin et al., Essential role of mda-5 in type I IFN responses to polyriboinosinic:polyribocytidylic acid and encephalomyocarditis picornavirus. Proc Natl Acad Sci USA. 2006 May 30; 103(22):8459-64. Epub 2006 May 19.
79. M. E. Perkus, et al., Vaccinia virus host genes. Virology 179(1):276-286 (1990).
80. G. Sivan, et al., Identification of Restriction Factors by Human Genome-Wide RNA Interference Screening of Viral Host Range Mutants Exemplified by Discovery of SAMD9 and WDR6 as Inhibitors of the Vaccinia Virus K1L⁻ C7L⁻ Mutant. mBio 6(4):e01122-15 (July/August 2015).
81. X. Meng, et al., C7L Family of Poxvirus Host Range Genes Inhibits Antiviral Activities Induced by Type I Interferons and Interferon Regulatory Factor 1. J. Virol. 86(8):4538-4547 (2012).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. An engineered E3LΔ83N-TK⁻-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding an anti-cytotoxic T lymphocyte-associated antigen (CTLA-4) antibody heavy chain (HC), and an anti-CTLA-4 antibody light chain (LC), wherein the HC and LC are separated by a nucleotide sequence that encodes, in the 5′ to 3′ direction, a protease cleavage site and a 2A peptide (Pep2A) sequence.

2. The engineered E3LΔ83N-TK⁻-anti-CTLA-4 vaccinia virus of claim 1, wherein the protease cleavage site is a furin cleavage site.

3. The engineered E3LΔ83N-TK⁻-anti-CTLA-4 vaccinia virus of claim 1 or claim 2, wherein the expression cassette further comprises a promoter that is capable of directing expression of the open reading frame.

4. The engineered E3LΔ83N-TK⁻-anti-CTLA-4 vaccinia virus of any one of claims 1-3, wherein the heterologous nucleic acid sequence further comprises an additional expression cassette comprising an open reading frame that encodes a selectable marker operably linked to a promoter that is capable of directing expression of the selectable marker.

5. The engineered E3LΔ83N-TK⁻-anti-CTLA-4 vaccinia virus of claim 4, wherein the selectable marker is a xanthine-guanine phosphoribosyl transferase (gpt) gene, a bioluminescent protein, a fluorescent protein, a chemiluminescent protein, or any combination thereof.

6. The engineered E3LΔ83N-TK⁻-anti-CTLA-4 vaccinia virus of any one of claims 1-5, wherein the virus does not produce a full-length thymidine kinase (TK) gene product.

7. The engineered E3LΔ83N-TK⁻-anti-CTLA-4 vaccinia virus of any one of claims 1-6, wherein the open reading frame comprises the nucleotide sequence set forth in SEQ ID NO: 1.

8. The engineered E3LΔ83N-TK⁻-anti-CTLA-4 vaccinia virus of any one of claims 1-6, wherein the open reading frame encodes an anti-CTLA-4 antibody or antigen binding fragment thereof comprising a heavy chain immunoglobulin variable domain (V_{H}) and a light chain immunoglobulin variable domain (V_{L}), wherein:
(a) the V_{H} comprises a V_{H}-CDR1 sequence of GYTFTDY (SEQ ID NO: 27), a V_{H}-CDR2 sequence of PYNG (SEQ ID NO: 28), and aV_{H}-CDR3 sequence of YGSWFA (SEQ ID NO: 29), and
(b) the V_{L} comprises a V_{L}-CDR1 sequence of SQSIVHSNGNTY (SEQ ID NO: 30), a V_{L}-CDR2 sequence of KVS (SEQ ID NO: 31), and a V_{L}-CDR3 sequence of GSHVPY (SEQ ID NO: 32); and
wherein the open reading frame is at least 95% identical to the nucleotide sequence set forth in SEQ ID NO: 1.

9. The engineered E3LΔ83N-TK⁻-anti-CTLA-4 vaccinia virus of any one of claims 1-6 or claim 8, wherein the open reading frame encodes (a) the heavy chain CDR regions of an anti-human CTLA-4 antibody (anti-huCTLA-4) and the light chain CDR regions of an anti-huCTLA-4, or (b) encodes the heavy chain variable regions of an anti-human CTLA-4 antibody (anti-huCTLA-4) and the light chain variable regions of an anti-huCTLA-4, wherein the anti-huCTLA-4 is optionally ipilimumab.

10. The engineered E3LΔ83N-TK⁻-anti-CTLA-4 vaccinia virus of any one of claims 1-9, wherein mice infected with the engineered virus have an increased post-infection lifespan compared to mice infected with a vector control (E3LΔ83N-TK⁻) or E3LΔ83N-TK⁻ co-administered with anti-CTLA-4 (E3LΔ83N-TK⁻+ anti-CLTA-4).

11. An immunogenic composition comprising the engineered E3LΔ83N-TK⁻-anti-CTLA-4 vaccinia virus of any one of claims 1-10.

12. The immunogenic composition of claim 11, further comprising a pharmaceutically acceptable carrier.

13. The immunogenic composition of claim 11 or claim 12, further comprising a pharmaceutically acceptable adjuvant.

14. A method for treating a solid tumor in a subject in need thereof, the method comprising delivering to a tumor a composition comprising an effective amount of an engineered E3LΔ83N-TK⁻-anti-CTLA-4 vaccinia virus of any one of claims 1-10 or the immunogenic composition of any one of claims 11-13.

15. The method of claim 14, wherein treatment comprises one or more of the following:
inducing an immune response in the subject against the tumor or enhancing or promoting an ongoing immune response against the tumor in the subject, inducing increased cytotoxic CD8⁺ T cells and/or CD4⁺ T effector cells within the tumor;
inducing increased cytotoxic CD8⁺ T cells within the spleen; reducing the volume of the tumor, eradicating the tumor, inhibiting growth of the tumor, inhibiting metastatic growth of the tumor, inducing apoptosis of tumor cells, or prolonging survival of the subject as compared to an untreated control subject.

16. The method of claim 14 or claim 15, wherein the tumor includes tumor cells located at the site of the E3LΔ83N-TK⁻-anti-CTLA-4 vaccinia virus delivery, or tumor cells located both at the site of delivery and elsewhere in the body of the subject.

17. The method of any one of claims 14-16, wherein the composition is administered to the subject intratumorally, intravenously, or any combination thereof.

18. The method of any one of claims 14-16, wherein the tumor is melanoma, colon carcinoma, breast carcinoma, or prostate carcinoma.

19. The method of any one of claims 14-18, further comprising simultaneously or sequentially delivering one or more immune checkpoint blocking agents or immune stimulating agents to the subject, wherein the one or more immune checkpoint blocking agents is administered to the subject intratumorally, intravenously, or any combination thereof.

20. The method of claim 19, wherein the one or more immune checkpoint blocking agents or immune stimulating agents is selected from the group consisting of: anti-PD-1 antibody, anti-PD-L1 antibody, anti-PD-L2 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, anti-GITR antibody, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, and any combination thereof.

21. An engineered E3LΔ83N-TK⁻- hFlt3L-anti-CTLA-4 vaccinia virus comprising an insertion of a heterologous nucleotide sequence into the coding sequence of a thymidine kinase (TK) gene, wherein the heterologous nucleotide sequence comprises an expression cassette comprising an open reading frame encoding human Fms-like tyrosine kinase 3 ligand (hFlt3L), an anti-cytotoxic T lymphocyte-associated antigen (CTLA-4) antibody heavy chain (HC), and an anti-CTLA-4 antibody light chain (LC), wherein the hFlt3L and the HC nucleotide sequences are separated by a nucleotide sequence that encodes, in the 5′ to 3′ direction, a protease cleavage site and a 2A peptide (Pep2A) sequence, and wherein the HC and LC are separated by a nucleotide sequence that encodes, in the 5′ to 3′ direction, a protease cleavage site and a Pep2A sequence.

22. The engineered E3LΔ83N-TK⁻- hFlt3L-anti-CTLA-4 vaccinia virus of claim 21, wherein the protease cleavage site is a furin cleavage site.

23. The engineered E3LΔ83N-TK⁻- hFlt3L-anti-CTLA-4 vaccinia virus of claim 21 or claim 22, wherein the expression cassette further comprises a promoter that is capable of directing expression of the open reading frame.

24. The engineered E3LΔ83N-TK⁻- hFlt3L-anti-CTLA-4 vaccinia virus of any one of claims 21-23, wherein the heterologous nucleic acid sequence further comprises an additional expression cassette comprising an open reading frame that encodes a selectable marker operably linked to a promoter that is capable of directing expression of the selectable marker.

25. The engineered E3LΔ83N-TK⁻- hFlt3L-anti-CTLA-4 vaccinia virus of claim 24, wherein the selectable marker is a xanthine-guanine phosphoribosyl transferase (gpt) gene, a bioluminescent protein, a fluorescent protein, a chemiluminescent protein, or any combination thereof.

26. The engineered E3LΔ83N-TK⁻- hFlt3L-anti-CTLA-4 vaccinia virus of any one of claims 21-25, wherein the virus does not produce a full-length thymidine kinase (TK) gene product.

27. The engineered E3LΔ83N-TK⁻- hFlt3L-anti-CTLA-4 vaccinia virus of any one of claims 21-26, wherein the open reading frame comprises the nucleotide sequence set forth in SEQ ID NO: 5.

28. The engineered E3LΔ83N-TK⁻- hFlt3L-anti-CTLA-4 vaccinia virus of any one of claims 21-26, wherein the open reading frame encodes an anti-CTLA-4 antibody or antigen binding fragment thereof comprising a heavy chain immunoglobulin variable domain (V_{H}) and a light chain immunoglobulin variable domain (V_{L}), wherein:
(a) the V_{H} comprises a V_{H}-CDR1 sequence of GYTFTDY (SEQ ID NO: 27), a V_{H}-CDR2 sequence of PYNG (SEQ ID NO: 28), and aV_{H}-CDR3 sequence of YGSWFA (SEQ ID NO: 29), and
(b) the V_{L} comprises a V_{L}-CDR1 sequence of SQSIVHSNGNTY (SEQ ID NO: 30), a V_{L}-CDR2 sequence of KVS (SEQ ID NO: 31), and a V_{L}-CDR3 sequence of GSHVPY (SEQ ID NO: 32); and
wherein the open reading frame is at least 95% identical to the nucleotide sequence set forth in SEQ ID NO: 5.

29. The engineered E3LΔ83N-TK⁻-anti-CTLA-4 vaccinia virus of any one of claims 21-26 or claim 28, wherein the open reading frame encodes (a) the heavy chain CDR regions of an anti-human CTLA-4 antibody (anti-huCTLA-4) and the light chain CDR regions of an anti-huCTLA-4, or (b) encodes the heavy chain variable regions of an anti-human CTLA-4 antibody (anti-huCTLA-4) and the light chain variable regions of an anti-huCTLA-4, wherein the anti-huCTLA-4 is optionally ipilimumab.

30. An immunogenic composition comprising the engineered E3LΔ83N-TK⁻- hFlt3L-anti-CTLA-4 vaccinia virus of any one of claims 21-29.

31. The immunogenic composition of claim 30, further comprising a pharmaceutically acceptable carrier.

32. The immunogenic composition of claim 30 or claim 31, further comprising a pharmaceutically acceptable adjuvant.

33. A method for treating a solid tumor in a subject in need thereof, the method comprising delivering to a tumor a composition comprising an effective amount of an engineered E3LΔ83N-TK⁻- hFlt3L-anti-CTLA-4 vaccinia virus of any one of claims 21-29 or the immunogenic composition of any one of claims 30-32.

34. The method of claim 33, wherein treatment comprises one or more of the following:
inducing an immune response in the subject against the tumor or enhancing or promoting an ongoing immune response against the tumor in the subject, inducing increased cytotoxic CD8⁺ T cells and/or CD4⁺ T effector cells within the tumor;
inducing increased cytotoxic CD8⁺ T cells within the spleen; reducing the volume of the tumor, eradicating the tumor, inhibiting growth of the tumor, inhibiting metastatic growth of the tumor, inducing apoptosis of tumor cells, or prolonging survival of the subject as compared to an untreated control subject.

35. The method of claim 33 or claim 34, wherein the tumor includes tumor cells located at the site of the E3LΔ83N-TK⁻- hFlt3L-anti-CTLA-4 vaccinia virus delivery, or tumor cells located both at the site of delivery and elsewhere in the body of the subject.

36. The method of any one of claims 33-35, wherein the composition is administered to the subject intratumorally, intravenously, or any combination thereof.

37. The method of any one of claims 33-35, wherein the tumor is melanoma, colon carcinoma, breast carcinoma, or prostate carcinoma.

38. The method of any one of claims 33-37, further comprising simultaneously or sequentially delivering one or more immune checkpoint blocking agents or immune stimulating agents to the subject, wherein the one or more immune checkpoint blocking agents or immune stimulating agents is administered to the subject intratumorally, intravenously, or any combination thereof.

39. The method of claim 38, wherein the one or more immune checkpoint blocking agents or immune stimulating agents is selected from the group consisting of: anti-PD-1 antibody, anti-PD-L1 antibody, anti-PD-L2 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, anti-GITR antibody, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, and any combination thereof.

40. A recombinant E3LΔ83N-TK⁻-anti-CTLA-4 virus nucleic acid sequence, wherein the nucleic acid sequence between position 80,962 and 81,032 of the corresponding wild type vaccinia genome as set forth in SEQ ID NO: 7 is replaced with the heterologous nucleic acid sequence of any one of claim 1, 4, 5, 6, 7, 8, or 9.

41. A recombinant E3LΔ83N-TK⁻- hFlt3L-anti-CTLA-4 vaccinia virus nucleic acid sequence, wherein the nucleic acid sequence between position 80,962 and 81,032 of the corresponding wild type vaccinia genome as set forth in SEQ ID NO: 7 is replaced with the heterologous nucleic acid sequence of any one of claim 21, 24, 25, 26, 27, 28, or 29.


SEQUENCE LISTING (32 sequences)

SEQ ID NO: 1 - DNA, 2199 bp, Artificial Sequence
  Description of Artificial Sequence Synthetic polynucleotide
atggaatggtcctttgtctttctttttttcttgtccgcagctgccggagtacattcggaggcgaagttgcaagagtccggacctgtacttgttaagcccggagcttcagtgaaaatgtcctgtaaagcatccggatatacctttacagattattatatgaattgggtgaagcaaagtcatggaaagagtcttgaatggataggagtaattaatccttataacggagatacatcttataatcaaaagttcaaaggaaaagctacactaactgttgataaatcctcaagtactgcttatatggaactaaactcactaactagtgaagattctgcagtttattattgtgctcgttattatggttcgtggtttgcatattggggacagggaaccttaataactgtaagtacagcaaaaacaacggcgccttctgtttatccattagcgcctgtatgtggagatacaactggttcttctgttacattaggatgtctagtcaaaggatatttcccagaacctgttacattaacctggaactccggttcgctatcatcaggtgtacacactttcccggcggttctacaatctgatttgtatacattatcatcttccgttacagttacttcttccacttggccatcgcaaagtatcacatgtaacgtagcgcacccagcttcatcaacaaaagtcgataaaaaaatagagccgcgaggtcccactataaagccgtgtccaccttgtaaatgtccagctcctaatttattaggaggacccagtgtatttattttccctcctaaaattaaagatgtattgatgatttctttatctccaattgttacatgcgtggttgtagatgtatccgaagacgatccggatgtgcaaatatcgtggttcgttaataatgtggaagttcacaccgcgcaaactcaaactcacagagaggattacaattctaccttgcgtgtagtgtcggctctacctatacaacaccaagattggatgtctggaaaagaatttaaatgcaaagttaataacaaagaccttccagcgccaatagaaagaacaatatccaaacctaaaggtagtgtaagagctcctcaagtatacgttttaccgcctcctgaagaagaaatgacgaaaaaacaagttacattaacctgtatggtgacagattttatgccagaggatatttatgtggagtggactaataatggaaaaacggaattgaattacaaaaatactgaacctgtattagatagtgatggatcatattttatgtacagtaaattgagagtggaaaaaaagaattgggttgaaagaaattcgtactcttgttcagttgtacatgagggactacataatcatcataccactaagagtttttcaagaacccctggtaaacgtagaaggcgtaggagatctggtgctactaatttctccttgttaaaacaagccggtgacgtcgaagaaaaccctggtcctatgatgacatggactctactattccttgccttccttcatcacttaacagggtcatgtgccgacatcgttatgacccaaactactctttcattacctgtgtcattaggagaccaggcatccatctcatgccgatcctctcaatcgatagtacactctaatggaaatacctatttagaatggtatttgcagaagccgggtcaatctccgaagttactaatttataaagtttctaacagattttcaggagttccagatagatttagtggttcaggatccggtactgactttacattaaagatctctcgtgtagaagctgaggatcttggtgtatattattgtttccaaggatctcacgtcccatatactttcggaggaggaaccaaactagaaattaagcgtgctgatgcggctcccacagtaagtatattcccaccgtcgtcagaacaattaacctcgggaggagcctcggtcgtttgtttcttaaataacttttatcctaaggatatcaacgttaaatggaaaattgatggttctgaacgtcagaatggagtgttgaattcatggactgatcaagattccaaagactctacttattcgatgtcgagtacacttactttaacaaaagatgaatatgaacgacataactcatatacttgtgaagcaacccataagacatctacttcaccaattgtaaagtcttttaatcgaaacgaatgctaa

SEQ ID NO: 2 - protein, 732 aa, Artificial Sequence
  Description of Artificial Sequence Synthetic polypeptide
MEWSFVFLFFLSAAAGVHSEAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRRRRRRSGATNFSLLKQAGDVEENPGPMMTWTLLFLAFLHHLTGSCADIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - DNA, 2232 bp, Artificial Sequence
  Description of Artificial Sequence Synthetic polynucleotide
atggaatggagctttgtctttctcttcttcctgtcagcagctgcaggtgtccactcccaggtgcagctgcaggagtcgggcccaggactggtgaagcctttacagaccctgtccctcacctgcactgtctctggtggctccatcagcagtggtggttattactggagctggatccgccagcacccagggaagggcctggagtggattgggtacatctattacagtaggagcacctactacaacccgtccctcaagagtcgagttaccatatcagtagacacgtctaagaaccagttctccctgaagctgagctctgtgacagccgcggacacggccgtgtattactgtgcgagaaccgggtatagcagtggctggtacccttttgactactggggccagggaaccctggtcaccgtctctagtgcaaaaacaacggcgccttctgtttatccattagcgcctgtatgtggagatacaactggttcttctgttacattaggatgtctagtcaaaggatatttcccagaacctgttacattaacctggaactccggttcgctatcatcaggtgtacacactttcccggcggttctacaatctgatttgtatacattatcatcttccgttacagttacttcttccacttggccatcgcaaagtatcacatgtaacgtagcgcacccagcttcatcaacaaaagtcgataaaaaaatagagccgcgaggtcccactataaagccgtgtccaccttgtaaatgtccagctcctaatttattaggaggacccagtgtatttattttccctcctaaaattaaagatgtattgatgatttctttatctccaattgttacatgcgtggttgtagatgtatccgaagacgatccggatgtgcaaatatcgtggttcgttaataatgtggaagttcacaccgcgcaaactcaaactcacagagaggattacaattctaccttgcgtgtagtgtcggctctacctatacaacaccaagattggatgtctggaaaagaatttaaatgcaaagttaataacaaagaccttccagcgccaatagaaagaacaatatccaaacctaaaggtagtgtaagagctcctcaagtatacgttttaccgcctcctgaagaagaaatgacgaaaaaacaagttacattaacctgtatggtgacagattttatgccagaggatatttatgtggagtggactaataatggaaaaacggaattgaattacaaaaatactgaacctgtattagatagtgatggatcatattttatgtacagtaaattgagagtggaaaaaaagaattgggttgaaagaaattcgtactcttgttcagttgtacatgagggactacataatcatcataccactaagagtttttcaagaacccctggtaaaggtagttccgactacaaagacgatgacgacaagcgtagaaggcgtaggagatctggtgctactaatttctccttgttaaaacaagccggtgacgtcgaagaaaaccctggtcctatgatgacatggactctactattccttgccttccttcatcacttaacagggtcatgtgccgacatccagatgaccctgtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagtataatagttccccgtggacgttcggccaagggaccgaggtggaaatcaaacgtgctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 4 - protein, 743 aa, Artificial Sequence
  Description of Artificial Sequence Synthetic polypeptide
MEWSFVFLFFLSAAAGVHSQVQLQESGPGLVKPLQTLSLTCTVSGGSISSGGYYWSWIRQHPGKGLEWIGYIYYSRSTYYNPSLKSRVTISVDTSKNQFSLKLSSVTAADTAVYYCARTGYSSGWYPFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGSSDYKDDDDKRRRRRRSGATNFSLLKQAGDVEENPGPMMTWTLLFLAFLHHLTGSCADIQMTLSPSSLSASVGDRVTITCRASQGIRNDLGWYQQKPGKAPKRLIYAASSLQSGVPSRFSGSGSGTEFTLTISSLQPEDFATYYCLQYNSSPWTFGQGTEVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 3021 bp, Artificial Sequence
  Description of Artificial Sequence Synthetic polynucleotide
atgacagtgctggcgccagcctggagcccaacgacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagccccctcccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgcctcccgtccccagtccccaggacctgctgcttgtggagcacagacggcggagaagaagaagcggcgccaccaatttcagcctgctgaaacaggctggcgacgtggaagagaaccccggacctatgatggaatggtcctttgtctttctttttttcttgtccgcagctgccggagtacattcggaggcgaagttgcaagagtccggacctgtacttgttaagcccggagcttcagtgaaaatgtcctgtaaagcatccggatatacctttacagattattatatgaattgggtgaagcaaagtcatggaaagagtcttgaatggataggagtaattaatccttataacggagatacatcttataatcaaaagttcaaaggaaaagctacactaactgttgataaatcctcaagtactgcttatatggaactaaactcactaactagtgaagattctgcagtttattattgtgctcgttattatggttcgtggtttgcatattggggacagggaaccttaataactgtaagtacagcaaaaacaacggcgccttctgtttatccattagcgcctgtatgtggagatacaactggttcttctgttacattaggatgtctagtcaaaggatatttcccagaacctgttacattaacctggaactccggttcgctatcatcaggtgtacacactttcccggcggttctacaatctgatttgtatacattatcatcttccgttacagttacttcttccacttggccatcgcaaagtatcacatgtaacgtagcgcacccagcttcatcaacaaaagtcgataaaaaaatagagccgcgaggtcccactataaagccgtgtccaccttgtaaatgtccagctcctaatttattaggaggacccagtgtatttattttccctcctaaaattaaagatgtattgatgatttctttatctccaattgttacatgcgtggttgtagatgtatccgaagacgatccggatgtgcaaatatcgtggttcgttaataatgtggaagttcacaccgcgcaaactcaaactcacagagaggattacaattctaccttgcgtgtagtgtcggctctacctatacaacaccaagattggatgtctggaaaagaatttaaatgcaaagttaataacaaagaccttccagcgccaatagaaagaacaatatccaaacctaaaggtagtgtaagagctcctcaagtatacgttttaccgcctcctgaagaagaaatgacgaaaaaacaagttacattaacctgtatggtgacagattttatgccagaggatatttatgtggagtggactaataatggaaaaacggaattgaattacaaaaatactgaacctgtattagatagtgatggatcatattttatgtacagtaaattgagagtggaaaaaaagaattgggttgaaagaaattcgtactcttgttcagttgtacatgagggactacataatcatcataccactaagagtttttcaagaacccctggtaaaggtagttccgactacaaagacgatgacgacaagcgtagaaggcgtaggagatctggtgctactaatttctccttgttaaaacaagccggtgacgtcgaagaaaaccctggtcctatgatgacatggactctactattccttgccttccttcatcacttaacagggtcatgtgccgacatcgttatgacccaaactactctttcattacctgtgtcattaggagaccaggcatccatctcatgccgatcctctcaatcgatagtacactctaatggaaatacctatttagaatggtatttgcagaagccgggtcaatctccgaagttactaatttataaagtttctaacagattttcaggagttccagatagatttagtggttcaggatccggtactgactttacattaaagatctctcgtgtagaagctgaggatcttggtgtatattattgtttccaaggatctcacgtcccatatactttcggaggaggaaccaaactagaaattaagcgtgctgatgcggctcccacagtaagtatattcccaccgtcgtcagaacaattaacctcgggaggagcctcggtcgtttgtttcttaaataacttttatcctaaggatatcaacgttaaatggaaaattgatggttctgaacgtcagaatggagtgttgaattcatggactgatcaagattccaaagactctacttattcgatgtcgagtacacttactttaacaaaagatgaatatgaacgacataactcatatacttgtgaagcaacccataagacatctacttcaccaattgtaaagtcttttaatcgaaacgaatgctaa

SEQ ID NO: 6 - protein, 1006 aa, Artificial Sequence
  Description of Artificial Sequence Synthetic polypeptide
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEHRRRRRRSGATNFSLLKQAGDVEENPGPMMEWSFVFLFFLSAAAGVHSEAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGSSDYKDDDDKRRRRRRSGATNFSLLKQAGDVEENPGPMMTWTLLFLAFLHHLTGSCADIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - DNA, 194711 bp, Vaccinia virus WR
atttaaaatataatattaatgtactaaaacttatatattattaatttatctaactaaagttagtaaattatatatataattttataattaatttaattttactaattttatttagtgtctagaaaaaaatgtgtgacccatgactgtaggaaactctagagtgtaagaaagatcgatcgctttatagagaccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcgaaggagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaaggagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagaaagagatagttgatctagatatttttcttagtacaaaagtcaatgttttaaaatatatggacaagaatttgtctgtataaaaacttgtgtgaaattttgtaccaaagaaaaaatgtgagcagtatcccctacatggattttactagatcatttatataccaaaaaatattatacgatctacgttttattatatgattttaacgtgtaaattataaacattattttatgatatacaattgtctggtaacctagatgggcataggggatgttgataagctcgacgagtatatgttgttggacgttattgtttaagaaatagttgatgcatcagaaagagaataaaaaatattttagtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagataaaacttttttacgactccatcagaaagaggtttaatatttttgtgagaccatcgaagagagaaagagaataaaaatattttatgactccattgaagagagaaagagaaaatgagaatgagaataaaaatattttagtgacaccatcagaaagaggtttaatatttttgtgagaccatcgaagagagaaagagaataaaaatattttatgactccattgaagagagaaagagaaaatgagaatgagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcagaaagaggtttaatatttttgtgataccctgaaaggaaataggaataggaataggaatagtgtcataatcgtatcacactattgagacagaaaaagaagaagtcgcgagaggtaactttttgtgaatgtagttaagaacatttttgttttgcaaaccggaatatagtgtccggtacacttttttaattcgtggtgtgcctgaatcgttcgattaaccctactcatccaatttcagatgaatagagttatcgattcagacacacgctttgagttttgttgaatcgatgagtgaagtatcatcggttgcaccttcagatgccgatccgtcgacatacttaaatccatccttgacctcaagttcagatgattccttgcacatgtctccgatacgaacgctaaactctagattcttgacacattttgtatcgacgatcgttgaaccgatgatatcttcgtaactcactttcttatgagagatgttagacccgagtactggatgggtcttgatgtcgctgtctttctcttcttcgctacatctgatgtcgatagacacctcacagtctttgatcatagccagagcttcttcatgagtgatcgcgggagagtccttaccttgtcctggggacacgctggacaatctagcattcactgtgtttccatcagcggattctgagatggatttaatctgaggacatttggtgaatccaaagttcattctcagacctccaccgatgatggagtaataagtggtaggaggatctacatcctcgactgatgtggaatcatcttctgattccacctcgggatctggatctgactcggactctgtaatttccgttacggattggcaaatcttatcattggtcggtgtttggtcttgctttgtgactttgataataacatcgattcccatatgatgtttgttttcttcttccgtacacgaggaggaggatgaggatgattgctgaagactggcaggcacatgcatgccaggacgatatattgtttcatgattgctattgattgagtactgttctttatgattctacttccttaccgtgcaataaattagaatatattttctacttttacgagaaattaattattgtatttattatttatgggtgaaaaacttactataaaaagcgggtgggtttggaattagtgatcagtttatgtatatcgcaactaccgggcatatggctatcgacatcgagaacattacccacatgataagagattgtatcagtttcgtagtcttgagtattggtattactatatagtatatagatgtcgacgctagatagacagtctccgaatgcggcatgataccgtcatcattctttgctttcgttaactgtttggaggaaaaatttttgttattgcatttaatctcgaaattcagagtgcacacctttctcctgtaaagaaacctgaagttgctaccttattaaggacggagaagtattcctcacgaaatacgggattacagtctttatgattcatagtaatagttagttccgacgttgagatggattcgctgagaccggtagtggtcgttaactggatacagattaatttccacatcgatatagttaaaggtattactgggtacgggttcgcatttatctgcggaagagacggtgtgagaatatgttccgagaccacacggagaacagatgacgtctccggatactccgtatcctattccacattttgtttgggaaacacatgccttgcatccggatgatcctttgagaagacaataatatccgggagagcattcacagattctattgtgagtcgtgttacacggtcgcgtcttccgttacaacttagacaagcgggtaaatgattattgcgagatgtgaaggtacccgaaccacacggcgtacattgtgtgttagtcttgctatcgcataatctggaagcgtatgttcccggacacaaattatggcgtttgtattcgttgtctttacactttccatcggatggtgcatgcggtgctatatctcttccgtttattattatacatgagagaaacaatatatacgagtataatacggacttcatgatttaataatgtagtaatcgtcgtcttgttcctgtttcctacttctccaatcatatagatattttctttctatcatggataatatttgtaatggttcttttcgtacaacatactgtttagatgatattgcgcataatttccggaggcaaatacgatagtctagattgaccgatggtagactctaatttattgagtgctttgtcgacgagtttacttttacgctccatcgatagatggcactgttctatgagatcgtcgtacatgggaaatgaaatgtgactgtctgaatgtatggctttaagatagctgtgataccgtatacaggtcggtgtcggagattcgaatctctttaaggcgacttatgtcacgatgatggaatctatcttatcgaatgatatatttttcataaatacacttttatagtcctcgtttaaacagaatttactatgtagttccgcgaatgactcgtcccttaataggcagtaggctattatcttctttacgtagtaatcgtcgtagggagagacatcttgtagaacaacgatttaatcataggtagagatactttcagtctgtggtggatgatgtcattcacaacatccgccttgtatatgatgtttctgttttcaaacaccaagtcgaataccgtctttagtcggaaggttgatgtcgtatccgatgtatgaggcaacattgttgttacaattttgaaaggcggtattatagtattcgtctttctgaatgtcgaacctatctagtagataccgtagtatattgagagtgtatccttgattatgttttatgaatagataaagtagatgttgtccttcttccttttgttcgtgccaattgagtaacattatgagaatatgacctgttgcacaatcgttccatgatgggtgtacaatcaagattattacgtatcctcgtatcggctcctcgagataaaagagcatacaccacacgaggactatgtttggtatactgttgaaggtaagtgtgtaaccgcgttaatgtttgctccataatctattatcgcgtagatgaatcgcttctcggctcgcatcttagtgtgacttaacttgtaataattgcttttgtagaacgtggatatgtgtttacagtagtaatgaagagaagtgagttcatcctcgtcggatcctttgtacagaacgtaatagtttaagctcccattgaatttatatctaagataacacagcaatagatcggatgatttactaaagtcatcaatggtgtccgttagtatatcaaagatcttgttatcgattgatagtggtgtcctttttcatccttgctatcaaagttacgcatgccgtggtgtaacaatatctttaatacagatggattaaatcgtgtattcatcgtatagcaatgtaatggagagttacctcgtttattcagatcgcagtgtttaataactagcttaaacagatgagacgatgtatccacatcaaagaacgtgaaatacatatgacagacattgttgacagaaacgtgaccttcattcttaccgtcgtccataaatacgttaggtatgtaccacatactgtcgcgaacgatgcgtacaatctcgtccatctcataatgatttactttttcataattaaagatgtgaaagaaaaacagaacaatatatttttttagtaatgtttatgcgagacatataaaataaactccgtgtttatgatcatttttaacagcaacacattcaatattgtattgttatttttatattatttacacaattaacaatatattattagtttatattactgaattaataatataaaattcccaatcttgtcataaacacacactgagaaacagcataaacacaaaatccatcaaaaatgtcgatgaaatatctgatgttgttgttcgctgctatgataatcagatcattcgccgatagtggtaacgctatcgaaacgacatcgccagaaattacaaacgctacaacagatattccagctatcagattatgcggtccagagggagatggatattgtttacacggtgactgtatccacgctagagatattgacggtatgtattgtagatgctctcatggttatacaggcattagatgtcagcatgtagtattagtagactatcaacgttcagaaaacccaaacactacaacgtcatatatcccatctcccggtattatgcttgtattagtaggcattattattattacgtgttgtctattatctgtttataggttcactcgacgaactaaactacctatacaagatatggttgtgccataatttttataaatttttttatgagtatttttacaaaaaaaatgtataaagtgtatgtcttatgtatatttataaaaatgctaagtatgcgatgtatctatgttatttgtatttatctaaacaatacctctacctctagatattatacaaaaattttttatttcggcatattaaagtaaaatctagttaccttgaaaatgaatacagtgggtggttccgtatcaccagtaagaacataatagtcgaatacagtatccgattgagattttgcatacaatactagtctagaaagaaatttgtaatcatcttctgtgacgggagtccatatatctgtatcatcgtctagtttatcagtgtcccatgctatattcctgttatcatcattagttaatgaaaataactctcgtgcttcagaaaagtcaaatattgtatccatacatacatctccaaaactatcgcttatacgtttatctttaacgatacctatacctagatggttatttactaacagacattttccagatctattgactataactcctatagtttccacatcaaccaagtaatgatcatctattgttatataacaataacataactcttttccatttttatcagtatgtatatctatatcaacgtcgtcgttgtagtgaatagtagtcattgatctattatatgaaacggatatgtctagaacggcaattgttttacgtccagttaacactttctttgatttaaagtctagagtctttgcaaacataatatccttatccgactttatatttcctgtagggtggtataattttattttgcctccacatatcggtgtttccaaatatattactagacaatattccatatagttattagttaagggtacccaattagaacacgtacgcttattatcatcatttggatcgtatttcataaaagttattgtactatcgatgtcaacacattctacattttttaatcgtctatatagtatttttctgatattttctataatatcagaattgtcttccatcggaagttgtatactatcggaatcagttacatgtttaaataattctctgatgtcattccttatacaatcaaattcattattaaacagtttaatagtctgtagacctttatcgtcgtaaatatccattgtcttattagttacgcttatttttatgtgttttacgttgctttattatattttataagaatgattgtttgacgaatcacgagaactattaagacacattattaggtatatattataaaaaagtttttgattacgatgttataagaggaaagaggacacattaacatcatacatcaattaactacattcttataacatcgtaatcaaaagaattgcaattttgatgtataacaactgtcaatgggttatggaattgtatattacatattatacggtatgttggtaacgacaaataccgatcggtaattgtctgccggtgtaatagaattatatatatctatctattacaccggctgagtatgcataataataagttgtggtagtatgatctccatatttataatttaggactttgtattcagtatttttggaatcataaaaaataaaaaaaagttttactaatttaaaatttaaaaagtatttacatttttttcactgtttagtcgcggatatggaattcgatcctgccaaaatcaatacatcatctatagatcatgtaacaatattacaatacatagatgaaccaaatgatataagactaacagtatgcattatccgaaatattaataacattacatattatatcaatatcacaaaaataaatacacatttggctaatcaatttcgggcttggaaaaaacgtatcgccggaagggactatatgactaacttatctagagatacaggaatacaacaatcaaaacttactgaaactatacgtaactgtcaaaaaaatagaaacatatatggtctatatatacactacaatttagttattaatgtggttattgattggataaccgatgtgattgttcaatcaatattaagagggttggtaaattggtacatagctaataatacctatacacccaataatacaacaaccatttctgagttggatatcatcaaaatactggataaatacgaggacgtgtatagagtaagtaaagaaaaagaatgtggaatttgctatgaagttgtttactcaaaacgatagatactttggtttattggattcgtgtactcatatattttgcataacatgcatcaatatatggcataaaacacgaagagaaaccggtgcgtcggataattgtcctatatgtcgtacccgttttagaaacataacaatgagcaagttctataagctagttaactaataaataaaaagtttaatttgttgacgacgtatgtcgttatttttctcgtatgaaagattaaattcaattcaattcgttgtttctaatataatctgccgtattggatggattctcaagacaattgcatttagattatattatcatgaataaaaatagtagcacgcactacttcagccaaatattcttttttgaaacgccatctatcgtagtgaggacacaagtgaacctataattatcaaatttattagtatcagtcacatgaaggactttctgtagagtgacgattctaccatctatggtactaacggtttcatcctccttgataccctcacccaaatgttctataaatttagcatcctcgtccgatctcatatcctttgccaaccaatacatgtagctaaaattaggcataaatttcacacatccagtgcaacgaaattctccagaagatgttacgatgtttaggttaggacatttgatttcgtcggcattaacatatgggtgaacacacccatacatgaaagcgatgagaaataggattctcatcttgccaaaatatcactagaaaaaatttatttatcaattttaaaggtataaaaaatacttattgttgctcgaatattttgtatttgatggtatacggaagattagaaatgtaggtattatcatcaactgattctatggttttatgtattctatcatgtttcactattgcgtcggaaataatatcatatgcttccacatatattttattttgttttaactcataatactcacgtaattctggattattggcatatctatgaataattttagctccatgatcagtaaatattaatgagaacatagtattaccacctaccattatttttttcatttcgttcaattcttgattgcaaagatctatataatcattatagcgttgacttatggactctggaatcttagacgatgtacagtcatctataatcatggcatatttaatacattgttttatagcatagtagttatctacgatgttagatatttctctcaatgaatcaatcacacaatctaatgtaggtttatgacataatagcattttcagcagttcaatgtttctagattcgttgatggcaatggctatacatgtatatccgttatttgatctaatgttgacatctgaaccggattctagcagtaaagatactagagattgtttattatatctaacagccttgtgaagaagtgtttctcctcgtttgtcaatcatgttaatgtctttaagataaggtaggcaaatgtttatagtactaagaattgggcaagcataagacatgtcacaaagaccctttttgtatgtataagtgtaaaaattataacattcatagttggatttacataggtgtccaatcgggatctctccatcatcgagataattgatggcatctcccttccttttttagtagatatttcatcgtgtaagaatcaatattaatatttctaaagtattcgtgtatagcctctttatttaccacagttccatattccactagagggatatcgccgaatgtcatatactcaattagtatatgttggaggacatccgagttcattgttttcaatatcaaaaagatggtttccttatcatttctccatagtggtacaatactacacattattccgtgcggctttccattttccaaaaacaatttgaccaaatctaaatctacatctttattgtatctataatcactatttagataatcagccataattactcgagtgcaacatgttagatcgtctatatatgaataagcagtgttatctattcctttcattaacaatttaacgatgtctatatctatatgagatgacttaatataatattgaagagctgtacaatagtttttatctatagaagacggcttgattccgtgattaattagacatttaacaacttccggacgcacatatgctctcgtatccgactttgaatacagatgagagatgatatacagatgcaatacggtaccgcaatttcgtagttgataatcatcatacgcgtatcagtactcgtcctcataaagaacactgcagccattttctatgaacaaatcaataattttaggaacaggatcattgtcattacataattttctataactgaacgatggttttcacatttaacactcaagtcaaatccatgttctaccaacacctttatcaagtcaacgtctacatttttggatttcatatagctgaatatattaaagtcatttatgttgctaaatccagtggcttctagtagagccatcgctatatcctttaactttaacatgtctactatttgtgtattcttctaatggggtagctgtctccaatttttgcgtaatggattagtgccactgtctagtagtagtttgacgacctcgacattattacaatgctcattaaaaaggtatgcgtgtaaagcattattcttgaattggttcctggtatcattaggatctctgtctctcaacatctgtttaagttcatcgagagccacctcctcattttccagatagtcaaacattttgactgaatgagctactgtgaactctatacacccacacaactaatgtcattaaatattatttttttgaatgtatttataccatgtcaaaaacttgtacaattattaataaaaataatttagtgtttaaattttaccagttccagattttacacctccgttaaccccactttttacaccactggacgatcctcctccccacattccaccgccaccagatgtataagttttagatcctttattactaccatcatgtccatggataaagacactccacatgccgccactactaccccctttagaagacatattaataagacttaaggacaagtttaacaataaaattaatcacgagtaccctactaccaacctacactattatatgattatagtttctatttttacagtaccttaactaaagtctctagtcacaagagcaatactaccaacctacactattatatgattatagtttctatttttataggaacgcgtacgagaaaatcaaatgtctaatttctaacggtagtgttgataaacgattatcgtcaatggatacctcctctatcatgtcgtctattttcttactttgttctattaacttattagcattatatattatttgattataaaacttatattgcttattagcccaatctgtaaatatcggattattaacatatcgtttctttgtaggtttatttaacatgtacatcactgtaagcatgtccgtaccatttattttaatttgacgcatatccgcaatttctttttcgcagtcggttataaattctatatatgatggatacatgctacatgtgtacttataatcgactaatatgaagtacttgatacatattttcagtaacgatttattattaccacctatgaataagtacctgtgatcgtctaggtaatcaactgttttcttaatacattcgatggttggtaatttactcagaataatttccaatatcttaatatataattctgctatttctgggatatatttatctgccagtataacacaaatagtaatacatgtaaacccatattttgttattatattaatgtctgcgccattatctattaaccattctactaggctgacactatgcgacttaatacaatgataaagtatactacatccatgtttatctattttgtttatatcatcaatatacggcttacaaagttttagtatcgataacacatccaactcacgcatagagaaggtagggaataatggcataatatttattaggttatcatcattgtcattatctacaactaagtttccattttttaaaatatactcgacaactttaggatctctattgccaaatttttgaaaatatttatttatatgcttaaatctatataatgtagctccttcatcaatcatacatttaataacattgatgtatactgtatgataagatacatattctaacaatagatcttgtatagaatctgtatatcttttaagaattgtggatattaggatattattacgtaaactattacacaattctaaaatataaaacgtatcacggtcgaataatagttgatcaactatataattatcgattttgtgatttttcttcctaaactgtttacgtaaatagttagatagaatattcattagttcatgaccactatagttactatcgaataacgcgtcaaatatttcccgtttaatatcgcatttgtcaagataataatagagtgtggtatgttcacgataagtataataacgcatctcttttttgtgtgaaattaaatagtttatcacgtccaaagatgtagcataaccatcttgtgacctagtaataatataataatagagaactgttttacccattctatcatcataatcagtggtgtagtcgtaatcgtaatcgtctaattcatcatcccaattataatattcaccagcacgtctaatctgttctattttgatcttgtatccatactgtatgttgctacatgtaggtattcctttatccaataatagtttaaacacatctacattgggatttgatgttgtagcgtatttctctacaatattaataccatttttgatactatttatttctatacctttcgaaattagtaatttcaataagtctatatcgatgttatcagaacatagatattcgaatatatcaaaatcattgatatttttatagtcgactgacgacaataacaaaatcacaacatcgtttttgatattattatttttcttggtaacgtatgcctttaatggagtttcaccatcatactcatataatggatttgcaccactttctatcaatgattgtgcactgctggcatcgatgttaaatgttttacaactatcatagagtatcttatcgttaaccatgattggttgttgatgctatcgcattttttggtttctttcatttcagttatgtatggatttagcacgtttgggaagcatgagctcatatgatttcagtactgtagtgtcagtactattagtttcgatcagatcaatgtctagatctatagaatcaaaacacgataggtcagaagataatgaatatctgtacgcttctttttgtactgtaacttctggttttgttagatggttgcatcgtgctttaacatcaatggtacaaattttatcctcgctttgtgtatcatattcgtctctagtataaaattctatattcagattatcatgcgatgtgtatacgctaacggtatcaataaacggagcacaccatttagtcataacagtaatccaaaattttttaaagtatatcttaacgaaagaagttgtgtcattgtctacggtgtatggtactagatcctcataagtgtatatatctagagtaatgtttaatttattaaatggttgataatatggatcctcatgacaatttccgaagatggaaatgagatatagacatgcaataaatctaatcgaagacatggttactccttaaaaaaatacgaataatcaccttggctatttagtaagtgtcatttaacactatactcatattaatccatggactcataatctctatacgggattaacggatgttctatatacggggatgagtagttttcttctttaactttatactttttactaatcatatttagactgatgtatgggtaatagtgtttaaagagttcgttctcatcatcagaataaatcaatatctctgtttttttgttatacagatgtattacagcctcatatattacgtaatagaacgtgtcatctaccttattaactttcaccgcatagttgtttgcaaatacggttaatcctttgacctcgtcgatttccgaccaatctgggcgtataatgaatctaaactttaatttcttgtaatcattcgaaataatttttagtttgcatccgtagttatcccctttatgtaactgtaaatttctcaacgcgatatctccattaataatgatgtcgaattcgtgctgtatacccatactgaatggatgaactaacgaatatcaacggcgttaatagtaatttactttttcatctttacatattgggtactagttttactatcataagtttataaattccacaagctactatggaataagccaaccatcttagtataccacacatgtcttaaagtttattaattaattacatgttgttttatatatatcgctacgaatttaaagagaaatcagtttaggaagaaaaaaattatctatctacatcatcacgtctctgtattctacgatagagtgctactttaagatgagacatatccgtgtcatcaaaaatatactccattaaaatgattattccggcagcgaacttgatattggatatatcacaacctttgttaatatctacgacaatagacagcagtcccatggttccataaacagtgagtttatctttctttgaagcgatagtttgtagagatcttataaaaccgtcaaacgacatcgcatttatatctttagctaattcatatatgttaccatcgtaatatctaaccgcgtctatcttaaacgtttccatcgctttaaagacgtttccgatagatggtctcatttcatcagtcatactgagccaacaaatataatcgtgtataacatctttgatagaatcagactctaaagaaaacgaatcggctttattatacgcattcatgataaacttaatgaaaaatgtttttcgttgtttaagttggatgaatagtatgtcttaataattgttattatttcattaattaatatttagtaacgagtacactctataaaaacgagaatgacataactagttatcaaagtgtctaggacgcgtaattttcatatggtatagatcctgtaagcattgtctgtattctggagctattttctttatcgcattagtaagttcagaatatgttataaatttaaatcgaataacgaacatgactttagtaaagtcgtctatattaactcttttattttctagccatcgtaataccatgtttaagatagtatattctctagttactacgatctcatcgttgtctagaatatcacatactgaatctacatccaattttagaaattggtctgtgttacatatctcttctatattattgttgatgtattgtcgtagaaaactattacgtagaccattttctttataaaacgaatatatagtactccaattatctttaccgatatatttgcacacataatccattctctcaatcactacatctttaagattttcgttgttaagatatttggctaaactatataattctattagatcatcaacagaatcagtatatatttttctagatccaaagacgaactctttggcgtcctctataatattcccagaaaagatattttcgtgttttagtttatcgagatctgatctgttcatatacgccatgattgtacggtacgttatgataaccgcataaaataaaaatccattttcatttttaaccaatactattcataattgagattgatgtaatactttgttactttgaacgtaaagacagtacacggatccgtatctccaacaagcacgtagtaatcaaatttggtgttgttaaacttcgcaatattcatcaatttagatagaaacttatactcatcatctgttttaggaatccatgtattattaccactttccaacttatcattatcccaggctatgtttcgtccatcatcgttgcgcagagtgaataattcttttgtattcggtagttcaaatatatgatccatgcatagatcggcaaagctattgtagatgtgatttttcctaaatctaatataaaactcgtttactagcaaacactttcctgatttatcgaccaagacacatatggtttctaaatctatcaagtggtggggatccatagttatgacgcagtaacatagattattacattcttgactgtcgctaatatctaaatatttattgttatcgtattggattctgcatatagatggcttgtatgtcaaagatatagaacacataaccaatttatagtcgcgctttacattctcgaatctaaagttaagagatttagaaaacattatatcctcggatgatgttatcactgtttctggagtaggatatattaaagtctttacagatttcgtccgattcaaataaatcactaaataatatcccacattatcatctgttagagtagtatcattaaatctattatattttatgaaagatatatcactgctcacctctatatttcgtacatttttaaactgtttgtataatatctctctgatacaatcagatatatctattgtgtcggtagacgataccgttacatttgaattaatggtgttccattttacaacttttaacaagttgaccaattcatttctaatagtatcaaactctccatgattaaatattttaatagtatccattttatatcactacggacacaaagtagctgacataaaccattgtataatttttatgttttatgtttattagcgtacacattttggaagttccggcttccatgtatttcctggagagcaagtagatgatgaggaaccagatagtttatatccgtacttgcacttaaagtctacattgtcgttgtatgagtatgatcttttaaacccgctagacaagtatccgtttgatattgtaggatgtggacatttaacaatctgacacgtgggtggatcggaccattctcctcctgaacacaggacaccagagttaccaatcaacgaatatccactattgcaactataagttacaacgctcccatcggtataaaaatcctcgtatccgttatgtcttccgttggatatagatggaggggattggcatttaacagattcacaaataggtgcctcgggattccataccatagatccagtagatcctaattcacaatacgatttagattcaccgatcaaatgatatccgctattacaagagtacgttatactagagccaaagtctactccaccaatatcaagttggccattatcgatatctcgaggcgatgggcatctccgtttaatacattgattaaagagtgtccatccagtacctgtacatttagcatatataggtcccattttttgctttctgtatccaggtagacatagatattctatagtgtctcctatgttgtaattagcattagcatcagtctccacactattcttaaatttcatattaatgggtcgtgacggaatagtacagcatgatagaacgcatcctattcccaacaatgtcaggaacgtcacgctctccaccttcatatttatttatccgtaaaaatgttatcctggacatcgtacaaataataaaaagcccatatatgttcgctattgtagaaattgtttttcacagttgctcaaaaacgatggcagtgacttatgagttacgttacactttggagtctcatctttagtaaacatatcataatattcgatattacgagttgacatatcgaacaaattccaagtatttgattttggataatattcgtattttgcatctgctataattaagatataatcaccgcaagaacacacgaacatctttcctacatggttaaagtacatgtataattctatccatttgtcttccttaactatatatttgtatagataattacgagtctcgtgagtaattccagtaattacatagatgtcgccgtcgtactctacagcataaactatactatgatgtctaggcatgggagacttttttatccaacgatttttagtgaaacattccacatcgtttaatactacatatttttcatacgtggtataaactccacccattacatatatatcatcgtttacgaataccgacgcgcctgaatatctaggagtaattaagtttggaagtcttatccatttcgaagtgccgtgtttcaaatattctgccacacccgttgaaatagaaaattctaatcctcctattacatataactttccatcgttaacacaagtactaacttctgattttaacgacgacatattagtaaccgttttccattttttcgtttcaagatctacccgcgatacggaataaacatgtctattgttaatcatgccgccaataatgtatagacaattatgtaaaacatttgcattatagaattgtctatctgtattaccgactatcgtccaatattctgttctaggagagtaatgggttattgtggatatataatcagagtttttaatgactactatattatgttttataccatttcgtgtcactggctttgtagatttggatatagttaatcccaacaatgatatagcattgcgcatagtattagtcataaacttgggatgtaaaatgttgatgatatctacatcgtttggatttttatgtatccactttaataatatcatagctgtaacatcctcatgatttacgttaacgttttcgtgggataagatagttgtcagttcatcctttgataattttccaaattctggatcggatgtcaccgcagtaatattgttgattatttctgacatcgacgcattatatagttttttaattccatatcttttagaaaagttaaacatccttatacaatttgtggaattaatattatgaatcatagtttttacacatagatctactacaggcggaacatcaattattatggcagcaactagtatcatttctacattgtttatggtgatgtttatcttcttccagcgtatatagtctaatagcgattcaaacgcgtgatagtttataccattcaatataatcgcttcatcctttagatggtgatcctgaattcgtttaaaaaaattatacggagatgccgtaataatttccttattcacttgtataatttccccattgatagaaaatatcacgctttccattcttgaagtactataagtaattatagtataatgtaaaggtttatatattcaatattttttataaaaaaatcattttgacattaattcctttttaaatttccgtctatcatctatagaaacgtattctatgaatttataaaatgcttttacgtgtcctatcgtaggcgatagaaccgctaaaaagcctatcgaatttctacaaaagaatctgttatatggtatagggagagtataaaacattaaatgtccgtacttattaaagtattcagtagccaatcctaactctttcgaatacttattaatggctcttgttctgtacgaatctatttttttgaacaacggacctagtggtatatcttgttctatgtatctaaaataatgtctgactagatccgttagtttaatatcctcagtcatcttgtctagaatggcaaatctaactgcgggtttaggctttagtttagtttttatatctacatctatgtctttatctaacaccaaaaatataatagctaatattttattacaatcatccggatattcttctacgatctcactaactaatgtttctttggttatactagtatagtcactatcggacaaataaagaaaatcagatgatcgatgaataatacatttaaattcatcatctgtaagatttttgagatgtctcattaaaatattattagggtcagtactcattatcattcggcagctattacttattttatttttctgtattttattatttttcaccatatagatcaatcattagatcatcaaaatatgtttcaatcatcctaaagagtatggtgaatgactcttcccatctaatttctgaacgttcaccaatgtctctagccactttggcactaatagcgatcattcgcttagcgtcttctatattattaactggttgattcaatctatctagcaatggaccgtcggacagcgtcattctcatgttcttaatcaatgtacatacatcgccgtcatctaccaattcatccaacaacataagctttttaaaatcatcattataataggtttgatcgttgtcatttctccaaagaatatatctaataagtagagtcctcatgcttagtaatttaactattttagttaacaactattttttatgttaaatcaattagtacaccgctatgtttaatacttattcatattttagtttttaggattgagaatcaatacaaaaaattaatgcatcattaattttagaaatacttagtttccacgtagttaatgaaacatttgaactcatcgtacaggacgttctcgtacaggacgtaactataaaccggtttatatttgttcaagatagatacaaatccgataactttttttacgaattctacgggatccactttaaaagtgtcataccgggttctttttattcttttaaacagatcaatggtgtgatgttgattaggtcttttacgaatttgatatagaatagcgttcacatatcctccataatggtcaatcgccatttgttcgtatgtcataaattctttaattatatgacactgtgtattatttagttcatccttgttcatcattaggaatctatccaaaatggcaattatactagaactataggtgcgttgtatacacatattgatgtgtctgtttatacaatccatgatatttggatccatgctactaccttcgggtaaaattgtagcatcatataccatttctagtactttaggttcattattatccattgcagaggacgtcatgatcgaatcctaaaaaaatatattatttttatgttattttgttaaaaataatcatcgaatacttcgtaagatactccttcatgaacataatcagttacaaaacgtttatatgaagtaaagtatctacgatttttacaaaagtccggatgcataagtacaaagtacgcgataaacggaataataatagatttatctagtctatctttttctatagctttcatagttagatacatggtctcagaagtaggattatgtaacatcagcttcgataaaatgactgggttatttagtcttacacattcgctcatacatgtatgaccgttaactacaaagtctacactaaaatgattgaacaatagatagtctaccattgtttcgtattcagatagtacagcgtagtacatagcatcttcacaaattatatcattgtctaatagatatttgacgcatcttatggatcccacttcaacagccatcttaaaatcggtagaatcatattgctttcctttatcattaataatttctaaaacatcatctctatcataaaagatacaaatattaactgtttgatccgtaataacattgctagtcgatagcaatttgttaataagatgcgctgggctcaatgtcttaataagaagtgtaagaggactatctccgaatttgttttgtttattaacatccgttgatggaagtaaaagatctataatgtctacattcttgactgttttagagcatacaatatggagaggtgtatttccatcatgatctggttttgagggactaattcctagtttcatcatccatgagattgtagaagcttttggattgtctgacataagatgtctatgaatatgatttttgccaaatttatccactatcctggcttcgaatccgatggacattatttttttaaacactctttctgaaggatctgtacacgccaacaacggaccacatccttcttcatcaaccgagttgttaatcttggctccatactgtaccaataaatttattctctctatgacttcatcatctgttcccgagagataatatagaggtgttttattatgtttatcacacgcgtttggatctgcgccgtgcgtcagcagcatcgcgactattctattattattaattttagaagctatatgcaatggataatttccatcatcatccgtctcatttggagagtatcctctatgaagaagttcttcgacaaatcgttcatctagtcctttaattccacaatacgcatgtagaatgtgataattatttccagaaggttcgatagcttgtagcatattcctaaatacatctaaatttttactattatatttggcataaagagatagataatactcggccgacataatgttgtccattgtagtataaaaattaatatttctatttctatttctgtatatttgcaacaatttactctctataacaaatatcataacttagttcttttatgtcaagaaggcactggtttagttcatctataaatgtcacgccataactaccacgcatgctatactcagaattatgataaagatatttatccttggggtgtaggtaatggggattaatctttgttggatcagtctctaagttaacacatgtcacacatgatccatttatagttatatcacacgatgatgatttatgaattgattccggaagatcgctatcgtattttgtggttccacaattcatttccatacatgttattgtcacactaatattatgatgaactttatctagccgctgagtggtaaacaacagaacagatagtttattatctttaccaacaccctcagccgctgccacaaatctctgatccgtatccatgatggtcatgtttatttctagtccgtatccagtcaacactatgttagcatttctgtcgatatagctttcactcatatgacactcaccaataatagtagaattaatgtcgtaatttacaccaatagtgagttcggcggcaaagtaccaataccggtaatcttgtcgaggaggacatatagtattcttgtattctaccgaatacccgagagatgcgatacaaaagagtaagactaatttgtaaaccatcttactcaaaatatgtaacaatagtacgatgcaatgagtaagacaataggaaatctatcttatatacacataattattctatcaattttaccaattagttagtgtaatgttaacaaaaatgtgggagaatctaattagtttttctttacacaattgacgtacatgagtctgagttccttgtttttgctaattatttcatccaatttattattcttgactatatcgagatcttttgtataggagtcagacttgtattcaacatgcttttctataatcattttagctatttcggcatcatccaatagtacattttccagattagcagaatagatattaatgtcgtatttgaacagagcctgtaacatctcaatgtctttattatctatagccaatttaatgtccggaatgaagagaagggaattattggtgtttgtcgacgtcatatagtcgagcaagagaatcatcatatccacgtgtccattttttatagtgatgtgaatacaactaaggagaatagccagatcaaaagtagatggtatctctgaaagaaagtaggaaacaatacttacatcattaagcatgacggcatgataaaatgaagttttccatccagttttcccatagaacatcagtctccaatttttcttaacaaacagttttaccgtttgcatgttaccactatcaaccgcataatacaatgcggtgtttcccttgtcatcaaattgtgaatcatccagtccactgaatagcaaaatctttactattttggtatcttccaatgtggctgcctgatgtaatggaaattcattctctagaagatttttcaatgctccagcgttcaacaacgtacatactagacgcacgttattatcagctattgcataatacaaggcactatgtccatggacatccgccttaaatgcatctttgctagagagaaagcttttcagctgcttagacttccaagtattaattcgtgacagatccatgtctgaaacgagacgctaattagtgtatattttttcattttttataattttgtcatattgcaccagaattaataatatctctaatagatctgattagtagatacatggctatcgcaaaacaacatatacacatttaataaaaataatatttattaagaaaattcagatttcacgtacccatcaatataaataaaataatgattccttacaccgtacccatattaaggagattccaccttacccataaacaatataaatccagtaatatcatgtctgatgatgaacacaaatggtgtattaaattccagtttttcaggagatgatctcgccgtagctaccataatagtagatgcctctgctacagttccttgttcgtcgacatctatctttgcattctgaaacattttataaatatataatgggtccctagtcatatgtttaaacgacgcattatctggattaaacatactaggagccatcatttcggctatcgacttaatatccctcttattttcgatagaaaatttagggagtttaagattgtacactttattccctaattgaaacgaccaatagtctaattttgcagccgtaatagaatctgtgaaatgggtcatattatcacctattgccaggtacatactaatattagcatccttatacggaaggcgtaccatatcatattcttcgtcatcgattgtgattgtatttccttgcaatttagtaactacgttcatcatgggaaccgttttcgtaccgtacttattagtaaaactagcattgcgtgttttagtgatatcaaacggatattgccatatacctttaaaatatatagtattaatgattgcccatagagtattattgtcgagcatattagaatctactacattagacataccggatctacgttctactatagaattaattttattaaccgcatctcgtctaaagtttaatctatataggccgaatctatgatattgttgataatacgacggtttaatgcacacagtattatctacgaaactttgataagttagatcagtgtacgtatatttagatgttttcagcttagctaatcctgatattaattctgtaaatgctggacccagatctctttttctcaaatccatagtcttcaataattctattctagtattacctgatgcaggcaatagcgacataaacatagaaaacgaataaccaaacggtgagaagacaatattatcatcttgaatatttttatacgctactataccggcattggtaaatccttgcagacgataggtagacactgaacacgttaacgatagtatcaataacgcaatcatgattttatggtattaataattaaccttatttttatgttcggtataaaaattattgatgtctacacatccttttgtaattgacatctatatatccttttgtataatcaactctaatcactttaacttttacagttttccctaccagtttatccctatattcaacatatctatccatatgcatcttaacactctctgccaagatagcttcaaagtgaggatagtcaaaaagataaatatatagagcataatccttctcgtatactctgccctttattacatcacccgcattgggcaacgaataacaaaatgcaagcatcttgttaacgggctcgtaaattgggataaaaattatgtttttatatctattttattcaagagaatattcaggaatttctttttccggttgtatctcatcgcagtatatatcatttgtacattgtttcatattttttaatagtctacaccttttagtaggactagtatcgtacaattcatagctgtattttgaattccaatcacgcataaaaatatcttccaattgttgacgaagacctaatccatcatccggtgtaatattaatagatgctccacatgtatccgtaaagtaatttcctgtccaatttgaggtacctatatacgccgttttatcggttaccatatatttggcatggtttaccctagaatacggaatgggaggatcagcatctggtacaataaatagctttacttctatatttatgtttttagattttagcatagcgatagatcttaaaaagtttctcatgataaacgaagatcgttgccagcaactaatcaatagcttaactgacacttgtctgtctatagcggctcttcttaattcatcttctatataaggccaaaacaaaatattgcctgccttcgaataaataatagggataaagttcataacagatacataaacgaatttactcgcatttctgatacatgacaataaagcggttaaatcattggttctttccatagtacatagttgttgcggtgcagaagcaataaatacagagtgtggaacgccgcttacgttaatactaagaggatgatctgtattataatacgacggataaaagtttttccaattatatggtagattgttaactccaagataccagtatacctcaaaaatttgagtgagatccgctgccaagttcctattattgaagatcgcaatacccaattctttgacctgagttagtgatctccaatccatgttagcgcttcctaaataaatatgtgtattatcagatatccaaaattttgtatgaagaactcctcctaggatatttgtaatatctatgtatcgtacttcaactccggccatttgtagtctttcaacatcctttaatggtttgttagatttattgacggctactctaactcgtactcctcttttgggtaattgtacaatcttgtttaatattatcgtgccgaaattcgtacccacttcatccgataaactccaataaaaagatgatatatctagtgtttttgtggtattggatagaatttccctccacatgttaaatgtagacaaatatactttatcaaattgcatacctataggaatagtctctgtaatcactgcgattgtattatccggattcattttatttgttaaaagaataatcctatatcacttcactctattaaaaatccaagtttctatttctttcatgactgattttttaacttcatccgtttccttatgaagatgatgtttggcaccttcataaatttttatttctctattacaatttgcatgttgcatgaaataatatgcacctaaaacatcgctaatcttattgtttgttccctggagtatgagagtcggggggtgttaatcttggaaattatttttctaaccttgttggtagccttcaagacctgactagcaaatccagccttaattttttcatgattgattaatgggtcgtattggtatttataaactttatccatatctctagatactgattctggacatagctttccgactggcgcatttagtgtgatggttcccataagtttggcagctagcagattcagttttgaaacagcatctgcattaactagaggagacattagaatcattgctgtaaacaagtttggattatcgtaagaggctagctcccatggaatgacccaataagtagatttaatagttaccacgtgctgtaccaaagtcatcaatcatcattttttcaccattacttcttccatgtccaatatgatcatgtgagaatactaaaattcctaacgatgatatgttttcagctagttcgtcataacgtccagaatgtttaccagctccatgacttatgaatactaatgccttaggatatgtaataggtttccaatatatgtaatcattgtccagattgaacatacagtttgcactcatgattcacgttatataactatcaatattaacagttcgtttgatgatcatattatttttatgttttattgataattgtaaaaacatacaattaaatcaatatagaggaaggagacggctactgtcttttgtgagatagtcatggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtttattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaattgatgatataggggtcttcataacgcataattattacgttagcattctatatccgtgttaaaaaaaattatcctatcatgtatttgagagttttatatgtagcaaacatgatagctgtgatgccaataagctttagatattcacgcgtgctagtgttagggatggtattatctggtggtgaaatgtccgttatataatctacaaaataatcatcgcatatagtatgcgatagtagagtaaacatttttatcgtttctactgggttcatacatcgtctacccaattcggttataaatgaaattgtcgccaatcttacacccaaccccttgttatccattagtatagtattaacttcgttatttatgtcataaactgtaaatgattttgtagatgccatatcatacatgatattcatgtccctattataatcattactaactttatcacaatatatgttgataatatctatatatgatctagtctttgtgggcaactgtctatacaagtcgtctaaacgttgtttactcatatagtatcgaacagccatcattacatggtcccgttccgttgatagataatcgagtatgttagtggacttgtcaaatctatataccatattttctggaagtggatatacatagtcgtgatcaacattattgctagcctcatcttctatatcctgtactataccattatctatatcatctacataatctacgatattattacacataaacatcgacaacatactattgtttattatctaagtcctgttgatccaaacccttgatctcctctatttgtactatctagagattgtacttcttccagttctggataatatatacgttgatagattagctgagctattctatctccagtatttacattaaacgtacattttccattattaataagaatgactcctatgtttcccctataatcttcgtctattacaccgcctcctatatcaatgccttttagggacagaccagacctaggagctattctaccatagcagaacttaggcatggacatactaatatctgtcttaattaactgtcgttctcctggagggatagtataatcgtaagcgctatataaatcatatccggcggcgtaaggtgattgcctagtaggagatttagctctgttagtttccttaacaaatctaactggtgagttaatattcatgttgaacataaaactaatattttatttcaaaattatttaccatcccatatattccatgaataagtgtgatgattgtacacttctatagtatctatatacgattcacgataaaatcctcctatcaatagcagtttattatccactatgatcaattctggattatccctcggataaataggatcatctatcagagtccatgtattgctggattcacaataaaattccgcatttctaccaaccaagaataaccttctaccgaacactaacgcgcatgatttataatgaggataataagtggatggtccaaactgccactgatcatgattgggtagcaaatattctgtagttgtatcagtttcagaatgtcctcccattacgtatataacattgtttatggatgccactgctggattacatctaggtttcagaagactcggcatattaacccaagcagcatccccgtggaaccaacgctcaacagatgtgggatttggtagacctcctactacgtataatttattgttagcgggtatcccgctagcatacagtctggggctattcatcggaggaattggaatccaattgtttgatatataatttaccgctatagcattgttatgtatttcattgttcatccatccaccgatgagatatactacttctccaacatgagtacttgtacacatatggaatatatctataatttgatccatgttcataggatactctatgaatggatacttgtatgatttgcgtggttgtttatcacaatgaaatattttggtacagtctagtatccattttacattatttatacctctgggagaaagataatttgacctgattacatttttgataaggagtagcagatttcctaatttatttcttcgctttatataccacttaatgacaaaatcaactacataatcctcatctggaacatttagttcatcgctttctagaataagtttcatagatagataatcaaaattgtctatgatgtcatcttccagttccaaaaagtgtttggcaataaagtttttagtatgacataagagattggatagtccgtattctatacccatcatgtaacactcgacacaatattcctttctaaaatctcgtaagataaagtttatacaagtgtagatgataaattctacagaggttaatatagaagcacgtaataaattgacgacgttatgactatctatatatacctttccagtatacgagtaaataactatagaagttaaactgtgaatgtcaaggtctagacaaacccttgtaactggatctttatttttcgtgtatttttgacgtaaatgtgtgcgaaagtaaggagataactttttcaatatcgtagaattgactattatattgccacctatagcatcaataattgttttgaatttcttagtcatagacaatgctaatatattcttacagtacacagtattaacaaatatcggcatttatgtttctttaaaagtcaacatctaaagaaaaatgattatcttcttgagacataactcccattttttggtattcacccacacgtttttcgaaaaaattagtttttccttccaatgatatattttccatgaaatcaaacggattggtaacattataaatttttttaaatcccaattcagaaatcaatctatccgcgacgaattctatatatgttttcatcatttcacaattcattcctataagtttaactggaagagccgcagtaagaaattcttgttcaatggataccgcatctgttataatagatctaacggtttcttcactcggtggatgcaataaatgtttaaacatcaaacatgcgaaatcgcagtgcagaccctcgtctctactaattagttcgttggaaaacgtgagtccgggcattaggccacgctttttaagccaaaatatggaagcgaatgatccagaaaagaagattccttctactgcagcaaaggcaataagtctctctccataaccggcgctgtcatgtatccacttttgagcccaatcggccttcttttttacacaaggcatcgtttctatggcattaaagagatagtttttttcattactatctttaacataagtatcgatcaaaagactatacatttccgaatgaatgttttcaatggccatctgaaatccgtagaaacatctagcctcggtaatctgtacttctgtacaaaatcgttccgccaaattttcattcactattccgtcactggctgcaaaaaacgccaatacatgttttataaaatatttttcgtctggtgttagtttattccaatcattgatatctttagatatatctacttcttccactgtccaaaatgatgcctctgcctttttatacatgttccagatgtcataatattggattgggaaaataacaaatctatttggatttggtgcaaggatgggttccataactaaattaacaataacaataaattttttttcagttatctatatgcctgtacttggatcttttgtacatcgatatcgccgcaatcactacaataattacaagtattattgatagcattgttattagtactatcataattaaattatcgacattcatgggtgctgaataatcgttattatcatcattatcattttgtaattgtgacatcatactagataaatcgtttgcgagattgttgtgggaagcgggcatggaggatgaattatcgttattattatttaacgcctcccattcggattcacaaatgttacgcacattcaacattttatggaaactataattttgtgaaaacagataacaagaaaactcgtcatcgttcaaatttttaacgatagtaaaccgattaaacgtcgagctaatttctaacgctagcgactctgttggatatgggtttccagatatatatcttttcagttcccctacgtatctataatcatctgtaggaaatggaagatatttccatttatctactgttcctaatatcatatgtggtggtgtagtagaaccattaagcgcgaaagatgttatttcgcatcgtattttaacttcgcaataatttctggttagataacgcactctaccagtcaagtcaatgatattagcctttacagatatattcatagtagttgtaacgatgactccatcttttagatgcgatactcctttgtatgtaccagaatcttcgtaccgcaaactcgatatatttaaacaagttaatgagatattaacgcgttttatgaatgatgatatataaccagaagttttatcctcggtggctagcgctataaccttatcattataataccaactagtgtgattaatatgtgacacgttagtgtgggtacaaatatgtacattatcgtctacgtcgtattcgatacatccgcatacagccaacaaatataaaatgacaaatactctaacgccgttcgtacccatcttgatgcggtttaataaatgttttgatttcaatttattgtaaaaaaagattcggttttatactgttcgatattctcattgcttatattttcatctatcatctccacacagtcaaatccgtggttagcatgcacctcatcaaccggtaaaagactatcggactcttctatcattataactctagaatatttaatttggtcattattaatcaagtcaattatcttatttttaacaaacgtgagtattttactcattttttataaaaacttttagaaatatacagactctatcgtgtgtctatatcttctttttatatccaatgtatttatgtctgatttttcttcatttatcatatataatggtccaaattctacacgtgcttcggattcatccagatcattaaggttcttataattgtaacatccttctcttccctcttctacatcttccttcttattcttattcttagcgtcacagaatctaccacagcaggatcccatgacgagcgtcatattaaactaatccattttcaattataatatacgattagtaatgaccattaaaataaaaaatattcttcataaccggcaagaaagtgaaaagttcacattgaaactatgtcagtagtatacatcatgaaatgatgatatatatatactctattttggtggaggattatatgatataattcgtggataatcattcttaagacacatttcttcattcgtaaatcttttcacgttaaatgagtgtccatattttgcaatttcttcatatgatggcggtgtacgtggacgaagctgctcctgttcttgttgtagtcgccgactgtcgtgtttgcgtttagatccctccattatcgcgattgcgtagatggagtactattatataccttgtaattaaatttttttattaattaaacgtataaaaacgttccgtatctgtatttaagagccagatttcgtctaatagaacaaatagctacagtaaaaataactagaataattgctacacccactagaaaccacggatcgtaatacggcaatcggttttcgataataggtggaacgtatattttatttaaggacttaacaattgtctgtaaaccacaatttgcttccgcggatcctgtattaactatctgtaaaagcatatgttgaccgggcggagccgaacattctccgatatctaatttctgtatatctataatattattaacctccgcatacgcattacagttcttttctagcttggataccgcactaggtacatcgtctagatctattcctatttcttcagcgatagctcttctatccttttccggaagcaatgaaatcacttcaataaatgattcaaccatgagtgtgaaactaagtcgagaattactcatgcatttgttagttattcggagcgcgcaatttttaaactgtcctataacctctcctatatgaatagcacaagtgacattagtagggatagaatgttgagctaatttttgtaaataactatctataaaaagattatacaaagttttaaactctttagtttccgccatttatccagtctgagaaaatgtctctcataataaatttttccaagaaactaattgggtgaagaatggaaacctttaatctatatttatcacagtctgttttggtacacatgatgaattcttctaatgctgtactaaattcgatatctttttcgatttctggatatgtttttaataaagtatgaacaaagaaatggaaatcgtaataccagttatgttcaactttgaaattgttttttattttcttgttaatgattccagccacttgggaaaagtcaaagtcgtttaatgccgatttaatacgttcattaaaaacaaactttttatcctttagatgaattattattggttcattggaatcaaaaagtaagatattatcgggtttaagatctgcgtgtaaaaagttgtcgcaacagggtagttcgtagattttaatgtataacagagccatctgtaaaaagataaactttatgtattgtaccaaagatttaaatcctaatttgatagctaactcggtatctactttatctgccgaatacagtgctaggggaaaaattataatatttcctctttcgtattcgtagttagttctcttttcatgttcgaaaaagtgaaacatgcggttaaaatagtttataacattaatattactgttaataactgccggataaaagtgggatagtaatttcacgaatttgatactgtcctttctctcgttaaacgcctttaaaaaaactttagaagaatatctcaatgagagttcctgaccatccatagtttgtatcaataatagcaacatatgaagaacccgtttatacagagtatgtaaaaatgttaatttatagtttaatcccatggcccacgcacacacgattaattttttttcatctccctttagattgttgtatagaaatttgggtactgtgaactccgccgtagtttccatgggactatataattttgtggcctcgaatacaaattttactacatagttatctatcttaaagactataccatatcctcctgtagatatgtgataaaaatcgtcgtttataggataaaatcgtttatccttttgttggaaaaaggatgaattaatgtaatcattctcttctatctttagtagtgtttccttattaaaattcttaaaataatttaacaatctaactgacggagcccaattttggtgtaaatctaattgggacattatgttgttaaaatacaaacagtctcctaatataacagtatctgataatctatggggagacatccattgatattcaggggatgaatcattggcaacacccatttattgtacaaaaagccccaatttacaaacgaaagtccaggtttgatagagacaaacaattaactattttgtctctgtttttaatttctttggtaatgaaattattcacaatatcagtatcttctttatctaccagagattttactaacttgataaccttggctgtctcattcaatagggtagtaatatttgtatgtgtgatattgatatctttttgaattgtttcttttagaagtgattctttgatggtgccagcatacgaattacaataatgcagaaactcagttaacatgcaggaattatagtaagccaattccaattgttgcctgtattgtattagagtattaatatgcgcaatggtgtccttgcgtttctctgatagaatgcgagcagcgattttggcgttatcatttgacgatatttctggaatgacgaatcctgtttctactaactttttggtaggacaaagtgaaacaatcaagaagatagcttctcctcctatttgtggaagaaattgaactcctctagatgatctactgacgatagtatctccttgacagatattggaccgaattacagaagtacctggaatgtaaagccctgaaaccccctcattttttaagcagattgttgccgtaaatcctgcactgtgaccaagatagagagctcctttggtgaatccatctctatgtttcagtttaaccaagaaacagtcagctggtctaaaatttccatctctatctaatacagcatctaacttgatgtcaggaactatgaccggtttaatgttatatgtaacattgagtaaatccttaagttcataatcatcactgtcatcagttatgtacgatccaaacaatgtttctactggcatagtggatacgaagatgctatccatcagaatgtttccctgattagtattttctatatagctattcttctttaaacgattttccaaatcagtaactatgttcatttttttaggagtaggacgcctagccagtatggaagaggattttctagatcctctcttcaacatctttgatctcgatggaatgcaaaaccccatagtgaaacaaccaacgataaaaataatattgtttttcactttttataattttaccatctgactcatggattcattaatatctttataagagctactaacgtataattctttataactgaactgagatatatacaccggatctatggtttccataattgagtaaatgaatgctcggcaataactaatggcaaatgtatagaacaacgaaattatactagagttgttaaagttaatattttctatgagctgttccaataaattatttgttgtaactgcgttcaagtcataaatcatcttgatactatccagtaaaccgtgtttaagttctggaatattatcatcccattgtaaagcccctaattcgactatcgaatatcctgctctgatagcagtttcaatatcgacggacgtcaatactgtaataaaggtggtagtattgtcatcatcgtgataaactacgggaatatggtcgttagtaggtacggtgactttacacaacgcgatatataactttccttttgtaccatttttaacgtagttgggacgtcctgcagggtattgttttgaagaaatgatatcgagaacagatttgatacgatatttgttggattcctgattattcactataatataatctagacagatagatgattcgataaatagagaaggtatatcgttggtaggataatacatccccattccagtattctcggatactctattgatgacactagttaagaacatgtcttctattctagaaaacgaaaacatcctacatggactcattaaaacttctaacgctcctgattgtgtctcgaatgcctcgtacaaggatttcaaggatgccatagattctttgaccaacgatttagaattgcgtttagcatctgatttttttattaaatcgaatggtcggctctctggtttgctaccccaatgataacaatagtcttgtaaagataaaccgcaagaaaatttatacgcatccatccaaataaccctagcaccatcggatgatattaatgtattattatagattttccatccacaattattgggccagtatactgttagcaacggtatatcgaatagattactcatgtaacctactagaatgatagttcgtgtactagtcataatatctttaatccaatctaagaaatttaaaattagattttttacactgttaaagttaacaaaggtattacccggatacgtggatatcatatatggcattggtccattatcagtaatagctccataaactgatacggcgatggtttttatatgtgtttgatctaacgaggaagaaattcgcgcccacaattcatctctagatatgtatttaatatcaaacggtaacacatcaatttcgggacgcgtatatgtttctaaatttttaatccaaatataatgatgacctatatgccctattatcatactgtcaactatagtacacctagagaacttacgatacatctgtttcctataatcgttaaattttacaaatctataacatgctaaaccttttgacgacagccattcattaatttctgatatggaatctgtattctcgataccgtattgttctaaagccagtgctatatctccctgttcgtgggaacgctttcgtataatatcgatcaacggataatctgaagtttttggagaataatatgactcatgatctatttcgtccataaacaatctagacataggaattggaggcgatgatcttaattttgtgcaatgagtcgtcaatcctataacttctaatcttgtaatattcatcatcgacataatactatctatgttatcatcgtatattagtataccacggccttcttcatttcgtgccaaaataatatacagtcttaaataattacgcaatatctcaatagtttcataattgttagctgttttcatcaagatttgtaccctgtttaacatgatggcgttctatacgtctctattttcttttttttaaatttttaacgatttactgtggctagatacccaatctctctcaaatatttttttagcctcgcttacaagctgtttatctatactattaaaactgacgaatccgtgattttggtaatgggttccgtcgaaatttgccgaagtgatatgaacatattcgtcgtcgactatcaacaattttgtattattctgaatagtgaaaaccttcacagatagatcattttgaacacacaacgcgtctagacttctggcggttgccatagaatatacgtcgttcttatcccaattaccaactagaagtctgatcttaactcctctattaatggctgcttctataatggagttgtaaatgtcgggccaatagtagctattaccgtcgacacgtgtagtgggaactatggccaaatgttcaatatctatactagtcttagccgacttgagtttatcaataactacatcggtatctagatctctagaatatcccaataggtgttccggagaatcagtaaagaacactccacctataggattcttaatatgatacgcagtgctaactggcaaacaacaagccgcagagcataaattcaaccatgaattttttgcgctattaaaggctttaaaagtatcaaatcttctacgaagatctgtggccagcgggggataatcagaatatacacctaacgttttaatcgtatgtatagatcctccagtaaatgacgcgtttcctacataacatctttcatcatctgacacccaaaaacaaccgagtagtagtcccacattattttttttatctatattaacggttataaaatttatatccgggcagtgactttgtagctctcccagatttcttttccctcgttcatctagcaaaactattattttaatccctttttcagatgcctcttttagtttatcaaaaataagcgctcccctagtcgtactcagaggattacaacaaaaagatgctatgtatatatatttcttagctagagtgataatttcgttaaaacattcaaatgttgttaaatgatcggatctaaaatccatattttctggtagtgtttctaccagcctacattttgctcccgcaggtaccgatgcaaatggccacatttagttaacataaaaacttatacatcctgttctatcaacgattctagaatatcatcggctatatcgctaaaattttcatcaaagtcgacatcacaacctaactcagtcaatatattaagaagttccatgatgtcatcttcgtctatttctatatccgtatccattgtagattgttgaccgattatcgagtttaaatcattactaatactcaatccttcagaatacaatctgtgtttcattgtaaatttataggcggtgtatttaagttggtagattttcaattatgtatcaatatagcaacagtagttcttgctcctccttgattctagcatcctcttcattattttcttctacgtacataagcatgtccaatacgttagacaacacaccgacgatggcggccgccacagacacgaatatgactagaccgatgaccatttaaaaacccctctctagctttcacttaaactgtatcgattattcttttagaacatgtataatataaaaacattattctatttcgaatttaggcttccaaaaatttttcatccgtaaaccgataataatatatatagacttgttaatagtcggaataaatagattaatgcttaaactatcatcatctccacgattagagatacaatatttacattttttttgctgtttcgaaactttatcaatacacgttaatacaaacccaggaaggagatattgaaactgaggctgttgaaaatgaaacggtgaatacaataattcagataatgtaaaatcatgattccgtattctgatgatattagaactgctaatggatgtcgatggtatgtatctaggagtatctattttaacaaagcatcgatttgctaatatacaattatccttttgattaattgttattttattcatattcttaaaaggtttcatatttatcaattcttctacattaaaaatttccatttttaatttatgtagcccccgcaatactcctcattacgtttcattttttgtctataatatccattttgttcatctcggtacatagattatccaattgagaagcgcatttagtagttttgtacattttaagtttattgacgaatcgtcgaaaactagttatagttaacattttattatttgataccctgatattaatacccctgccgttactattatttataactgatgtaatccacgtaacattagaattaattatcgatagtaatgcatcgacgcttccaaaattgtctattataaactcaccgataatttttttattgcatgttttcatattcattaggattatcaaatctttaatcttactacgattgtatgcgttgatattgcaagacgtcattctaaaagacggaggatctccatcaaatgccagacaatcacgtacaaagtacatggaaataggttttgttctattgcgcatcatagatttatatagaacacccgtagaaatactaatttgttttactctataaaatactaatgcatctatttcatcgttttgtataacgtctttccaagtgtcaaattccaaatttttttcattgatagtaccaaattcttctatctctttaactacttgcatagataggtaattacagtgatgcctacatgccgttttttgaaactgaatagatgcgtctagaagcgatgctacgctagtcacaatcaccactttcatatttagaatatatgtatgtaaaaatatagtagaatttcattttgtttttttctatgctataaatgaattctcattttgcatctgctcatactccgttttatatcaataccaaagaaggaagatatctggttctaaaagccgttaaagtatgcgatgttagaactgtagaatgcgaaggaagtaaagcttcctgcgtactcaaagtagataaaccctcatcgcccgcgtgtgagagaagaccttcgtccccgtccagatgcgagagaatgaataaccctggaaaacaagttccgtttatgaggacggacatgctacaaaatatgttcgcggctaatcgcgacaacgtggcgtcgagacttttgaactaaaatacaattatatccttttcgatattaataaatccgtgtcgtccaggttttttatctctttcagtatgtgaatagataggtattttatctctattcatcatcgaatttaagagatccgataaacattgtttgtattctccagatgtcagcatctgatacaacaatatatgtgcacataaacctctggcacttatttcatgtaccttccccttatcactaaggagaatagtatttgagaaatatgtatacatgatattatcatgaattagatatacagaatttgtaacactctcgaaatcacacgatgtgtcggcgttaagatctaatatatcactcgataacacattttcatctagatacactagacattttttaaagctaaaatagtctttagtagtgacagtaactatgcgattattttcatcgatgatacatttcatcggcatattattacgcttaccatcaaagactataccatgtgtatatctaacgtattctagcatggttgccatacgcgcattaaacttttcaggatctttggatagatcttccaatctatctatttgagaaaacatttttatcatgttcaatagttgaaacgtcggatccactatatagatattatctataaagattttaggaactacgttcatggtatcctggcgaatattaaaactatcaatgatatgattatcgttttcatcttttatcaccatatagtttctaagatatgggattttacttaatataatattatttcccgtgataaattttattagaaaggccaaatctataagaaaagttctagaattagtctgaagaatatctatatcgccgtaccgtatatttggattaattagatatagagaatatgatccgtaacatatacaacttttattatggcgtctaagatattcttccatcaacttattaacatttttgactagggaagatacattatgacgtcccattacttttgccttgtctattactgcgacgttcatagaatttagcatatctcttgccaattcttccattgatgttacattataagaaattttagatgaaattacatttggagctttaatagtaagaactcctaatatgtccgtgtatgtggtcactaatacagattgtagttctataatcgtaaataatttacctatattatatgtttgagtctgtttagaaaagtagctaagtatacgatcttttatttctgatgcagatgtattaacatcggaaaaaaatctttttttattcttttttactaaagatacaaatatgtctttgttaaaaacagttattttttgaatatttctagcttgtaattttaacatatgatattcgttcacactaggtactctgcctaaataggtttctataatctttaatgtaatattaggaagagtattctgatcaggattcctattcattttgaggatttaaaactctgattattgtctaatatggtctctacgcaaactttttcacagagcgatagagtttttgataactcgtttttcttaagaaatataaaactactgtctccagagctcgctctatcttttattttatttaattcgatacaaactcctgatactggttcagaaagtaattcattaattttcagtcctttatagaagatatttaatatagataatacaaaattttcagttcttgatatcgatctgattgatcctagaactagatatattaataacgtgctcattaggcagtttatggcagcttgataattagatatagtatattccagttcatatttattagataccgcattgcccagattttgatattctatgaattcctctgaaaataaatccaaaataactaaacattctattttttgtggattagtgtactctcttccctctatcatgttcactactggtgtccacgatgataaatatctagagggaatataatatagtccataggatgccaatctagcaatgtcgaataactgtaatttgattcttcgttcttcattatgaattgattcttgaggtataaacctaacacaaattatattattagacttttcgtatgtaatgtctttcatgttataagtttttaatcctggaatagaatctattttaatgaggcttttaaacgcagagttctccaacgagtcaaagcataatactctgttgtttttcttatatacgatgttacgattttcttctttgaatggaataggtttttgaattagtttataattacaacataatagataaggaagtgtgcaaatagtacgcggaaaaaacataatagctcccctgttttcatccatggttttaagtaaatgatcactggcttctttagtcaatggatattcgaacattaaccgtttcatcatcattggacagaatccatatttcttaatgtaaagagtgatcaaatcattgtgtttattgtaccatcttgttgtaaatgtgtattcggttatcggatctgctcctttttctattaaagtatcgatgtcgatctcgtctaagaattcaactatatcgacatatttcatttgtatacacataaccattactaacgtagaatgtataggaagagatgtaacgggaacagggtttgttgattcgcaaactattctaatacataattcttctgttaatacgtcttgcacgtaatctattatagatgccaagatatctatataattattttgtaagatgatgttaactatgtgatctatataagtagtgtaataattcatgtatttcgatatatgttccaactctgtctttgtgatgtctagtttcgtaatatctatagcatcctcaaaaaatatattcgcatatattcccaagtcttcagttctatcttctaaaaaatcttcaacgtatggaatataataatctattttacctcttctgatatcattaatgatatagtttttgacactatcttctgtcaattgattcttattcactatatctaagaaacggatagcgtccctaggacgaactactgccattaatatctctattatagcttctggacataattcatctattataccagaattaatgggaactattccgtatctatctaacatagttttaagaaagtcagaatctaagacctgatgttcatatattggttcatacatgaaatgatctctattgatgatagtgactatttcattctctgaaaattggtaactcattctatatatgctttccttgttgatgaaggatagaatatactcaatagaatttgtaccaacaaactgttctcttatgaatcgtatatcatcatctgaaataatcatgtaaggcatacatttaacaattagagacttgtctcctgttatcaatatactattcttgtgataatttatgtgtgaggcaaatttgtccacgttctttaattttgttatagtagatatcaaatccaatggagctacagttcttggcttaaacagatatagtttttctggaacgaattctacaacattattataaaggactttgggtagataagtgggatgaaatcctattttaattaatgcgatagccttgtcctcgtgcagatatccaaacgcttttgtgatagtatggcattcattgtctagaaacgctctacgaatatctgtgacagatatcatctttagagaatatactagtcgcgttaatagtactacaatttgtattttttaatctatctcaataaaaaaattaatatgtatgattcaatgtataactaaactactaactgttattgataactagaatcagaatctaatgatgacgtaaccaagaagtttatctactgccaatttagctgcattatttttagcatctcgtttagattttccatctgccttatcgaatactcttccgtcgatgtctacacaggcataaaatgtaggagagttactaggccccactgattcaatacgaaaagaccaatctctcctagtaatttggcagtactcattaataacggtgacagggttagcacctttccaatcaataatttttttagccggaataacatcatcaaaagacttatgatcctctctcattgatttttcgcgggatacatcatctattatgacgtcagccatagcatcagcatccggcttatccgcctccgttgtcataaaccaacgaggaggaatatcgtcggagctgtacaccatagcactacgttgaagatcgtacagagctttattaacttctcgcttctccatattaagttgtctagttagttgtgcagcagtagctccttcgattccaatggttttaatagcctcacacacaatctctgcgttagaacgctcgtcgatatagattttagacatttttagagagaactaacgcaatcagtaataaaactaatttattttatcattttttttattcatcatcctctggtggttcgtcgtttctatcgaatgtagctctgattaacccgtcatctataggtgatgctggttctggagattctggaggagatggattattatctggaagaatctctgttatttccttgttttcatgtatcgattgcgttgtaacattaagattgcgaaatgctctaaatttgggaggcttaaagtgttgtttgcaatctctacacgcgtgtctaactagtggaggttcgtcagcggctctagtttgaatcatcatcggcgtagtattcctacttttacagttaggacacggtgtattgtatttctcgtcgagaacgttaaaataatcgttgtaactcacatcctttattttatctatattgtattctactcctttcttaatgcattttataccgaataagagatagcgaaggaattctttttcggtgccgctagtacccttaatcatatcacatagtgttttatattccaaatttgtggcaatagacggtttatttctatacgatagtttgtttctggaatcctttgagtattctataccaatattattctttgattcgaatttagtttcttcgatattagattttgtattacctatattcttgatgtagtactttgatgatttttccatggcccattctattaagtcttccaagttggcatcatccacatattgtgatagtaattctcggatatcagtagcggttaccgccattgatgtttgttcattggatgagtaactactaatgtatacattttccatttataacacttatgtattaactttgttcatttatattttttcattattatgttgatattaacaaaagtgaatatatatatgttaataattgtattgtggttatacggctacaattttataattagtgaaagtcagtgtccgatgatcaatgacgatagctttactctgaaaagaaagtatcaaatcgatagtgcggagtcaacaataaaaatggataagaagaggacaaagtttcagaatagagccaaaatggtaaaagaaataaatcagacaataagagcagcacaaactcattacgagacattgaaactaggatacataaaatttaagagaatgattaggactactactctagaagatatagcaccatctattccaaataatcagaaaacttataaactattctcggacatttcagccatcggcaaagcatcacggaatccaagtaagatggtatatgctctgctgctttacatgtttcccaatttgtttggagatgatcatagattcattcgttatagaatgcatccaatgagtaaaatcaaacacaagatcttctctcctttcaaacttaatcttattagaatattagtggaagaaagattctataataatgaatgcagatctaataaatggagaataattggaacacaagttgataaaatgttgatagctgaatctgataaatatacaatagatgcaaggtataacctaaaacccatgtatagaatcaagggaaaatctgaagaagataccctctttatcaaacagatggtagaacaatgtgtgacatcccaggaattggtggaaaaagtgttgaagatactgtttagagatttgttcaagagtggagaatacaaagcgtacagatacgatgatgatgtagaaaatggatttattggattggatacactaaaattaaacattgttcatgatatagttgaaccatgtatgcctgttcgtaggccagtggctaagatactgtgtaaagaaatggtaaataaatactttgagaatccgctacatattattggtaaaaatcttcaagagtgcattgactttgttagtgaataggcatttcatctttctccaatactaattcaaattgttaaattaataatggatagtataaatagttattagtgataaaatagtaaaaataattattagaataagagtgtagtatcatagataactctcttctataaaaatggattttattcgtagaaagtatcttatatacacagtagaaaataatatagattttttaaaggatgatacattaagtaaagtaaacaattttaccctcaatcatgtactagctctcaagtatctagttagcaattttcctcaacatgttattactaaggatgtattagctaataccaatttttttgttttcatacatatggtacgatgttgtaaagtgtacgaagcggttttacgacacgcatttgatgcacccacgttgtacgttaaagcattgactaagaattatttatcgtttagtaacacaatacaatcgtacaaggaaaccgtgcataaactaacacaagatgaaaaatttttagaggttgccaaatacatggacgaattaggagaacttataggcgtaaattatgacttagttcttaatccattatttcacggaggggaacccatcaaagatatggaaatcatttttttaaaactgtttaagaaaacagacttcaaagttgttaaaaaattaagtgttataagattacttatttgggcttacctaagcaagaaagatacaggcatagagtttgcggataatgatagacaagatatatatactctatttcaacaaactggtagaatcgtccatagcaatctaacagaaacgtttagagattatatctttcccggagataagactagctattgggtgtggttaaacgaaagtatagctaatgatgcggatattgttcttaatagacacgccattaccatgtatgataaaattcttagttatatatactctgagataaaacaaggacgcgttaataaaaacatgcttaagttagtttatatctttgagcctgaaaaagatatcagagaacttctgctagaaatcatatatgatattcctggagatatcctatctattattgatgcaaaaaacgacgattggaaaaaatattttattagtttttataaagctaattttattaacggtaatacatttattagtgatagaacgtttaacgaggacttattcagagttgttgttcaaatagatcccgaatatttcgataatgaacgaattatgtctttattctctacgagtgctgcggacattaaacgatttgatgagttagatattaataacagttatatatctaatataatttatgaggtgaacgatatcacattagatacaatggatgatatgaagaagtgtcaaatctttaacgaggatacgtcgtattatgttaaggaatacaatacatacctgtttttgcacgagtcggatcccatggtcatagagaacggaatactaaagaaactgtcatctataaaatccaagagtagacggctgaacttgtttagcaaaaacattttaaaatattatttagacggacaattggctcgtctaggtcttgtgttagatgattataaaggagacttgttagttaaaatgataaaccatcttaagtctgtggaggatgtatccgcattcgttcgattttctacagataaaaaccctagtattcttccatcgctaatcaaaactattttagctagttataatatttccatcatcgtcttatttcaaaggtttttaagagataatctatatcatgtagaagaattcttggataaaagcatccatctaaccaagacggataagaaatatatacttcaattgataagacacggtagatcatagaacagaccaaatatattattaataatttgtatatacatagatataattatcacatattaaaaattcacacatttttgataaatgggaactgctgcaacaattcagactcccaccaaattaatgaataaagaaaatgcagaaatgattttggaaaaaattgttgatcatatagttatgtatattagtgacgaatcaagtgattcagaaaataatcctgaatatattgattttcgtaacagatacgaagactatagatctctcattataaaaagtgatcacgagtttgtaaagctatgtaaaaatcatgcagagaaaagttctccagaaacgcaacaaatgattatcaaacacatatacgaacaatatcttattccagtatctgaagtactattaaaacctataatgtccatgggtgacataattacatataacggatgtaaagacaatgaatggatgctagaacaactctctaccctaaactttaacaatctccgcacatggaactcatgtagcataggcaatgtaacgcgtctgttttatacattttttagttatctgatgaaagataaactaaatatataagtataatcccattctaatactttaacctgatgtattagcatcttattagaatattaacctaactaaaagacataacataaaaactcattacatagttgataaaaagcggtaggatataaatattatggctgccaccgttccgcgttttgacgacgtgtacaaaaatgcacaaagaagaattctagatcaagaaacattttttagtagaggtctaagtagaccgttaatgaaaaacacatatctatttgataattacgcgtatggatggataccagaaactgcaatttggagtagtagatacgcaaacttagatgcaagtgactattatcccatttcgttgggattacttaaaaagttcgagtttctcatgtctctatataaaggtcctattccagtatacgaagaaaaagtaaatactgaattcattgctaatggatcgttctctggtagatacgtatcatatcttcgaaagttttctgctcttccaacaaacgagtttattagttttttgttactgacttccattccaatctataatatcttgttctggtttaaaaatactcagtttgatattactaaacacacattattcagatacgtctatacagataatgccaaacacctggcgttggctaggtatatgcatcaaacaggagactataagcctttgtttagtcgtctcaaagagaattatatatttaccggtcccgttccaataggtatcaaagatataaatcaccctaatcttagtagagcaagaagtccatccgattatgagacattagctaatattagtactatattgtactttaccaagtatgatccggtattaatgtttttattgttttacgtacctgggtattcaattactacaaaaattactccagccgtagaatatctaatggataaactgaatctaacaaagagcgacgtacaactgttgtaaattattttatgcttcgtaaaatgtaggttttgaaccaaacattctttcaaagaatgagatgcataaaactttattatccaatagattgactatttcggacgtcaatcgtttaaagtaaacttcgtaaaatattctttgatcactgccgagtttaaaacttctatcgataattgtttcatatgttttaatatttacaagttttttggtccatggtacattagccggacaaatatatgcaaaataatatcgttctccaagttctatagtttctggattatttttattatattcagtaaccaaatacatattagggttatctgcggatttataatttgagtgatgcattcgactcaacataaataattctagaggagacgatctactatcaaattcggatcgtaaatctgtttctaaagaacggagaatatctatacatacctgattagaattcatccgtccttcagacaacatctcagacagtctggtcttgtatgtcttaatcatattcttatgaaacttggaaacatctcttctagtttcactagtacctttattaattctctcaggtacagattttgaattcgacgatgccgagtatttcatcgttgtatatttcttcttcgattgcataatcagattcttatataccgcctcaaactctattttaaaattattaaacaatactctattattaatcagtcgttctaactcctttgctatttctatggacttatctacatcttgactgtctatctctgtaaacacggagtcggtatctccatacacgctacgaaaacgaaatctgtaatctataggcaacgatgttttcacaatcggattaatatctctatcgtccatataaaatggattacttaatggattggcaaaccgtaacataccgttagataactctgctccatttagtaccgattctagatacaagatcattctacgtcctatggatgtgcaactcttagccgaagcgtatgagtatagagcactatttctaaatcccatcagaccatatactgagttggctactatcttgtacgtatattgcatggaatcatagatggccttttcagttgaactggtagcctgttttaacatctttttatatctggctctctctgccaaaaatgttcttaatagtctaggaatggttccttctatcgatctatcgaaaattgctatttcagagatgaggttcggtagtctaggttcacaatgaaccgtaatatatctaggaggtggatatttctgaagcaagagctgattatttatttcttcttccaatctattggtactaacaacgacaccgactaatgtttccggagatagatttccaaagatacacacattaggatacagactgttataatcaaagattaatacattattactaaacattttttgttttggagcaaataccttaccgccttcataaggaaacttttgttttgtttctgatctaactaagatagttttagtttccaacaatagctttaacagtggacccttgatgactgtactcgctctatattcgaataccatggattgaggaagcacatatgttgacgcacccgcgtctgtttttgtttctactccataatactcccacaaatactgacacaaacaagcatcatgaatacagtatctagccatatctaaagctatgtttagattataatccttatacatctgagctaaatcaacgtcatcctttccgaaagataatttatatgtatcattaggtaaagtaggacataatagtacgactttaaatccattttcccaaatatctttacgaattactttacatataatatcctcatcaacagtcacataattacctgtggttaaaacctttgcaaatgcagcggctttgcctttcgcgtccgtagtatcgtcaccgatgaacgtcatttctctaactcctctatttaatactttacccatgcaactgaacgcgttcttggatatagaatccaatttgtacgaatccaatttttcagatttttgaatgaatgaatatagatcgaaaaatatagttccattattgttattaacgtgaaacgtagtattggccatgccgcctactcccttatgactagactgatttctctcataaatacagagatgtacagcttcctttttgtccggagatctaaagataatcttctctcctgttaataactctagacgattagtaatatatctcagatcaaagttatgtccgttaaaggtaacgacatagtcgaacgttagttccaacaattgtttagctattcgtaacaaaactatttcagaacataaaactagttctcgttcgtaatccatttccattagtgactgtatcctcaaacatcctctatcgacggcttcttgtatttcctgttccgttaacatctcttcattaatgagcgtaaacaataatcgtttaccacttaaatcgatataacagtaacttgtatgcgagattgggttaataaatacagaaggaaacttcttatcgaagtgacactctatatctagaaataagtacgatcttgggatatcgaatctaggtatttttttagcgaaacagttacgtggatcgtcacaatgataacatccattgttaatctttgtcaaatattgctcgtccaacgagtaacatccgtctggagatatcccgttagaaatataaaaccaactaatattgagaaattcatccatggtggcattttgtatgctgcgtttctttggctcttctatcaaccacatatctgcgacggagcattttctatctttaatatctagattataacttattgtctcgtcaatgtctatagttctcatctttcccaacggcctcgcattaaatggaggaggagacaatgactgatatatttcgtccgtcactacgtaataaaagtaatgaggaaatcgtataaatacggtctcaccatttcgacatctggatttcagatataaaaatctgttttcaccgtgactttcaaaccaattaatgcaccgaacatccatttatagaatttagaaatatattttcatttaaatgaatcccaaacattggggaagagccgtatggaccattatttttatagtactttcgcaagcgggtttagacggcaacatagaagcgtgtaaacgaaaactatatactatagttagcactcttccatgtcctgcatgtagacggcacgcgactatcgctatagaggacaataatgtcatgtctagcgatgatctgaattatatttattattttttcatcagattatttaacaatttggcatctgatcccaaatacgcgatcgatgtgacaaaggttaaccctttataaacttaacccattataaaacttatgattagtcacgactgaaataaccgcgtgattattttttggtataattctacacggcatggtttctgtgactatgaattcaacccccgttacattagtgaaatctttaacaaacagcaagggttcgtcaaagacataaaactcattgtttacaatcgaaatagaccccctatcacacttaaaataaaaaatatccttatcctttaccaccaaataaaattctgattggtcaatgtgaatgtattcacttaacagttccacaaatttatttattaactccgaggcacatacatcgtcggtattttttatggcaaactttactcttccagcatccgtttctaaaaaaatattaacgagttccatttatatcatccaatattattgaaatgacgttgatggacaaatgatacaaataagaaggtacggtacctttgtccaccatctcctccaattcatgctctattttgtcattaactttaatgtatgaaaacagtacgccacatgcttccatgacagtgtgtaacactttggatacaaaatgtttgacattagtataattgttcaagactgtcaatctataatagatagtagctataatatattctatgatggtattgaagaagatgacaaccttggcatattgatcatttaacacagacatggtatcaacagatagcttgaatgaaagagaatcagtaattggaataagcgtcttctcgatggagtgtccgtataccaacatgtctgatattttgatgtattccattaaattatttagttttttctttttattctcgttaaacagcatttctgtcaacggaccccaacatcgttgaccgattaagttttgattgatttttccgtgtaaggcgtatctagtcagatcgtatagcctatccaataatccatcgtctgtgtgtagatcacatcgtacactttttaattctctatagaagagcgacagacatctggagcaattacagacagcaatttctttattctctacagatgtaagatacttgaagacattcctatgatgatgcagaattttggataacacggtattgatggtatctgttaccataattcctttgatggctgatagtgtcagagcacaagatttccaatctttgacaatttttagcaccattatctttgttttgatatctatatcagacagcatggtgcgtctgacaacacaaggattaagacggaaagatgaaatgattctctcaacatcttcaatggataccttgctattttttctggcattatctatatgtgcgagaatatcctctagagaatcagtatcctttttgatgatagtggatctcaatgacatgggacgtctaaaccttcttattctatcaccagattgcatggtgatttgtcttctttcttttatcataatgtaatctctaaattcatcggcaaattgtctatatctaaaatcataatatgagatgtttacctctacaaatatctgttcgtccaatgttagagtatttacatcagttttgtattccaaattaaacatggcaacggatttaattttatattcctctattaagtcctcgtcgataataacagaatgtagataatcatttaatccatcgtacatggttggaagatgctcgttgacaaaatctttaattgtcttgatgaaggtgggactatatctaacatcttgattaataaaatttataacattgtccataggatactttgtaactagttttatacacatctcttcatcggtaagtttagacagaatatcgtgaacaggtggtatattatattcatcagatatacgaagaacaatgtccaaatctatattgtttaatatattatatagatgtagtgtagctcctacaggaatatctttaactaagtcaatgatttcatcaaccgttagatctattttaaagttaatcatataggcattgatttttaaaaggtatgtagccttgactacattctcattaattaaccattccaagtcactgtgtgtaagaagattatattctatcataagcttgactacatttggtcccgataccattaaagaattcttatgatataaggaaacagattttaggtactcatctactctacaagaattttggagagccttaacgatatcagtgacgtttattatttcaggaggaaaaaacctaacattgagaatatcggaattaatagcttccagatacagtgattttggcaatagtccgtgtaatccataatccagtaacacgagctggtgcttgctagacaccttttcaatgtttaatttttttgaaataagctttgataaagccttcctcgcaaattccggatacatgaacatgtcggcgacatgattaagtattgttttttcattatttttatattttctcaacaagttctcaataccccaatagatgatagaatatcacccaatgcgtccatgttgtctatttccaacaggtcgctatatccaccaatagaagttttcccaaaaaagattctaggaacagttctaccaccagtaatttgttcaaaatagtcacgcaattcattttcgggtttaaattctttaatatcgacaatttcatacgctcctcttttgaaactaaacttatttagaatatccagtgcatttctacaaaaaggacatgtatacttgacaaaaattgtcactttgttattggccaacctttgttgtacaaattcctcggccattttaatatttaagtgatataaaactatctcgacttatttaactctttagtcgagatatatggacgcagatagctatatgatagccaactacagaaggcaaacgctataaaaaacataattacgacgagcatatttataaatatttttattcagcattacttgatatagtaatattaggcacagtcaaacattcaaccactctcgatacattaactctctcattttctttaacaaattctgcaatatcttcgtaaaaagattcttgaaactttttagaatatctatcgactctagatgaaatagcgttcgtcaacatactatgttttgtatacataaaggcgcccattttaacagtttctagtgacaaaatgctagcgatcctaggatcctttagaatcacatagattgacgattcgtctctcttagtaactctagtaaaataatcatacaatctagtacgcgaaataatattatccttgacttgaggagatctaaacaatctagttttgagaacatcgataagttcatcgggaatgacatacatactatctttaatagaactcttttcatccagttgaatggattcgtccttaaccaactgattaatgagatcttctattttatcattttccagatgatatgtatgtccattaaagttaaattgtgtagcgcttctttttagtctagcagccaatactttaacatcactaatatcgatatacaaaggagatgatttatctatggtattaagaattcgtttttcgacatctgtcaaaaccaattcctttttgcctgtatcatccagttttccatcctttgtaaagaaattattttctactagactattaataagactgataaggattcctccataattgcacaatccaaactttttcacaaaactagactttacaagatctacaggaatgcgtacttcaggtttcttagcttgtgattttttcttttgtggacattttcttgtgaccaactcatctaccatttcattgattttagcagtgaaataagctttcaatgcacgggcactgatactattgaaaacgagttgatcttcaaattccgccatttaagttcaccaaacaacttttaaatacaaatatatcaatagtagtagaataagaactataaaaaaaataataattaaccaataccaaccccaacaaccggtattattagttgatgtgactgttttctcatcacttagaacagatttaacaatttctataaagtctgtcaaatcatcttccggagaccccataaatacaccaaatatagcggcgtacaacttatccatttatacattgaatattggcttttctttatcgctatcttcatcatattcatcatcaatatcaacaagtcccagattacgagccagatcttcttctacattttcagtcattgatacacgttcactatctccagagagtccgataacgttagccaccacttctctatcaatgattagtttcttgagtgcgaatgtaatttttgtttccgttccggatctatagaaaactacaggtgtgataattgccttggccaattgtctttctcttttactgagtgattctagttcaccttctatagatctgagaatggatgattctccagtcgaaacatattctaccatggctccgtttaatttgttgatgaagatggattcatccttaaatgttttctctgtaatagtttccaccgaaagactatgcaaagaatttggaatgcgttccttgtgcttaatgtttccatagacggcttctagaagttgatacaacataggactagccgcggtaacttttatttttagaaagtatccatcgcttctatcttgtttagatttatttttataaagtttagtctctccttccaacataataaaagtggaagtcatttgactagataaactatcagtaagttttatagagatagacgaacaattagcgtattgagaagcatttagtgtaacgtattcgatacattttgcattagatttactaatcgattttgcatactctataacacccgcacaagtctgtagagaatcgctagatgcagtaggtcttggtgaagtttcaactctcttcttgattaccttactcatgattaaacctaaataattgtactttgtaatataatgatatatattttcactttatctcatttgagaataaaaatgtttttgtttaaccactgcatgatgtacagatttcggaatcacaaaccaccggtggttttattttatccttgtccaatgtgaattgaatgggagcggatgcgggtttcgtacgtagatagtacattcccgtttttagaccgagactccatccgtaaaaatgcatactcgttagtttggaataactcggatctgctatatggatattcatagattgactttgatcgatgaaggctcccctgtctgcagccatttttatgatcgtcttttgtggaatttcccaaatagttttataaactcgcttaatatcttctggaaggtttgtattctgaatggatccaccatctgccataatcctattcttgatctcatcattccataattttctctcggttaaaactctaaggagatgcggattaactacttgaaattctccagacaatactctccgagtgtaaatattactggtatacggttccaccgactcattatttcccaaaatttgagcagttgatgcagtcggcataggtgccaccaataaactatttctaagaccgtatgttctgattttatcttttagaggttcccaattccaaagatccgacggtacaacattccaaagatcatattgtagaataccgttactggcgtacgatcctacatatgtatcgtatggtccttccttctcagctagttcacaactcgcctctaatgcaccgtaataaatggtttcgaagatcttcttatttagatcttgtgcttccaggctatcaaatggataatttaagagaataaacgcgtccgctaatccttgaacaccaataccgataggtctatgtctcttattagagatttcagcttctggaataggataataattaatatctataattttattgagatttctgacaattactttgaccacatccttcagtttgagaaaatcaaatcgcccatctattacaaacatgttcaaggcaacagatgccagattacaaacggctacctcattagcatccgcatattgtattatctcagtgcaaagattactacacttgatagttcctaaattttgttgattactctttttgttacacgcatccttataaagaatgaatggagtaccagtttcaatctgagattctataatcgctttccagacgactcgagcctttattatagatttgtatctcctttctctttcgtatagtgtatacaatcgttcgaactcgtctccccaaacattgtccaatccaggacattcatccggacacatcaacgaccactctccgtcatccttcactcgtttcataaagagatcaggaatccaaagagctataaatagatctctggttctatgttcctcgtttcctgtattctttttaagatcgaggaacgccataatatcagaatgccacggttccaagtatatggccataactccaggccgtttgtttcctccctgatctatgtatctagcggtgttattataaactctcaacattggaataataccgtttgatataccattggtaccggagatatagcttccactggcacgaatattactaattgatagacctattccccctgccattttagagattaatgcgcatcgttttaacgtgtcatagataccctctatgctatcatcgatcatgttaagtagaaaacagctagacatttggtgacgactagttcccgcattaaataaggtaggagaagcgtgcgtaaaccatttttcagaaagtagattgtacgtctcaatagctgagtctatatcccattgatgaattcctactgcgacacgcattaacatgtgctgaggtctttcaacgatcttgttgtttattttcaacaagtaggatttttccaaagttttaaaaccaaaatagttgtatgaaaagtctcgttcgtaaataataaccgagttgagtttatccttatatttgttaactatatccatggtgatacttgaaataatcggagaatgtttcccatttttaggattaacatagttgaataaatcctccatcacttcactaaatagtttttttgtttccttgtgtagatttgatacggctattctggcggctagaatggcataatccggatgttgtgtagtacaagtggctgctatttcggctgccagagtgtccaattctaccgttgttactccattatatattccttgaataaccttcatagctattttaataggatctatatgatccgtgtttaagccataacataattttctaatacgagacgtgattttatcaaacatgacattttccttgtatccatttcgtttaatgacaaacatttttgttggtgtaataaaaaaattatttaacttttcattaatagggatttgacgtacgtagcgtacaaaatgattgttcctggtatatagataaagagtcctatatatttgaaaatcgttacggctcgattaaactttaatgattgcatagtgaatatatcattaggatttaactccttgactatcagggcggcaccagaaattaccatcaaaagcattaatacagttatgcctatcgcagttagaacggttatagcatccaccatttatatctaaaaattagatcaaagaatatgtgacaaagtcctagttgtatattgagaattgacaaaacaatgtttcttacatattttttttttattagtaaccgacttaatagtaggaactggaaaactagacttgattattctataagtatagatacccttccaaataatattctctttgataaaagttccagaaaatgtagaattttttaaaaagttatcttttgctattaccaagattgtgtttagacgcttattattaatatgagtgatgaaatccacaccgcctctagatatcgcctttatttccacattagatggtaaatccaatagtgaaactatctttttaggaatgtatggactcgcgtttagaggagtgaacgtcttgggcgtcggaaaggatgattcgtcaaacgaataaacaatttcacaaatggatgttaatgtattagtaggaaattttttgacgctagtggaattgaaaattctaatggatgatgttctacctatttcatccgataacatgttaatttccgacaccaacggttttaatatttcgatgatatacggtagtctctctttcggacttatatagcttattccacaatacgagtcattatatactccaaaaaacaaaataactagtataaaatctgtatcgaatgggaaaaacgaaattatcgacataggtatagaatccggaacattgaacgtattaatacttaattctttttctgtggtaagtaccgataggttattgacattgtatggttttaaatattctataacttgagacttgatagatattagtgatgaattgaaaattatttttatcaccacgtgtgtttcaggatcatcgtcgacgcccgtcaaccaaccgaacggagtaaaataaatatcattaatatatgctctagatattagtatttttatcaatcctttgattatcatcttctcgtaggcgaatgattccatgatcaagagtgatttaagaacatcctccggagtattaatgggcttagtaaacagtccatcgttgcaataataaaagttatccaagttaaaggatattatgcattcgtttaaagatatcacctcatctgacggagacaattttttggtaggttttagagactttgaagctacttgtttaacaaagttattcatcgtcgtttactattctatttaattttgtagttaatttatcacatatcacattaattgactttttggtccatttttccatacgtttatattcttttaatcctgcgttatccgtttccgttatatccagtgatagatcgtgcaggttaaatagaatgctcttaaataatgtcatttttttatccgctaaaaatttaaagaatgtataaacttttttcaaagatttaaaacttttaggtggtgtcctagtacacaatatcataaacaaactaataaacatcccgcattcagattccaacagctgattaacttccacattaatacagcctattttcgctccaaatgtacattcgaaaaatctgaataaaacatcaatgtcgcaatttgtattatccaatacagaatgtttgtgattcgtgttaaaaccatcggagaaggaatagaaataaaaattattatagtggtggaattcagttggaatattgcctccggagtcataaaaggatactaaacattgttttttatcataaattacacatttccaatgagacaaataacaaaatccaaacattacaaatctagaggtagaacttttaattttgtctttaagtatatacgataagatatgtttattcataaacgcgtcaaatttttcatgaatcgctaaggagtttaagaatctcatgtcaaattgtcctatataatccacttcggatccataagcaaactgagagactaagttcttaatacttcgattgctcatccaggctcctctctcaggctctattttcatcttgacgacctttggattttcaccagtatgtattcctttacgtgataaatcatcgattttcaaatccatttgtgagaagtctatcgccttagatactttttcccgtagtcgaggtttaaagaaatacgctaacggtatactagtaggtaactcaaaaacatcatatatagaatggtaacgcgtctttaactcgtcggttaactctttcttttgatcgagttcgtcgctactattgggtctgctcaggtgccccgactctactagttccaacatcataccgataggaatacaagacactttgccggcggttgtagatttatcatatttttccactacatatccgttacaatttgttaaaaatttagatacatctatattgctacataatccagctagtgaatatatatgacataataaattggtaaatcctagttctggtattttactaattactaaatctgtatatctttccatttatcatggaaaagaatttaccagatatcttcttttttccaaactgcgttaatgtattctcttacaaatattcacaagatgaattcagtaatatgagtaaaacggaacgtgatagtttctcattggccgtgtttccagttataaaacatagatggcataacgcacacgttgtaaaacataaaggaatatacaaagttagtacagaagcacgtggaaaaaaagtatctcctccatcactaggaaaacccgcacacataaacctaaccgcgaagcaatatatatacagtgaacacacaataagctttgaatgttatagttttctaaaatgtataacaaatacagaaatcaattcgttcgatgagtatatattaagaggactattagaagctggtaatagtttacagatattttccaattccgtaggtaaacgaacagatactataggtgtactagggaataagtatccatttagcaaaattccattggcctcattaactcctaaagcacaacgagagatattttcagcgtggatttctcatagacctgtagttttaactggaggaactggagtgggtaagacgtcacaggtacccaagttattgctttggtttaattatttatttggtggattctctactctagataaaatcactaactttcacgaaagaccagtcattctatctcttcctaggatagctttagttagattgcatagcaataccattttaaaatcattgggatttaaggtactagatggatctcctatttctttacggtacggatctataccggaagaattaataaacaaacaaccaaaaaaatatggaattgtattttctacccataagttatctctaacaaaactatttagttatggcactcttattatagacgaagttcatgagcatgatcaaataggagatattattatagcagtagcgagaaagcatcatacgaaaatagattctatgtttttaatgactgccacgttagaggatgaccgagaacggctaaaagtatttttacctaatcccgcatttatacatattcctggagatacactgtttaaaattagcgaggtatttattcataataagataaatccatcttccagaatggcatacatagaagaagaaaagagaaatttagttactgctatacagatgtatactcctcctgatggatcatccggtatagtctttgtggcatccgttgcacagtgtcacgaatataaatcatatttagaaaaaagattaccgtatgatatgtatattattcatggtaaggtcttagatatagacgaaatattagaaaaagtgtattcatcacctaatgtatcgataattatttctactccttatttggaatccagcgttactatacgcaatgttacacacatttatgatatgggtaaagtttttgtccccgctccttttggaggatcgcaagaatttatttctaaatctatgagagatcaacgaaaaggaagagtaggaagagttaatcctggtacatacgtctatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataactttgagaggacgggagaattaactagtattgtacgagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaaatcaaactctaatgaccacatctttttttagagatgaaaaattttctacatctccttttgtagacacgactaaacattttgcaaaaaaaagtttattagtgtttagataatcgtatacttcatcagtgtagatagtaaatgtgaacaaataaaaggtattcttactcaatagattggtaaattccatagaatatattaatcctttcttcttgagatcccacatcatttcaaccagagacgttttatccaatgatttacctcgtactataccacatacaaaactagattttgcagtgacgtcgtacctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaactactatcggatgttgttgggtatctctagtgttacacatggccttactaaagtttgggtaaataactatgatatctctattaattatagatgcatatatttcattcgtcaaggatattagtatcgacttgctatcgtcattaatacgtgtaatgtaatcatataaatcatgcgatagccaaggaaaattcaaatagatgttcatcatataatcgtcgctataattcatattaatacgttgacattgactaatttgtaatatagcctcgccacgaagaaagctctcgtattcagtttcatcgataaaggataccgttaaatataactggttgccgatagtctcatagtctattaagtggtaagtttcgtacaaatacagaatccctaaaatattatctaatgttggattaatctttaccataactgtataaaatggagacggagtcataactattttaccgtttgtacttactggaatagacgaaggaataatctccggacatgctggtaaagacccaaatgtctgtttgaagaaatccaatgttccaggtcctaatctcttaacaaaaattacgatattcgatcccgatatcctttgcattctatttaccagcatatcacgaactatattaagattatctatcatgtctattctcccaccgttatataaatcgcctccgctaagaaacgttagtatatccatacaatggaatacttcatttctaaaatagtattcgttttctaattctttaatgtgaaatcgtatactagaaagggaaaaattatctttgagttttccgttagaaaagaaccacgaaactaatgttctgattgcgtccgattccgttgctgaattaatggatttacaccaaaaactcatataacttctagatgtagaagcattcgctaaaaaattagtagaatcaaaggatataagtagatgttccaacaagtgagcaattcccaagatttcatctatatcattctcgaatccgaaattagaaattcccaagtagatatcctttttcatccgatcgttgatgaaaatacgaactttattcggtaagacaatcatttactaaggagtaaaataggaagtaatgttcgtatgtcgttatcatcgtataaattaaaggtgtgttttttaccattaagtgacattataattttaccaatattggaattataatataggtgtatttgcgcactcgcgacggttgatgcatcggtaaatatagctgtatctaatgttctagtcggtatttcatcatttcgctgtctaataatagcgttttctctatctgtttccattacagctgcctgaagtttattggtcggataatatgtaaaataataagaaatacatacgaataacaaaaataaaataagatataataaagatgccatttagagatctaattttgttcaacttgtccaaattcctacttacagaagatgaggaatcgttggagatagtgtcttccttatgtagaggatttgaaatatcttatgatgacttgataacttactttccagataggaaataccataaatatatttctaaagtatttgaacatgtagatttatcggaggaattaagtatggaattccatgatacaactttgcgagatttagtctatcttagattgtacaagtattccaagtgtatacggccgtgttataaattaggagataatctaaaaggcatagttgttataaaggacaggaatatttatattagggaagcaaatgatgacttgatagaatatctcctcaaggaatacactcctcagatttatacatattctaatgagcgcgtccccataactggttcaaaattaattctttgtggattttctcaagttacatttatggcgtatacaacgtcgcatataacaacaaataaaaaggtagatgttctcgtttccaaaaaatgtatagatgaactagtcgatccaataaattatcaaatacttcaaaatttatttgataaaggaagcggaacaataaacaaaatactcaggaagatattttattcggtaacaggtggccaaactccataggtagctttttctatttcggattttagaatttccaaattcaccagcgatttatctgttttggtgaaatccaaggatttattaatgtccacaaatgccatttgttttgtctgtggattgtatttgaaaatggaaacgatgtagttagatagatgcgctgcgaagtttcctattagggttccgcgcttcacgtcacccagcatacttgaatcaccatcctttaaaaaaaatgataagatatcaacatggagtatatcatactcggattttaattcttctactgcatcactgacattttcacaaatactacaatacggtttaccgaaaataatcagtacgttcttcatttatgggtatcaaaaacttaaaatcgttactgctggaaaataaatcactgacgatattagatgataatttatacaaagtatacaatggaatatttgtggatacaatgagtatttatatagccgtcgccaattgtgtcagaaacttagaagagttaactacggtattcataaaatacgtaaacggatgggtaaaaaagggagggcatgtaaccctttttatcgatagaggaagtataaaaattaaacaagacgttagagacaagagacgtaaatattctaaattaaccaaggacagaaaaatgctagaattagaaaagtgtacatccgaaatacaaaatgttaccggatttatggaagaagaaataaaggcagaaatgcaattaaaaatcgataaactcacatttcaaatatatttatctgattctgataacataaaaatatcattgaatgagatactaacacatttcaacaataatgagaatgttacattattttattgtgatgaacgagacgcagaattcgttatgtgtctcgaggctaaaacacatttctctaccacaggagaatggccgttgataataagtaccgatcaggatactatgctatttgcatctgctgataatcatcctaagatgataaaaaacttaactcaactgtttaaatatgttccatctgcagaggataactatttagcaaaattaacggcgttagtgaatggatgtgatttctttcctggactctatggggcatctataacacccaacaacttaaacaaaatacaattgtttagtgattttacaatcgataatatagtcactagtttggcaattaaaaattattatagaaagactaactctaccgtagacgtgcgtaatattgttacgtttataaacgattacgctaatttagacgatgtctactcgtatattcctccttgtcaatgcactgttcaagaatttatattttccgcattagatgaaaaatggaatgaatttaaatcatcttatttagaaagcgtgccgttaccctgccaattaatgtatgcgttagaaccacgcaaggagattgatgtttcagaagttaaaactttatcatcttatatagatttcgaaaatactaaatcagatatcgatgttataaaatctatatcctcgatcttcggatattctaacgaaaactgtaacacgatagtattcggcatctataaggataatttactactgagtataaatagttcattttactttaacgatagtctgttaataaccaatactaaaagtgataatataataaatataggttactagattaaaaatggtgttccaactcgtgtgctctacgtgcggcaaagatatttctcacgaacgatataaattgattatacgaaaaaaatcattaaaggatgtactcgtcagtgtaaagaacgaatgttgtaggttaaaattatctacacaaatagaacctcaacgtaacttaacagtgcaacctctattggatataaactaatatggatccggttaattttatcaagacatatgcgcctagaggttctattatttttattaattataccatgtcattaacaagtcatttgaatccatcgatagaaaaacatgtgggtatttattatggtacgttattatcggaacacttggtagttgaatcaacatatagaaaaggagttcgaatagtcccattggatagtttttttgaaggatatcttagtgcaaaagtatacatgttagagaatattcaagttatgaaaatagcagctgatacgtcattaactttattgggtattccgtatggatttggtcatgatagaatgtattgttttaaattggtagctgactgttataaaaatgccggtattgatacatcgtctaaacgaatattaggtaaagatatttttctgagccaaaacttcacagacgataatagatggataaagatatatgattctaataatttaacattttggcaaattgattaccttaaagggtgagttaatatgcataactactcctccgttgttttttccctcgttctttttcttaacgttgtttgccatcactctcataatgtaaagatattctaaaatggtaaacttttgcatatcggacgcagaaattggtataaatgttgtaattgtattatttcccgtcaatggactagtcacagctccatcagttttatatcctttagagtatttctcactcgtgtctaacattctagagcattccatgatctgtttatcgttgatattggccggaaagatagattttttattttttattatattactattggcaattgtagatataacttctggtaaatatttttctaccttttcaatctcttctattttcaagccggctatatattctgctatattgttgctagtatcaataccttttctggctaagaagtcatatgtggtattcactatatcagttttaactggtagttccattagcctttccacttctgcagaataatcagaaattggttctttaccagaaaatccagctactataataggctcaccgatgatcattggcaaaatcctatattgtaccagattaatgagagcatatttcatttccaataattctgctagttcttgagacattgatttatttgatgaatctagttggttctctagatactctaccatttctgccgcatacaataacttgttagataaaatcagggttatcaaagtgtttagcgtggctagaatagtgggcttgcatgtattaaagaatgcggtagtatgagtaaaccgttttaacgaattatatagtctccagaaatctgtggcgttacatacatgagccgaatgacatcgaagattgtccaatatttttaatagctgctctttgtccattatttctatatttgactcgcaacaattgtagataccattaatcaccgattcctttttcgatgccggacaatagcacaattgtttagctttggactctatgtattcagaattaatagatatatctcttaatacagattgcactatacattttgaaactatgtcaaaaattgtagaacgacgctgttctgcagccatttaactttaaataatttacaaaaatttaaaatgagcatccgtataaaaatcgataaactgcgccaaattgtggcatatttttcagagttcagtgaagaagtatctataaatgtagactcgacggatgagttaatgtatatttttgccgccttgggcggatctgtaaacatttgggccattatacctctcagtgcatcagtgttctaccgcggagccgaaaatattgtgtttaatcttcctgtgtccaaggtaaaatcgtgtttgtgtagttttcacaatgatgccatcatagatatagaacctgatctggaaaataatctagtaaaactttctagttatcatgtagtaagtgtcgattgtaataaggaactgatgcctattaggacagatactactatttgtctaagtatagatcaaaagaaatcttacgtgtttaattttcacaagtatgaagaaaaatgttgtggtagaaccgtcattcatttagaatggttgttgggctttatcaagtgtattagtcagcatcagcatttggctattatgtttaaagatgacaatattattatgaagactcctggtaatactgatgcattttccagggaatattctatgactgaatgttctcaagaactacaaaagttttctttcaaaatagctatctcgtctctcaacaaactacgaggattcaaaaagagagtcaatgtttttgaaactagaatcgtaatggataatgacgataacattttaggaatgttgttttcggatagagttcaatcctttaagatcaacatctttatggcgtttttagattaatactttcaatgagataaatatgggtggcggagtaagtgttgagctccctaaacgggatccgcctccgggagtacccactgatgagatgttattaaacgtggataaaatgcatgacgtgatagctcccgctaagcttttagaatatgtgcatataggaccactagcaaaagataaagaggataaagtaaagaaaagatatccagagtttagattagtcaacacaggacccggtggtctttcggcattgttaagacaatcgtataatggaaccgcacccaattgctgtcgcacttttaatcgtactcattattggaagaaggatggaaagatatcagataagtatgaagagggtgcagtattagaatcgtgttggccagacgttcacgacaccggaaaatgcgatgttgatttattcgactggtgtcagggggatacgttcgatagaaacatatgccatcagtggatcggttcagcctttaataggagtaatagaactgtagagggtcaacaatcgttaataaatctgtataataagatgcaaacattatgtagtaaagatgctagtgtaccaatatgtgaatcatttttgcatcatttacgcgcacacaatacagaagatagcaaagagatgatcgattatattctaagacaacagtctgcggactttaaacagaaatatatgagatgtagttatcccactagagataagttagaagagtcattaaaatatgcggaacctcgagaatgttgggatccagagtgttcgaatgccaatgttaatttcttgctaacacgtaattataataatttaggactttgcaatattgtacgatgtaatactagcgtgaacaacttacagatggataaaacttcctcattaagattgtcatgtggattaagcaatagtgatagattttctactgttcccgtcaatagagcaaaagtagttcaacataatattaaacactcgttcgacctaaaattgcatttgatcagtttattatctctcttggtaatatggatactaattgtagctatttaaatgggtgccgcggcaagcatacagacgacggtgaatacactcagcgaacgtatctcgtctaaattagaacaagaagcgaatgctagtgctcaaacaaaatgtgatatagaaatcggaaatttttatatccgacaaaaccatggatgtaacctcactgttaaaaatatgtgctctgcggacgcggatgctcagttggatgctgtgttatcagccgctacagaaacatatagtggattaacaccggaacaaaaagcatacgtgccagctatgtttactgctgcgttaaacattcagacgagtgtaaacactgttgttagagattttgaaaattatgtgaaacagacttgtaattctagcgcggtcgtcgataacaaattaaagatacaaaacgtaatcatagatgaatgttacggagccccaggatctccaacaaatttggaatttattaatacaggatctagcaaaggaaattgtgccattaaggcgttgatgcaattgacgactaaggccactactcaaatagcacctaaacaagttgctggtacaggagttcagttttatatgattgttatcggtgttataatattggcagcgttgtttatgtactatgccaagcgtatgttgttcacatccaccaatgataaaatcaaacttattttagccaataaggaaaacgtccattggactacttacatggacacattctttagaacttctccgatggttattgctaccacggatatgcaaaactgaaaatatattgataatattttaatagattaacatggaagttatcactgatcgtctagacgatatagtgaaacaaaatatagcggatgaaaaatttgtagattttgttatacacggtctagagcatcaatgtcctgctatacttcgaccattaattaggttgtttattgatatactattatttgttatagtaatttatatttttacggtacgtctagtaagtagaaattatcaaatgttgttggcgttggtggcgctagtcatcacattaactattttttattactttatactataatagtactagactgacttctaacaaacatctcacctgccataaataaatgcttgatattaaagtcttctatttctaacactattccatctgtggaaaataatactctgacattatcgctaattgacacatcggtgagtgatatgcctataaagtaataatcttctttgggcacatataccagtgtaccaggttctaacaacctatttactggtgctcctatagcatactttttctttaccttgagaatatccatcgtttgcttggtcaatagcgatatgtgattttttatcaaccactcgaaaaagtaattggagtgttcatatcctctacgggctattgtctcatggccgtgtatgaaatttaagtaacacgactgtggtagatttgttctatagagccggttgccgcaaatagatagaactaccaatatgtctgtacaaatgttaaacattaattgattaacagaaaaaacaatgttcgttctgggaatagaaaccagatcaaaacaaaattcgttagaatatatgccacgtttatacattgaatataaaataactacagtttgaaaaataacagtatcatttaaacatttaacttgcggggttaatctcacaactttactgtttttgaactgttcaaaatatagcatagatccgtgagaaatacgtttagccgcctttaatagaggaaatcccaccgcctttctggatctcaccaacgacgatagttctgaccagcaactcatttcttcatcatccacctgttttaacatataataggcaggagatagatatccgtcattgcaatattccttctcgtaggcacacaatctaatattgataaaatctccattctcttctctgcatttattatcttgtttcggtggctgattaggctgtagtcttggtttaggctttggtatatcgttgttgaatctattttggtcattaaatctttcatttcttcctggtatatttttatcacctcgtttggttggatttttgtctatattatcgtttgtaacatcggtacgggtattcatttatcacaaaaaaaacttctctaaatgagtctactgctagaaaacctcatcgaagaagataccatattttttgcaggaagtatatctgagtatgatgatttacaaatggttattgccggcgcaaaatccaaatttccaagatctatgctttctatttttaatatagtacctagaacgatgtcaaaatatgagttggagttgattcataacgaaaatatcacaggagcaatgtttaccacaatgtataatataagaaacaatttgggtctaggagatgataaactaactattgaagccattgaaaactatttcttggatcctaacaatgaagttatgcctcttattattaataatacggatatgactgccgtcattcctaaaaaaagtggtaggagaaagaataagaacatggttatcttccgtcaaggatcatcacctatcttgtgtattttcgaaactcgtaaaaagattaatatttataaagaaaatatggaatccgcgtcgactgagtatacacctatcggagacaacaaggctttgatatctaaatatgcgggaattaatatcctaaatgtgtattctccttccacatccataagattgaatgccatttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacgtaaaatatctagcaaaaattctatgtctaaaaacagaaatagcaagagatccgtacgcagtaataaatagaaacgtactactgagatatactaccgatatagagtataatgatttagttactttaataaccgttagacataaaattgattctatgaaaactgtgtttcaggtatttaacgaatcatccataaattatactccggttgatgatgattatggagaaccaatcattataacatcgtatcttcaaaaaggtcataacaagtttcctgtaaattttctatacatagatgtggtaatatctgacttatttcctagctttgttagactagatactacagaaactaatatagttaatagtgtactacaaacaggcgatggtaaaaagactcttcgtcttcccaaaatgttagagacggaaatagttgtcaagattctctatcgccctaatataccattaaaaattgttagatttttccgcaataacatggtaactggagtagagatagccgatagatctgttatttcagtcgctgattaatcaattagtagagatgagataagaacattataataatcaataatatatcttatatctcgtttagaaaaatgctaatattaaaatagctaacgctagtaatccaatcggaagccatttgatatctataatagggtatctaatttcctgattcagatagcggacagctatattctcggtagctactcgtttggaatcacaaacattatttacatctaatttactatctgtaatggaaacgtttcccaatgaaatggtacaatccgatacattgcattttgttatatttttttttaaagaggctggtaacaacgcatcgcttcgtttacatggctcgtaccaacaataatagggtaatcttgtatctattcctatccgtactatgcttttatcaggataaatacatttacatcgtatatcgtctttgttagcatcacagaatgcataaatttgttcgtccgtcatgataaaaatttaaagtgtaaatataactattatttttatagttgtaataaaaagggaaatttgattgtataccttcggttctttaaaagaaactgacttgataaaaatggctgtaatctctaaggttacgtatagtctatatgatcaaaaagagattaatgctacagatattatcattagtcatgttaaaaatgacgacgatatcggtaccgttaaagatggtagactaggtgctatggatggggcattatgtaagacttgtgggaaaacggaattggaatgtttcggtcactggggtaaagtaagtatttataaaactcatatagttaagcctgaatttatttcagaaattattcgtttactgaatcatatatgtattcactgcggattattgcgttcacgagaaccgtattccgacgatattaacctaaaagagttatcgggacacgctcttaggagattaaaggataaaatattatccaagaaaaagtcatgttggaacagtgaatgtatgcaaccgtatcaaaaaattactttttcaaagaaaaaggtttgtttcgtcaacaagttggatgatattaacgttcctaattctctcatctatcaaaagttaatttctattcatgaaaagttttggccattattagaaattcatcaatatccagctaacttattttatacagactactttcccatccctccgttgattattagaccggctattagtttttggatagatagtatacccaaagaaaccaatgaattaacttacttattaggtatgatcgttaagaattgtaacttgaatgctgatgaacaggttatccagaaggcggtaatagaatacgatgatattaaaattatttctaataacacttccagtatcaatttatcatatattacatccggcaaaaataatatgattagaagttatatcgtcgcccgacgaaaagatcagacggctagatctgtaattggtcccagtacatctatcaccgttaatgaggtaggaatgcccgcatatattagaaatacacttacagaaaagatatttgttaatgcctttacagtggataaagttaaacaactattagcatcaaaccaagttaaattttactttaataaacgattaaaccaattaacaagaatacgccaaggaaagtttatcaaaaataaaatacatttattgcctggtgattgggtagaagtagctgttcaagaatatacaagtattatttttggaagacagccgtctctacatagatacaacgtcatcgcttcatctatcagagctaccgaaggagatactatcaaaatatctcccggaattgccaactctcaaaatgctgatttcgacggagatgaagaatggatgatattagaacaaaatcctaaagctgtaattgaacaaagtattcttatgtatccgacgacgttactcaaacacgatattcatggagcccccgtttatggatctattcaagatgaaatcgtagcagcgtattcattgtttaggatacaagatctttgtttagatgaagtattgaacatcttggggaaatatggaagagagttcgatcctaaaggtaaatgtaaattcagcggtaaagatatctatacttacttgataggtgaaaagattaattatccgggtctcttaaaggatggtgaaattattgcaaacgacgtagatagtaattttgttgtggctatgaggcatctgtcattggctggactcttatccgatcataagtcgaacgtggaaggtatcaactttattatcaagtcatcttatgtttttaagagatatctatctatttacggttttggggtgacattcaaagatctgagaccaaattcgacgttcactaataaattggaggccatcaacgtagaaaaaatagaacttatcaaagaagcatacgccaaatatctcaacgatgtaagagacgggaaaatagttccattatctaaagctttagaggcggactatgtggaatccatgttatccaacttgacaaatcttaatatccgagagatagaagaacatatgagacaaacgctgatagatgatccagataataacctcctgaaaatggccaaagcgggttataaagtaaatcctacagaactaatgtatattctaggtacgtatggacaacaaaggattgatggtgaaccagcagagactcgagtattgggtagagtcttaccttactatcttccagactctaaggatccagaaggaagaggttacattcttaattctttaacaaaaggattaacaggttctcaatattacttttcgatgctggttgcaagatctcaatctactgatatcgtctgtgaaacatcacgtaccggaacactggctagaaaaatcattaaaaagatggaggatatggtggtcgacggatacggacaagtagttataggtaatacgctcatcaagtacgccgccaattataccaaaattctaggctcagtatgtaaacctgtagatcttatctatccagatgagtccatgacttggtatttggaaattagtgctctgtggaataaaataaaacagggattcgtttactctcagaaacagaaacttgcaaagaagacattggcgccgtttaatttcctagtattcgtcaaacccaccactgaggataatgctattaaggttaaggatctgtacgatatgattcataacgtcattgatgatgtgagagagaaatacttctttacggtatctaatatagattttatggagtatatattcttgacgcatcttaatccttctagaattagaattacaaaagaaacggctatcactatctttgaaaagttctatgaaaaactcaattatactctaggtggtggaactcctattggaattatttctgcacaggtattgtctgagaagtttacacaacaagccctgtccagttttcacactactgaaaaaagtggtgccgtcaaacaaaaacttggtttcaacgagtttaataacttgactaatttgagtaagaataagaccgaaattatcactctggtatccgatgatatctctaaacttcaatctgttaagattaatttcgaatttgtatgtttgggagaattaaatccagacatcactcttcgaaaagaaacagataggtatgtagtagatataatagtcaatagattatacatcaagagagcagaaattaccgaattagtcgtcgaatatatgattgaacgattcatctcctttagcgtcattgtaaaggaatggggtatggaaacattcattgaggacgaggataatattagatttactgtctatctaaatttcgttgaaccagaggaattgaatcttagtaagtttatgatggttcttccgggggcagccaacaagggaaagattagtaaattcaagattcctatctctgattatacgggttatgacgacttcaatcaaacaaaaaagctcaataagatgactgtagaactcatgaatctaaaagaattaggttctttcgatttggaaaacgtcaacgtgtatcctggagtatggaatacatacgatatcttcggtatcgaggccgctcgtgaatacttgtgcgaagccatgttaaacacctatggagaagggttcgattatctgtatcagccttgtgatcttctcgctagtttactatgtgctagttacgaaccagaatcagtgaataaattcaagttcggcgcagctagtactcttaagagagctacgttcggagacaataaagcattgttaaacgcggctcttcataaaaagtcagaacctattaacgataatagtagctgccacttttttagcaaggtccctaatataggaactggatattacaaatactttatcgacttgggtcttctcatgagaatggaaaggaaactatctgataagatatcttctcaaaagatcaaggaaatggaagaaacagaagacttttaattcttatcaataacatatttttctatgatctgtcttttaaacgatggattttccacaaatgcgcctctcaagtccctcatagaatgatacacgtataaaaaatatagcataggcaatgactccttatttttagacattagatatgccaaaatcatagccccgcttctatttactcccgcagcacaatgaaccaacacgggctcgtttcgttgatcacatttagataaaaaggcggttacgtcgtcaaaatatttactaatatcggtagttgtatcatctaccaacggtatatgaataatattaatattagagttaggtaatgtatatttatccatcgtcaaatttaaaacatatttgaacttaacttcagatgatggtgcatccatagcatttttataatttcccaaatacacattattggttacccttgtcattatagtgggagatttggctctgtgcatatctccagttgaacgtagtagtaagtatttatacaaacttttcttatccatttataacgtacaaatggataaaactactttatcggtaaacgcgtgtaatttagaatacgttagagaaaaggctatagtaggcgtacaagcagccaaaacatcaacacttatattctttgttattatattggcaattagtgcgctattactctggtttcagacgtctgataatccagtctttaatgaattaacgagatatatgcgaattaaaaatacggttaacgattggaaatcattaacggatagcaaaacaaaattagaaagtgatagaggtagacttctagccgctggtaaggatgatatattcgaattcaaatgtgtggatttcggcgcctattttatagctatgcgattggataagaaaacatatctgccgcaagctattaggcgaggtactggagacgcgtggatggttaaaaaggcggcaaaggtcgatccatctgctcaacaattttgtcagtatttgataaaacacaagtctaataatgttattacttgtggtaatgagatgttaaatgaattaggttatagcggttattttatgtcaccgcattggtgttccgattttagtaatatggaatagtgttagataaatgcggtaacgaatgttcctgtaaggaaccataacagtttagatttaacgttaaagatgagcataaacataataaacaaaattacaatcaaacctataacattaatatcaaacaatccaaaaaatgaaatcagtggagtagtaaacgcgtacataactcctggataacgtttagtagctgccgttcctattctagaccaaaaattcggtttcatgttttcgaaacggtgttctgcaacaagtcggggatcgtgttctacatatttggcggcattatccagtatctgcctattgatcttcatttcgttttcaattctggctatttcaaaataaaatcccgatgatagacctccagactttataatttcatctacgatgttcagcgccgtagtaactctaataatataggctgataagctaacatcataccctcctgtatatgtgaatatggcatgatttttgtccattacaagctcggttttaactttattgcctgtaataatttctctcatctgtaggatatctatttttttgtcatgcattgccttcaagacgggacgaagaaacgtaatatcctcaataacgttatcgttttctacaataactacatattctacctttttattttctaactcggtaaaaaaattagaatcccatagggctaaatgtctagcgatatttcttttcgtttcctctgtacacatagtgttacaaaaccctgaaaagaagtgagtatacttgtcatcatttctaatgtttcctccagtccactgtataaacgcataatccttgtaatgatctggatcatccttgactaccacaacatttcttttttctggcataacttcgttgtcctttacatcatcgaacttctgatcattaatatgctcatgaacattaggaaatgtttctgatggaagtctatcaataactggcacaacaataacaggagttttcgccgccgccatttagttattgaaattaatcatatacaactctttaatacgagttatattttcgtctatccattgtttcacatttacatatttcgacaaaaagatataaaatgcgtattccaatgcttctctgtttaatgaattactaaaatatacaaacacgtcactgtctggcaataaatgatatcttagaatattgtaacaatttattttgtattgcacatgttcgtgatctatgagttcttcttcgaatggcataggatctccgaatctgaaaacgtataaataggagttagaataataatatttgagagtattggtaatatataaactctttagcggtataattagtttttttctctcaatttctatttttagatgtgatggaaaaatgactaattttgtagcattagtatcatgaactctaatcaaaatcttaatatcttcgtcacacgttagctctttgaagtttttaagagatgcatcagttggttcgaccgatggagtaggtgcaacaattttttgttcgatgtatgtatgtactggagccattgttttaactataatggtgcttgtatcgaaaaactttaatgcagatagcggaagctcttcgccgcgactttctacatcgtaattgggttctaacgccgatctctgaatggatactagttttctaagttctaatgtgattctctgaaaatgtaaatccaattcctccggcattatagatgtgtatacatcggtaaataaaactatagtatccaacgatcccttctcgcaaattctagtcttaaccaaaaaatcgtatataaccacggagatggcgtatttaagagtggattcttctaccgttttgttcttggatgtcatataggaaactataaagtccgcactactgttaagaatgattactaacgcaactatatagttcaaattaagcattttggaaacataaaataactctgtagacgatacttgactttcgaataagtttgcagacaaacgaagaaagaacagacctctcttaatttcagaagaaaactttttttcgtattcctgacgtctagagtttatatcaataagaaagttaagaattagtcggttaatgttgtatttcattacccaagtttgagatttcataatattatcaaaagacatgataatattaaagataaagcgctgactatgaacgaaatagctatatggttcgctcaaaaatatagtcttgttaaacgtggaaacgataactgtatttttaatcacgtcagcggcatctaaattaaatataggtatatttattccacacactctacaatatgccacaccatcttcataataaataaattcgttagcaaaattattaattttagtgaaatagttagcgtcaactttcatagcttccttcaatctaatttgatgctcacacggtgcgaattccactctaacatcccttttccatgcctcaggttcatcgatctctataatatctagttttttgcgtttcacaaacacaggctcgtctctcgcgatgagatctgtatagtaactatgtaaatgataactagatagaaagatgtagctatatagatgacgatcctttaagagaggtataataactttaccccaatcagatagactgttgttatggtcttcggaaaaagaatttttataaatttttccagtattttccaaatatacgtacttaacatctaaaaaatccttaatgataataggaatggataatccgtctattttataaagaaatacatatcgcacattatacttttttttggaaatgggaataccgatgtgtctacataaatatgcaaagtctaaatattttttagagaatcttaattggtccaaattcttttccaagtacggtaatagatttttcatattgaacggtatcttcttaatctctggttctagttccgcattaaatgatgaaactaagtcactatttttataactaacgattacatcacctctaacatcatcatttaccagaatactgatcttcttttgtcgtaaatacatgtctaatgtgttaaaaaaaagatcatacaagttatacgtcatttcatctgtggtattcttgtcattgaaggataaactcgtactaatctcttctttaacagcctgttcaaatttatatcctatatacgaaaaaatagcaaccagtgtttgatcatccgcgtcaatattctgttctatcgtagtgtataacaatcgtatatcttcttctgtgatagtcgatacgttataaaggttgataacgaaaatatttttatttcgtgaaataaagtcatcgtaggattttggacttatattcgcgtctagtagatatgcttttatttttggaatgatctcaattagaatagtctctttagagtccatttaaagttacaaacaactaggaaattggtttatgatgtataatttttttagtttttatagattctttattctatacttaaaaaatgaaaataaatacaaaggttcttgagggttgtgttaaattgaaagcgagaaataatcataaattatttcattatcgcgatatccgttaagtttgtatcgtaatggcgtggtcaattacaaataaagcggatactagtagcttcacaaagatggctgaaatcagagctcatctaaaaaatagcgctgaaaataaagataaaaacgaggatattttcccggaagatgtaataattccatctactaagcccaaaaccaaacgagccactactcctcgtaaaccagcggctactaaaagatcaaccaaaaaggaggaagtggaagaagaagtagttatagaggaatatcatcaaacaactgaaaaaaattctccatctcctggagtcagcgacattgtagaaagcgtggccgctgtagagctcgatgatagcgacggggatgatgaacctatggtacaagttgaagctggtaaagtaaatcatagtgctagaagcgatctctctgacctaaaggtggctaccgacaatatcgttaaagatcttaagaaaattattactagaatctctgcagtatcgacggttctagaggatgttcaagcagctggtatctctagacaatttacttctatgactaaagctattacaacactatctgatctagtcaccgagggaaaatctaaagttgttcgtaaaaaagttaaaacttgtaagaagtaaatgcgtgcacttttttataaagatggtaaactctttaccgataataattttttaaatcctgtatcagacgataatccagcgtatgaggttttgcaacatgttaaaattcctactcatttaacagatgtagtagtatatgaacaaacgtgggaggaggcgttaactagattaatttttgtgggaagtgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataagccgctattgaaactgacggatgattctagtcccgaagaatttctgttcaacaaactaagtgaacgaaaggtatatgaatgtatcaaacagtttggtattagaatcaaggatctccgaacgtatggagtcaattatacgtttttatataatttttggacaaatgtaaagtccatatctcctcttccatcaccaaaaaagttaatagcgttaactatcaaacaaactgctgaagtggtaggtcatactccatcaatttcaaaaagagcttatatggcaacgactattttagaaatggtaaaggataaaaattttttagatgtagtatctaaaactacgttcgatgaattcctatctatagtcgtagatcacgttaaatcatctacggatggatgatatagatctttacacaaataattacaaaaccgataaatggaaatggataagcgtatgaaatctctcgcaatgaccgctttctttggggagctaagcacattagatattatggcattgataatgtctatatttaaacgccatccaaacaataccattttttcagtggataaggatggtcagtttatgattgatttcgaatacgataattataaggcttctcaatatttggatctgaccctcactccgatatctggagatgaatgcaagactcacgcatcgagtatagccgaacaattggcgtgtgtggatattattaaagaggatattagcgaatatatcaaaactactccccgtcttaaacgatttataaaaaaataccgcaatagatcagatactcgcatcagtcgagatacagaaaagcttaaaatagctctagctaaaggcatagattacgaatatataaaagacgcttgttaataagtaaatgaaaaaaaactagtcgtttataataaaacacgatatggatgccaacgtagtatcatcttctactattgcgacgtatatagacgctttagcgaagaatgcttcggaattagaacagaggtctaccgcatacgaaataaataatgaattggaactagtatttattaagccgccattgattactttgacaaatgtagtgaatatctctacgattcaggaatcgtttattcgatttaccgttactaataaggaaggtgttaaaattagaactaagattccattatctaaggtacatggtctagatgtaaaaaatgtacagttagtagatgctatagataacatagtttgggaaaagaaatcattagtgacggaaaatcgtcttcacaaagaatgcttgttgagactatcgacagaggaacgtcatatatttttggattacaagaaatatggatcctctatccgactagaattagtcaatcttattcaagcaaaaacaaaaaactttacgatagactttaagctaaaatattttctaggatccggtgcccagtctaaaagttctttattacacgctattaatcatccaaagtcaaggcctaatacatctctggaaatagaatttacacctagagacaatgaaacagttccatatgatgaactaataaaggaattgacgactctctcgcgtcatatatttatggcttctccagagaatgtaattctttctccgcctattaacgcgcctataaaaacctttatgttgcctaaacaagatatagtaggtttggatctggaaaatctatatgccgtaactaagactgacggcattcctataactatcagagttacatcaaacgggttgtattgttattttacacatcttggttatattattagatatcctgttaagagaataatagattccgaagtagtagtctttggtgaggcagttaaggataagaactggaccgtatatctcattaagctaatagagcctgtgaatgcaatcaatgatagactagaagaaagtaagtatgttgaatctaaactagtggatatttgtgatcggatagtattcaagtcaaagaaatacgaaggtccgtttactacaactagtgaagtcgtcgatatgttatctacatatttaccaaagcaaccagaaggtgttattctgttctattcaaagggacctaaatctaacattgattttaaaattaaaaaggaaaatactatagaccaaactgcaaatgtagtatttaggtacatgtccagtgaaccaattatctttggagagtcgtctatctttgtagagtataagaaatttagcaacgataaaggctttcctaaagaatatggttctggtaagattgtgttatataacggcgttaattatctaaataatatctattgtttggaatatattaatacacataatgaagtgggtattaagtccgtggttgtacctattaagtttatagcagaattcttagttaatggagaaatacttaaacctagaattgataaaaccatgaaatatattaactcagaagattattatggaaatcaacataatatcatagtcgaacatttaagagatcaaagcatcaaaataggagatatctttaacgaggataaactatcggatgtgggacatcaatacgccaataatgataaatttagattaaatccagaagttagttattttacgaataaacgaactagaggaccgttgggaattttatcaaactacgtcaagactcttcttatttctatgtattgttccaaaacatttttagacgattccaacaaacgaaaggtattggcgattgattttggaaacggtgctgacctggaaaaatacttttatggagagattgcgttattggtagcgacggatccggatgctgatgctatagctagaggaaatgaaagatacaacaaattaaactctggaattaaaaccaagtactacaaatttgactacattcaggaaactattcgatccgatacatttgtctctagtgtcagagaagtattctattttggaaagtttaatatcatcgactggcagtttgctatccattattcttttcatccgagacattatgctaccgtcatgaataacttatccgaactaactgcttctggaggcaaggtattaatcactaccatggacggagacaaattatcaaaattaacagataaaaagacttttataattcataagaatttacctagtagcgaaaactatatgtctgtagaaaaaatagctgatgatagaatagtggtatataatccatcaacaatgtctactccaatgactgaatacattatcaaaaagaacgatatagtcagagtgtttaacgaatacggatttgttcttgtagataacgttgatttcgctacaattatagaacgaagtaaaaagtttattaatggcgcatctacaatggaagatagaccatctacaagaaactttttcgaactaaatagaggagccattaaatgtgaaggtttagatgtcgaagacttacttagttactatgttgtttatgtcttttctaagcggtaaataataatatggtatgggttctgatctcccagttctaaatgcattaaataattccaatagagcgatttttgttcctataggaccttccaactgtggatactctgtattgttaatagatatattaatacttttgtcgggtaacagaggttctacgtcttttaaaaataaaagtttgataacatctggcctgttcataaataaaaacttggcgattctatatatactcttattatcaaatctagccattgtcttatagatgtgagctactgtaggtgtaccatttgattttctttctaatactatatatttctctcgaagaagttcttgcacatcatctgggaataaaatactactgttgagtaaatcagttattttttttatatcgatattgatggacatttttatagttaaggataataagtatcccaaagtagataacgacgataacgaagtatttatacttttaggaaatcacaatgactttatcagattaaaattaacaaaattaaaggagcatgtatttttttctgaatatattgtgactccagatacatatggatctttatgcgtcgaattaaatgggtctagttttcagcacggcggtagatatatagaggtggaggaatttatagatgctggaagacaagttagatggtgttctacatccaatcatatatctaaagatatacccgaagatatgcacactgataaatttgtcatttatgatatatacacttttgacgctttcaagaataaacgattggtattcgtacaggtacctccgtcgttaggagatgatagtcatttgactaatccgttattgtctccgtattatcgtaattcagtagccagacaaatggtcaatgatatgatttttaatcaagattcatttttaaaatatttattagaacatctgattagaagccactatagagtttctaaacatataacaatagttagatacaaggataccgaagaattaaatctaacgagaatatgttataatagagataagtttaaggcgtttgtattcgcttggtttaacggcgtttcggaaaatgaaaaggtactagatacgtataaaaaggtatctaatttgatataatgaattcagtgactgtatcacacgcgccatatactattacttatcacgatgattgggaaccagtaatgagtcaattggtagagttttataacgaagtagccagttggctgctacgagacgagacgtcgcctattcctgataagttctttatacagttgaaacaaccgcttagaaataaacgagtatgtgtgtgcggtatagatccgtatccgaaagatggaactggtgtaccgttcgaatcaccaaattttacaaaaaaatcaattaaggagatagcttcatctatatctagattaaccggagtaattgattataaaggttataaccttaatataatagacggggttataccctggaattattacttaagttgtaaattaggagaaacaaaaagtcacgcgatctactgggataagatttccaagttactgctgcagcatataactaaacacgttagtgttctttattgtttgggtaaaacagatttctcgaatatacgggccaagttagaatccccggtaactaccatagtcggatatcatccagcggctagagaccgccaattcgagaaagatagatcatttgaaattatcaacgttttactggaattagacaacaaggcacctataaattgggctcaagggtttatttattaatgctttagtgaaattttaacttgtgttctaaatggatgcggctattagaggtaatgatgttatctttgttcttaagactataggtgtcccgtcagcgtgcagacaaaatgaagatccaagatttgtagaagcatttaaatgcgacgagttagaaagatatattgagaataatccagaatgtacactattcgaaagtcttagggatgaggaagcatactctatagtcagaattttcatggatgtagatttagacgcgtgtctagacgaaatagattatttaacggctattcaagattttattatcgaggtgtcaaactgtgtagctagattcgcgtttacagaatgcggcgccattcatgaaaatgtaataaaatccatgagatctaatttttcattgactaagtctacaaatagagataaaacaagttttcatattatctttttagacacgtataccactatggatacattgatagctatgaaacgaacactattagaattaagtagatcatctgaaaatccactaacaagatcgatagacactgccgtatataggagaaaaacaactcttcgggttgtaggtactaggaaaaatccaaattgcgacactattcatgtaatgcaaccaccgcatgataatatagaagattacctattcacttacgtggatatgaacaacaatagttattacttttctctacaacaacgattggaggatttagttcctgataagttatgggaaccagggtttatttcattcgaagacgctataaaaagagtttcaaaaatattcattaattctataataaactttaatgatctcgatgaaaataattttacaacggtaccactggtcatagattacgtaacaccttgtgcattatgtaaaaaacgatcgcataaacatccgcatcaactatcgttggaaaatggtgctattagaatttacaaaactggtaatccacatagttgtaaagttaaaattgttccgttggatggtaataaactgtttaatattgcacaaagaattttagacactaactctgttttattaaccgaacgaggagaccatatagtttggattaataattcatggaaatttaacagcgaagaacccttgataacaaaactaattttgtcaataagacatcaactacctaaggaatattcaagcgaattactctgtccaagaaaacgaaagactgtagaagctaacatacgagacatgttagtagattcagtagagaccgatacctatccggataaacttccgtttaaaaatggtgtattggacctggtagacggaatgttttactctggagatgatgctaaaaaatatacgtgtactgtatcaaccggatttaaatttgacgatacaaagttcgtcgaagacagtccagaaatggaagagttaatgaatatcattaacgatatccaaccattaacggatgaaaataagaaaaatagagagctatatgaaaaaacattatctagttgtttatgcggtgctaccaaaggatgtttaacattcttttttggagaaactgcaactggaaagtcgacaaccaaacgtttgttaaagtctgctatcggtgacctgtttgttgagacgggtcaaacaattttaacagatgtattggataaaggacctaatccatttatcgctaacatgcatttgaaaagatctgtattctgtagcgaactacctgattttgcctgtagtggatcaaagaaaattagatctgacaatattaaaaagttgacagaaccttgtgtcattggaagaccgtgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaatatatgctcatatatttatagaagatatcacatatctaaatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaattcagacacttcttaacgaacgcggtgtgaatgtacttaaggatttgcttaaaggaagaatatcatattacgaaatgcctgataaagatctaccaacgataagatatcacggacgtaagtttctagatactagagtagtatattgtcacatgtctaaacttcaagagagagattatatgattactagacgacagctatgttatcatgaaatgtttgataaaaatatgtataacgtgtcaatggcagtattgggacaacttaatctgatgaataatttagatactttatttcaggaacaggataaggaattgtacccaaatctgaaaataaataatggcgtgttatacggagaagaattggtaacgttaaacattagttccaaatttaaatactttattaatcggatacagacactcaacggaaaacattttatatacttttctaattctacatatggcggattggtaattaaatatatcatgctcagtaatggatattctgaatataatggttctcagggaactaatccacatatgataaacggcaaaccaaaaacatttgctatcgttactagtaaaatgaaatcgtctttagaggatctattagatgtgtataattctcctgaaaacgatgatggtagtcaattgatgtttttgttttcgtcaaacattatgtccgaatcctatactctgaaagaggtaaggcatatttggtttatgactatcccagatactttttctcaatacaaccaaattcttggacgatctattagaaaattctcttacgccgatatttctgaaccagttaatgtatatcttttagccgccgtatattccgatttcaatgacgaagtaacgtcattaaacgattacacacaggatgaattgattaatgttttaccatttgacatcaaaaagctgttatatctaaaatttaagactaaagaaacgaatagaatatactctattcttcaagagatgtctgaaacgtattctcttccaccacatccatcaattgtaaaagttttattgggagaattggtcagacaatttttttataataattctcgtattaagtataacgactccaagttacttaaaatggttacatcagttataaaaaataaagaagacgctaggaattacatagatgatattgtaaacggtcacttctttgtatcgaataaagtatttgataaatctcttttatacaaatacgaaaacgatattattacagtaccgtttagactttcctacgaaccatttgtttggggagttaactttcgtaaagaatataacgtggtatcttctccataaaactgatgaaatatataaagaaataaatgtcgagctttgttaccaatggataccttccagttacattggaaccacacgagctgacgttagacataaaaactaatattaggaatgccgtatataagacgtatctccatagagaaattagtggtaaaatggccaagaaaatagaaattcgtgaagacgtggaattacctctcggcgaaatagttaataattctgtagttataaacgttccgtgtgtaataacctacgcgtattatcacgttggggatatagtcagaggaacattaaacatcgaagatgaatcaaatgtaactattcaatgtggagatttaatctgtaaactaagtagagattcgggtactgtatcatttagcgattcaaagtactgcttttttcgaaatggtaatgcgtatgacaatggcagcgaagtcactgccgttctaatggaggctcaacaaggtatcgaatctagttttgtttttctcgcgaatatcgtcgactcataaaaaagagaatagcggtaagtataaacacgaatactatggcaataattgcgaatgttttattctcttcgatatatttttgataatatgaaaaacatgtctctctcaaatcggacaaccatctcataaaatagttctcgcgcgctggagaggtagttgctgctcgtataatctccccagaataatatacttgcgtgtcgtcgttcaatttatacggatttctatagttctctgttatataatgcggttttccatcatgattagacgacgacaatagtgttctgaatttagatagttgatcagaatgaatgtttattggcgttggaaaaattatccatacagcgtctgcagagtggttgatagttgttcctagatatgtaaaataatccaacttactaggcagcaaattgtctagataaaatactgaatcaaacggtgcagacgtattggcggatctaatggaatccaattgattaactatcttttgaaaatatacatttttatgatccaatacttgtaagaatatagaaataatgataagtccatcatcgtgtttttttgcctcttcataagaactatattttttcttattccaatgaacaagattaatctctccagagtatttgtacacatctatcaagtgattggatccataatcgtcttcctttccccaatatatatgtagtgatgataacacatattcattggggagaaaccctccacttatatatcctcctttaaaattaatccttactagttttccagtgttctggatagtggttggtttcgactcattataatgtatgtctaacggcttcaatcgcgcgttagaaattgcttttttagtttctatattaataggagatagttgttgcggcatagtaaaaatgaaatgataactgtttaaaaatagctcttagtatgggaattacaatggatgaggaagtgatatttgaaactcctagagaattaatatctattaaacgaataaaagatattccaagatcaaaagacacgcacgtgtttgctgcgtgtataacaagtgacggatatccgttaataggagctagaagaacttcattcgcgttccaggcgatattatctcaacaaaattcagattctatctttagagtatccactaaactattacggtttatgtactacaatgaactaagagaaatctttagacggttgagaaaaggttctatcaacaatatcgatcctcactttgaagagttaatattattgggtggtaaactagataaaaaggaatctattaaagattgtttaagaagagaattaaaagaggaaagtgatgaacgtataacagtaaaagaattcggaaatgtaattctaaaacttacaacgcgcgataaattatttaataaagtatatataggttattgcatggcgtgttttattaatcaatcgttggaggatttatcgcatactagtatttacaatgtagaaattagaaagattaaatcattaaatgattgtattaacgacgataaatacgaatatctgtcttatatttataatatgctagttaatagtaaatgaacttttacagatctagtataattagtcagattattaagtataatagacgactagctaagtctattatttgcgaggatgactctcaaattattacactcacggcattcgttaaccaatgcctatggtgtcataaacgagtatccgtgtccgctattttattaactactgataacaaaatattagtatgtaacagacgagatagttttctctattctgaaataattagaactagaaacatgtttagaaagaaacgattatttctgaattattccaattatttgaacaaacaggaaagaagtatactatcgtcatttttttctctagatccagctactgctgataatgatagaatagacgctatttatccgggtggcatacccaaaaggggtgagaatgttccagagtgtttatccagggaaattaaagaagaagttaatatagacaattcttttgtattcatagacactcggttttttattcatggcatcatagaagataccattattaataaattttttgaggtaatcttctttgtcggaagaatatctctaacgagtgatcaaatcattgatacatttaaaagtaatcatgaaatcaaggatctaatatttttagatccgaattcaggtaatggactccaatacgaaattgcaaaatatgctctagatactgcaaaactcaaatgttatggccatagaggatgttattacgaatcattaaaaaaattaactgaggatgattgattagaaaatataaattaatttaccatcgtgtatttttataacgggattgtccggcatatcatgtagatagttaccgtctacatcgtatactcgaccatctacgcctttaaatcctctatttattgacattaatctattagaattggaataccaaatattagtaccctcaattagtttattggtaatatttttgttagacgatagatcgatggctcttgaaaccaaggttttccaaccggactcattgtcgatcggtgagaagtctttttcattagcatgaatccattctaatgatgtatgtttaaacactctaaacaattggacaaattcttttgatttgctttgaatgatttcaaataggtcttcgtctacagtaggcataccattagataatctagccattataaagtgcacgtttacatatctacgttctggaggagtaagaacgtgactattgagacgaatggctcttcctactatctgacgaagagacgcctcgttccatgtcatatctaaaatgaagatatcattgattgagaagaagctaataccctcgcctccactagaagagaatacgcatgttttaatgcattctccgttagtgtttgattcttggttaaactcagccaccgccttgattctagtatcttttgttctagatgagaactctatattagagataccaaagactttgaaatatagtaataagatttctattcctgactgattaacaaatggttcaaagactagacatttaccatgggatgctaatattcccaaacatacatctataaatttgacgcttttctcttttaattcagtaaatagagagatatcagccgcactagcatcccctcccaatagttctccccttttaaaggtatctaatgcagatttagaaaattctctatctcttaatgaatttttaaaatcattatatagtgttgctatctcttgcgcgtattcgcccggatcacgattttgtctttcaggaaagctatcgaacgtaaacgtagtagccatacgtctcagaattctaaatgatgatatacctgtttttatttcagcgagtttagccttttgataaatttcttcttgctttttcgacatattaacgtatcgcattaatactgttttcttagcgaatgatgcagacccttctacgtcatcaaaaatagaaaactcgttattaactatgtacgaacataggcctcctagtttggagactaattctttttcatcaactagacgtttattctcaaatagcgattggtgttgtaaggatcctggtcgtagtaagttaaccaacatggtgaattcttgcacactattgacgataggtgtagccgataaacaaatcatcttatggttttttaatgcgatggtcttagataaaaaattatatactgaacgagtaggacggatcttaccatcttctttgattaatgatttagaaatgaagttatgacattcatcaataatgacgcatattctactcttggaattaatagttttgatattagtaaaaaatttatttctaaaattttgatcatcgtaattaataaaaatacaatccttcgttatctctggagcgtatctgagtatagtgttcatccaaggatcttctatcaaagcctttttcaccaataagataatagcccaattcgtataaatatccttaagatgtttgagaatatatacagtagtcattgttttaccgacacccgtttcatggaacaataaaagagaatgcatactgtctaatcctaagaaaactcttgctacaaaatgttgataatccttgaggcgtactacgtccgaccccatcatttcaacaggcatattagtagttctgcgcaatgcataatcgatataggccgcgtgtgatttactcatttatgagtgataagtaataactatgttttaaaaatcacagcagtagtttaactagtcttctctgatgtttgttttcgatactttttgaatcagaagtcatactagaataaagcaacgagtgaacgtaatagagagcttcgtatactctattcgaaaactctaagaacttattaatgaattccgtatccactggattgtttaaaatactaaattgaacactgttcacatccttccaagaagaagacttagtgacggacttaacatgagacataaataaatccaaattttttttacaaacatcactagccaccataatggcgctatcttttaaccagctatcgcttacgcattttagcagtctaacatttttaaagagactacaatatattctcatagtatcgattacacctctaccgaataaagttggaagtttaataatacaatatttttcgtttacaaaatcaaataatggtcgaaacacgtcgaaggttaacatcttataatcgctaatgtatagattgttttcagtgagatgattattagatttaatagcatctcgttcacgtttgaacagtttattgcgtgcgctgaggtcggcaactacggcgtccgctttagtactcctcccataatactttacgctattaatctttaaaatttcatagactttatctagatcgctttctggtaacatgatatcatgtgtaaaaagttttaacatgtcggtcggcattctatttagatcattaactctagaaatctgaagaaagtaattagctccgtattccagactaggtaatgggcttttacctagagacagattaagttctggcaatgtttcataaaatggaagaaggacatgcgttccctcccggatattttttacaatttcatccatttacaactctatagtttgttttcattattattagttattatctcccataatcttggtaatacttaccccttgatcgtaagataccttatacaggtcattacatacaactaccaattgtttttgtacataatagattggatggttgacatccatggtggaataaactactcgaacagatagtttatctttccccctagatacattagccgtaatagttgtcggcctaaagaatatctttggtgtaaagttaaaagttagggttcttgttccattattgctttttgtcagtagttcattataaattctcgagatgggtccgttctctgaatatagaacatcatttccaaatctaacttctagtctagaaataatatcggtcttattcttaaaatctattcccttgatgaagggatcgttaatgaacaaatccttggcctttgattcggctgatctattatctccgttatagacgttacgttgactagtccaaagacttacaggaatagatgtatcgatgatgttgatactatgtgatatgtgagcaaagattgttctcttagtggcatcactatatgttccagtaatggcggaaaactttttagaaatgttatatataaaagaattttttcgtgttccaaacattagcagattagtatgaagataaacactcatattatcaggaacattatcaatttttacatacacatcagcatcttgaatagaaacgataccatcttctggaacctctacgatctcggcagactccggataaccagtcggtggaccatcgctaacaataactagatcatccaacaatctactcacatatgcatctatataatctttttcatcttgtgagtaccctggatacgaaataaatttattatccgtatttccataataaggtttagtataaacagagagcgatgttgccgcatgaacttcagttacagtcgccgttggttggtttatttgacctattactctcctaggtttctctataaatgatggtttaatttgtacattcttaaccatatatccaataaagctcaattcaggaacataaacaaattctttgttgaacgtttcaaagtcgaacgaagagtcacgaataacgatatcggatactggattgaaggttaccgttacggtaatttttgaatcggatagtttaagactgctgaatgtatcttccacatcaaacggagttttaatataaacgtatactgtagatggttctttaatagtgtcattaggagttaggccaatagaaatatcattaagttcactagaatatccagagtgtttcaaagcaattgtattattgatacaattattatataattcttcgccctcaatttcccaaataacaccgttacacgaagagatagatacgtgattaatacatttatatccaacatatggtacgtaaccgaatcttcccatacctttaacttctggaagttccaaactcagaaccaaatgattaagcgcagtaatatactgatccctaatttcgaagctagcgatagcctgattgtctggaccatcgtttgtcataactccggatagagaaatatattgcggcatatataaagttggaatttgactatcgactgcgaagacattagaccgtttaatagagtcatccccaccgatcaaagaattaatgatagtattattcattttctatttaaaatggaaaaagcttacaataaactccgtagagaaatatctataatttgtgagttttccttaaagtaacagcttccgtaaacgccgtctttatctcttagtaagtttattgtatttataaccttttccttatcttcatagaatactaaaggcaacaaagaaatttttggttcttctctaagagctacgtgagacttaaccatagacgccaacgaatccctacatattttagaacagaaatacccaacttcaccacccttgaatgtctcaatactaataggtttaaaaaccaaatcttgattacaaaaccaacacttatcaattacactatttgtcttaatagacacatctgccatagatttataatactttggtagtatacaagcgagtgcttcttctttagcgggcttaaagactgctttaggtgctgaaataaccacatctggaaggcttactcgcttagccatttaattacggaactatttttttatacttctaatgagcaagtagaaaacctctcatctacaaaaacatactcgtgtccataatcctctaccatagttacacgttttttagatctcatatgtgctaaaaagttttcccatactaattggttactattatttttcgtataatttttaacagtttgaggttttagatttttagttacagaagtgatatcgaatattttatccaaaaagaatgaataattaattgtcttagaaggagtgttttcttggcaaaagaataccaagtgcttaaatatttctactacttcattaatcttttctgtactcagattcagtttctcatcttttacttgattgattatttcaaagactaacttataatcctttttatttattctctcgttagccttaagaaaactagatacaaaatttgcatctacatcatccgtggatatttgatttttttccatgatatccaagagttccgagataatttctccagaacattgatgagacaataatctccgcaatacatttctcaaatgaataagtttattagacacatggaagtttgactttttttgtacctttgtacatttttgaaataccgactcgcaaaaaatacaatattcatatccttgttcagatactataccgttgtgtctacaaccgctacataatcgtagattcatgttaacactctacgtatctcgtcgtccaatattttatataaaaacattttatttctagacgttgccagaaaatcctgtaatatttttagttttttgggctgtgaataaagtatcgccctaatattgttaccgtcttccgccaatatagtagttaaattatccgcacatgcaaaagaacaccgcttaggcggattcagtacaatgttatatttttcgtaccaactcatttaaatatcataatctaaaatagttctgtaatatgtctagcgctaatatattgatcataatcctgtgcataaattaagatacaacaatgtctcgaaatcatcgacatggcttcttccatagttagaagatcgtcgtcaaagttagcaacgtgattcatcaacatttgctgttttgaggcagcaaatactgaaccgtcgccattcaaccattcataaaaaccatcgtctgaatccattgataatttcttgtactggtttttgagagctcgcatcaatctagcatttctagctcccggattgaaaacagaaagaggatcgtacatccagggtccattttctgtaaatagaatcgtataatgtcccttcaagaagatatcagacgatccacaatcaaagaattggtctccgagtttgtaacaaactgcggactttaacctatacatgataccgtttagcatgatttctggtgatacgtcaatcggagtatcatctattagagatctaaagccggtgtaacattctccaccaaacatattcttattctgacgtcgttctacataaaacatcattgctccattaacgataacaggggaatgaacagcactacccatcacattagttcccaatggatcaatgtgtgtaactccagaacatcttccatatcctatgttaggaggagcgaacaccactcttccactattgccatcgaatgccatagaataaatatccttggaattgatagaaatcggactgtcggatgttgtgatcatcttcataggattaacaactatgtatggtgccgcctgaagtttcatatcgtaactgatgccgtttataggtctagccacagaaaccaacgtaggtctaaatccaactatagacaaaatagaagccaatatctgttcctcatctgtcataacttgagagcatccagtatgaataatcttcattagatggggatctaccgcatcatcatcgttacaataaaaaattcccattctaatgttcataattgcttttctaatcatggtatgcatgtttgctctctgaatctctgtggaaattagatctgatacacctgtaatcactatcggattatcctccgtaagacgattaaccaacaacatataattataagactttacttttctaaattcataaagttgctggattaggctataggtgtctccatgtacatacgcgttctcgagcgcaggaagtttaataccgaatagtgccatcagaataggatgaatatagtaattagtttctggttttctataaataaaagacaaatcttgtgaactagacatatcggtaaaatgcatggattggaatcgtgtagtcgacagaagaatatgatgattagatggagagtatattttatctaactctttgagttggtcaccgattctaggactagctcgagaatgaataagtactaaaggatgagtacatttcacagaaacactagcattgttcaatgtgctctttacatgggtaaggagttgaaatagctcgtttctatttgttctgacaatatttagtttattcataatgttaagcatatcctgaatagtaaagttagatgtgtcatacttgttagtagttagatatttagcaattgcattcccatcatttctcaatctcgtactccaatcatgtgtagatgctacttcgtcgatggaaaccatacaatcctttttgataggctgttgagattgattatttcctgcacgtttaggtttggtacgttgatttctagcccctgcggatataaagtcatcgtctacaatttgggacaatgaattgcatacactacaagacaaagatttatcagaagtgtgaatatgatcttcatctaccaaagaaagagtttgattagtataactagattttagtcctgcgttagatgttaaaaaaacatcgctattgaccacggcttccattatttatattcgtagtttttactcgaaagcgtgattttaatatccaatcttattacttttggaatcgttcaaaacctttgactagttgtagaatttgatctattgccctacgcgtatactcccttgcatcatatacgttcgtcaccagatcgtttgtttcggcctgaagttggtgcatatctctttcaacattcgacatgagatccttaagggccatatcgtctagattttgttgagatgctgctcctggatttggattttgttgtgctgttgtacatactgtaccaccagtaggtgtaggagtacatacagtggccacaataggaggttgaggaggtgtaaccgttggagtagtacaagaaatatttccatccgattgttgtgtacatgtagttgttggtaacgtctgagaaggttgggtagatggcggtgtcgtcgtcttttgatctttattaaatttagagataatatcctgaacagcattgctcggcgtcaacgctggaaggagtgaactcgccggcgcatcagtatcttcagacagccaatcaaaaagattagacatatcagatgatgtattagtttgttgtcgtggttttggtgtaggaacagtactactaggtagaagaataggagccggtgtagctgttggaaccggctgtggagttatatgaatagttggttgtagcggttggataggctgtctgctggcgaccgtcatattatctctagctagttgttctcgcaactgtctttgataatacgactcttgagactttagtcctatttcaatcgcttcatcctttttcgtatccggatccttttcttcagaataatagattgacgactttggtgtagaggattctgccagcccctgtgagaacttgttaaagaagtccatttaaggctttaaaattgaattgcgattataagattaaatggcagacacagacgatattatcgactatgaatccgatgatctcaccgaatacgaggatgatgaagaagaggaagaagatggagagtcactagaaactagtgatatagatcccaaatcttcttataagattgtagaatcagcatccactcatatagaagatgcgcattccaatcttaaacatatagggaatcatatatctgctcttaaacgacgctatactagacgtataagtctatttgaaatagcgggtataatagcagaaagctataacttgcttcaacgaggaagattacctctagtttcagaattttctgacgaaacgatgaagcaaaatatgctacatgtaattatacaagagatagaggagggttcttgtcctatagtcatcgaaaagaacggagaattgttgtcggtaaacgattttgacaaagatggtctaaaattccatctagactatattatcaaaatttggaaacttcaaaaacgatattagaatttatacgaatatcgttctctaaatgtcacaatcaagtctcgcatgttcagcaatttattgtcgtactttatatcgtgttcattaacgatatcttgcaaaatagtaatgattctatcttccttcgatagatattcttcagagattattgtcttatattttttcttgttatccgatatgaatttgataagactttgaacattattgatacccgtctgtttaattttttctacagatattttagttttggcagattctatcgtatctgtcaatagacatccaacatcgacattcgacgtcaattgtctataaatcaacgtataaattttagaaataacattagcgaattgttgtgcattgatgtcgttattctgaaacagtatgattttaggtagcattttcttaacaaagagaacgtatttattgttactcagttgaacagatgatatatccagattactaacgcatctgattccgtataccaaactttcagaagaaatggtgtacaattgtttgtattcattcaatgtctctttttcagaaattagtttagagtcgaatactgcaataattttcaagagatagttttcatcagataagattttatttagtgtagatatgataaaactattgttttgttggagaacttgatacgccgcgttctctgtagtcgacgctctcaaatgggaaacaatctccattatttttttggaatcggatactatatcttcggtatcttgacgcagtctagtatacatagagttaagagagattagagtttgtacattaagcaacatgtctctaaatgtggctacaaacttttcctttttcacataatctagtttattatataccgatttcacaacggcaccagatttaaggaaccagaatgaaaaactctgataactacaatatttcatcatagttacgattttatcatcttctatagttggtgtaatagcgcatacctttttctccaagactggaaccaacgtcataaaaatgtttaaatcaaaatccatatcaacatctgatgcgctaagaccagtctcgcgttcaagattatctttactaatggtgacgaactcatcatatagaactctaagtttgtccattatttatttacagatttagttgtttaatttatttgtgctcttccagagttgggatagtatttttctaacgtcggtattatattattaggatctacgttcatatgtatcataatattaatcatccacgttttgataaatctatctttagcttctgaaataacgtatttaaacaaaggagaaaaatatttagctacggcatcagacgcaataacattttttgtaaatgtaacgtatttagacgacagatcttcgttaaaaagttttccatctatgtagaatccatcggttgttaacaccattcccgcgtcagattgaataggagtttgaatagtttgttttggaaatagatccttcaataacttatagttgggtgggaaaaaatcgattttatcactagactctttcttttttactatcattacctcatgaactatttcttgaatgagtatatgtattttctttcctatatcggacgcgttcattggaaaatataccatgtcgttaactataagaatatttttatcctcgtttacaaactgaataatatcagatgtagttcgtaaacgaactatatcatcaccagcacaacatctaactatatgatatccactagtttcctttagccgtttattatcttgttccatattagcagtcattccatcatttaagaaggcgtcaaagataatagggagaaatgacattttggattctgttacgactttaccaaaattaaggatatacggacttactatctttttctcaacgtcgatttgatgaacacacgatgaaaatgtgcttctatgagattgatcatgtagaaaacaacaagggatacaatatttccgcatatcatgaaatatattaagaaatcccaccttattatatttccccaaaggatccatgcatgtaaacattatgccgttatcattaataaagacttctttctcatcggatttgtaaaagttgttactgatttttttcattccaggatctagataattaataatgatgggttttctattcttattctttgtattttggcatatcctagaccagtaaacagtttccactttggtaaaatcagcagacttttgaacgctattaaacatggcattaatggcaataactaaaaatgtaaaatatttttctatgttaggaatatggtttttcactttaatagatatatggtttttggccaaaatgatagatatttttttatccgaggatagtaaaatattattagtcgccgtctctataaaaatgaagctagtctcgatatccaattttattctagaattgataggagtcgccaaatgtaccttatacgttatatctcccttgatgcgttccatttgtgtatctatatcggacacaagatctgtaaatagttttacgttattaatcatcacggtatcgccgtcgctagataacgctaatgtaccatccaagtcccaaatggagagatttaactgttcatcgtttagaataaaatgattaccggtcatattaataaagtgttcatcgtatctagataacaacgacttataattaatgtccaagtcttgaactcgctgaatgatcttttttaacccagttagttttagattggtacgaaatatattgttaaactttgattctacagtaatgtccaaatctagttgtggaaatacttccatcaacattgtttcaaacttgataatattattatctacatcttcatacgatccaaattccggaatagatgtatcacatgctcttgccacccagataaccaaaaagtcacacgctccaggatatacattgtataaaaagctatcgttttttagtagtgtttttttctgagtatatacgaagggattaaaaatagtattatcaacgtaactatattccaaattattcttatgagaatagataataatatcgtccttaatatctaacaaatttcctaaatatccctttaattgagtcattcgaagcgtcaatagaatatgtctcttaactatttccggctgttgtatatttaaatgacttcgtaaaaaataatatatgggcgacttctcatctatgtaatcatatggagtgagatatagggctcgttctacctcctgccccttacccacctgtaataccaattgcggacttactatatatcgcatatttatatcgtggggtaaagtgaaaatctactaccgatgatgtaagtcttacaatgttcgaaccagtaccagatcttaatttggaggcctccgtagaactaggggaggtaaatatagatcaaacaacacctatgataaaggaaaatagcggttttatatcccgtagtagacgtctattcgcccatagatctaaggatgatgagagaaaactagcactacgattctttttacaaagactttattttttagatcatagagagattcattatttgttcagatgcgttgacgctgtaaaagacgtcactattaccaaaaaaaataacattatcgtggcgccttatatagcacttttaactatcgcatcaaaaggatgcaaacttacagaaacaatgattgaagcattttttccagaactatataatgaacatagtaagaaatttaaattcaactctcaagtatccatcatccaagaaaaactcggataccagtttggaaactatcacgtttatgattttgaaccgtattactctacagtagctctggctattcgagatgaacattcatctggcatttttaatatccgtcaagagagttatctggtaagttcattatctgaaataacatatagattttatctaattaatctaaaatctgatcttgttcaatggagtgctagtacgggcgctgtaattaatcaaatggtaaatactgtattgattacagtgtatgaaaagttacaactggtcatagaaaatgattcacaatttacatgttcattggctgtggaatcaaaacttccaataaaattacttaaagatagaaatgaattatttacaaaattcattaacgagttaaaaaagaccagttcattcaagataagcaaacgcgataaggatacgctactaaaatattttacttaggactggagttagaatttatagacgactcatttcgtttatcattgttactattattactattactatcattattagtgttggcattattagtattcttcttgtcatcttgttcagaaatatacagcaatgctatacctaatactaaatacattatcatgctcgcaatggctctaacaacaacgaaccaaaatgaatttggtcgtagcttttgttcacaaaaatacataaagaaatgtctacataaatctatggcgccattggctacttgaaatagcgccagtcctcctacagattttaatatagctgtataacatgacatttattcatcatcaaaagagacagagtcaccatctgtcatatttagattttttttcatgtgttcaaagtatcctctactcatttcattataatagtttatcatacttagaattttaggacggatcaatgagtaagacttgactagatcgtcagtagtaatttgtgcatcgtctattctgcatccgcttcgtcgaataatgtatagcatcgctttgagattctccatagctatcaagtctttatacaatgacatggaaatatctgtgaatactttatacttctccaacatcgatgccttaacatcatcgcctactttagcattgaaaatacgttctattgtgtagatggatgtagcaagatttttaaacaacaatgccatcttacacgatgattgcctcaagtctccaatcttttgtttagaacgattagctacagagttcaacgcttggctgactagcatattattatctttagaaattgtattcttcaatgaggcgtttatcatatctgtgatttcgttagtcatattacagtctgactgggttgtaatgttatccaacatatcacctatggatacggtacacgtaccagcatttgtaataatcctatctaagatgttgtatggcattgcgcagaaaatatcttctcctgtaatatctccactctcgataaatctactcagattattcttaaatgccttattctctggagaaaagatatcagtgtccatcatttcattaatagtatacgcagaaaagataccacgagtatcaattctatccaagatacttatcggttccgagtcacagataatggtttcctctccttcgggagatcctgcatagaaatatctaggacaatagtttctatactgtctgtaactctgataatctctaaagtcactaactgataccatgaaattgagaagatcaaacgctgaagtaatcaatttttctgcctcgtttttactacaactagttttcatcaatgtagcgacgatgtattgtttagttactcttggtctaatactgatgatagagatattattgcttcccataatggatcttctagtagtcaccttaaagcccattgatgcgaatagcagatagataaagtcttggtatgactcctttctaatatagtacggactacctttgtcacccaactttatacccacataagccataacaacctctttaatagccgtttcatgaggtttatcagccatgagcctgagtagttggaagaatctcatgaatcccgtctcagaaagtcctatatgcatgatagatttatctttcctgggaaactctcgtatagtcatagatgaaatactcttcaaagtttctgaaataagattagtaacagtcttacctccgactactctgggtaacaaacatactctaataggtgttttctctgcggagataatatcagaaaggatagagcaataagtagtattattgtgattataaagaccgaatacataacaggtagaatttataaacatcatgtcctgaaggtttttagacttgtattcctcgtaatccataccgtcccaaaacatggatttggtaactttgatagccgtagatctttgttccttcgccaacaggttaaagaaattaataaagaatttgttgtttctatttatgtccacaaattgcacgtttggaagcgccacggttacattcactgcagcattttgaggatcgcgagtatgaagtacgatgttattgtttactggtatatctggaaagaaatctaccagtctaggaataagagattgatatcgcatagaaatagtaaagtttataatctcatcatcgaagagcattttgttaccattgtaataaatatccactctgtcatatgtataaatgaagtactgttcaaacatgatgagatgtttatatgttggcatagtagtgagatcgacgtttggtaatggcaatgtattaagattaactccataatgtctagcagcatctgcgatgttataagcgtcgtcaaagcggggtcgatcttgtattgttatatattgtctaacacctataagattatcaaaatcttgtctgcttaatacaccgttaacaatttttgccttgaattcttttattggtgcattaataacatccttatagaggatgttaaacaaataagtgttatcaaagttaagatctggatatttcttttctgctagaacatccattgagtcggagccatctggtttaatataaccaccgataaatctagctctgtattctgtatccgtcaatctaatattaagaaggtgttgagtgaaaggtggaagatcgtaaaagctgtgagtattaatgataggattagtttccgaactaatgttaattggggtattaataatatctatatttccagcgttaagtgtaacattaaacagttttaattcacgtgacgtggtatcaattaaataattaatgcccaatttggatatagcagcctgaagctcatcttgtttagttacggatcctaatgagttattaagcaatatatcgaacggatgaacgaaggttgttttaagttggtcacatactttgtaatctagacatagatgcggaagaacggtagaaactatacgaaataaatattcagagtcctctaattgatcaagagtaactattgacttaataggcatcatttatttagtattaaatgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattcaccggccactattatcgaacgagtacaacctcatactactattattgacgatactccacctcctacttttcgtagagagttattgatatcggaacaacgtcaacaacgagaaaaaagatttaatattacagtatcgaaaaatgctgaagcaataatggaatctagatctatgataacttctatgccaacacaaacaccatccttgggagtagtttatgataaagataaaagaattcagatgttggaggatgaagtggttaatcttagaaatcaacgatctaatacaaaatcatctgataatttagataattttaccaaaatactatttggtaagactccgtataaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacaaaaaattatcgtcactaacgtgattattattgtcataaacattatcgagcaagcattgctaaaactcggatttgaagaaatcaaaggactgagtaccgatatcacttcagaaattatcgatgtggagatcggagatgactgcgatgctgtagcatcaaaactaggaatcggtaacagtccggttcttaatattgtattgtttatactcaagatattcgttaaacgaattaaaattatttaatttaatacattcccatatccagacaacaatcgtctggattaatctgttcctgtcgtctcataccggacgacatattaatctttttattagtaggcatctttttagatggtttctttttcccagcattaactgagtcgatacctagaagatcgtgattgatctctccgaccattccacgaacttctaattggccgtctctaacggtaccataaactattttaccagcattagtaacagcttggacaatctgaccatccatcgcattgtacgatgtagtagtaactgttgttctacgtctaggagcaccagaagtatttttggagcccttggaggctgatgtagaagaagacgaggattttgattttggtttacatgtaatacattttgaactctttgattttgtatcacatgcgccggcagtcacatctgtttgagaattaagattattgttgcctcctttgacggctgcatctccaccgatttgcgctagtagatttttaagctgtggtgtaatcttattaactgtttcgatataatcatcgtaactgcttctaacggctaaattttttttatccgccatttagaagctaaaaatatttttatttatacagaagatttaactagattatacaatgaactaatatgatccttttccagattatttacaaacttggtattttttggttctggaggaggcgaatttaaattcggacttggattcggattttgtgagttcttgatcttattatacatcgagtataggatggcgacggtaactgctacacaaataccgatcaacaaaagaataccaatcatttattgacaataacttcactattgatcaagtatgcaatatatcatcttttcactaaataagtagtaataatgattcaacaatgtcgagatatatggacgataataatttagttcatggaaatatcgctatgattggtatgaatgactccgctaactctgtggggcgcgcagtgctttccccacatagaataaattagcattccgactgtgataataataccaagtataaacgccataatactcaatactttccatgtacgagtgggactggtagacttactaaagtcaataaaggcgaagatacacgaaagaatcaaaagaatgattccagcgattagcacgccggaaaaataatttccaatcataagcatcatgtccatttaactaataaaaattttaaatcgccgaatgaacaaagtggaatataaaccatataaaaacaatagtttgtactgcaaaaataatatctatttttgttttcgaagatatggtaaaattaaatagtagtacacagcatgttataactaacagcagcaacggctcgtaattacttatcatttactagacgaaaaggtggtgggatattttcttgctcaaataatacgaatatatcacccatccattttatgcgatgtttatatactctaatctttaatagatctatagacgacgggtttaccaacaatatagattttatcgattcatctaatttaaacccttccttaaacgtgaatgatctattatctggcataacgatgaccctacctgatgaatcggacaatgtactgggccatgtagaataaattatcaacgaattatcgtctacgaacatttatatcatttgttttaattttaggacgcgaataaatagatataaaatagaaaataacagatattacaaccagtgttatggccgcgcccaaccaggtaggcagttttattttatcttttactacaggttctcctggatgtacgtcaccaacggcggacgtagttctagtacaattagacgtaagttccgcttgggaattttttaacgctaaagagttaacgttaatcgtgcacccaacgtatttacatctagttctttgaacatcttgattataatataaccattttctatctctagattcgtcggtgcactcatgtaaccaacataccctaggtcctaaatatttatctccggaattagattttggataattcgcgcaccaacaatttctatttcctttatgatcgttacaaaagacgtataatgccgtatccccaaaagtaaaataatcaggacgaataattctaataaactcagaacaatatctcgcatccatatgtttggagcaaatatcggaataagtagacatagccggtttccgttttgcacgtaaccattctaaacaattggggtttccaggatcgtttctacaaaatccagtcatgaaatcgtcacaatgttctgtcttgtaattattattaaatatttttggacagtgtttggtatttgtcttagaacaacattttgccacgctatcactatcgcccaggagataatccttttttataaaatgacatcgttgcccggatgctatataatcagtagcgtgttttaaatccttaatatattcaggagttacctcgttctgataatagattaatgatccaggacgaaatttgaaagaactacatggttctccatgaattaatacatattgtttagcaaattcaggaactataaaactactacaatgatctatcgacataccatctatcaaacaaaacttgggtttaatttctcccggagatgtttcataatagtacgtataactttcttctgcaaacttaacagctctattatattcaggataattaaaacctaattccatatatttgtctcgtatatctgctattcctggtgctattttgattctattaagagtaacagctgcccccattcttaataatcgtcagtatttaaactgttaaatgttggtatatcaacatctaccttatttcccgcagtataaggtttgttgcaggtatactgttcaggaatggttacatttatacttcttctatagtcctgtctttcgatgttcatcacatatgcaaagaacagaataaacaaaataatgtaagaaataatattaaatatctgtgaattcgtaaatacattgattgccataataattacagcagctacaatacacacaatagacattcccacagtgttgccattacctccacgatacatttgagttactaagcaataggtaataactaagctagtaagaggcaatagaaaagatgagataaatatcatcaatatagagattagaggagggctatatagagccaagacgaacaaaatcaaaccgagtaacgttctaacatcattatttttgaagattcccaaataatcattcattcctccataatcgttttgcatcatacctccatctttaggcataaacgattgctgctgttcctctgtaaataaatctttatcaagcactccagcacccgcagagaagtcgtcaagcatattgtaatatcttaaataactcatttatatattaaaaaatgtcactattaaagatggagtataatctttatgccgaactaaaaaaaatgacttgtggtcaacccctaagtctttttaacgaagacggggatttcgtagaagttgaaccgggatcatcctttaagtttctgatacctaagggattttacgcctctccttccgtaaagacgagtctagtatttgaaacattaacaacgaccgataataaaatcactagtatcaatccaacaaatgcgccaaagttatatcctcttcaacgcaaagtcgtatctgaagtagtttctaatatgaggaaaatgatcgaatcaaaacgtcctctatacattactcttcacttggcgtgtggatttggtaagactattaccacgtgttatcttatggctacacacggtagaaaaaccgtcatttgcgtacccaataaaatgttaatacatcaatggaagacacaggtagaggcagtcggattggaacataagatatccatagatggagtaagtagtctattaaaggaactaaagactcaaagtccggatgtattaatagtagtcagtagacatctgacaaacgatgccttttgtaaatatatcaataagcattatgatttgttcatcttggatgaatcacatacgtataatctgatgaacaatacagcagttacaagatttttagcgtattatcctccgatgatgtgttattttttaactgctacacctagaccagctaaccgaatttattgtaacagtattattaatattgccaagttatccgatctaaaaaaaactatctatgcggtagatagtttttttgagccatattccacagacaatattagacatatggtaaaacgactagatggaccatctaataaatatcatatatataccgagaagttattatctgtagacgagcctagaaatcaacttattcttaataccctggtagaagaattcaagtcaggaactattaatcgcattttagttattactaaactacgtgaacatatggtattattctacaaacgattattagatcttttcggaccagaggttgtatttataggagacgcccaaaatagacgtactccagatatggtcaaatcaatcaaggaactaaatagatttatattcgtatccaccttattttattccggtactggtttagatattcctagtttggattcgttgttcatttgctcggcagtaatcaacaatatgcaaatagagcaattactagggagggtatgtcgagaaacagaactattagataggacggtatatgtatttcctaacacatccatcaaagaaataaagtacatgataggaaatttcatgcaacgaattattagtctgtctgtagataaactaggatttaaacaagaaagttatcggaaacatcaagaatccgatcccacttctgtatgtacaacatcctccagagaagaacgtgtattaaatagaatatttaactcgcaaaatcgttaagaagtttaagcgacgatccgcatgctgcgcaggccagtgtattacccctcatagtattaatataatccaatgatacttttgtgatgtcggaaatcttaaccaatttagactgacaggcagaacacgtcatgcaatcatcatcgtcatcgataactgtagtcttgggcttctttttgcggctcttcattccggaacgcacattggtgctatccatttaggtagtaaaaaataagtcagaatatgccctataacacgatcgtgcaaaacctggtatatcgtctctatctttatcacaatatagtgtatcgacatctttattattattgacctcgtttatcttggaacatggaatgggaacatttttgttatcaacggccacctttgccttaattccagatgttgtaaaattataactaaacagtctatcatcgacacaaatgaaattcttgtttagacgtttgtagtttacgtatgcggctcgttcgcgtctcattttttcagatattgcaggtactataatattaaaaataagaatgaaataacataggattaaaaataaagttatcatgacttctagcgctgatttaactaacttaaaagaattacttagtctgtacaaaagtttgaaattttcagattctgcggctatagaaaagtataattctttggtagaatggggaacatctacttactggaaaataggcgtgcaaaaggtagctaatgtcgagacgtcaatatctgattattatgatgaggtaaaaaataaaccgtttaatattgatccgggctattacattttcttaccggtatattttgggagcgtctttatttattcgaagggtaaaaatatggtagaacttggatctggaaactcttttcaaataccagatgatatgcgaagtgcgtgtaacaaagtattagacagcgataacggaatagactttctgagatttgttttgttaaacaatagatggataatggaagatgctatatcaaaatatcagtctccagttaatatatttaaactagctagtgagtacggattaaacatacccaaatatttagaaattgaaatagaggaagacacattatttgacgacgagttatactctattatagaacgctcttttgatgataaatttccaaaaatatccatatcgtatattaagttgggagaacttaggcggcaagttgtagactttttcaaattctcgttcatgtatattgagtccatcaaggtagatcgtataggagataatatttttattcctagcgttataacaaaatcaggaaaaaagatattagtaaaagatgtagaccatttaatacgatctaaggttagagaacatacatttgtaaaagtaaaaaagaaaaacacattttccattttatacgactatgatgggaacggaacagaaactagaggagaagtaataaaacgaattatagacactataggacgagactattatgttaacggaaagtatttctctaaggttggtagtgcaggcttaaagcaattgactaataaattagatattaatgagtgcgcaactgtcgatgagttagttgatgagattaataaatccggaactgtaaaacgaaaaataaaaaaccaatcagcatttgatttaagcagagaatgtttgggatatccagaagcagattttataacgttagttaataacatgcggttcaaaatagaaaattgtaaggttgtaaatttcaatattgaaaatactaattgtttaaataacccgagtattgaaactatatatggaaactttaaccagttcgtctcaatctttaatatcgtcaccgatgtcaaaaaaagattattcgagtgaaataatatgcgcctttgatataggtgcaaaaaatcctgccagaactgttttagaagtcaaggataactccgttagggtattggatatatcaaaattagactggagttctgattgggaaaggcgcatagctaaagatttgtcacaatatgaatacactacagttcttctagaacgtcagcctagaaggtcgccgtatgttaaatttatctattttattaaaggctttttatatcatacatcggctgccaaagttatttgcgtctcgcctgtcatgtctggtaattcatatagagatcgaaaaaagagatcggtcgaagcatttcttgattggatggacacattcggattgcgagactccgttccggatagacgcaaattagacgatgtagcggatagtttcaatttggctatgagatacgtattagataaatggaatactaattatacaccttataataggtgtaaatctagaaattacataaaaaaaatgtaataacgttagtaacgccattatggataatctatttacctttctacatgaaatagaagatagatatgccagaactatttttaactttcatctaataagttgcgatgaaataggagatatatatggtcttatgaaagaacgcatttcctcagaggatatgtttgataatatagtatataataaagatatacatcctgccattaagaaactagtgtattgcgacatccaacttactaaacacattattaatcagaatacgtatccggtatttaacgattcttcacaagtgaaatgttgtcattatttcgacataaactcagataatagcaatattagctctcgtacagtagagatatttgagagggaaaagtcatctcttgtatcatatattaaaactaccaataagaagagaaaggtcaattacggcgaaataaagaaaactgttcatggaggcactaatgcaaattacttttccggtaaaaagtctgacgagtatctgagtactacagttagatccaacattaatcaaccttggatcaaaaccatttctaagagaatgagagtagatatcattaatcactctatagtaacgcgtggaaaaagctctatattacaaactatagaaattatttttactaatagaacatgtgtgaaaatattcaaggattctactatgcacattattctatccaaggacaaggatgaaaaggggtgtatacacatgattgacaaattattctatgtctattataatttatttctgttgttcgaagatatcatccaaaacgagtactttaaagaagtagctaatgttgtaaaccacgtacttacggctacggcattagatgagaaattattcctaattaagaaaatggctgaacacgatgtttatggagttagcaatttcaaaatagggatgtttaacctgacatttattaagtcgttggatcataccgttttcccctctctgttagatgaggatagcaaaataaagttttttaaggggaaaaagctcaatattgtagcattacgatctctggaggattgtataaattacgtgactaaatccgagaatatgatagaaatgatgaaggaaagatcgactattttaaatagcatagatatagaaacggaatcggtagatcgtctaaaagaattgcttctaaaatgaaaaaaaacactgattcagaaatggatcaacgactagggtataagtttttggtgcctgatcctaaagccggagttttttatagaccgttacatttccaatatgtatcgtattctaattttatattgcatcgattgcatgaaatcttgaccgtcaagcggccactcttatcgtttaagaataatacagaacgaattatgatagaaattagcaatgttaaagtgactcctccagattactcacctataatcgcgagtattaaaggtaagagttatgatgcattagccacgttcactgtaaatatctttaaagaggtaatgaccaaagagggtatatccatcactaaaataagtagttatgagggaaaagattctcatttgataaaaattccgctactaataggatacgggaataaaaatccacttgatacagccaagtatcttgttcctaatgtcataggtggagtctttatcaataaacaatctgtcgaaaaagtaggaattaatctagtagaaaagattacaacatggccaaaatttagggttgttaagccaaactcattcactttctcgttttcctccgtatcccctcctaatgtattaccgacaagatatcgccattacaagatatctctggatatatcacaattggaagcgttgaatatatcatcgacaaagacatttataacggtcaatattgttttgctgtctcaatatttatctagagtgagtctagaattcattagacgtagtttatcatacgatatgcctccagaagttgtctatctagtaaacgcgataatagatagtgctaaacgaattactgaatctattactgactttaatattgatacatacattaatgacctggtggaagctgaacacattaaacaaaaatctcagttaacgatcaacgagttcaaatatgaaatgctgcataactttttacctcatatgaactatacacccgatcaactaaagggattttatatgatatctttactaagaaagtttctctactgtatctaccacacttctagatatccagatagagattcgatggtttgtcatcgcatcctaacgtacggcaaatattttgagacgttggcacatgatgaattagagaattacataggcaacatccgaaacgatatcatgaacaatcacaagaacagaggcacttacgcggtaaacattcatgtactaacaactcccggacttaatcacgcgttttctagcttattgagtggaaagttcaaaaagtcagacggtagttatcgaacacatcctcactattcatggatgcagaatatttctattcctaggagtgttggattttatccggatcaagtaaagatttcaaagatgttttctgtcagaaaataccatccaagtcaatatctttacttttgttcatcggacgttccggaaagaggtcctcaggtaggtttagtatctcaattgtctgtcttgagttccattacaaatatactaacgtctgagtatttggatttggaaaagaaaatttgtgagtatatcagatcatattataaagatgatataagttactttgaaacaggatttccaatcactatagaaaatgctctagtcgcatctcttaatccaaatatgatatgtgattttgtaactgactttagacgtagaaaacggatgggattcttcggtaacttggaggtaggtattactttagttagggatcacatgaatgaaattcgcattaatattggagcgggaagattagtcagaccattcttggttgtggataacggagagctcatgatggatgtgtgtccggagttagaaagcagattagacgacatgacattctctgacattcagaaagagtttccgcatgtcatcgaaatggtagatatagaacaatttacttttagtaacgtatgtgaatcggttcaaaaatttagaatgatgtcaaaggatgaaagaaagcaatacgatttatgtgactttcctgccgaatttagagatggatatgtggcatcttcattagtgggaatcaatcacaattctggacccagagctattcttggatgtgctcaagctaaacaagctatctcttgtctgagttcggatatacgaaataaaatagacaatggaattcatttgatgtatccagagaggccaatcgtgattagtaaggctttagaaacttcaaagattgcggctaattgcttcggccaacatgttactatagcattaatgtcgtacaaaggtatcaatcaagaggatggaattatcatcaaaaaacaatttattcagagaggcggtctcgatatagttaccgcaaagaaacatcaagtagaaattccgttggaaaactttaataacaaagaaagagataggtctaacgcctattcaaaattagaaagtaatggattagttagactgaatgctttcttggaatccggagacgctatggcacgaaatatctcatcaagaactcttgaagatgattttgctagagataatcagattagcttcgatgtttccgagaaatataccgatatgtacaaatctcgcgttgaacgagtacaagtagaacttactgacaaagttaaggtacgagtattaaccatgaaagaaagaagacccattctaggagataaatttaccactagaacgagtcaaaagggaacagtcgcgtatgtcgcggatgaaacggaacttccatacgacgaaaatggtatcacaccagatgtcattattaattctacatccatcttctctagaaaaactatatctatgttgatagaggttattttaacagccgcatattctgctaagccgtacaacaataagggagaaaaccgacctgtctgttttcctagtagtaacgaaacatccatcgatacatatatgcaattcgctaaacaatgttatgagcattcaaatccgaaattgtccgatgaagaattatcggataaaatcttttgtgaaaagattctctatgatcctgaaacggataagccttatgcatccaaagtattttttggaccaatttattacttgcgtctgaggcatttaactcaggacaaggcaaccgttagatgtagaggtaaaaagacgaagctcattagacaggcgaatgagggacgaaaacgtggaggaggtatcaagttcggagaaatggagagagactgtttaatagcgcatggcgcagccaatactattacagaagttttaaaagactcagaagaggattatcaagatgtgtatgtttgtgaaaattgtggagacatagcagcacaaatcaagggtattaatacatgtcttagatgttcaaaacttaatctctctcctctcttaacaaaaattgataccacgcacgtatctaaagtatttcttactcaaatgaacgccagaggcgtaaaagtcaaattagatttcgaacgaaggcctccttcgttttataaaccattagataaagttgatctcaagccgtcttttctggtgtaatattctagtttggtagtagatacatatcaatatcatcaaattcgagatccgaattataaaatgggcgtggattgttaactatagaatcggacgtctgatattcgaaaatctgtggagttttaggttttggtggaggtgtaactgctacttgggatactgaagtctgatattcagaaagctgggggatgttctggttcgacatccaccgatggtgtcacatcactaatcggttcggtaacgtctgtggatggaggtgctacttctacagaacctgtagcctcagttgtcaacggagatacatttttaatgcgaggaaatgtataatttggtaatggtttctcatgtggatctgaagaagaggtaagatatctactagaaagataccgatcacgttctagttctcttttgtagaacttaactttttctttctcagcatctagttgatattccaacctcttcacgttactacgttcagattccaattcacgttcgcatgggttacctccgcagtttttacgagcgatttcacgttcagccttcatgcgtctctccttctctctatcgagtttatcagagcagtctttctgaaggcgatcgaactccataaatttctccaacgctttgattgtttccatagatttccgaagttcagcttttaggactgtgattctttttctttcgaattcacagctggatgtgcaaccgtttccattaccgccatctctaagtttcttttctagatcggcaacatttcatccccatgccttttacattcctcgagtctactgtcgtcgaaatatcgttccagctccttttcgacatcaataactttagcacgttgtctctcaagctctcttttgtagttatctgattccctggcacgtttaagatcttcatgcaattgagtcagctcttaacacaatctcttgcttcttcgtcatagtacttacaatcactatgggatccattgttaccacgtctacactcggcgagctcgcgtttaagagattcaatttcccgtttgtattggtccatgtttccattgctaccaccattagatttacaggctgctagttgtcgttcgagatcagaaatacgggttttcttggaattgatttcgtcgatgtacttggcatcgaaacacttattaagttctttttccaattctacgattttatttctttcgcgagtcaattccctcctgtagtaactatctgttttgtcagattcacgctctctacgtagactttcttgcaagttactaatttgttccctagcacgtccgagtttagttttatatgctgaatagagttctgattcatcctttgagcagatctctagcgatcgtttaagattcctgattctagtctttagcctatttacctcctcagaagatgttccgttaccgttgcgtttacactcgttaagctgtctatcaagatccatgattctatctctaagacgttgcatctctctttccatatcagcattgctttcattattacgtctgcagtcactcaactgtctttcaatatctgagattctatctctaagacgtcgcatctctctctgtttcggcattggtttcattattacgtctacagtcgttcaactgtctttcaagatctgatattctagattggagtctgctaatctctgtagcattttcacggcattcactcagttgtctttcaagatctgagattttagattggagtctgctaatctctgtaagatttcctcctccgctctcgatgcagtcggtcaacttattctctagttctctaatacgcgaacgcagtgcatcaacttcttgcgtgtcttcctggttgcgtgtacattcatcgagtctagattcgagatctctaacgcgtcgtcgttcttcctcaagttctctgcgtactacagaaagcgtgtccctatcttgttgatatttagcaatttctgattctagagtactgattttgcttacgtagttactaatagttgtcttggccttatcaagatcctccttgtatttgtcgcattccttgatatccctacgaagtctggacagttcccattcgacattacgacgtttatcgatttcagctcggagatcgtcatcgcgttgttttagccacatacgactgagttcaagttctcgttgacaagatccatctacttttccattcctaatagtatccagttccttttctagttctgaacgcatttctcgttccctatcaagcgattctctcaattctcggatagtcttcttatcaatttctaataaatctgaaccatcatctgtcccattttgaatatccctgtgttctttgatctcttttgtaagtcggtcgattctttcggttttataaacagaatccctttccaaagtcctaatcttactgagtttatcactaagttctgcattcaattcggtgagttttctcttggcttcttccaactctgttttaaactctccactatttccgcattcttcctcgcatttatctaaccattcaattagtttattaataactagttggtaatcagcgattcctatagccgttcttgtaattgtgggaacataattaggatcttctaatggattgtatggcttgatagcatcatctttatcattattagggggatggacaaccttaattggttggtcctcatctcctccagtagcgtgtggttcttcaataccagtgttagtaataggcttaggcaaatgcttgtcgtacgcgggcacttcctcatccatcaagtatttataatcgggttctacgtctgaatattcttttctaagagacgcgacttcgggagttagtagaagaactctgtttctgtatctatcaacgctggaatcaatactcaagttaaggatagcgaatacctcatcgtcatcatccgtatcttctgaaacaccatcatatgacatttcatgaagtctaacgtattgataaatagaatcagatttagtattaaacagatccttaacctttttagtaaacgcatatgtatattttagatctccagatttcataatatgatcacatgccttaaatgtcagtgcttccatgatataatctggaacactaatgggtgacgaaaaagatacagcaccatatgctacgttgataaataaatctgaaccactaagtagataatgattaatgttaaggaagaggaaatattcagtatatagatatgccttagcatcatatcttgtactaaacacgctaaacagtttattgatgtgatcaatttccaacagaataattagagcagcgggaataccaacaaacatattaccacatccgtattttctatgaatatcacatatcatgttaaaaaatcttgatagaagagcgaatatctcgtctgacttaatgagtcgtagttcagcagcaacataagtcataactgtaaatagaacatactttcctgtagtgttgattctagactccacatcaacaccattattaaaaatagttttatatacatctttaatctgctctccgttaatcgtcgaacgttctagtatacggaaacactttgatttcttatctgtagttaatgacttagtgatatcacgaagaatattacgaattacatttcttgtttttcttgagagacctgattcagaactcaactcatcgttccatagtttttttacctcagtggcgaaatctttggagtgcttggtacatttttcaataaggttcgtgacctccatttattataaaaaattttattcaaaacttaactacaatcgggtaattataaaatcgtagatctcccatgtggtggaatactaccatctatcgcatgtggatggacagtaggtaatggccatgggaacagtaatgtttgcatatttatctttcttgccagtattactgcatattgtcccaatgtttcgatgtgatgttctaacctatcaactgccgctgtatcacaacaatagtgtccgatgaaattaagattatgatccaatgtgtttaatatatgattatcaagtcttatacgatccgcgtcttttttgacaggatcaggttcttctacaggaagaagtttcggcctcttatgatattcatgtctgggaaacggtggtctagggtgaggctccggtatcggagtgggttttggattataatcatcatcgtctatgacatcatcttcgacttcgatatttattttgctatcttgatgatgtcctgtatcagttgcattttcagcactcgactgaatattagcgcattcattgtctattattaccatatttctaaacccaaaatgtatgtgttgaacatcagtactatcgttgatgagtcttatagcatgaattcgcttatcgttatcgggtttatcttctgtcaccttagcaattccttttttattaaactctacataatcatatccatttctattgtttgttctaatataaacgagtatagcatcattgctaaatttttcaatagtatcgaaaacagaatatcctaaaccatataatatatattcagggacactcaaactaaatgtccaggattctcctaaatacgtaaactttaatagtgcgaaatcattcaaaaatctaccacttatagatagatagtacataaatgcgtatagtagtctacctatctctttattatgaaaaccggcattacgatcatatatgtcgtgatatacctgtgatccgtttacgttaaaccataaatacatgggtgatcctataaacatgaatttatttctaattctcagagctatagttaattgaccgtgtaatatttgcttacatgcatacttgatacgctcattaataaaatttttatcattgctcgttatctcagaatcgtatatataaggagtaccatcgtgattcttaccagatattatacaaaatactatatataaaatatattgaccaacgttagtaatcatataaatgtttaacgttttaaattttgtattcaatgatccattatcatacgctagcatggtcttatgatattcattctttaaaatataatattgtgttagccattgcattggggctcctaatggagattttttattctcatccattttaggataggctttcataaagtccctaataacttcgtgaataatgtttctatgttttctactgatgcatgtatttgcttcgatttttttatcccatgtttcatctatcatagatttaaacgcagtaatgctcgcaacattaacatcttgaaccgttggtacaattccgttccataaatttataatgttcgccatttatataactcattttttgaatatacttttaattaacaaaagagttaagttactcatatgggcgccgtccagtctgaacatcaatctttttagccagagatatcatagccgctcttagagtttcagcgtgattttccaacctaaatagaacttcatcgttgcgtttacaacacttttctatttgttcaaactttgttgttacattagtaatctttttttccaaattagttagccgttgtttgagagtttcctcattgtcgtcttcatcggctttaacaattgcttcgcgtttagcctctggctttttagcagcctttgtagaaaaaaattcagttgctggaattgcaagatcgtcatctccggggaaaagagttccgtccatttaaagtacagattttagaaactgacactctgcgttatttatatttggtacaacacatggattataaatatcgatgttaataacatcagaaaatgtaaagtctatacattgttgcatcgtgttaaattttctaatggatctagtattattgggtccaacttctgcctgaaatccaaatatggaagcggatacaaaaccgtttcctggataaaccacacatctccacttttgctttacatcagaaattgtgtcgttgacatcttgaactctcctatctaatgccggtgttccacctatagattttgaatattcgaatgctgcatgagtagcattaaattccttaatattgccataattttcatatattgagtaaccctggataaaaagtaaacacaccgcagccgtcgctaccacaataaaaaaaattgatagagagttcatttataatctattagaagctgacaaaatttttttacacgcatcagacaatgctttaataaatagttcaacatctacttttgtcatatcgaaccgatggtatgattctaacctagaattacatccgaaaaagttgactatgttcatagtcattaagtcattaacaaacaacattccagactctggattataagacgatactgtttcgtcacaattacctaccttaatcatgtgattatgaatattggctattagagcaccttctaagaaatctataatatctttgaaacacgatttaaaatcaaaccacgaatatacttctacgaagaaagttagtttacccataggagaaataactataaatggagatctaaatacaaaatccggatctatgatagttttaacattattatattctctattaaatacctccacatctaaaaatgttaattttgaaactatgtcttcgtttattaccgtacctgaactaaacgctataagctctattgtttgagaactctttaaacgatattcttgaaatacatgtaacaaagtttcctttaactcggtcggtttatctaccatagttacagaatttgtatccttatctataatataataatcaaaatcgtataaagttatataattatcgcgttcagattgggatcttttcaaatagactaaaaaccccatttctctagtaagtatcttatgtatatgtttgtaaaatatcttcatggtgggaatatgctctaccgcagttagccattcctcattgacagcggtagatgtattagacaaaactattccaatgtttaacaagggccattttacgagattattaaatccttgtttgataaatgtagccaatgagggttcgagttcaacgacgattgaattctcttcccgcggatgctgcatgatgaacgacgggatgttgttcgattgatttggaattctttttcgactttttgtttatattaaatattttaaaatttatagcggatagcaattcatgtaccacggataatgtagacgcgtattgcgcatcgatatctttattattagataaatttatcaataaatgtgagaagtttgcctcgttaaggtcttccatttaaatattatataaacatttgtgtttgtatcttattcgtcttttatggaatagttttttactagtaaagctgcaattacacactttgtccgtaaaacataaatataaacaccagcttttatcaatcgttccaaaaagtcgacggcggacatttttaacatggcatctattttaaatacacttaggtttttggaaaaaacatcattttataattgtaacgattcaataactaaagaaaagattaagattaaacataagggaatgtcatttgtattttataagccaaagcattctaccgttgttaaatacttgtctggaggaggtatatatcatgatgatttggttgtattggggaaggtaacaattaataatctaaagatgatgctattttacatggatttatcatatcatggagtgacaagtagtggagcaatttacaaattgggatcgtctatcgatagactttctctaaataggactattgttacaaaagttaataattatgatgatacattttttgacgacgatgattgatcgctattgcacaattttgtttttttactttctaatatagcgtttagattctttttcatgtgcgaatattgatttactaaaatatcgatgtttaacttttgttctatgacgtccttatcagcggtatcggtacatatacgtaattcaccttcacaaaatacggagtcttcgataataatagccaatcgattattggatctagctgtctgtatcatattcaacatgtttaatatatcctttcgtttcccctttacaggcatcgatcgtagcatattttccgcgtctgagatggaaatgttaaaactacaaaaatgcgtaatgttagcccgtcctaatattggtacgtgtctataagtttggcatagtagaataatagacgtgtttaaatgccttccaaagtttaagaattctattagagtattgcattttgatagtttatcacctacatcatcaaaaataagtaaaaagtgtgctgattttttatgattttgtgcgacagcaatacatttttctatgttacttttagttcgtatcagattatattctagagattcctgactactaacgaaattaatatgatttggccaaatgtatccatcataatctgggttataaacgggtgtaaacaagaatatatgtttatattttttaactagtgtagaaaacagagatagtaaatagatagtttttccagatccagatcctcccgttaaaaccattctaaacggcatttttaataaattttctcttgaaaattgtttttcttggaaacaattcataattatatttacagttactaaattaatttgataataaatcaaaatatggaaaactaaggtcgttagtagggaggagaacaaagaaggcacatcgtgacataaataacatttattatcatgatgacaccagaaaacgacgaagagcagacatctgtgttctccgctactgtttacggagacaaaattcaaggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaactaatatttatttttgtacattaataaatgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtgacaacaaaaaatgaattaataataagtcgttaacgtacgccgccatggacgccgcgtttgttattactccaatgggtgtgttgactataacagatacattgtatgatgatctcgatatctcaatcatggactttataggaccatacattataggtaacataaaaactgtccaaatagatgtacgggatataaaatattccgacatgcaaaaatgctactttagctataagggtaaaatagttcctcaggattctaatgatttggctagattcaacatttatagcatttgtgccgcatacagatcaaaaaataccatcatcatagcatgcgactatgatatcatgttagatatagaagataaacatcagccattttatctattcccatctattgatgtttttaacgctacaatcatagaagcgtataacctgtatacagctggagattatcatctaatcatcaatccttcagataatctgaaaatgaaattgttgtttaattcttcattctgcatatcagacggcaatggatggatcataattgatgggaaatgcaatagtaattttttatcataaaagttgtaaagtaaataataaaacaataaatattgaactagtagtacgtatattgagcaatcagaaatgatgctggtacctcttatcacggtgaccgtagttgcgggaacaatattagtatgttatatattatatatttgtaggaaaaagatacgtactgtctataatgacaataaaattatcatgacaaaattaaaaaagataaagagttctaattccagcaaatctagtaaatcaactgatagcgaatcagactgggaggatcactgtagtgctatggaacaaaacaatgacgtagataatatttctaggaatgagatattggacgatgatagcttcgctggtagtttaatatgggataacgaatccaatgtcatggcgcctagcacagaacacatttacgatagtgttgctggaagcacgctgctaataaataatgatcgtaatgaacagactatttatcagaacactacagtagtaattaatgagacggagactgttgaagtacttaatgaagataccaaacagaatcctaactattcatccaatcctttcgtaaattataataaaaccagtatttgtagcaagtcaaatccgttcattacagaactcaacaataaatttagtgagaataatccgtttagacgagcacatagcgatgattatcttaataagcaagaacaagatcatgaacacgatgatatagaatcatcggtcgtatcattggtgtgattagtttcctttttataaaattgaagtaatatttagtattattgctgccgtcacgttgtacaaatggagatattccctgtattcggcatttctaaaattagcaattttattgctaataatgactgtagatattatatagatacagaacatcaaaaaattatatctgatgagatcaatagacagatggatgaaacggtacttcttaccaacatcttaagcgtagaagttgtaaatgacaatgagatgtaccatcttattccccatagactatcgactattatactctgtattagttctgtcggaggatgtgttatctctatagataatgacatcaatgacaaaaatattctaacatttcccattgatcatgctgtaatcatatccccactgagtaaatgtgtcgtagttagcaagggtcctacaaccatattggttgttaaagcggatatacccagcaaacgattggtaacatcgtttacaaacgacatactgtatgtaaacaatctgtcactgattaattatttgccgttgtctgtattcattattagacgagtcaccgactatttggatagacacatatgcgatcagatatttgcgaataataagtggtattccattataaccatcgacgataagcaatatcctattccatcaaactgtataggtatgtcctctgccaagtacataaattctagcatcgagcaagatactttaatacatgtttgtaacctcgagcatccattcgacttagtatacaaaaaaatgcagtcgtacaattctgtacctatcaaggaacaaatattgtacggtagaattgataatataaatatgagcattagtatttctgtggattaatagatttctagtatggggatcattaatcatctctaatctctaaatacctcataaaacgaaaaaaaagctattatcaaatactgtacggaatggattcattctcttctctttttatgaaactctgttgtatatctactgataaaactggaagcaaaaaatctgataaaaagaataagaataagatcaaggattatatggaacacgattattataaaataacaatagttcctggttcctcttccacgtctactagctcgtggtattatacacatgcctagtaatagtctctttgcgttgacggaaagcagactagaaataacaggctaaaatgttcagacaccataatagttcccaacccagataataacagagtaccatcaacacattcctttaaactcaatcccaaacccaaaaccgttaaaatgtatccggccaattgatagtagataatgaggtgtacagcgcatgataatttacacagtaaccaaaatgaaaatactttagtaattataagaaatatagatggtaacgtcatcatcaacaatccaataatatgccggagagtaaacattgacggataaaacaaaaatgctccgcataactctatcatggcaataacacaaccaaatacttgtaagattcctaaattagtagaaaatacaacggatatcgatgtataagtgatctcgagaaataataagaataaagtaatgcccgtaaagataaacatcaacattgtttggtaatcattaaaccaattagtatgaagttgaactaatttcacagtagattttattccagtgttatcctcgcatgtatacgtacctggtaagatatctttatattctataatcaatgagacatcactatccgataacgaatgaagtctagcactagtatgccatttacttaatatggtcgtcttggaagttttattataagttaaaatatcatggttgtccaatttccatctaatatactttgtcggattatctatagtacacggaataatgatggtattattacatgctgtatactctatagtctttgtagatgttataatcataaaagtacagaggtatatcaacgatattctaactcttgacattttttatttatttaaaatgatacctttgttatttattttattctattttgctaacggtatcgaatggcataagtttgaaacgagtgaagaaataatttctacttacttattagacgacgtattatacacgggtgttaatggggcggtatacacattttcaaataataaactaaacaaaactggtttagctaatactaattatatcacaacatctataaaagtagaggatgcggataaggatacattagtatgcggaaccaataacggaaatcccaaatgttggaaaatagacggttcagacgacccaaaacatagaggtagaggatacgctccttatcaaaatagcaaagtaacgataatcagtcacaacggatgtgtactatctgacataaacatatcaaaagaaggaattaaacgatggagaagatttgacggaccatgtggttatgatttatacacggcggataacgtaattccaaaagatggtttacgaggagcattcgtcgataaagatggtacttatgacaaagtttacattcttttcactgatactatcggctcaaagagaattgtcaaaattccgtatatagcacaaatgtgcctaaacgacgaaggtggtccatcatcattgtctagtcatagatggtcgacgtttctcaaagtcgaattagaatgtgatatcgacggaagaagttatagacaaattattcattctagaactataaaaacagataatgatacgatactatatgtattcttcgatagtccgcattatgtacctattctatgaataccattaaacaatctttttctacgtcaaaattggaaggatatacaaagcaattgccgtctccagctcctggtatatgtctaccagctggaaaagttgttccacataccacgtttgaagtcatagaaaaatataatgtactagatgatattataaagcctttatctaaccaacctatcttcgaaggaccgtctggtgttaaatggttcgatataaaggagaaggaaaatgaacatcgggaatatagaatatacttcataaaagaaaattctatatattcgttcgatacaaaatctaaacaaactcgtagctcgcaagtcgatgcgcgactattttcagtaatggtaacttcgaaaccgttatttatagcagatatagggataggagtaggaatgccacaaatgaaaaaaatacttaaaatgtaatcttaatcgagtacaccacacgacaatgaacaaacataagacagattatgctggttatgcttgctgcgtaatatgcggtctaattgttggaattatttttacagcgacactattaaaagttgtagaacgtaaattagttcatacaccatcaatagataaaacgataaaagatgcatatattagagaagattgtcctactgactggataagctataataataaatgtatccatttatctactgatcgaaaaacctgggaggaaggacgtaatgcatgcaaagctctaaatccaaattcggatctaattaagatagagactccaaacgagttaagttttttaagaagcattagacgcggatattgggtaggagaatccgaaatattaaaccagacaaccccatataattttatagctaagaatgccacgaagaatggaactaaaaaacggaaatatatttgtagcacaacgaatactcccaaactgcattcgtgttacactatataacaattacactacatttttatcataccactacttcggttagatgttttagaaaaaaataaatatcgccgtaccgttcttgtttttataaaaataacaattaacaattatcaaattttttctttaatattttacgtggttgaccattcttggtggtaaaataatctcttagtgttggaatggaatgctgtttaatgtttccacactcatcgtatattttgacgtatgtagtcacatcgtttacgcaatagtcagactgtagttctatcatgcttcctacattagaaggaggaacagttttaaagtctcttggttttaatctattaccgttagttttcatgaaatcctttgttttatccacttcacattttaaataaatgtccactatacattcttctgttaattttactagatcgtcatgggtcatagaatttataggttccgtagtccatggatccaaactagcaaacttcgcgtatacggtatcgcgattagtgtatacaccaactgtatgaaaattaagaaaacagtttaatagatcaacagaaatatttaatcctccgtttgatacagatgcaccatatttatggattttggattcacacgttgtttgtctgaggggttcgtctagcgttgcttctacataaacttctattcccatatattctttattgtcagaatcgcataccgatttatcatcatacactgtttgaaaactaaatggtatacacatcaaaataacaaatactaacgagtacattctgcaatattgttatcgtaattggaaaaatagtgttcgagtgagttggattatgtgagtattggattgtatattttattttatattttgtaataagaataaaatgctaatgtcaagtttattccaatagatgtcttattaaaaacatatataataaataacaatggctgaatggcataaaattatcgaggatatctcaaaaaataataagttcgaggatgccgccatcgttgattacaagactacaaagaatgttctagctgctattcctaacagaacatttgccaagattaatccgggtgaaattattcctctcatcactaatcgtaatattctaaaacctcttattggtcagaaatattgtattgtatatactaactctctaatggatgagaacacgtatgctatggagttgcttactgggtacgcccctgtatctccgatcgttatagcgagaactcataccgcacttatatttttgatgggtaagccaacaacatccagacgtgacgtgtatagaacgtgtagagatcacgctacccgtgtacgcgcaactggtaattaaaataaaaagtaatattcatatgtagtgtcaattttaaatgatgatgatgaaatggataatatccatattgacgatgtcaataatgccggtattggcatacagctcatcgatttttagatttcattcagaggatgtggaattatgttatgggcatttgtattttgataggatctataatgtagtaaatataaaatataatccgcatattccatatagatataattttattaatcgcacgttaaccgtagatgaactagacgataatgtcttttttacacatggttattttttaaaacacaaatatggttcacttaatcctagtttgattgtctcattatcaggaaacttaaaatataatgatatacaatgctcagtaaatgtatcgtgtctcattaaaaatttggcaacgagtacatctactatattaacatctaaacataagacttattctctacatcggtccacgtgtattactataataggatacgattctattatatggtataaagatataaatgacaagtataatggcatctatgattttactgcaatatgtatgctaatagcgtctacattgatagtgaccatatacgtgtttaaaaaaataaaaatgaactcttaattatgctatgctattagaaatggataaaatcaaaattacggttgattcaaaaattggtaatgttgttaccatatcgtataacttggaaaagataactattgatgtcacacctaaaaagaaaaaagaaaaggatgtattattagcgcaatcagttgctgtcgaagaggcaaaagatgtcaaggtagaagaaaaaaatattatcgatattgaagatgacgatgatatggatgtagaaagcgcataatacgatctataaaaataagtatataaatactttttatttactgtactcttactgtgtagtggtgataccctactcgattatttttttaaaaaaatacttattctgattcttctagccatttccgtgttcgttcgaatgccacatcgacgttaaagataggggagtagttgaaatctagttctgcattgttggtacgcacctcaaatgtagtgttggatatcttcaacgtatagttgttgagtagtgatggttttctaaatagaattctcttcatatcattcttgcacgcgtacatttttagcatccatcttggaattctagatccttgttctattcccaatggtttcatcaatagaagattaaacatatcgtacgaacacgatggagagtaatcgtagcaaaagtaagcatttcctttaatctcagatcccggatactggatatattttgcagccaacacgtgcatccatgcaacatttcctacatatacccggctatgcaccgcgtcatcatcgactgtacgatacataatgttaccgtgttgcttacattgctcgtaaaagactttcgtcaatttgtctccttctccgtaaattccagtgggtcttaggcaacaagtatacaattttgctccattcatgattacggaattattggctttcataaccagttgctcggccatacgtttactttttgcgtatacatgtcctggtgatatatcataaagggtatgctcatggccgatgaatggatcaccgtgtttatttggtcctattgcttccatgctactagtatagatcaaatacttgattcctaggtccacacaagctgccaatatagtctgtgttccataatagtttactttcatgatttcattatcggtgtattttccaaatacatccactagagcagccgtatgaataatcagatttaccccatctagcgcttctctcaccttatcaaagtcgtttatatcacattgtatatagtttataaccttaactttcgaggttattggttgtggatcttctacaatatctatgactctgatttcttgaacatcatctgcactaattaacagttttactatatacctgcctagaaatccggcaccaccagtaaccgcgtacacggccattgctgccactcataatatcagactacttattctattttactaaataatggctgtttgtataatagaccacgataatatcagaggagttatttactttgaaccagtccatggaaaagataaagttttaggatcagttattggattaaaatccggaacgtatagtttgataattcatcgttacggagatattagtcaaggatgtgattccataggcagtccagaaatatttatcggtaacatctttgtaaacagatatggtgtagcatatgtttatttagatacagatgtaaatatatctacaattattggaaaggcgttatctatttcaaaaaatgatcagagattagcgtgtggagttattggtatttcttacataaatgaaaagataatacattttcttacaattaacgagaatggcgtttgatatatcagttaatgcgtctaaaacaataaatgcattagtttacttttctactcagcaaaataaattagtcatacgtaatgaagttaatgatacacactacactgtcgaatttgatagggacaaagtagttgacacgtttatttcatataatagacataatgacaccatagagataagaggggtgcttccagaggaaactaatattggttgcgcggttaatacgccggttagtatgacttacttgtataataagtatagttttaaactgattttagcagaatatataagacacagaaatactatatccggcaatatttattcggcattgatgacactagatgatttggctattaaacagtatggagacattgatctattatttaatgagaaacttaaagtagactccgattcgggactatttgactttgtcaactttgtaaaggatatgatatgttgtgattctagaatagtagtagctctatctagtctagtatctaaacattgggaattgacaaataaaaagtataggtgtatggcattagccgaacatatatctgatagtattccaatatctgagctatctagactacgatacaatctatgtaagtatctacgcggacacactgagagcatagaggataaatttgattattttgaagacgatgattcgtctacatgttctgccgtaaccgacagggaaacggatgtataattttttttatagcgtgaaggatatgataaaaaatataattgttgtatttatcccattccaatcaccttatatgattctgtaacacaatgaaggagtctcatagatgtatagaggtcagatactggtttgataaactgtttattccacatgagtatgtttgactttatggttagacccgcatactttaacaaatcactgaaaattggagttaggtattgacctctcagaatcagttgccgttctggaacattaaatgtattttttatgatatactccaacgcatttatgtgggcatacaacaagtcattactaatggagtattccaagagttttagttgtctagtatttaacaagagaagagatttcaacagactgtttatgaactcgaatgccgcctcattgtcgcttatattgatgatgtcgaattctcccaatatcatcaccgatgagtagctcatcttgttatcgggatccaagttttctaaagatgtcattaaaccctcgatcatgaatggatttatcatcatcgtttttatgttggacatgagcttagtccgtttgtccacatctatagacgacgatttctgaattattttatatatccctctctttaactccaggaacttgtcaggatggtctactttaatatgttctcgtctaagagatgaaaatctttggatggttgcacgcgacttttctctaaaggatgacgttgcccaagatcctctcttaaatgaatccatcttatccttggacaagatggacagtctattttccttagatggtttaatatttttgttacccatgatctataaaggtagacctaatcgtctcggatgacctatatatttattttcagttttattatacgcataaattgtaaaaaatatgttaggtttacaaaaatgtctcgtggggcattaatcgtttttgaaggattggacaaatctggaaaaacaacacaatgtatgaacatcatggaatctataccggcaaacacgataaaatatcttaactttcctcagagatccactgtcactggaaagatgatagatgactatctaactcgtaaaaaaacctataatgatcatatagttaatctattattttgtgcaaatagatgggagtttgcatcttttatacaagaacaactagaacagggaattactttaatagttgatagatacgcgttctctggagtagcgtatgccgccgctaaaggcgcgtcaatgactctcagtaagagttatgaatctggattgcctaaacccgacttagttatattcttggaatctggtagcaaagaaattaatagaaacgtcggcgaggaaatttatgaagatgttacattccaacaaaaggtattacaagaatataaaaaaatgattgaagaaggagatattcattggcaaattatttcttctgaattcgaggaagatgtaaagaaggagttgattaagaatatagttatagaggctatacacacggttactggaccagtggggcaactgtggatgtaatagtgaaattacattttttataaatagatgttagtacagtgttataaatggatgaagcatattactctggcaacttggaatcagtactcggatacgtgtccgatatgcataccgaactcgcatcaatatctcaattagttattgccaagatagaaactatagataatgatatattaaacaaggacattgtaaattttatcatgtgtagatcaaacttggataatccatttatctctttcctagatactgtatatactattatagatcaagagaactatcagaccgaattgattaattcattagacgacaatgaaattatcgattgtatagttaataagtttatgagcttttataaggataacctagaaaatatagtagatgctatcattactctaaaatatataatgaataatccagattttaaaactacgtatgccgaagtactcggttccagaatagccgatatagatattaaacaagtgatacgtgagaatatactacaattgtctaatgatatccgcgaacgatatttgtgaaaaatattaaaaaaaaatactttttttattaaatgacgtcgcttcgcgaatttagaaaattatgctgtgatatatatcacgcatcaggatataaagaaaaatctaaattaattagagactttataacagatagggatgataaatatttgatcattaagctattgcttcccggattagacgatagaatttataacatgaacgataaacaaattataaaattatatagtataatatttaaacaatctcaggaagatatgctacaagatttaggatacggatatataggagacactattaggactttcttcaaagagaacacagaaatccgtccacgagataaaagcattttaactttagaagacgtggatagtttcttaactacgttatcatccgtaactaaagaatcgcatcaaataaaattattgactgatatcgcatccgtttgtacatgtaatgatttaaaatgtgtagtcatgcttattgataaagatctaaaaattaaagcgggtcctcggtacgtacttaacgctattagtcctaatgcctatgatgtgtttagaaaatctaataacttgaaagagataatagaaaatgcatctaaacaaaatctagactctatatctatttctgttatgactccaattaatcccatgttagcggaatcgtgtgattctgtcaataaggcgtttaaaaaatttccatcaggaatgtttgcggaagtcaaatacgatggtgaaagagtacaagttcataaaaataataacgagtttgccttctttagtagaaacatgaaaccagtactctctcataaagtggattatctcaaagaatacataccgaaagcatttaaaaaagctacgtctatcgtattggattctgaaattgttcttgtagacgaacataatgtaccgctcccgtttggaagtttaggaatacacaaaaagaaagaatataaaaactctaacatgtgtttgttcgtgtttgactgtttgtactttgatggattcgatatgacggacattccattgtacgaacgaagatcttttctcaaagatgttatggttgaaatacccaatagaatagtattctcagagttgacgaatattagtaacgagtctcagttaactgacgtattggatgatgcactaacgagaaaattagaaggattggtcttaaaagatattaatggagtatacgaaccgggaaagagaagatggttaaaaataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggttttgcaagaccaattaacgatggttaaaattaacaaggatcccaaaaaaattccagagtggttggtagttaataaaatctatattcccgattttgtagtagaggatccgaaacaatctcagatatgggaaatttcaggagcagagtttacatcttccaagtcccataccgcaaatggaatatccattagatttcctagatttactaggattagagaagataaaacgtggaaagaatctactcatctaaacgatttagtaaacttgactaaatcttaatagttacatacaaactgaaaattaaaataacactatttagttggtggtcgccatggatggtgttattgtatactgtctaaacgcgttagtaaaacatggcgaggaaataaatcatataaaaaatgatttcatgattaaaccatgttgtgaaagagtttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgttattcctgttatcatatctataggaaaggataaagatgtctgtgaactattaatctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcaccatggacataaagatagatattagtatttctggtgataaatttacggtgactactaggagggaaaatgaagaaagaaaaaaatatctacctctccaaaaagaaaaaactactgatgttatcaaacctgattatcttgagtacgatgacttgttagatagagatgagatgtttactattctagaggaatattttatgtacagaggtctattaggcctcagaataaaatatggacgactctttaacgaaattaaaaaattcgacaatgatgcggaagaacaattcggtactatagaagaactcaagcagaaacttagattaaattctgaagagggagcagataactttatagattatataaaggtacaaaaacaggatatcgtcaaacttactgtatacgattgcatatctatgataggattgtgtgcatgcgtggtagatgtttggagaaatgagaaactgttttctagatggaaatattgtttacgagctattaaactgtttattaatgatcacatgcttgataagataaaatctatactgcagaatagactagtatatgtggaaatgtcatagaaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaatggtaacgataacacaaaatgtgaacgctgcccacctcatacatataccacaatcccaattattctaatggatgtcatcaatgtagaaaatgcccaaccggatcatttgataaggtaaagtgtaccggaacacagaacagtaaatgttcgtgtcttcctggttggtattgtgctactgattcttcacagactgaagattgttgaaattgtgtaccaaaaaggagatgtccatgcggatactttggtggaatagatgaacaaggaaatcctatttgtaaatcgtgttgtgttggtgaatattgcgactacctacgtaattatagacttgatccatttcctccatgcaaactatctaaatgtaattaattatgattttgatgataatgttaccatacattatatcgctacttggttagtgtattattcagtatgaagacctattaataattacttatcttttgacgatcttgttataattataatataaaaatacttatggcatagtaactcataattgctgacgcgataaattcgtaataatctgttttgttcaaatttttataaggaatctacaggcataaaaataaaaatataatttataatatactcttacagcgcgccatcatgaataacagcagtgaattgattgctgttattaatggatttagaaatagtggacgattttgtgatattagtatagttattaatgatgaaaggataaacgctcataaactcatcctatctggagcctccgaatatttttccattctgttttccaataattttatcgattctaatgaatacgaagttaatctaagtcatttagattatcaaagcgttaacgatttgatcgattacatttatgggatacctttgagcctaactaacgataacgtgaaatatattctttcaaccgctgattttttacaaattggatctgctattacggagtgtgaaaattacatacttaaaaatctttgttctaaaaactgtatcgatttctacatatacgctgataaatataataacaagaaaatagaatcagcgtcgtttaacacaatattacaaaatattttgagactcatcaacgatgaaaactttaaatacttaacagaggaatcaatgataaaaattttaagcgatgatatgttaaatataaaaaatgaggattttgcaccactaattctcattaaatggttagagagtactcaacaatcatgcaccgtcgagttacttagatgcctcagaatatcattgctttccccacaagttataaaatcactttatagtcatcaactggttagttcaatctacgaatgtataacattcttaaacaatatagcattcttggatgaatcatttcctagataccatagcatcgagttgatatctatcggtataagtaattcgcatgataagatttccataaactgctacaatcataaaaaaaatacatgggaaatgatatcttcacgtagatataggtgtagtttcgcagtggccgtcctggataatattatctatatgatgggtggatatgatcagtccccgtatagaagttcaaaggttatagcgtacaatacatgtacaaattcttggatatatgatataccagagctaaaatatcctcgttctaattgtgggggactggctgatgacgaatacatttattgtataggcggcatacgcgatcaggattcatcgttgacatctagtattgataaatggaagccatcaaaaccatattggcagaagtatgctaaaatgcgcgaaccaaaatgtgatatgggggttgcgatgttaaacggattaatatatgtcatgggtggaatcgttaaaggtgacacgtgtaccgacgcactagagagtttatcagaagatggatggatgaagcatcaacgtcttccaataaaaatgtccaatatgtcgacgattgttcatgatggcaagatttatatatctggaggttacaacaatagtagtgtagttaatgtaatatcgaatctagtccttagctataatccgatatatgatgaatggaccaaattatcatcattaaacattcctagaattaatcccgctctatggtcagcgcataataaattatatgtaggaggaggaatatctgatgatgttcgaactaatacatctgaaacatacgataaagaaaaagattgttggacattggataatggtcacgtgttaccacgcaattatataatgtataaatgcgaaccgattaaacataaatatccattggaaaaaacacagtacacgaatgattttctaaagtatttggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcaactacaaatgacactgataaagtagattatgaagaatactccacagagttgattgtaaatacagatagtgaatcgactatagacataatactatctggatctacacattcaccggaaactagttctaagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagatcatacagttacagacactgtctcatacactacagtaagtacatcatctggaattgtcactactaaatcaaccaccgatgatgcggatctttatgatacgtacaatgataatgatacagtaccaccaactactgtaggcggtagtacaacctctattagcaattataaaaccaaggactttgtagaaatatttggtattaccgcattaattatattgtcggccgtggcaattttctgtattacatattatatatataataaacgttcacgtaaatacaaaacagagaacaaagtctagatttttgacttacataaatgtctgggatagtaaaatctatcatattgagcggaccatctggtttaggaaagacagccatagccaaaagactatgggaatatatttggatttgtggtgtcccataccactagatttcctcgtcctatggaacgagaaggtgttgattaccattacgttaacagagaggccatctggaagggaatagccgccggaaactttctagaacatactgagtttttaggaaatatttacggaacttctaaaacagctgtgaatacagcggctattaataatcgtatttgtgtgatggatctaaacatcgacggtgttagaagtcttaaaaatacgtacctaatgccttactcggtgtatataagacctacctctcttaaaatggttgagaccaagcttcgttgtagaaacactgaagctaacgatgagattcatcgtcgcgtgatattggcaaaaacggatatggatgaggccaacgaagcaggtctattcgacactattatcattgaagatgatgtgaatttagcatatagtaagttaattcagatactacaggaccgtattagaatgtattttaacactaattagagacttaagacttaaaacttgataattaataatataactcgtttttatatgtggctatttcaacgtctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggattcggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtctaatggagaacatgttccatttataagaaatccaaataaaatggataacggtactctagaatttacacctatagattcgcataaaggatacgttgtatctagacgtggagatctagaaacacttggatattgtatgattagatggttgggaggtatcttgccatggactaagatatctgaaacaaagaattgtgcattagtaagtgccacaaaacagaaatatgttaacaatactgcgactttgttaatgaccagtttgcaatatgcacctagagaattgctgcaatatattaccatggtaaactctttgacatattttgaggaacccaattatgacgagtttcggcacatattaatgcagggtgtatattattaagtgtggtgtttggtcgatgtaaaatttttgtcgataaaaattaaaaaataacttaatttattattgatctcgtgtgtacaaccgaaatcatggcgatgttttacgcacacgctctcggtgggtacgacgagaatcttcatgcctttcctggaatatcatcgactgttgccaatgatgtcaggaaatattctgttgtgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcaaatctctatcatcacatatcgccacaaaaactactatgctctaagcggaatcgggtacgagagtctagacttgtgtttggaaggagtagggattcatcatcacgtacttgaaacaggaaacgctgtatatggaaaagttcaacatgattattctactatcaaagagaaggccaaagaaatgaatgcacttagtccaggacctatcattgattaccacgtctggataggagattgtatctgtcaagttactgctgtggacgtacatggaaaggaaattatgagaatgagattcaaaaagggtgcggtgcttccgatcccaaatctggtaaaagttaaacttggggagaatgatacagaaaatctttcttctactatatcggcggcaccatcgaggtaaccacctctctggaagacagcgtgaataatgtactcatgaaacgtttggaaactatacgccatatgtggtctgtcgtatatgatcattttgatattgtgaatggtaaagaatgctgttatgtgcatacgcatttgtctaatcaaaatcttataccgagtactgtaaaaacaaatttgtacatgaagactatgggatcatgcattcaaatggattccatggaagctctagagtatcttagcgaactgaaggaatcaggtggatggagtcccagaccagaaatgcaggaatttgaatatccagatggagtggaagacactgaatcaattgagagattggtagaggagttcttcaatagatcagaacttcaggctggtgaatcagtcaaatttggtaattctattaatgttaaacatacatctgtttcagctaagcaactaagaacacgtatacggcagcagcttcctttatactctcatcttttaccaacacaaagggtggatatttgttcattggagttgataataatacacacaaagtaattggattcacggtgggtcatgactacctcagactggtagagaatgatatagaaaagcatatcaaaagacttcgtgttgtgcatttctgtgagaagaaagaggacatcaagtacacgtgtcgattcatcaaggtatataaacctggggatgaggctacctcgacatacgtgtgcgctatcaaagtggaaagatgctgttgtgctgtgtttgcagattggccagaatcatggtatatggatactaatggtatcaagaagtattctccagatgaatgggtgtcacatataaaattttaattaatgtaatagagaacaaataataaggttgtaatatcatatagacaataactaacaattaattagtaactgttatctcttttttaactaaccaactaactatatacctattaatacatcgtaattatagttcttaacatctattaatcattaattcgcttctttaattttttataaactaacattgttaattgaaaagggataacatgttacagaatataaattatatatggatttttttaaaaaggaaatacttgactggagtatatatttatctcttcattatatagcacgcgtgttttccaatttttccacatcccatataatacaggattataatctcgttcgaacatacgagaaagtggataaaacaatagttgattttttatctaggttgccaaatttattccatattttagaatatggggaaaatattctacatatttattctatggatgatgctaatacgaatattataattttttttctagatagagtattaaatattaataagaacgggtcatttatacacaatctcaggttatcatcatccattaatataaaagaatatgtatatcaattagttaataatgatcatccagataataggataagactaatgcttgaaaatggacgtagaacaagacattttttgtcctatatatcagatacagttaatatctatatatgtattttaataaatcatggattttatatagatgccgaagacagttacggttgtacattattacatagatgtatatatcactataagaaatcagaatcagaatcatacaatgaattaattaagatattgttaaataatggatcagatgtagataaaaaagatacgtacggaaacacaccttttatcctattatgtaaacacgatatcaacaacgtggaattgtttgagatatgtttagagaatgctaatatagactctgtagactttaatagatatacacctcttcattatgtctcatgtcgtaataaatatgattttgtaaagttattaatttctaaaggagcaaatgttaatgcgcgtaatagattcggaactactccattttattgtggaattatacacggtatctcgcttataaaactatatttggaatcagacacagagttagaaatagataatgaacatatagttcgtcatttaataatttttgatgctgttgaatctttagattatctattatccagaggagttattgatattaactatcgtactatatacaacgaaacatctatttacgacgctgtcagttataatgcgtataatacgttggtctatctattaaacagaaatggtgattttgagacgattactactagtggatgtacatgtatttcggaagcagtcgcaaacaacaacaaaataataatggaagtactattgtctaaacgaccatctttgaaaattatgatacagtctatgatagcaattactaaacataaacagcataatgcagatttattgaaaatgtgtataaaatatactgcgtgtatgaccgattatgatactcttatagatgtacagtcgctacagcaatataaatggtatattttaaaatgtttcgatgaaatagatatcatgaagagatgttatataaaaaataaaactgtattccaattagttttttgtatcaaagacattaatactttaatgagatacggtaaacatccttctttcgtgaaatgcactagtctcgacgtatacggaagtcgtgtacgtaatatcatagcatctattagatatcgtcagagattaattagtctattatccaagaagctggatcctggagataaatggtcgtgttttcctaacgaaataaaatataaaatattggaaaactttaacgataacgaactatccacatatctaaaaatcttataaacactattaaaatataaaatctaagtaggataaaatcacactacatcattgtttccttttagtgctcgacagtgtatactatttttaacactcataaataaaaatgaaaacgatttccgttgttacgttgttatgcgtactacctgctgttgtttattcaacatgtactgtacccactatgaataacgctaaattaacgtctaccgaaacatcgtttaatgataaacagaaagttacgtttacatgtgatcagggatatcattcttcggatccaaatgctgtctgcgaaacagataaatggaaatacgaaaatccatgcaaaaaaatgtgcacagtttctgattacatctctgaattatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatatgccgtctctatctaatggattaatttccggatctacattttctatcggtggcgttatacatcttagttgtaaaagtggttttacactaacggggtctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaaatataaatccgttaaaataatgaataattaataacgaacaagtatcaaaagattaaagaattatagctagaatcaattgagatgtcttcttcagtggatgttgatatctacgatgccgttagagcatttttactcaggcactattataacaagagatttattgtgtatggaagaagtaacgccatattacataatatatacaggctatttacaagatgcgccgttataccgttcgatgatatagtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtataccattgactcgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacaccatgagatatattataattctcgcagttttgttcattaatagtatacacgctaaaataactagttataagtttgaatccgtcaattttgattccaaaattgaatggactggggatggtctatacaatatatcccttaaaaattatggcatcaagacgtggcaaacaatgtatacaaatgtaccagaaggaacatacgacatatccgcatttccaaagaatgatttcgtatctttctgggttaaatttgaacaaggcgattataaagtggaagagtattgtacgggactatgcgtcgaagtaaaaattggaccaccgactgtaacattgactgaatacgacgaccatatcaatttgtacatcgagcatccgtatgctactagaggtagcaaaaagattcctatttacaaacgcggtgacatgtgtgatatctacttgttgtatacggctaacttcacattcggagattctaaagaaccagtaccatatgatatcgatgactacgattgcacgtctacaggttgcagcatagactttgtcacaacagaaaaagtgtgcgtgacagcacagggagccacagaagggtttctcgaaaaaattactccatggagttcgaaagtatgtctgacacctaaaaagagtgtatatacatgcgcaattagatccaaagaagatgttcccaatttcaaggacaaaatggccagagttatcaagagaaaatttaataaacagtctcaatcttatttaactaaatttctcggtagcacatcaaatgatgttaccacttttcttagcatgcttaacttgactaaatattcataactaatttttattaatgatacaaaaacgaaataaaactgcatattatacactggttaacgcccttataggctctaaccattttcaagatgaggtccctgattatagtccttctgttcccctctatcatctactccatgtctattagacaatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtgcatggaagttagtacatgatcgtctccccgctataaaggcattatcaacttctccttattgattgaaaatgaaaatataaatagtttttatgtatagcagtattaccctatagttttattgcttactactaacatggatacagatgttacaaatgtagaagatatcataaatgaaatagatagagagaaagaagaaatactaaaaaatgtagaaattgaaaataataaaaacattaacaagaatcatccaagtggatatattagagaagcactcgttattaataccagtagtaatagtgattccattgataaagaagttatagaatgtatctgtcacgatgtaggaatatagatcatatctactaatttttataatcgatacaaaacataaaaaacaactcgttattacatagcaggcatggaatccttcaagtattgttttgataacgatggcaagaaatggattatcggaaatactttatattctggtaattcaatactatataaggtcagaaaaaatttcactagttcgttctacaattacgtaatgaagatagatcacaaatcacacaagccattgttgtctgaaatacgattctatatatctgtattggatcctttgactatcgacaactggacacgggaacgtggtataaagtatttggctattccagatctgtatggaattggagaaaccgatgattatatgttcttcgttataaagaatttgggaagagtattcgccccaaaggatactgaatcagtcttcgaagcatgcgtcactatgataaacacgttagagtttatacactctcaaggatttacccatggaaaaatagaaccgaggaatatactgattagaaataaacgtctttcactaattgactattctagaactaacaaactatacaagagtggaaactcacatatagattacaacgaggacatgataacttcaggaaatatcaattatatgtgtgtagacaatcatcttggagcaacagtttcaagacgaggagatttagaaatgttgggatattgcatgatagaatggttcggtggcaaacttccatggaaaaacgaaagtagtataaaagtaataaaacaaaaaaaagaatataaaaaatttatagctactttctttgaggactgttttcctgaaggaaatgaacctctggaattagttagatatatagaattagtatacacgttagattattctcaaactcctaattatgacagactacgtaaactgtttatacaagattgaaattatattcttttttttatagagtgtggtagtgttacggatatttaatattagactatctctatcgcgctacacgaccaatatcgattactatggatatcttcagggaaatcgcatcttctatgaaaggagagaatgtattcatttctccagcgtcaatctcgtcagtattgacaatactgtattatggagctaatggatccactgctgaacagctatcaaaatatgtagaaaaggaggagaacatggataaggttagcgctcaaaatatctcattcaaatccataaataaagtatatgggcgatattctgccgtgtttaaagattcctttttgagaaaaattggcgataagtttcaaactgttgacttcactgattgtcgcactatagatgcaatcaacaagtgtgtagatatctttactgaggggaaaatcaatccactattggatgaaccattgtctcctgatacctgtctcctagcaattagtgccgtatactttaaagcaaaatggttgacgccattcgaaaaggaatttaccagtgattatcccttttacgtatctccgacggaaatggtagatgtaagtatgatgtctatgtacggcaaggcatttaatcacgcatctgtaaaggaatcattcggcaacttttcaatcatagaactgccatatgttggagatactagtatgatggtcattcttccagacaagattgatggattagaatccatagaacaaaatctaacagatacaaattttaagaaatggtgtaactctctggaagctacgtttatcgatgttcacattcccaagtttaaggtaacaggctcgtataatctggtggatactctagtaaagtcaggactgacagaggtgttcggttcaactggagattatagcaatatgtgtaattcagatgtgagtgtcgacgctatgatccacaaaacgtatatagatgtcaatgaagagtatacagaagcagctgcagcaacttgtgcactggtgtcagactgtgcatcaacaattacaaatgagttctgtgtagatcatccgttcatctatgtgattaggcatgttgatggaaaaattcttttcgttggtagatattgctctccgacaactaattgttaaccattttttttaaaaaatagaaaaaacatgtggtattagtgcaggtcgttattcttccaattgcaattggtaagatgacggccaactttagtacccacgtcttttcaccacagcactgtggatgtgacagactgaccagtattgatgacgtcagacaatgtttgactgaatatatttattggtcgtcctatgcataccgcaacaggcaatgcgctggacaattgtattccacactcctctcttttagagatgatgcggaattagtgttcatcgacattcgcgagctggtaaaaaatatgccgtgggatgatgtcaaagattgtgcagaaatcatccgttgttatataccggatgagcaaaaaaccatcagagagatttcggccatcatcggactttgtgcatatgctgctacttactggggaggtgaagaccatcccactagtaacagtctgaacgcattgtttgtgatgcttgagatgctcaattacgtggattataacatcatattccggcgtatgaattgatgagttgtacatcttgacattttcttctttcttctcttctccctttcccagaaacaaactttttttacccactataaaataaaatgagtatactacctgttatatttctttctatatttttttattcttcattcgttcagacttttaacgcgcctgaatgtatcgacaaagggcaatattttgcatcattcatggagttagaaaacgagccagtaatcttaccatgtcctcaaataaatacgctatcatccggatataatatattagatattttatgggaaaaacgaggagcggataatgatagaattataccgatagataatggtagcaatatgctaattctgaacccgacacaatcagactctggtatttatatatgcattaccacgaacgaaacctactgtgacatgatgtcgttaaatttgacaatcgtgtctgtctcagaatcaaatatagatcttatctcgtatccacaaatagtaaatgagagatctactggcgaaatggtatgtcccaatattaatgcatttattgctagtaacgtaaacgcagatattatatggagcgggcatcgacgccttagaaataagagacttaaacaacggacacctggaattattaccatagaagatgttagaaaaaatgatgctggttattatacatgtgttttagaatatatatacggtggcaaaacatataacgtaaccagaattgtaaaattagaggtacgggataaaataataccttctactatgcaattaccagatggcattgtaacttcaataggtagtaatttgactattgcatgcagagtatcgttgagacctcccacaacggatgcagacgtcttttggataagtaatggtatgtattacgaagaagatgatggggacggaaacggtagaataagtgtagcaaataaaatctatatgaccgataagagacgtgttattacatcccggttaaacattaatcctgtcaaggaagaagatgctacaacgtttacgtgtatggcgtttactattcctagcatcagtaaaacagttactgttagtataacgtgaatgtatgttgttacatttccatgtcaattgagtttataagaatttttatacattatcttccaacaagcaattgacgaacgtattgctatgattaactcccacgatactatgcatattattaatcattaacttgcagactatacctagagctattttgacatactcgtgttcttgtgtaattgcagtatctatattattaaagtacgtaaatctagctatagttttattatttaattttagataatataccgtctccttatttttaaaaattgccacatcctttattaaatcatgaatgggaatttctatgtcatagttaatatattgtgaacaacaagagcagatatctataggaaagggtggaatgcgatacattgatctatgtagttttaaaacacacgcaaactttgaagaatttatataaatcattccatcgatacatccttctatgttgagatgtatatatccaggaattcgtttattaatatcgggaaatgtataaactaaaacattgcccggaagcggagcttctaccggagttatatcagtttttaacttacaaaatgtaaccaatacctttgcatgacttgttttgttcggcaacgttagtttaaacttgacgaatggattaattacaatagcatgatccgcgcatctattaagtttttttactttaacgcccttgtatgtttttacagagactttatctaaatttctagtgcttgtatgtgttataaatataacgggatatagaactgaatcacctaccttagatacccaattacattttatcagatccagataataaacaaattttgtcgccctaactaattctatattgttatatattttacaattggttatgatatcatgtaataacttggaatctaacgcacatcgtcgtacgtttatacaattgtgatttagtgtagtatatctacacatgtatttttccgcgctatagtattctggactagtgataaaactatcgttatatctgtcttcaatgaactcatcgagatattgctctctgtcatattcatacacctgcataaactttctagacatcttacaatccgtgttattttaggatcatatttacatatttacgggtatatcaaagatgttagattagttaatgggaatcgtctataataatgaatattaaacaattatatgaggacttttaccacaaagcatcataaaaatgagtcgtcgtctgatttatgttttaaatatcaaccgcgaatcaactcataaaatacaagagaatgaaatatatacatattttagtcattgcaatatagaccatacttctacagaacttgattttgtagttaaaaactatgatctaaacagacgacaacctgtaactgggtatactgcactacactgctatttgtataataattactttacaaacgatgtactgaagatattattaaatcatggagtggatgtaacgatgaaaaccagtagcggacgtatgcctgtttatatattgcttactagatgttgcaatatttcacatgatgtagtgatagatatgatagacaaagataaaaaccacttattacatagagactattccaacctattactagagtatataaaatctcgttacatgttattaaaggaagaggatatcgatgagaacatagtatccactttattagataagggaatcgatcctaactttaaacaagacggatatacagcgttacattattattatttgtgtctcgcacacgtttataaaccaggtgagtgtagaaaaccgataacgataaaaaaggccaagcgaattatttctttgtttatacaacatggagctaatctaaacgcgttagataattgtggtaatacaccattccatttgtatcttagtattgaaatgtgtaataatattcatatgactaaaatgctgttgacttttaatccgaatttcgaaatatgtaataatcatggattaacgcctatactatgttatataacttccgactacatacaacacgatattcttgttatgttaatacatcactatgaaacaaatgttggagaaatgccgatagatgagcgtcgtataatcgtattcgagtttatcaaaacatattctacacgtcctgcagattcgataacttatttgatgaataggtttaaaaatatagatatttatacccgctatgaaggaaagacattattacacgtagcatgtgaatataataatacacacgtaatagattatcttatacgtatcaacggagatataaatgcgttaaccgacaataacaaacacgctacacaactcattatagataacaaagaaaattccccatataccattaattgtttactgtatatacttagatatattgtagataagaatgtgataagatcgttggtggatcaacttccatctctacctatcttcgatataaaatcatttgagaaattcatatcctactgtatacttttagatgacacattttacaatagacacgttaggaatcgcgattctaaaacgtatcgatacgcattttcaaaatacatgtcgtttgataaatacgatggtataataactaaatgtcataaagaaacaatattgctcaaactatccactgttctagacactacactatatgcagttttaagatgccataattcgaaaaagttaagaagatacctcaccgagttaaaaaaatataataacgataagtcctttaaaatatattctaatattatgaatgagagataccttaatgtatattataaagatatgtacgtgtcaaaggtatatgataaactatttcctgttttcacagataaaaattgtctactaacattactaccttcagaaattatatacgaaatattatacatgctgacaattaacgatctttataatatatcgtatccacctaccaaagtatagttgtatttttctcatgcgatgtgtgtaaaaaaactgatattatataaatattttagtgccgtataataaagatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaaatatacaatgcatttttatatacattactgaataattattattattatttatatcgtatttgtgctataacgcgactatctaggtatttgtatctcaccgatagagaacatataaatgtagactctattaaacagttgtgtaaaatatcagatcctaatgcatgttatagatgtggatgtacggctttacatgagtacttttataattatagatcagtcaacggaaaatacaagtatagatacaacggttactatcaatattatttatctagcgattatgaaaattataatgaatattattatgatgattatgatagaactggtatgaacagtgagagtgataatatatcaatcaaaacagaatatgaattctatgatgaaacacaagatcaaagtacacaactagtaggttacgacattaaactcaaaaccaatgaggatgattttatggctatgatagatcagtgggtgtccatgattatatagatgaatcaattaataaagtagtatatggaagagagtctcacgtaagatggcgggatatatggcaagaacataatgatggcgtatacagtataggaaaggagtgcatagataatatatacgaagacaaccataccgtagacgaattctacaagatagacagcgtatcagatgtagatgacgcggaacacatatctccgataactaatgatgtatctacacaaacatgggaaaagaaatcagagttagatagatacatggaaatgtatcctcgtcatagatatagtaagcattctgtctttaagggattttctgacaaagttagaaaaaatgatttagacatgaatgtggtaaaagaattactttctaacggtgcatctctaacaattaaggatagcagtaataaggatccaataaccgtttattttcgaagaacgataatgaatttagaaatgattgatgaacgaaagtatatagtacactcctatctaaaaaattataaaaatttcgattatccatttttcaggaagttagttttgactaataaacattgtctcaacaattattataatataagcgacagcaaatatggaacaccgctacatatattggcgtctaataaaaaattaataactcctaattacatgaagttattagtgtataacggaaatgatataaacgcacgaggtgaagatacacaaatgcgaactccattacacaaatatttgtgtaaatttgtatatcataatattgaatatggtatccgatactataatgaaaagattatagacgcatttatagagttaggagccgatctaactattccaaatgacgatggaatgataccagtagtttactgtatacactcaaatgccgaatatggttataacaatattactaacataaagataatacgtaaactacttaatcttagtagacgtgcgtcacataatctatttagagatcgagtcatgcacgattatataagtaatacatatattgatcttgagtgtttagatattattagatcgttggatggattcgatatcaatggttactttgaaggacgtacaccacttcattgcgctatacaacataacttcactcagattgctaagtacttattagatcgaggagctgatatagtcgtacccaacacattgattatacatcagtacatacagtaaatagcatagatatggaggaggatacaaatatttcaaataaagttataaggtacaacactgtcaataatatatgggaaacattacctaacttctggactggaactataaatccaggcgtggtctcgcataaagatgatatatatgttgtatgcgacatcaaagatgaaaaaaatgttaaaacttgtatatttagatataacacgaatacgtataacggatgggaattggtcacgacgacagaaagcagattatcagctctgcatactattctttataacaataccataatgatgttacattgttatgaatcgtatatgttacaagatacatttaatgtgtacactcgcgaatggaatcatatgtgtcatcaacattcgaatagttatatcatgtacaatatactacccatctactaaatataatagaataaaataaatgagtatgatcattttagataacgattgattttatcattaccgcttcattcttatattctttgcttacggaacctatatttagaaacatctactaacgattttttatgcttgcattattaatggtatgtaatatgattgattgtgtacgcaataccaatttgttaagtatgaatacggggtacaaacataaactgaagtttaacattatttatttatgatatatatcgttattgtttggtctataccatggatatctttaaagaactaatcttaaaacacacggatgaaaatgttttgatttctccagtttctattttatctactttatctattctaaatcatggagcagctggttctacagctgaacaactatcaaaatatatagagaatatgaatgagaatacacccgatgacaataatgacatggacgtagatattccgtattgtgcgacactagctaccgcaaataaaatatacggtagcgatagtatcgagttccacgcctccttcctacaaaaaataaaagacgattttcaaactgtaaactttaataatgctaaccaaacaaaggaactaatcaacgaatgggttaagacaatgacaaatggtaaaattaattccttattgactagtccgctatccattaatactcgtatgacagttgttagcgccgtccattttaaagcaatgtggaaatatccattttctaaacatcttacatatacagacaagttttatatttctaagaatatagttaccagtgttgatatgatggtgagcactgagaataacttgcaatatgtacatattaatgaattattcggaggattctctattatcgatattccatacgagggaaactctagtatggtaattatactaccggacgacatagaaggtatatataacatagaaaaaaatataacagatgaaaaatttaaaaaatggtgtggtatgttatctactaaaagtatagacttgtatatgccaaagtttaaagtggaaatgacagaaccgtataatctggtaccgattttagaaaatttaggacttactaatatattcggatattatgcagattttagcaagatgtgtaatgaaactatcactgtagaaaaatttctacatacgacgtttatagatgttaatgaggagtatacagaagcatcggccgttacaggagtatttatgactaacttttcgatggtatatcgtacgaaggtctacataaaccatccattcatgtacatgattaaagacaacacaggacgtatactttttatagggaaatactgctatccgcaataaatataaacaaatagacttttatcacgtttatctatgtctaaatattacaaatagtaatagtataaactaaagctgataatacttaaaaaaataataatatcatttacaattaatagtataaactaaaaattaaacaaatcgttattataagtaatatcaaaatgatgatatacggattaatagcgtgtcttatattcgtgacttcatccatcgctagtccactttatattcccgttattccacccatttcggaagataaatcgttcaatagtgtagaggtattagtttccttgtttagagatgaccaaaaagactatacggtaacttctcagttcaataactacactatcgataccaaagactggactatcggcgtactatccacacctgatggtttggatataccattgactaatataacttattggtcacggtttactataggtcgtgcattgttcaaatcagagtctgaggatattttccaaaagaaaatgagtattctaggtgtttctatagaatgtaagaagtcgtcgacattacttacttttttgaccgtgcgtaaaatgactcgagtatttaataaatttccagatatggcttattatcgaggagactgtttaaaagccgtttatgtaacaatgacttataaaaatactaaaactggagagactgattacacgtacctctctaatggggggttgcctgcatactatcgtaatggggtcgatggttgattattgattagtatattccttattctttttattcacacaaaaagaacatttttataaacatgaaaccactgtctaaatgtaattatgatcttgatttatagatgaagatcagcctttagaggattttaaccagtatgtttaatatgaaaaaaataaacataacatattttgagattaagcgctattgtgcaagattatattagaatcaaattaatctttcatacgagaaaaataacgacatacgtcgtcaacaaattaaactttttatttattagttaactagcttatagaacttgctcattgttatgtttctaaaacgggtacgacatataggacaattatccgacgcaccggtttctcttcgtgttttatgccatatattgatgcatgttatgcaaaatatatgagtacacgaatccaataaaccaaagtatctatcgttttgagtaaacaacttcatagcaaattccacattctttttctttacttactctatacacgtcctcgtatttatccagtattttgatgatatccaactcagaaatggttgttgtattattgggtgtattgggagtataggtattattagctatgtaccaatttaccaaccctcttaatattgattgaacaatcacatcggttatccaatcaataaccacattaataactaaattgtagtgtatatatagaccatatatgtttctatttttttgacagttacgtatagtttcagtaagttttgattgttgtattcctgtatctctagataagttagtcatatagtcccttccggcgatacgttttttccaagcccgaaattgattagccaaatgtgtatttatttttgtgatattgatataatatgtaatgttattaatatttcggataatgcatactgttagtcttatatcatttggttcatctatgtattgtaatattgttacatgatctatagatgatgtattgattttggcaggatcgaattccatatccgcgactaaacagtgaaaaaaatgtaaatactttttaaattttaaattagtaaaacttttttttattttttatgattccaaaaatactgaatacaaagtcctaaattataaatatggagatcatactaccacaacttattattatgcatactcagccggtgtaatagatagatatatataattctattacaccggcagacaattaccgatcggtatttgtcgttaccaacataccgtataatatgtaatatacaattccataacccattgacagttgttatacatcaaaattgcaattcttttgattacgatgttataagaatgtagttaattgatgtatgatgttaatgtgtcctctttcctcttataacatcgtaatcaaaaacttttttataatatatacctaataatgtgtcttaatagttctcgtgattcgtcaaacaatcattcttataaaatataataaagcaacgtaaaacacataaaaataagcgtaactaataagacaatggatatttacgacgataaaggtctacagactattaaactgtttaataatgaatttgattgtataaggaatgacatcagagaattatttaaacatgtaactgattccgatagtatacaacttccgatggaagacaattctgatattatagaaaatatcagaaaaatactatatagacgattaaaaaatgtagaatgtgttgacatcgatagtacaataacttttatgaaatacgatccaaatgatgataataagcgtacgtgttctaattgggtacccttaactaataactatatggaatattgtctagtaatatatttggaaacaccgatatgtggaggcaaaataaaattataccaccctacaggaaatataaagtcggataaggatattatgtttgcaaagactctagactttaaatcaaagaaagtgttaactggacgtaaaacaattgccgttctagacatatccgtttcatataatagatcaatgactactattcactacaacgacgacgttgatatagatatacatactgataaaaatggaaaagagttatgttattgttatataacaatagatgatcattacttggttgatgtggaaactataggagttatagtcaatagatctggaaaatgtctgttagtaaataaccatctaggtataggtatcgttaaagataaacgtataagcgatagttttggagatgtatgtatggatacaatatttgacttttctgaagcacgagagttattttcattaactaatgatgataacaggaatatagcatgggacactgataaactagacgatgatacagatatatggactcccgtcacagaagatgattacaaatttctttctagactagtattgtatgcaaaatctcaatcggatactgtattcgactattatgttcttactggtgatacggaaccacccactgtattcattttcaaggtaactagattttactttaatatgccgaaataaaaaatttttgtataatatctagaggtagaggtattgtttagataaatacaaataacatagatacatcgcatacttagcatttttataaatatacataagacatacactttatacattttttttgtaaaaatactcataaaaaaatttataaaaattatggcacaaccatatcttgtataggtagtttagttcgtcgagtgaacctataaacagataatagacaacacgtaataataataatgcctactaatacaagcataataccgggagatgggatatatgacgttgtagtgtttgggttttctgaacgttgatagtctactaatactacatgctgacatctaatgcctgtataaccatgagagcatctacaatacataccgtcaatatctctagcgtggatacagtcaccgtgtaaacaatatccatctccctctggaccgcataatctgatagctggaatatctgttgtagcgtttgtaatttctggcgatgtcgtttcgatagcgttaccactatcggcgaatgatctgattatcatagcagcgaacaacaacatcagatatttcatcgacatttttgatggattttgtgtttatgctgtttctcagtgtgtgtttatgacaagattgggaattttatattattaattcagtaatataaactaataatatattgttaattgtgtaaataatataaaaataacaatacaatattgaatgtgttgctgttaaaaatgatcataaacacggagtttattttatatgtctcgcataaacattactaaaaaaatatattgttctgtttttctttcacatctttaattatgaaaaagtaaatcattatgagatggacgagattgtacgcatcgttcgcgacagtatgtggtacatacctaacgtatttatggacgacggtaagaatgaaggtcacgtttctgtcaacaatgtctgtcatatgtatttcacgttctttgatgtggatacatcgtctcatctgtttaagctagttattaaacactgcgatctgaataaacgaggtaactctccattacattgctatacgatgaatacacgatttaatccatctgtattaaagatattgttacaccacggcatgcgtaactttgatagcaaggatgaaaaaggacaccactatcaatcgataacaagatctttgatatactaacggacaccattgatgactttagtaaatcatccgatctattgctgtgttatcttagatataaattcaatgggagcttaaactattacgttctgtacaaaggatccgacgaggatgaactcacttctcttcattactactgtaaacacatatccacgttctacaaaagcaattattacaagttaagtcacactaagatgcgagccgagaagcgattcatctacgcgataatagattatggagcaaacattaacgcggttacacacttaccttcaacagtataccaaacatagtcctcgtgtggtgtatgctcttttatctcgaggagccgatacgaggatacgtaataatcttgattgtacacccatcatggaacgattgtgcaacaggtcatattctcataatgttactcaattggcacgaacaaaaggaagaaggacaacatctactttatctattcataaaacataatcaaggatacactctcaatatactacggtatctactagataggttcgacattcagaaagacgaatactataataccgcctttcaaaattgtaacaacaatgttgcctcatacatcggatacgacatcaaccttccgactaaagacggtattcgacttggtgtttgaaaacagaaacatcatatacaaggcggatgttgtgaatgacatcatccaccacagactgaaagtatctctacctatgattaaatcgttgttctacaagatgtctctccctacgacgattactacgtaaagaagataatagcctactgcctattaagggacgagtcattcgcggaactacatagtaaattctgtttaaacgaggactataaaagtgtatttatgaaaaatatatcattcgataagatagattccatcatcgtgacataagtcgccttaaagagattcgaatctccgacaccgacctgtatacggtatcacagctatcttaaagccatacattcagacagtcacatttcatttcccatgtacgacgatctcatagaacagtgccatctatcgatggagcgtaaaagtaaactcgtcgacaaagcactcaataaattagagtctaccatcggtcaatctagactatcgtatttgcctccggaaattatgcgcaatatcatctaaacagtatgttgtacgaaaagaaccattacaaatattatccatgatagaaagaaaatatctatatgattggagaagtaggaaacaggaacaagacgacgattactacattattaaatcatgaagtccgtattatactcgtatatattgtttctctcatgtataataataaacggaagagatatagcaccgcatgcaccatccgatggaaagtgtaaagacaacgaatacaaacgccataatttgtgtccgggaacatacgcttccagattatgcgatagcaagactaacacacaatgtacgccgtgtggttcgggtaccttcacatctcgcaataatcatttacccgcttgtctaagttgtaacggaagacgcgaccgtgtaacacgactcacaatagaatctgtgaatgctctcccggatattattgtcttctcaaaggatcatccggatgcaaggcatgtgtttcccaaacaaaatgtggaataggatacggagtatccggagacgtcatctgttctccgtgtggtctcggaacatattctcacaccgtctcttccgcagataaatgcgaacccgtacccagtaatacctttaactatatcgatgtggaaattaatctgtatccagttaacgaccactaccggtctcagcgaatccatctcaacgtcggaactaactattactatgaatcataaagactgtaatcccgtatttcgtgaggaatacttctccgtccttaataaggtagcaacttcaggtttctttacaggagaaaggtgtgcactctgaatttcgagattaaatgcaataacaaaaatttttcctccaaacagttaacgaaagcaaagaatgatgacggtatcatgccgcattcggagactgtctatctagcgtcgacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtactcaatcaatagcaatcatgaaacaatatatcgtcctggcatgcatgtgcctgccagtcttcagcaatcatcctcatcctcctcctcgtgtacggaagaagaaaacaaacatcatatgggaatcgatgttattatcaaagtcacaaagcaagaccaaacaccgaccaatgataagatttgccaatccgtaacggaaattacagagtccgagtcagatccagatcccgaggtggaatcagaagatgattccacatcagtcgaggatgtagatcctcctaccacttattactccatcatcggtggaggtctgagaatgaactttggattcaccaaatgtcctcagattaaatccatctcagaatccgctgatggaaacacagtgaatgctagattgtccagcgtgtccccaggacaaggtaaggactctcccgcgatcactcatgaagaagctctggctatgatcaaagactgtgaggtgtctatcgacatcagatgtagcgaagaagagaaagacagcgacatcaagacccatccagtactcgggtctaacatctctcataagaaagtgagttacgaagatatcatcggttcaacgatcgtcgatacaaaatgtgtcaagaatctagagtttagcgttcgtatcggagacatgtgcaaggaatcatctgaacttgaggtcaaggatggatttaagtatgtcgacggatcggcatctgaaggtgcaaccgatgatacttcactcatcgattcaacaaaactcaaagcgtgtgtctgaatcgataactctattcatctgaaattggatgagtagggttaatcgaacgattcaggcacaccacgaattaaaaaagtgtaccggacactatattccggtttgcaaaacaaaaatgttcttaactacattcacaaaaagttacctctcgcgacttcttctttttctgtctcaatagtgtgatacgattatgacactattcctattcctattcctatttcctttcagggtatcacaaaaatattaaacctctttctgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctcattctcattttctctttctctcttcaatggagtcataaaatatttttattctctttctctcttcgatggtctcacaaaaatattaaacctctttctgatggtgtcactaaaatatttttattctcattctcattttctctttctctcttcaatggagtcataaaatatttttattctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcactaaaatattttttattctctttctgatgcatcaactatttcttaaacaataacgtccaacaacatatactcgtcgagcttatcaacatcccctatgcccatctaggttaccagacaattgtatatcataaaataatgtttataatttacacgttaaaatcatataataaaacgtagatcgtataatattttttggtatataaatgatctagtaaaatccatgtaggggatactgctcacattttttctttggtacaaaatttcacacaagtttttatacagacaaattcttgtccatatattttaaaacattgacttttgtactaagaaaaatatctagatcaactatctctttctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggagtcgtaaaaaagttttatctctttctccttcgatggtctcacaaaaatattaaacctctttctgatggagtcgtaaaaaagttttatctctttctccttcgatggtctcacaaaaatattaaacctctttctgatggagtcgtaaaaaagttttatctctttctctcttcgatggtctcacaaaaatattaaacctctttctgatggagtcgtaaaaaagttttatctctttctctcttcgatggtctcacaaaaatattaaacctctttctgatggtctctataaagcgatcgatctttcttacactctagagtttcctacagtcatgggtcacacatttttttctagacactaaataaaattagtaaaattaaattaattataaaattatatatataatttactaactttagttagataaattaataatatataagttttagtacattaatattatattttaaat

SEQ ID NO: 8 - DNA, 24 bp, Artificial Sequence
  Description of Artificial Sequence Synthetic primer
tgtgaagacgataaattaatgatc

SEQ ID NO: 9 - DNA, 20 bp, Artificial Sequence
  Description of Artificial Sequence Synthetic primer
acctgatggataaaaaggcg

SEQ ID NO: 10 - DNA, 20 bp, Artificial Sequence
  Description of Artificial Sequence Synthetic primer
ttgtcatcatgaacggcgga

SEQ ID NO: 11 - DNA, 20 bp, Artificial Sequence
  Description of Artificial Sequence Synthetic primer
tccttcgtttgccatacgct

SEQ ID NO: 12 - DNA, 20 bp, Artificial Sequence
  Description of Artificial Sequence Synthetic primer
aggagaccaggcatccatct

SEQ ID NO: 13 - DNA, 20 bp, Artificial Sequence
  Description of Artificial Sequence Synthetic primer
gttctgacgacggtgggaat

SEQ ID NO: 14 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 15 - protein, 330 aa, Homo sapiens [sequence omitted]

SEQ ID NO: 16 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 17 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 18 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 19 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 21 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 22 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 21 aa, Artificial Sequence
  Description of Artificial Sequence Synthetic peptide
  MISC_FEATURE (1)..(3): This region may or may not be present
GSGEGRGSLLTCGDVEENPGP

SEQ ID NO: 24 - protein, 22 aa, Artificial Sequence
  Description of Artificial Sequence Synthetic peptide
  MISC_FEATURE (1)..(3): This region may or may not be present
GSGATNFSLLKQAGDVEENPGP

SEQ ID NO: 25 - protein, 23 aa, Artificial Sequence
  Description of Artificial Sequence Synthetic peptide
  MISC_FEATURE (1)..(3): This region may or may not be present
GSGQCTNYALLKLAGDVESNPGP

SEQ ID NO: 26 - protein, 25 aa, Artificial Sequence
  Description of Artificial Sequence Synthetic peptide
  MISC_FEATURE (1)..(3): This region may or may not be present
GSGVKQTLNFDLLKLAGDVESNPGP

SEQ ID NO: 27 - protein, 7 aa, Artificial Sequence
  Description of Artificial Sequence Synthetic peptide
GYTFTDY

SEQ ID NO: 28 - protein, 4 aa, Artificial Sequence
  Description of Artificial Sequence Synthetic peptide
PYNG

SEQ ID NO: 29 - protein, 6 aa, Artificial Sequence
  Description of Artificial Sequence Synthetic peptide
YGSWFA

SEQ ID NO: 30 - protein, 12 aa, Artificial Sequence
  Description of Artificial Sequence Synthetic peptide
SQSIVHSNGNTY

SEQ ID NO: 31 - protein, 3 aa, Artificial Sequence
  Description of Artificial Sequence Synthetic peptide
KVS

SEQ ID NO: 32 - protein, 6 aa, Artificial Sequence
  Description of Artificial Sequence Synthetic peptide
GSHVPY